(19) United States
(12) Patent Application Publication
(10) Pub. No.: US 2025/0237606 A1
(43) Pub. Date: Jul. 24, 2025

(54) METHODS FOR FLUORESCENCE IMAGING MICROSCOPY AND NANO-FISH

(71) Applicant: Altius Institute for Biomedical Sciences, Seattle, WA (US)

(72) Inventors: John A. Stamatoyannopoulos, Seattle, WA (US); Shreeram Akilesh, Seattle, WA (US); Vivek Nandakumar, Seattle, WA (US)

(21) Appl. No.: 19/053,261

(22) Filed: Feb. 13, 2025

Related U.S. Application Data
(63) Continuation of application No. 17/736,684, filed on May 4, 2022, now Pat. No. 12,253,468.
Application No. 17/736,684 is a continuation of application No. 16/319,157, filed on Jan. 18, 2019, now Pat. No. 11,353,400.
(60) Provisional application No. 62/364,245, filed on Jul. 19, 2016.

(51) Int. Cl. G01N 21/64; A01N 1/125; C12N 5/078; C12N 5/09; C12P 19/34; C12Q 1/6837; C12Q 1/6841; C40B 30/04; C40B 40/06; G01N 1/30; G01N 15/00; G01N 21/78; G01N 33/569; G01N 33/574; G01N 33/58

(52) U.S. Cl. CPC G01N 21/64; A01N 1/125; C12N 5/0634; C12N 5/0694; C12P 19/34; C12Q 1/6837; C12Q 1/6841; C40B 40/06; G01N 1/30; G01N 21/6458; G01N 21/78; G01N 33/56966; G01N 33/574; G01N 33/582; C40B 30/04; G01N 2015/0038

(57) ABSTRACT

Disclosed herein are methods of detecting a target nucleic acid sequence, determining the localization of the target nucleic acid sequence, and/or quantifying the number of target nucleic acid sequences in a cell. This method may be used on small target nucleic acid sequences, and may be referred to as Nano-FISH.

### CROSS-REFERENCE TO RELATED APPLICATIONS

This application is a continuation of U.S. application Ser. No. 17/736,684, filed on May 4, 2022, which is a continuation of U.S. application Ser. No. 16/319,157 filed on Jan. 18, 2019, issued as U.S. Pat. No. 11,353,400, which application is a 371 of international Application No. PCT/US2017/042946 filed on Jul. 19, 2017 which application claims the benefit of U.S. Provisional Application No. 62/364,245, filed Jul. 19, 2016, which application is incorporated herein by reference in its entirety for all purposes.

### INCORPORATION BY REFERENCE OF A SEQUENCE LISTING PROVIDED AS A SEQUENCE LISTING XML FILE

A Sequence Listing is provided herewith as a Sequence Listing XML, “ALTI-712USCON2_SEQ_LIST.xml,” created on Jan. 22, 2025 and having a size of 935,061 bytes. The contents of the Sequence Listing XML are incorporated by reference herein in their entirety.

### BACKGROUND OF THE INVENTION

Imaging techniques such as fluorescence in situ hybridization (FISH) allows for visualization of DNA or RNA regions, and/or assessment of gene expression, chromosome position, and/or protein localization. In some instances, these imaging methods are limited by small field of view and/or limited resolution. As such, data acquisition from large number of cells requires multiple fields of view and thereby presents challenges in obtaining high throughput and high resolution imaging data. Additionally, for example, existing FISH techniques typically use fluorescent probes that span tens to hundreds of kilobases (kb). This precludes the use of existing FISH techniques for accurate spatial localization of smaller genomic sequences. As such, new FISH techniques are required to detect the localization of probes to small genomic sequences.

### SUMMARY OF THE INVENTION

In various aspects, the present disclosure provides a method for detecting a presence of a target nucleic acid sequence in an intact cell, the method comprising: a) providing a nucleic acid sequence comprising the target nucleic acid sequence in the intact cell, wherein the target nucleic acid sequence is less than 2.5 kilobases in length; b) contacting the intact cell with a first plurality of probes, wherein each probe comprises a first detectable label and a probe sequence that binds to a portion of the target nucleic acid sequence; and c) detecting a presence of the first detectable label in the intact cell, wherein the presence of the first detectable label indicates the presence of the target nucleic acid sequence. In some aspects, the detecting is within a period of not more than 48 hours.

In various aspects, a method comprises detecting a target nucleic acid sequence within a period of not more than 48 hours, wherein the target nucleic acid sequence is a non-amplified target nucleic acid sequence. In further aspects, the method comprises contacting the target nucleic acid sequence with a first plurality of probes, wherein each probe comprises a first detectable label and probe sequence that binds to a portion of the target nucleic acid sequence. In still further aspects, the method comprises detecting a presence of the first detectable label on the target nucleic acid sequence, wherein the presence of the first detectable label indicates the presence of the target nucleic acid sequence.

In various aspects, a method for quantifying a target nucleic acid sequence in an intact cell is provided herein, the method comprising: a) providing a nucleic acid sequence comprising the target nucleic acid sequence in the intact cell; b) contacting the intact cell with a first plurality of probes, wherein each probe comprises a first detectable label and a probe sequence that binds to a portion of the target nucleic acid sequence; and c) determining a number of the target nucleic acid sequences present in the intact cell within a period of not more than 48 hours.

In some aspects, the target nucleic acid sequence comprises a nucleic acid sequence of not more than 10 kilobases in length, not more than 8 kilobases in length, not more than 6 kilobases in length, not more than 4 kilobases in length, not more than 3 kilobases in length, not more than 2 kilobases in length, not more than 1.5 kilobases in length, or not more than 1 kilobases in length.

In some aspects, the method further comprises: d) contacting the intact cell with a second detectable label that binds to a portion of a cellular structure, and e) detecting a position of the first detectable label in the intact cell relative to the second detectable label, wherein the position is used to determine a spatial position of the target nucleic acid sequence.

In other aspects, the method further comprises: d) providing the intact cell further comprising a second target nucleic acid sequence; e) contacting the intact cell with a second plurality of probes, wherein each probe comprises a second detectable label and a probe sequence that binds to a portion of the second target nucleic acid sequence, and f) detecting a position of the first detectable label in the intact cell relative to the second detectable label, wherein the position is used to determine the spatial position of the target nucleic acid sequence.

In some aspects, the target nucleic acid sequence comprises an exogenous nucleic acid sequence. In some aspects, the target nucleic acid sequence comprises a nucleic acid sequence encoding somatic rearrangement. In some aspects, the target nucleic acid sequence comprises a nucleic acid sequence encoding a B cell receptor or a T cell receptor.

In some aspects, the method comprises the first plurality of probes bound to the nucleic acid sequence is less than 250 probes, less than 200 probes, less than 150 probes, less than 100 probes, less than 80 probes, less than 60 probes, less than 50 probes, less than 40 probes, less than 30 probes, less than 20 probes, less than 15 probes, less than 10 probes, or less than 8 probes.

In some aspects, the period is not more than 24 hours.

In some aspects, the method further comprises binding at least a portion of the first plurality of probes to the target nucleic acid sequence.

In some aspects, the method further comprises washing the intact cell after contacting the target nucleic acid sequence with the first plurality of probes. In some aspects, the method further comprises optically detecting the first detectable label. In some aspects, the method further comprises optically detecting the second detectable label.

In some aspects, the intact cell is obtained from a tissue. In some aspects, the intact cell is a mammalian or eukaryotic cell. In some aspects, the intact cell is a hematopoietic progenitor cell, a monocyte, a macrophage, a microglia, a neuron, or a T-cell.

In some aspects, the method further comprises introducing the exogenous nucleic acid sequence into the cell. In some aspects, the introducing comprises electroporation, lipofection, transfection, microinjection, viral transduction, or use of a gene gun. In some aspects, the exogenous nucleic acid sequence is integrated into the genome of the cell.

In some aspects, the probe sequence of at least one probe of the first plurality of probes comprises an oligonucleotide sequence. In some aspects, the probe sequence of at least one probe of the first plurality of probes comprises an amino acid sequence. In some aspects, the second plurality of probes bound to the second target nucleic acid sequence is less than 250 probes, less than 200 probes, less than 150 probes, less than 100 probes, less than 80 probes, less than 60 probes, less than 50 probes, less than 40 probes, less than 30 probes, less than 20 probes, less than 15 probes, less than 10 probes, or less than 8 probes. In some aspects, the probe sequence of at least one probe of the second plurality of probes comprises an oligonucleotide sequence. In some aspects, the probe sequence of at least one probe of the second plurality of probes comprises an amino acid sequence.

In various aspects, the present disclosure provides a method for assessing a phenotype of an intact genetically modified cell comprising: a) providing the intact genetically modified cell comprising a target nucleic acid sequence less than 2.5 kilobases in length; b) contacting the intact genetically modified cell with a first plurality of probes, wherein each probe comprises a first detectable label and a probe sequence that binds to a portion of the target nucleic acid sequence; c) detecting a presence of the first detectable label in the intact cell, wherein the presence of the first detectable label indicates the presence of the target nucleic acid sequence; d) determining a phenotype of the intact genetically modified cell; and e) correlating the phenotype of the intact genetically modified cell with the presence of the target nucleic acid sequence. In some aspects, the intact genetically modified cell is a eukaryotic intact genetically modified cell. In some aspects, the phenotype is a product expressed due to a genetic modification in the intact genetically modified cell, a quality of the product expressed due to the genetic modification in the genetically modified cell, or a combination thereof. In some aspects, the phenotype is an increased or decreased expression of the product, an increase or decrease in the quality of the product, or a combination thereof. In some aspects, the method further comprises determining a number or location of genetic modifications in the intact genetically modified cell. In some aspects, the product expressed is a transgene protein, RNA, or a secondary product of the genetic modification. In some aspects, the method further comprises: f) selecting a first intact genetically modified cell comprising a phenotype of interest; g) determining a set of conditions used for a genetic modification of the first intact genetically modified cell; and h) preparing a second genetically modified cell using the set of conditions for genetic modification.

In various aspects, the present disclosure provides an in situ method of determining the localization of a regulatory element, the method comprising: a) contacting a regulatory element with a first set of detection agents; b) photobleaching the first set of detection agents for a first time point at a first wavelength to generate a second set of detection agents capable of generating a light at a second wavelength; c) detecting at least one burst generated by the second set of detection agents to generate a detection profile; and d) based on step c), analyzing the detection profile to determine the localization of the regulatory element. In some aspects, the first set of detection agent is a first set of fluorescently labeled probes.

In some aspects, the method further comprises hybridizing the first set of fluorescently labeled probes to a set of target sites on a regulatory element.

In some aspects, the second set of detection agent is a second set of fluorescently labeled probes. In some aspects, the fluorescent moieties of the first set of fluorescently labeled probes are the same.

In some aspects, the method further comprises a third set of fluorescently labeled probes. In some aspects, the third set of fluorescently labeled probes is photobleached to generate a fourth set of fluorescently labeled probes. In some aspects, the fluorescent moieties of the first set of fluorescently labeled probes are the same as the fluorescent moieties of the third set of fluorescently labeled probes. In some aspects, wherein the fluorescent moieties of the first set of fluorescently labeled probes are different than the fluorescent moieties of the third set of fluorescently labeled probes. In some aspects, wherein the third set of fluorescently labeled probes hybridizes to different target sites of the regulatory element from the first set of fluorescently labeled probes. In some aspects, the third set of fluorescently labeled probes hybridizes to different target sites of a different regulatory element from the first set of fluorescently labeled probes. In some aspects, the fluorescent moieties of the first set of fluorescently labeled probes are different than the fluorescent moieties of the third set of fluorescently labeled probes and the first set of fluorescently labeled probes hybridizes to different target sites from the third set of fluorescently labeled probes.

In some aspects, the method further comprises photobleaching the first set of fluorescently labeled probes for a first time point at a first wavelength and a third wavelength to generate a fourth set of fluorescently labeled probes capable of fluorescence at a fourth wavelength. In some aspects, the first wavelength and the third wavelength is applied simultaneously. In some aspects, the first wavelength and the third wavelength is applied sequentially In some aspects, the first wavelength is at 491 nm light. In some aspects, the third wavelength is at 405 nm light. In some aspects, the fluorescent moiety comprises a fluorescent small molecule. In some aspects, the fluorescent moiety does not comprise a fluorescent protein. In some aspects, the detection profile comprises a chromatic aberration correction. In some aspects, the detection profile comprises less than 5%, 4%, 3%, 2%, 1%, 0.5%, 0.1%, or 0% chromatic aberration. In some aspects, the localization of the regulatory element provides activity state of the regulatory element. In some aspects, the localization of the regulatory element provides interaction state with at least one additional regulatory element. In some aspects, the regulatory element comprises DNA, RNA, polypeptides, or a combination thereof. In some aspects, the regulatory element is DNA. In some aspects, the regulatory element is RNA. In some aspects, the RNA is an enhancer RNA (eRNA). In some aspects, the regulatory element is a DNasel hypersensitive site (DHS).

In some aspects, the method further comprises: a) hybridizing a first set of fluorescently labeled probes to a set of target sites on a DHS in a cell sample; b) photobleaching the first set of fluorescently labeled probes for a first time point at a first wavelength to generate a second set of fluorescently labeled probes capable of fluorescence at a second wavelength; c) detecting at least one fluorescent burst generated by the second set of fluorescently labeled probes to generate a DHS profile; and d) comparing the DHS profile with a control set of DHS profiles, wherein a correlation between the DHS profile and the control set leads to identification of the cell type. In some aspects, the regulatory element is a polypeptide. In some aspects, the regulatory element comprises chromatin.

In various aspects, a kit is provided by the present disclosure, wherein the kit comprises a probe set and a set of instructions for the method of any of the methods as described herein.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 represents a conceptual illustration of methods described herein.

FIG. 2 illustrates a conceptual schematic of an exemplary computer server to be used for processing a method described herein.

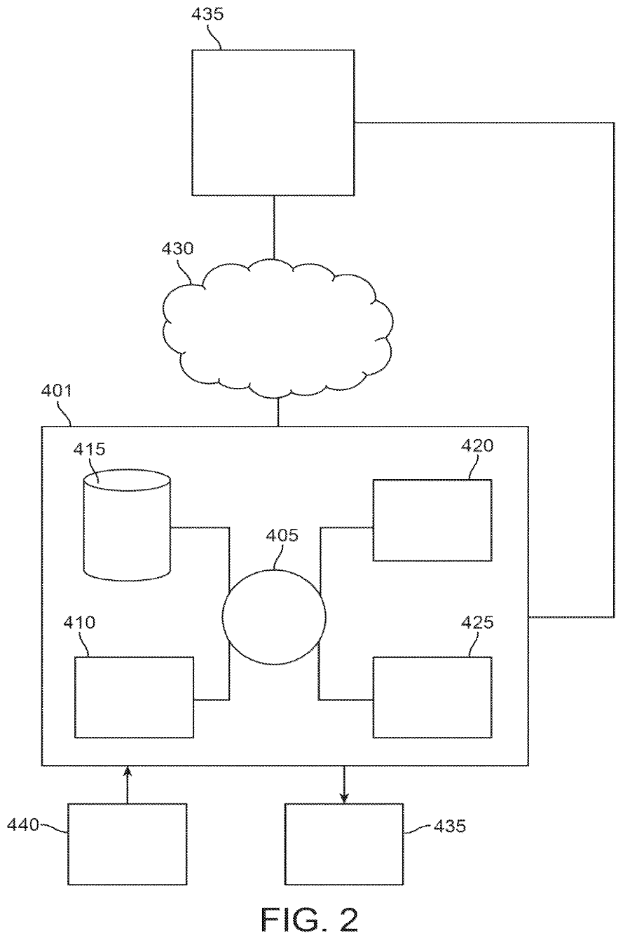

FIG. 3A shows a two color SPDM image (experimental) of chromatin (blue) with DNA sensitive element sites (red), showing anti-colocalization of the DNA sensitive element sites with chromatin. Scale bars: 1000 nm, inserts: 100 nm. The bottom right panel shows chromatin (blue), the middle right panel shows DNA sensitive element sites (red), and the top right panel shows the overlay and the anti-colocalization of the DNA sensitive element with chromatin. FIG. 3B is the inset of FIG. 3A.

FIG. 4A and FIG. 4B illustrate the localization precision and nearest neighbor distances for DNA and DNase sensitive elements.

FIG. 5A and FIG. 5B illustrate multi-omics imaging via encoding of molecular information with ssDNA tags. FIG. 5A shows a schematic of simultaneous labeling and multiplexed imaging of mRNA and protein targets with multicolor QDots via DNA encoding. In general, each molecular target is encoded by target-specific ssDNA-tagged affinity molecule (e.g., an antibody, aptamer, oligonucleotide, etc.). The resulting array of target-bound ssDNA tags can be sequentially or simultaneously labeled by complementary imaging probes, enabling multiplexed imaging of all targets of interest (e.g., via fluorescence microscopy with hyperspectral imaging, HSI). FIG. 5B shows an exemplary multiplexed labeling of GAPDH and HSP90-alpha mRNA and corresponding proteins with QDots. DNA encoding methodology enables ssDNA tagging of mRNA targets via in situ hybridization and protein targets via immunorecognition by antibody-ssDNA bioconjugates. All ssDNA tags are simultaneously converted into distinctive optical signals by hybridization with complementary QDot-ssDNA′ probes. Fluorescence microscopy with HSI is employed for cell imaging and unmixing of 4 individual QDot channels. Individual grayscale channels are false-colored and merged into a composite 4-color image. Scale bar, 50 μm.

FIG. 6 shows a workflow for target encoding and labeling via in situ hybridization, immunorecognition, and multi-omics procedures. DNA encoding methodology allows for labeling of different types of targets (mRNA and proteins in this proof-of-concept study) under conditions optimized for selective target binding in separate steps. As a result, all targets are converted into a uniform array of intermediate ssDNA tags, which are then simultaneously labeled by complementary QDot-ssDNA′ probes for multiplexed imaging.

FIG. 7A and FIG. 7B illustrate a schematic and characterization of QDot-ssDNA probe preparation. FIG. 7A shows that amine crosslinking by a homobifunctional reagent BS3 is used for covalent conjugation of 5′ amine-terminated ssDNA oligonucleotides and PEG-coated amine-functionalized QDots. ssDNA is activated by an excess BS3, purified by desalting, and reacted with QDots overnight. QDot-ssDNA probes are purified from excess unbound ssDNA by ultrafiltration. Agarose gel electrophoresis in FIG. 7B shows increase in QDot gel motility upon conjugation of negatively-charged ssDNA oligonucleotides, confirming successful preparation of QDot-ssDNA probes.

FIG. 8A and FIG. 8B show a schematic and characterization of antibody-ssDNA bioconjugate preparation via maleimide-mediated crosslinking. FIG. 8A shows rabbit anti-mouse IgG is partially reduced by treatment with TCEP to expose sulfhydryl groups for ssDNA conjugation. At the same time, 5′ amine-terminated ssDNA oligonucleotides are activated by sulfo-SMCC. Mixing and 4-hour incubation of activated ssDNA with reduced IgG yields 1/2IgG-ssDNA bioconjugates. PAGE analysis of bioconjugation products in FIG. 8B confirms formation of primarily 1/2IgG with one ssDNA along with smaller fractions of 1/2IgG conjugated to two and three ssDNA tags.

FIG. 9 illustrates evaluation of a 6-color QDot panel for protein labeling via DNA encoding. FIG. 9A shows specific staining of β-tubulin via incubation with mouse anti-β-tubulin primary antibody and ssDNA-conjugated rabbit anti-mouse secondary antibody followed by immuno-labeling with anti-rabbit QDot655-2′Ab probes indicates preserved functionality of 2′Ab-ssDNA bioconjugates. Consistent β-tubulin staining achieved via hybridization with complementary QDot-ssDNA probes in FIG. 9B confirms successful preparation of a functional 6-color QDot-ssDNA panel. A lack of non-specific binding in FIG. 9C by QDot-ssDNA probes in control experiments skipping incubation with primary and secondary antibodies corroborates utility of such probes for a highly specific target labeling via DNA encoding. True-color images for target staining (FIG. 9B) vs. control (FIG. 9C) are obtained at consistent exposure time for each QDot color. Scale bar, 50 μm.

FIG. 10A, FIG. 10B, and FIG. 10C show schematic and characterization of antibody-ssDNA bioconjugate preparation with Thunder-Link oligo conjugation system. A 2-step amine crosslinking strategy as illustrated in FIG. 10A is employed for preparation of covalent antibody-ssDNA bioconjugates with intact IgG. Antibody and 5′ amine-terminated ssDNA are simultaneously activated by respective activation reagents, purified via desalting, and reacted overnight, producing IgG with varying number of ssDNA tags attached. A reducing PAGE analysis in FIG. 10B highlights presence of multiple higher-MW bands corresponding to heavy and light chains conjugated to varying number of ssDNA tags. In 4 reaction conditions performed with goat anti-rabbit secondary antibodies, relative volume ratios of activated IgG to ssDNA are 1) 50+50, 2) 50+30, 3) 50+20, and 4) 50+10. As expected, increasing amount of ssDNA in the reaction leads to more ssDNA tags being conjugated to each IgG molecule. Staining of Lamin A in FIG. 10C via incubation with rabbit anti-Lamin A primary antibody and goat anti-rabbit 2′Ab-ssDNA bioconjugates followed by labeling with QDot605-ssDNA′ probes confirms preserved specificity of ssDNA-tagged antibodies and successful antibody-ssDNA bioconjugation. At the same time, increasing non-specific binding by 2′ Ab-ssDNA bioconjugates can be observed with increasing number of ssDNA tags per IgG in a control experiment skipping incubation with primary antibody. Thus, a volume ratio of Ab: ssDNA=2:1 in Thunder-Link reaction is considered optimal. All true-color images are obtained at consistent exposure for direct comparison of staining intensity. Scale bar, 250 μm.

FIG. 11A, FIG. 11B, FIG. 11C, FIG. 11D, and FIG. 11E show multiplexed protein labeling via DNA encoding with a panel of 1′ antibody-ssDNA bioconjugates. Primary antibodies against HSP90-alpha, GAPDH, Lamin A, and β-tubulin are conjugated to ssDNA tags using Thunder-Link oligo conjugation system. Reducing PAGE shows consistent formation of IgG-ssDNA bioconjugates for all antibodies (FIG. 11A). Conventional 2-step immunofluorescence with unmodified antibodies and QDot565-2′Ab probes shows characteristic staining pattern for the 4 proteins of interest (FIG. 11B). Protein labeling in FIG. 11C with 1′Ab-ssDNA bioconjugates and QDot565-2′Ab probes yields staining patterns consistent with unmodified antibodies in FIG. 11B, confirming preserved antigen-binding functionality of 1′Ab-ssDNA. Single-color staining with 1′Ab-ssDNA bioconjugates and complementary QDot-ssDNA′ probes further corroborates successful ssDNA conjugation and preparation of an antibody-ssDNA panel suitable for protein labeling via DNA encoding (FIG. 11D). Multiplexed staining via DNA encoding yields consistent staining patterns for all 4 proteins in respective spectral channels of the same hyperspectral image (FIG. 11E). Individual grayscale channels are false-colored for clarity. Scale bar, 50 μm.

FIG. 12 shows characterization of mRNA labeling intensity and specificity via DNA encoding. GAPDH mRNA is labeled via indirect FISH procedure with 41 nt FISH probe set (see TABLE 5) followed by staining with QDot605-ssDNA (left panels) or AlexaFluor555-labeled streptavidin-ssDNA (right panels) probes. Consistent characteristic punctuate staining pattern is observed with both complementary imaging probes (top row). At the same time, non-complementary probes (bottom row) fail to hybridize to mRNA ISH probes, confirming staining specificity of the DNA encoding methodology. “Match” and “mismatch” true-color images are obtained at consistent exposure for direct comparison of staining intensity. Scale bar, 50 μm.

FIG. 13A, FIG. 13B, FIG. 13C, and FIG. 13D illustrates the effect of dsDNA spacer in ISH probe on mRNA labeling intensity. Physical separation of mRNA-recognition and QDot-binding portions of 41 nt ssDNA ISH probes with a 16 bp dsDNA spacer prevents formation of secondary structures, promotes hybridization to target mRNA, and reduces steric hindrance to QDot binding. As a result, a substantial increase in mRNA staining intensity can be realized with such probes (FIG. 13A) in comparison to 41 nt ssDNA FISH probes (FIG. 13B). At the same time, longer 60 nt ssDNA probes without pre-hybridized dsDNA spacers experience greater degree of secondary structure formation, which interferes with mRNA and QDot binding and fails to produce robust mRNA staining (FIG. 13C) above non-specific QDot binding levels (FIG. 13D). All images are obtained with HSI and normalized for direct comparison of signal intensity. Scale bar, 50 μm.

FIG. 14 shows multi-omics QDot staining via DNA encoding. Protein and mRNA targets are encoded with ssDNA tags in separate steps, each using conditions optimal for binding of a specific target type. Consequently, DNA sequence code is converted into an optical signal by hybridization with complementary QDot-ssDNA probes. Specifically, GAPDH mRNA is labeled with a 41 nt ISH probe set followed by labeling of β-tubulin with Ab-ssDNA bioconjugates. Finally, both ssDNA tags are simultaneously hybridized with respective QDot-ssDNA′ probes. Clear microtubule staining pattern of β-tubulin (false-colored green) and punctuate pattern of GAPDH mRNA (false-colored red) are observed in dual-labeled specimen (top row), whereas only β-tubulin staining is present in a control specimen that was not hybridized with GAPDH FISH probe set (bottom row). Nuclei are counter-stained with DAPI (false-colored blue). Scale bar, 100 μm.

FIG. 15 illustrates the heterogeneity in GAPDH RNAi following forward transfection with siRNA. Cells are seeded into a 24-well plate, allowed to attach, grown overnight, and then transfected with GAPDH siRNA (or non-targeting control siRNA) for 24 Hrs. GAPDH mRNA is encoded via in situ hybridization with mRNA ISH probes and then labeled with QDot605-ssDNA′ probes. Imaging of different areas within the well highlights heterogeneity in GAPDH knock-down, likely resulting from heterogeneity in cell transfection with siRNA. Specifically, complete GAPDH mRNA degradation is observed throughout cells in the well center (top right panel), whereas cells at the crowded well edge still express regular levels of GAPDH mRNA (bottom right panel) consistent with GAPDH expression in cells transfected with control siRNA (left panels). Substantial number of non-transfected cells might explain an average silencing efficiency of 78% determined by RT-PCR. Insets: control experiments showing lack of QDot non-specific binding in the absence of complementary ssDNA probes. All images are obtained with true-color camera at the same exposure time for direct comparison of signal intensity. Scale bar, 250 μm.

FIG. 16 illustrates the heterogeneity in GAPDH RNAi following reverse transfection with siRNA. Cells are mixed with GAPDH siRNA (or non-targeting control siRNA) in suspension and then seeded to 24-well plate for transfection and growth for 24 Hrs. GAPDH mRNA is encoded via in situ hybridization with mRNA ISH probes and then labeled with QDot605-ssDNA′ probes. As evident from imaging of different areas within the well, reverse transfection achieves a more uniform transfection and GAPDH knock-down compared to forward transfection (see FIG. 12). Complete GAPDH mRNA degradation is observed throughout majority of cells, with only occasional colonies with full GAPDH expression forming from non-transfected cells, which is consistent with an improved average silencing efficiency of 95% determined by RT-PCR. Insets: control experiments showing lack of QDot non-specific binding in the absence of complementary ssDNA probes. All images are obtained with true-color camera at the same exposure time for direct comparison of signal intensity. Scale bar, 250 μm.

FIG. 17 shows the comparison of RNAi effect on GAPDH mRNA expression following forward vs. reverse transfection with siRNA. Both transfection methods have no effect on GAPDH expression when non-targeting control siRNA is used (left panels) and yield efficient GAPDH knock-down with GAPDH-targeting siRNA (middle panels), as evident from the lack of mRNA staining above non-specific QDot background (right panels). At the same time, small fraction of cells fails to get transfected and, as a result, expresses normal levels of GAPDH mRNA consistent with control experiments. This observation corroborates and all-on/all-off effect of RNAi regardless of transfection method used. All images are obtained with HSI and normalized for direct comparison of signal intensity. Scale bar, 50 μm.

FIG. 18 shows assessment of heterogeneity in cell transfection with siRNA. Dual-labeling of GAPDH and HSP90-alpha mRNA with QDots enables direct visualization of siRNA transfection effect at a single-cell level. Cells are either grown under regular culture conditions (FIG. 18A, FIG. 18B, and FIG. 18C), transfected with control non-targeting siRNA (FIG. 18D, FIG. 18E, and FIG. 18F), or transfected with GAPDH-targeting siRNA (FIG. 18G, FIG. 18H, and FIG. 18I). After a 24-hour treatment with GAPDH siRNA, the majority of cells had completely degraded GAPDH mRNA, as evident from the lack of GAPDH mRNA staining (FIG. 18G). At the same time, HSP90-alpha mRNA not targeted by RNAi machinery remained unperturbed (FIG. 18H). Interestingly, a single cell in the field of view failed to transfect with GAPDH siRNA (FIG. 18G, FIG. 18H, and FIG. 18I), expressing regular levels of GAPDH mRNA consistent with cells treated with control siRNA (FIG. 18D, FIG. 18E, and FIG. 18F) and reference cells not transfected with siRNA (FIG. 18A, FIG. 18B, and FIG. 18C), suggesting an all-on/all-off effect of RNAi. Dual-color images were obtained with hyperspectral imaging (HIS) and were unmixed in QDot channels. Panels for individual channels (FIG. 18A, FIG. 18B, FIG. 18D, FIG. 18E, FIG. 18G, and FIG. 18H) were normalized for direct comparison of signal intensity. In merged 2-color images (FIG. 18C, FIG. 18F, and FIG. 18I) The GAPDH channel was false-colored green and the HSP90-alpha channel was false-colored red. Scale bar, 50 μm.

FIG. 19 shows assessment of GAPDH RNAi heterogeneity at mRNA and protein levels with multi-omics imaging. Dual labeling of GAPDH mRNA and protein 24 Hrs post-transfection with GAPDH-targeting siRNA highlights heterogeneity in mRNA expression levels (bottom left panel) along with the lack of RNAi effect on the protein level (bottom middle panel) at this time point. Transfection with non-targeting control siRNA (top row) fails to affect GAPDH expression, yielding uniform mRNA and protein staining throughout all cells. Dual-color images are obtained with HSI, and individual channels are normalized for direct comparison of signal intensity. GAPDH mRNA channel is false-colored red and GAPDH protein channel is false-colored green in a composite 2-color image. Scale bar, 50 μm.

FIG. 20A and FIG. 20B show assessment of disparity in RNAi kinetics at mRNA and protein levels. HeLa cells are transfected with GAPDH siRNA for (FIG. 20A) 24 hours and (FIG. 20B) 48 hours. GAPDH and HSP90-alpha mRNA along with corresponding proteins are simultaneously assessed with QDot-based multi-omics imaging methodology. Consistent with mRNA-only analysis, multi-omics imaging highlights complete and selective degradation of GAPDH mRNA 24 hours post-transfection, whereas GAPDH protein level remains nearly unperturbed (FIG. 20A). Lagging mRNA knock-down, 48 hours post-transfection selective degradation of GAPDH protein can be observed (FIG. 20B). All grayscale images are normalized to HSP90 protein channel for direct comparison of staining intensities. In a merged 4-color image GAPDH protein channel is false-colored yellow, HSP90-alpha protein-blue, GAPDH mRNA-green, and HSP90-alpha mRNA-red. Scale bar, 50 μm.

FIG. 21A and FIG. 21B show multi-omics evaluation of GAPDH and HSP90-alpha expression at mRNA and protein levels under regular cell culture conditions. To provide a reference of normal GAPDH and HSP90 expression levels to RNAi experiments, cells are grown under regular cell culture conditions for (FIG. 21A) 24 Hrs and (FIG. 21B) 48 Hrs. All targets of interest are labeled via a 2+2 encoding procedure to produce a 4-plex staining. Consistent with expected fast growth of HeLa cells, cell density increases with time. However, GAPDH and HSP90 expression remains constant through 48 Hrs of incubation, as evident from consistent intensity of mRNA and protein labeling. Multiplex images are obtained with HSI, and individual channels are normalized for direct comparison of signal intensity. GAPDH mRNA channel is false-colored green, HSP90 mRNA-red, GAPDH protein-yellow, and HSP90 protein-blue in a composite 4-color image. Scale bar, 50 μm.

FIG. 22A and FIG. 22B show multi-omics evaluation of GAPDH and HSP90-alpha expression at mRNA and protein levels following transfection with a control (non-targeting) siRNA. To assess an effect of transfection on molecular expression profiles in reference to GAPDH RNAi experiments, cells are reverse transfected with non-targeting control siRNA for (FIG. 22A) 24 Hrs and (FIG. 22B) 48 Hrs. All targets of interest are labeled via a 2+2 encoding procedure to produce a 4-plex staining. Consistent with expected lack of RNAi with control siRNA, GAPDH and HSP90 expression remains constant through 48 Hrs of incubation, as evident from consistent intensity of mRNA and protein labeling. Multiplex images are obtained with HSI, and individual channels are normalized for direct comparison of signal intensity. GAPDH mRNA channel is false-colored green, HSP90 mRNA-red, GAPDH protein-yellow, and HSP90 protein-blue in a composite 4-color image. Scale bar, 50 μm.

FIG. 23A and FIG. 23B show direct visualization of GAPDH RNAi effect and kinetics via single-plex labeling of individual protein and mRNA targets. To eliminate any potential effect of multi-omics labeling methodology and artifacts of HSI analysis, GAPDH RNAi experiment along with reference and control is performed on separate specimens in parallel (different wells of the same 24-well plate), followed by a single-plex labeling of individual targets and direct true-color imaging under consistent imaging conditions. Cells are reverse transfected for (FIG. 23A) 24 Hrs and (FIG. 23B) 48 Hrs prior to fixation and staining. Consistent with multi-omics analysis, single-plex imaging confirms efficient and specific degradation of GAPDH mRNA within 24 Hrs post-transfection, whereas RNAi effect on GAPDH protein level can be observed only 48 Hrs post-transfection. Scale bar, 50 μm.

FIG. 24 shows a schematic of a detection agent comprising a probe, a detectable moiety, and a conjugating moiety.

FIG. 25 shows a flowchart for a method of detecting a nucleic acid sequence.

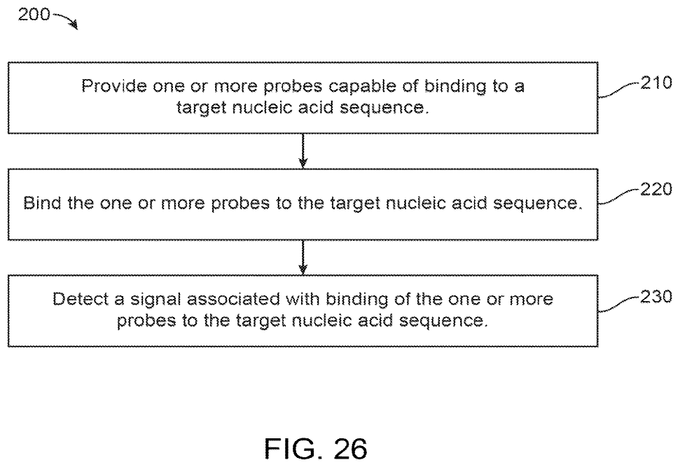

FIG. 26 shows a flowchart for a method of determining the spatial position of a nucleic acid sequence.

FIG. 27 shows a flowchart for a method of detecting the sequence position of a nucleic acid sequence.

FIG. 28 shows a flowchart for a method of detecting a nucleic acid in a sample relative to a control.

FIG. 29 shows a flowchart for a method of fluorescently detecting a target nucleic acid sequence.

FIG. 30 shows a flowchart for a method of analyzing a fluorescence image of one or more target nucleic acid sequences.

FIG. 31 shows a flowchart for a method of analyzing a fluorescence image of one or more target nucleic acid sequences.

FIG. 32 illustrates a conceptual schematic of an exemplary computer server to be used for processing one or more methods described herein.

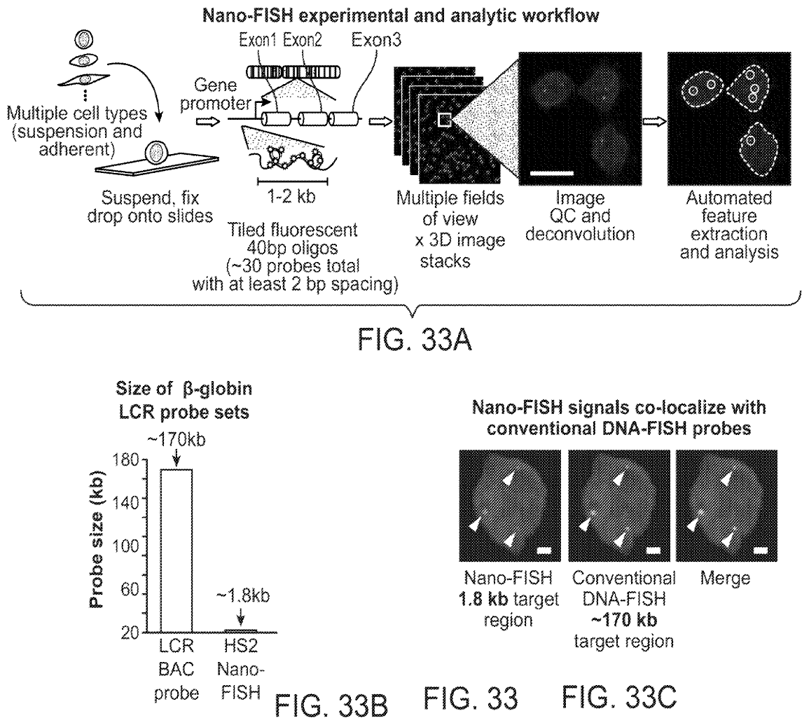

FIG. 33 shows the use of Nano-FISH to detect a 1.8 kb nucleic acid sequence. FIG. 33A shows a schematic of the Nano-FISH experiment. FIG. 33B shows the application of the Nano-FISH strategy to detect a 1.8 kb region encompassing the HS2 hypersensitive site of the β-globin locus control region (LCR) in triploid K562 erythroleukemia cells. FIG. 33C shows colocalization of the Nano-FISH signals (˜1.8 kb target region) with those from standard BAC-derived probes (conventional DNA-FISH; ˜170 kb target region), confirming the specificity of the detected Nano-FISH signal. FIG. 33D shows the efficiency and resolution of detection using Nano-FISH may be tuned according to the number of probes being used. FIG. 33E shows a comparison of the size of detected FISH spots between conventional FISH, pooled HS1-5 probes, and HS2 Nano-FISH. FIG. 33F shows a comparison of the intensity of detected FISH signals between conventional FISH, pooled HS 1-5 probes, and HS2 Nano-FISH. FIG. 33G shows Nano-FISH detected for genomic regions with varying size, such as a genomic region size ranging from about 800 bp to 2.1 kb.

FIG. 34 shows the use of Nano-FISH to perform fine structural analysis of specific genomic loci within the nucleus. FIG. 34A shows the distinct spots produced by Nano-FISH probes targeting specific loci on these chromosomes. To measure the relative localization of the detected loci, the relative radial distance (RRD), a normalized measure of the position of the detected spot with respect to the nuclear centroid, was calculated. FIG. 34B shows a schematic of the relative radial distance. FIG. 34C shows that the chromosome 18 Nano-FISH signals are closer to the nuclear periphery. The distributions were obtained across 2,396 chromosome 18 signals and 3,388 chromosome 19 signals. FIG. 34D shows radial histograms of the two target loci. The differences in the distribution of signals with respect to the nuclear centroid are readily apparent in the histograms.

FIG. 35 shows the use of Nano-FISH for examining the interaction of a gene enhancer with its target gene promoter. FIG. 35A shows two-color Nano-FISH in 786-O and MCF-7 cells. The normalized inter-spot distance (NID) between these two genomic loci were compared. FIG. 35B shows a schematic of the normalized inter-spot distance. FIG. 35C shows that, on average, the spots are situated closer together in 786-O cells compared to MCF-7 cells. FIG. 35D shows that, in spite of this, absolute colocalization (NID=0) was actually a rare event in both cell types.

FIG. 36 shows the use of Nano-FISH to detect small genomic structural variations such as small losses or gains of DNA. ZFN-mediated genome editing was used to generate a triploid homozygous deletion of the β-globin locus control region (LCR, ˜18 kb) in K562 cells, as shown in FIG. 36A. Cells imbued with this deletion are referred to as ALCR. Probes targeting either the HS2 or HS3 hypersensitive sites within the deleted region were utilized to detect loss of LCR in the genome edited cells, as shown in FIG. 36B and FIG. 36C. For the converse scenario, using TALEN-mediated homology directed repair, a sequence encoding for eGFP was inserted into the AAVS1 safe harbor locus on chromosome 19, as shown in FIG. 36D. This exogenously-derived sequenced was readily identified by Nano-FISH, as shown in FIG. 36E and FIG. 36F.

FIG. 37 shows the combination of Nano-FISH and super-resolution microscopy to obtain very fine-scale genome localization. FIG. 37A shows that these closely apposed loci are readily discernible as distinct spots by STED microscopy. Pair-wise measurements of other closely situated genomic segments such as HS1-HS4 (˜12 kb) and HS2-HGB2 (˜25 kb) were also readily obtained and revealed non-linear compaction of the β-globin locus control region and the surrounding genome which contains its target genes, as shown in FIG. 37B. Importantly, the high-throughput STED microscopy approach enables calculation of the distribution of actual distances between these various loci, as shown in FIG. 37C.

FIG. 38 shows a series of experiments to determine the optimal operating parameters for a Nano-FISH experiment. FIG. 38A shows how the labeling efficiency of the Nano-FISH procedure depends on denaturation temperature. With increasing temperature, the efficiency of Nano-FISH labeling increases, until a plateau is reached at a temperature of 78° C. FIG. 38B shows that the Nano-FISH labeling procedure is repeatable across experiments. FIG. 38C shows Nano-FISH detected for genomic regions with varying size, such as a genomic region size ranging from about 800 bp to 2.1 kb. FIG. 38D shows how the labeling efficiency of the Nano-FISH experiment depends on the number of oligo probes used. The labeling efficiency increases with the number of oligo probes used, attaining a maximum efficiency when 30 oligo probes are utilized. FIG. 38E shows how the detected fluorescence spot size depends on the number of oligo probes. FIG. 38F shows how the intensity of the fluorescence spot size depends on the number of oligo probes.

FIG. 39 shows a comparison of Nano-FISH and conventional FISH. FIG. 39A shows fluorescence images of β-globin lacking the LCR using conventional BAC probes (left panel), a pool of HS1-5 probes (middle panel), and the HS2 Nano-FISH technique (right panel). FIG. 39B shows the size of the probe sets used for the BAC, HS 1-5, and HS2. FIG. 38E shows the intensity of the FISH signals for the BAC, HS1-5, and HS2 experiments. As can be seen, the HS2 Nano-FISH experiment utilizes a significantly smaller nucleic acid sequence than conventional FISH techniques. FIG. 39C shows the labeling efficiency of the BAC, HS 1-5, and HS2 experiments. FIG. 39D shows the size of the FISH spots for the BAC, HS 1-5, and HS2 experiments. FIG. 39E shows the intensity of the FISH signals for the BAC, HS 1-5, and HS2 experiments.

### DETAILED DESCRIPTION OF THE INVENTION

Cellular activation and extinction patterns can encode information on cell identity, maturation state, cellular memory, and disease state. Tissues are composites of cells which can have one or more morphologically distinct cell types. In some instances, all of the cells in a tissue are processed simultaneously, yielding compounded information with limited sensitivity for cellular activities and/or rare cell types. Alternative approaches employ disaggregation and sorting of tissue components but in the process can destroy cellular architecture and potentially introduce artifacts such as biological stressors and perturbations.

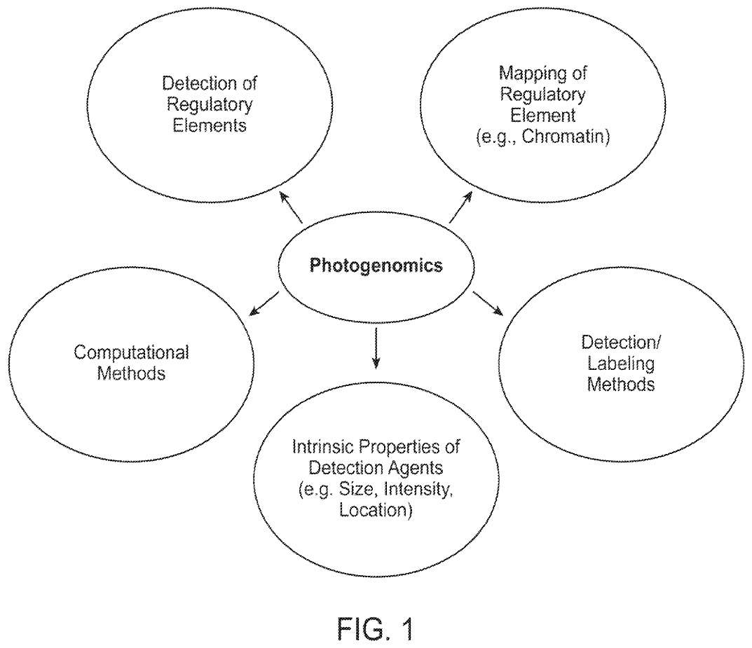

Described herein are methods of detecting a cellular regulatory element in situ utilizing a super-resolution microscopy technique to determine the presence, absence, and/or activity of a regulatory element. Also described herein are methods of detecting different types of regulatory elements simultaneously utilizing a heterogeneous set of detection agents, and translating the molecular information from the different types of regulatory elements to determine the activity state of a cell. The activity state of a cell may correlate to a localization, expression level, and/or interaction state of a regulatory element. One or more of the methods described herein may further interpolate 2-dimensional images to generate 3-dimensional maps which enable detection of localization, interaction states, and activity of one or more regulatory elements. Intrinsic properties such as size, intensity, and location of a detection agent further may enable detection of a regulatory element. Described herein are methods of determining the localization of a regulatory element and measuring the activity of a regulatory element. The methods provided herein may avoid the introduction of artifacts such as biological stressors and perturbations or destroys cellular architecture. Exemplary properties associated with the methods described herein are illustrated in FIG. 1.

One or more methods described herein may detect different types of regulatory elements, distinguish between different types of regulatory elements, and/or generate a map of a regulatory element (e.g., chromatin). For example, a regulatory element may be labeled by one or more different types of detection agents. The one or more different types of detection agents may include DNA detection agents, RNA detection agents, protein detection agents, or combinations thereof. The detection agent may comprise a probe portion, which may interact (e.g., hybridize) to a target site within the regulatory element, and optionally comprise a detectable moiety. The detectable moiety may include a fluorophore, such as a fluorescent dye or a quantum dot. The detection agent may be an unlabeled probe which can be further conjugated to an additional labeled probe. Upon labeling, the regulatory element may be detected by stochastic or deterministic super-resolution microscopy method. The stochastic super-resolution microscopy method may be a synthetic aperture optics (SAO) method. The SAO method may generate a detection profile, which can encompass fluorescent signal intensity, size, shape, or localization of the detection agent. Based on the detection profile, the activity state, the localization, expression level, and/or interaction state of the regulatory element may be determined. A map based on the detection profile of the regulatory element may also be generated, and may be correlated to cell type identification (e.g., cancerous cell identification). The regulatory element may be further analyzed in the presence of an exogenous agent or condition, such as a small molecule fragment or a drug, or under an environment such as a change in temperature, pH, nutrient, or a combination thereof. The perturbation of the activity state of the regulatory element in the presence of the exogenous agent or condition may be measured. A report may further be generated and provided to a user, such as a laboratory clinician or health care provider.

The systems and methods disclosed herein also relate to a novel FISH methodology (hereinafter referred to as “Nano-FISH”) to reliably label and detect localized small (less than 12 kb in size) DNA segments in cells. In some cases, Nano-FISH utilizes defined pools of synthetic fluorescent dye-labeled oligonucleotides to reliably detect small genomic regions in large numbers of adherent or suspension cells in situ. In some instances, Nano-FISH is conducted utilizing conventional wide-field microscopic imaging. In other embodiments, Nano-FISH is conducted using super-resolution imaging techniques.

In some cases, Nano-FISH is coupled with an automated image informatics pipeline to enable high-throughput detection and 2D and/or 3D spatial localization of small genomic DNA elements in situ in hundreds of, thousands of, or more individual cells per experiment. In some instances, to facilitate rigorous statistical analyses of the resulting large image data sets, a scalable image analysis software suite reliably identifies and quantitatively annotates labeled loci on a single-cell basis.

In some cases, Nano-FISH allows detection of the precise localization of specific regulatory genomic elements in 3D nuclear space, the identification of small-scale structural genomic variations (such as sequence gains or losses), the quantitation of spatial interactions between regulatory elements and their putative target gene(s), or the detection of genomic conformational changes that induce stimulus-dependent gene expression. In some instances, Nano-FISH allows the visualization of the precise localization of a target nucleic acid sequence. The target nucleic acid sequence can be an endogenous nucleic acid sequence, a nucleic acid sequence derived from an exogenous source, or a combination thereof. An exogenous target nucleic acid sequence may be introduced to a cell through electroporation, lipofection, transfection, microinjection, viral transduction, or a gene gun. Non-limiting examples of vector systems that can be used to introduce a target nucleic acid sequence into a cell may include viral vector, episomal vector, naked RNA (recombinant or natural), naked DNA (recombinant or natural), bacterial artificial chromosome (BAC), and RNA/DNA hybrid systems used separately or in combination. Vector systems may be used without additional reagents meant to aid in the incorporation and/or expression of desired mutations. A non-limiting list of reagents meant to aid in the incorporation and/or expression of desired mutations may include Lipofectamine, FuGENE, FuGENE HD, calcium phosphate, HeLaMONSTER, Xtreme Gene. An endogenous nucleic acid sequence may be a gene sequence or fragment thereof. An endogenous nucleic acid sequence may be a sequence in a chromosome. An endogenous nucleic acid sequence may be a nucleic acid sequence resulting from somatic chromosomal rearrangement, such as the nucleic acid sequence of a B cell receptor, T cell receptor, or fragment thereof. In some instances, Nano-FISH allows the detection of the precise localization of exogenous nucleic acids inserted or integrated into a genome. In some embodiments, Nano-FISH allows the detection of the precise localization of exogenous DNA inserted into a genome, as may be inserted by a genetic engineering technique. In some instances, Nano-FISH allows the detection of an episomal nucleic acid sequence.

The systems and methods described herein may be useful in detecting or determining the presence, absence, identity, or quantity of a target nucleic acid sequence in a sample. In particular, the methods, compositions, and systems described herein may be used to efficiently detect, to identify, and to quantify a target nucleic acid sequence that is a short nucleic acid sequences. In some cases, a short nucleic acid sequence that may be detected or quantified using the disclosures of the present application may be from 1 kilobase (kb) in length to about 12 kb in length. A short nucleic acid sequence may be less than 1 kb. A short nucleic acid sequence may be less than 12 kb, less than 11 kb, less than 10 kb, less than 9 kb, less than 8 kb, less than 7 kb, less than 6 kb, less than 5 kb, less than 4 kb, less than 3 kb, less than 2.5 kb, less than 2 kb, less than 1.5 kb, less than 1.2 kb, less than 0.8 kb, or less than 0.5 kb. A short nucleic acid sequence may be from 240 nucleotides to 1 kb in length. A short nucleic acid sequence may be from 360 nucleotides to 1 kb in length. A short nucleic acid sequence may be from 240 nucleotides to 2 kb, 15 nucleotides to 2.5 kb, 240 nucleotides to 3 kb, 15 nucleotides to 4 kb, 240 nucleotides to 5 kb, 15 nucleotides to 6 kb, 240 nucleotides to 7 kb, 240 nucleotides to 8 kb, 240 nucleotides to 9 kb, 15 nucleotides to 10 kb, 240 nucleotides to 11 kb, or 240 nucleotides to 12 kb in length. A short nucleic acid sequence may be from 360 nucleotides to 2 kb, 360 nucleotides to 2.5 kb, 360 nucleotides to 3 kb, 360 nucleotides to 4 kb, 360 nucleotides to 5 kb, 360 nucleotides to 6 kb, 360 nucleotides to 7 kb, 360 nucleotides to 8 kb, 360 nucleotides to 9 kb, 360 nucleotides to 10 kb, 360 nucleotides to 11 kb, or 360 nucleotides to 12 kb in length.

Methods for the detection, identification, and/or quantification of a short nucleic acid sequence of a sample may comprise contacting the short nucleic acid sequence with a probe comprising a detectable label and determining the presence, absence, or quantity of probes bound to the target nucleic acid sequence. Determination of the sequence position of the short nucleic acid sequence relative to other nucleotides or another short nucleic acid sequence (for instance, using a second probe capable of binding to a second target sequence of the nucleic acid) may be a step in the methods described herein. The methods described herein may also comprise determining the spatial position of the short nucleic acid sequence. For example, Nano-FISH may be used to measure the normalized inter-spot distance between a first short nucleic acid sequence encoding an enhancer or portion thereof and a second nucleic acid encoding a promoter of a gene or portion thereof, which may be used to study changes in genome conformation that may be associated with gene function.

The methods described herein may comprise comparing the presence, absence, spatial position, sequence position, or quantity of a short nucleic acid sequence of a sample to a reference value. A non-limiting example of quantifying detection of a short nucleic acid sequence in a cell may comprise quantifying the number of copies of a nucleic acid sequence that has been incorporated into a modified cell (for example, a cell modified by the introduction of a nucleic acid sequence into the cell by genetic editing), which may be used as quality control for modified cells produced by cell engineering strategies.

Also described herein are methods, compositions, and systems useful in characterizing and/or quantifying the presence, absence, position, or identity of a target nucleic acid sequence in a cell or sample derived therefrom relative to a reference nucleic acid sequence in the same cell or sample or relative to a control cell or sample. For example, improvements to the efficiency of detection and to a detection threshold, as described herein, may allow for the detection and characterization of short nucleic acid sequences (for instance, non-repeating nucleic acid sequence insertions) during analysis or validation of cell samples or cell lines.

Additionally, described herein, are methods, compositions, and systems for correlating protein expression with target nucleic acid sequence detection. For example, a target nucleic acid sequence may be associated with the expression of a target protein. Using Nano-FISH, the presence, absence, or quantity of the target nucleic acid sequence may be detected, and a detectable label may be used to detect a target protein expression, which therefore may allow for the correlation between the presence, absence, or quantity of the target nucleic acid sequence and the expression of the target protein.

### Types of Regulatory Elements

A regulatory element may be DNA, RNA, a polypeptide, or a combination thereof. A regulatory element may be DNA. A regulatory element may be RNA. A regulatory element may be a polypeptide. A regulatory element may be any combination of DNA, RNA, and/or polypeptide (e.g., protein-protein complexes, protein-DNA/RNA complexes, and the like).

A regulatory element may be DNA. A regulatory element may be a single-stranded DNA regulatory element, a double-stranded DNA regulatory element, or a combination thereof. The DNA regulatory element may be single-stranded. The DNA regulatory element may be double-stranded. The DNA regulatory element may encompass a DNA fragment. The DNA regulatory element may encompass a gene. The DNA regulatory element may encompass a chromosome. The DNA regulatory element may include endogenous DNA regulatory elements (e.g., endogenous genes). The DNA regulatory element may include artificial DNA regulatory elements (e.g., foreign genes introduced into a cell).

A regulatory element may be RNA. A regulatory element may be a single-stranded RNA regulatory element, a double-stranded RNA regulatory element, or a combination thereof. The RNA regulatory element may be single-stranded. The RNA regulatory element may be double-stranded. The RNA regulatory element may include endogenous RNA regulatory elements. The RNA regulatory element may include artificial RNA regulatory elements. The RNA regulatory element may include microRNA (miRNA), transfer RNA (tRNA), ribosomal RNA (rRNA), messenger RNA (mRNA), pre-mRNA, transfer-messenger RNA (tmRNA), heterogeneous nuclear RNA (hnRNA), short interfering RNA (siRNA), or short hairpin RNA (shRNA). The RNA regulatory element may be a RNA fragment. The RNA regulatory element may be an anti-sense RNA.

An RNA regulatory element may be an enhancer RNA (eRNA). An enhancer RNA may be a non-coding RNA molecule transcribed from an enhancer region of a DNA molecule, and may be from about 50 base-pairs (bp) in length to about 3 kilo base pairs in length (e.g., about 100 bp in length, about 200 bp in length, about 500 bp in length, about 1 kb in length, about 1.5 kb in length, about 2 kb in length, or about 2.5 kb in length). An enhancer RNA may be a 1D eRNA or an eRNA that may be unidirectionally transcribed. An enhancer RNA may also be a 2D eRNA or an eRNA that may be bidirectionally transcribed. An eRNA may be polyadenylated. Alternatively, an eRNA may be non-polyadenylated.

A regulatory element may be a DNasel hypersensitive site (DHS). DHS may be a region of chromatin unoccupied by transcription factors and which is sensitive to cleavage by the DNase I enzyme. The presence of DHS regions within a chromatin may demarcate transcription factory occupancy at a nucleotide resolution. The presence of DHS regions may further correlate with activation of cis-regulatory elements, such as an enhancer, promoter, silencer, insulator, or locus control region. DHS variation may be correlated to variation in gene expression in healthy or diseased cells (e.g., cancerous cells) and/or correlated to phenotypic traits.

A DHS pattern may encode memory of prior cell fate decisions and exposures. For example, upon differentiation, a DHS pattern of a progeny may encode transcription factor occupancy of its parent. Further, a DHS pattern of a cell may encode an environmentally-induced transcription factor occupancy from an earlier time point.

A DHS pattern may encode cellular maturity. An embryonic stem cell may encode a set of DHSs that may be transmitted combinatorially to a differentiated progeny, and this set of DHSs may be decreased with each cycle of differentiation. As such, the set of DHSs may be correlated with time, thereby allowing a DHS pattern to be correlated with cellular maturity.

A DHS pattern may also encode splicing patterns. Protein coding exons may be occupied by transcription factors, which may further be correlated with codon usage patterns and amino acid choice on evolutionary time scales and human fitness. A transcription factory occupancy may further modulate alternative splicing patterns, for example, by imposing sequence constraints at a splice junction. As such, a DHS pattern may encode transcription factor occupancy of one or more exons of interest and may provide additional information on alternative splicing patterns.

A DHS pattern may encode a cell type. For example, within each cell type, about 100,000 to about 250,000 DHSs may be detected. About 5% of the detected DHSs may be located within a transcription start site and the remaining DHSs may be detected at a distal site from the transcription start site. Each cell type may contain a distinct DHS pattern at the distal site and mapping the DHS pattern at the distal site may allow identification of a cell type. An overlap may further be present within two DHS patterns from two different cell types, for example, an overlap of a set of detected DHSs within the two DHS patterns. An overlap may be less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1% of the detected DHSs. The presence of an overlap may not affect the identification of a cell type.

A regulatory element may be a polypeptide. The polypeptide may be a protein or a polypeptide fragment. For example, a regulatory element may be a transcription factor, DNA-binding protein or functional fragment, RNA-binding protein or functional fragment, protein involved in chemical modification (e.g., involved in histone modification), or gene product. A regulatory element may be a transcription factor. A regulatory element may be a DNA or RNA-binding protein or functional fragment. A regulatory element may be a product of a gene transcript. A regulatory element may be a chromatin.

### Methods of Detecting a Regulatory Element

Described herein is a method of detecting a regulatory element. The detection may encompass identification of the regulatory element, determining the presence or absence of the regulatory element, and/or determining the activity of the regulatory element. A method of detecting a regulatory element may include contacting a cell sample with a detection agent, binding the detection agent to the regulatory element, and analyzing a detection profile from the detection agent to determine the presence, absence, or activity of the regulatory element.

The method may involve utilizing one or more intrinsic properties associated with a detection agent to aid in detection of the regulatory element. The intrinsic properties may encompass the size of the detection agent, the intensity of the signal, and the location of the detection agent. The size of the detection agent may include the length of the probe and/or the size of the detectable moiety (e.g., the size of a fluorescent dye molecule) may modulate the specificity of interaction with a regulatory element. The intensity of the signal from the detection agent may correlate to the sensitivity of detection. For example, a detection agent with a molar extinction coefficient of about 0.5-5×10⁶ M⁻¹ cm⁻¹ may have a higher intensity signal relative to a detection agent with a molar extinction coefficient outside of the 0.5-5×10⁶ M⁻¹ cm⁻¹ range and may have lower attenuation due to scattering and absorption. Further, a detection agent with a longer excited state lifetime and a large Stoke shift (measured by the distance between the excitation and emission peaks) may further improve the sensitivity of detection. The location of the detection agent may, for example, provide the activity state of a regulatory element. A combination of intrinsic properties of the detection agent may be used to detect a regulatory element of interest.

A detection agent may comprise a detectable moiety that is capable of generating a light, and a probe portion that is capable of hybridizing to a target site on a regulatory element. As described herein, a detection agent may include a DNA probe portion, an RNA probe portion, a polypeptide probe portion, or a combination thereof. Sometimes, a DNA or RNA probe portion may be between about 10 and about 100 nucleotides in length, between about 15 and about 100 nucleotides in length, between about 20 and about 100 nucleotides in length, between about 20 and about 80 nucleotides in length, between about 20 and about 60 nucleotides in length, between about 25 and about 55 nucleotides in length, between about 30 and about 50 nucleotides in length, between about 15 and about 80 nucleotides in length, between about 15 and about 60 nucleotides in length, between about 20 and about 40 nucleotides in length, or between about 20 and about 30 nucleotides in length. Sometimes, a DNA or RNA probe portion may be about 10, about 15, about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60, about 65, about 70, about 80, about 90, about 100, or more nucleotides in length. A DNA or RNA probe portion may be a TALEN probe, ZFN probe, or a CRISPR probe. A DNA or RNA probe portion may be a padlock probe. A polypeptide probe may comprise a DNA-binding protein, a RNA-binding protein, a protein involved in the transcription/translation process, a protein that detects the transcription/translation process, a protein that may detect an open or relaxed portion of a chromatin, or a protein interacting partner of a product of a regulatory element (e.g., an antibody or binding fragment thereof).

A detection agent may comprise a DNA or RNA probe portion which may be between about 10 and about 100 nucleotides in length, between about 15 and about 100 nucleotides in length, between about 20 and about 100 nucleotides in length, between about 20 and about 80 nucleotides in length, between about 20 and about 60 nucleotides in length, between about 25 and about 55 nucleotides in length, between about 30 and about 50 nucleotides in length, between about 15 and about 80 nucleotides in length, between about 15 and about 60 nucleotides in length, between about 20 and about 40 nucleotides in length, or between about 20 and about 30 nucleotides in length. A detection agent may comprise a DNA or RNA probe portion which may be about 10, about 15, about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60, about 65, about 70, about 80, about 90, about 100, or more nucleotides in length.

A detection agent may comprise a DNA or RNA probe selected from a TALEN probe, a ZFN probe, or a CRISPR probe.

A set of detection agents may be used to detect a regulatory element. The set of detection agents may comprise about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, or more detection agents. Each of the detection agents within the set of detection agents may recognize and interact with a distinct region of a regulatory element. Sometimes, about 1, about 2, about 3, about 4, about 5, about 6, about 7, about 8, about 9, about 10, about 11, about 12, about 13, about 14, about 15, about 16, about 17, about 18, about 19, about 20, or more detection agents may be used for detection of a regulatory element. About 1 or more detection agents may be used for detection of a regulatory element. About 2 or more detection agents may be used for detection of a regulatory element. About 3 or more detection agents may be used for detection of a regulatory element. About 4 or more detection agents may be used for detection of a regulatory element. About 5 or more detection agents as used for detection of a regulatory element. About 6 or more detection agents may be used for detection of a regulatory element. About 7 or more detection agents may be used for detection of a regulatory element. About 8 or more detection agents may be used for detection of a regulatory element. About 9 or more detection agents may be used for detection of a regulatory element. About 10 or more detection agents may be used for detection of a regulatory element. About 11 or more detection agents may be used for detection of a regulatory element. About 12 or more detection agents may be used for detection of a regulatory element. About 13 or more detection agents may be used for detection of a regulatory element. About 14 or more detection agents may be used for detection of a regulatory element. About 15 or more detection agents may be used for detection of a regulatory element. About 20 or more detection agents may be used for detection of a regulatory element.

A detection agent may comprise a polypeptide probe selected from a DNA-binding protein, a RNA-binding protein, a protein involved in the transcription/translation process, a protein that detects the transcription/translation process, a protein that may detect an open or relaxed portion of a chromatin, or a protein interacting partner of a product of a regulatory element (e.g., an antibody or binding fragment thereof).

A detectable moiety that is capable of generating a light may be directly conjugated or bound to a probe portion. A detectable moiety may be indirectly conjugated or bound to a probe portion by a conjugating moiety. As described herein, a detectable moiety may be a small molecule (e.g., a dye) which may be directly conjugated or bound to a probe portion. A detectable moiety may be a fluorescently labeled protein or molecule which may be attached to a conjugating moiety (e.g., a hapten group, an azido group, an alkyne group) of a probe.

A profile or a detection profile or signature may include the signal intensity, signal location, or size of the signal of the detection agent. The profile or the detection profile may comprise about 100 image frames, about 500 frames, about 1000 frames, about 2000 frames, about 5000 frames, about 10,000 frames, about 20,000 frames, about 30,000 frames, about 40,000 frames, about 50,000 frames, or more frames. Analysis of the profile or the detection profile may determine the activity of the regulatory element. The degree of activation may also be determined from the analysis of the profile or detection profile. Analysis of the profile or the detection profile may further determine the optical isolation and localization of the detection agents, which may correlate to the localization of the regulatory element.

In additional cases, a detection agent may comprise a polypeptide probe selected from a DNA-binding protein, a RNA-binding protein, a protein involved in the transcription/translation process or detects the transcription/translation process, a protein that may detect an open or relaxed portion of a chromatin, or a protein interacting partner of a product of a regulatory element (e.g., an antibody or binding fragment thereof).

Sometimes, a detectable moiety that is capable of generating a light is directly conjugated or bound to a probe portion. Other times, a detectable moiety is indirectly conjugated or bound to a probe portion by a conjugating moiety. As described elsewhere herein, a detectable moiety may be a small molecule (e.g., a dye) which may be directly conjugated or bound to a probe portion. Alternatively, a detectable moiety may be a fluorescently labeled protein or molecule which may be attached to a conjugating moiety (e.g., a hapten group, an azido group, an alkyne group) of a probe.

In some instances, a profile or a detection profile or signature may include the signal intensity, signal location, or size of the signal of the detection agent. Sometimes, the profile or the detection profile may comprise about 100 frames, 500 frames, 1000 frames, 2000 frames, 5000 frames, 10,000 frames, 20,000 frames, 30,000 frames, 40,000 frames, 50,000 frames or more images. Analysis of the profile or the detection profile may determine the activity of the regulatory element. In some cases, the degree of activation may also be determined from the analysis of the profile or detection profile. In additional cases, analysis of the profile or the detection profile may further determine the optical isolation and localization of the detection agents, which may correlate to the localization of the regulatory element.

Detection of DNA and/or RNA Regulatory Elements

A regulatory element may be DNA. Described herein is a method of detecting a DNA regulatory element, which may include contacting a cell sample with a detection agent, binding the detection agent to the DNA regulatory element, and analyzing a profile from the detection agent to determine the presence, absence, or activity of the DNA regulatory element.

A regulatory element may be RNA. Described herein is a method of detecting a RNA regulatory element, which may include contacting a cell sample with a detection agent, binding the detection agent to the RNA regulatory element, and analyzing a profile from the detection agent to determine the presence, absence, or activity of the RNA regulatory element.

A regulatory element may be an enhancer RNA (eRNA). The presence of an eRNA may correlate to an activated regulatory element. For example, the production of an eRNA may correlate to the transcription of a target gene. As such, the detection of an eRNA element may indicate that a target gene downstream of the eRNA element may be activated.

Provided herein is a method of detecting an eRNA regulatory element, which may include contacting a cell sample with a detection agent, binding the detection agent to the eRNA regulatory element, and analyzing a profile from the detection agent to determine the presence, absence, or activity of the eRNA regulatory element. Described herein is an in situ method of detecting an activated regulatory DNA site, which may include incubating a sample with a set of detection agents (e.g., fluorescently-labeled probes), hybridizing the set of detection agents to at least one enhancer RNA (eRNA), and analyzing a profile (e.g., a fluorescent profile) from the set of detection agents to determine the presence of an eRNA, in which the presence of eRNA correlates to an activated regulatory DNA site.

### Detection of a DNasel Hypersensitive Site, Generation of a DNasel Hypersensitive Site Map, and Determination of a Cell Type Based on a DNasel Hypersensitive Site Profile

A regulatory element may be a DNasel hypersensitive site (DHS). A DNasel hypersensitive site may be an inactivated DNasel hypersensitive site. A DNasel hypersensitive site may be an activated DNasel hypersensitive site. Described herein is a method of detecting a DHS, which may include contacting a cell sample with a detection agent, binding the detection agent to the DHS, and analyzing a profile from the detection agent to determine the presence, absence, or activity of the DHS.

The DHS may be an active DHS and may further contain a single stranded DNA region. The single stranded DNA region may be detected by S1 nuclease. A method of detecting a DHS may further be extended to detect the presence of a single stranded DNA region within a DHS. Such a method, for example, may comprise contacting a cell sample with a detection agent, binding the detection agent to a single stranded region of a DHS, and analyzing a profile from the detection agent to determine the presence or absence of the single stranded region within a DHS.

Also described herein is a method of determining the activity level of a regulatory element, which may include incubating a cell sample with a set of detection agents (e.g., fluorescently labeled probes), in which each detection agent hybridizes to a DHS, measuring a signature (e.g., a fluorescent signature) from the set of detection agents, and based on the signature, determining a DHS profile, and comparing the DHS profile with a control, in which a correlation with the control indicates the activity level of the regulatory element in the cell sample. The signature (e.g., the fluorescent signature) may further correlate to a signal intensity (or a peak height). A set of signal intensities may be compiled into a DHS profile and compared with a control to generate a second DHS profile which comprises a set of relative signal intensities (or relative peak heights). The set of relative signal intensities may correlate to the activity level of a regulatory element.

Also described herein is a method of generating a DHS map, which may provide information on cell-to-cell variation in gene expression, memory of early developmental fate decisions which establish lineage hierarchies, quantitation of embryonic stem cell DHS sites which decreases with cell passage, and presence of oncogenic elements.

The location of a set of DHS sites may be correlated to a cell type. For example, the location of about 1, about 5, about 10, about 15, about 20, about 25, about 30, about 35, about 40, about 45, about 50, about 55, about 60, or more DHS sites may be correlated to a cell type. The location of about 1, about 5, about 10, about 15, about 20, about 25, about 30, about 35, about 40, about 45, about 50, about 55, about 60, or more DHS may be used to determine a cell type. The cell may be a normal cell or a cancerous cell. DHS variation may be used to determine the presence of cancerous cells in a sample. A method of determining a cell type (e.g., a cancerous cell) may include incubating a cell sample with a set of detection agents (e.g., fluorescently labeled probes), in which each detection agent hybridizes to a DHS, measuring a signature (e.g., a fluorescent signature) from the set of detection agents, and based on the signature, determining a DHS profile, and comparing the DHS profile with a control, in which a correlation with the control indicates the cell type of the sample.

A DHS site may be visualized through a terminal deoxynucleotidyl transferase (TdT) dUTP Nick-End labeling (TUNEL) assay. A TUNEL assay may utilize a terminal deoxynucleotidyl transferase (TdT) which may catalyze the addition of a dUTP at the site of a nick or strand break. A fluorescent moiety may further be conjugated to dUTP. A TUNEL assay may be utilized for visualization of a plurality of DHSs present in a cell. A TUNEL assay may be an assay as described in EXAMPLE 2.

The sequence of a DHS site may be detected in situ, by utilizing an in situ sequencing methodology. For example, the two ends of a padlock probe may be hybridized to a target regulatory element sequence and the two ends may be further ligated together by a ligase (e.g., T4 ligase) when bound to the target sequence. An amplification (e.g., a rolling circle amplification or RCA) may be performed utilizing a polymerase (e.g., $29 polymerase), which may result in a single stranded DNA comprising at least about 1, at least about 2, at least about 3, at least about 4, at least about 5, at least about 6, at least about 7, at least about 8, at least about 9, at least about 10, or more tandem copies of the target sequence. The amplified product at least about be sequenced by ligation in situ using partition sequencing compatible primers and labeled probes (e.g., fluorescently labeled probes). For example, each target sequence within the amplified product may bind to a primer and probe set resulting in a bright spot detectable by, e.g., an immunofluorescence microscopy. The labeled probe (e.g., the fluorescent label on the probe) may identify the nucleotide at the ligation site, thereby allowing the color detected to define the nucleotide at the respective ligation position. Sometimes, at least 1, at least 2, at least 3, at least 4, at least 5, at least 6, at least 7, at least 8, at least 9, at least 10, at least 15, at least 20, or more rounds of ligation and detection may occur for detection of a DHS site.

A control as used herein may refer to a DHS profile generated from a regulatory element those activity level is known. A control may also refer to a DHS profile generated from an inactivated regulatory element. A control may further refer to a DHS profile generated from an activated or inactivated regulatory element from a specific cell type. For example, the cell type may be an epithelial cell, connective tissue cell, muscle cell, or nerve cell type. The cell may be a cell derived from heart, lung, kidney, stomach, intestines, liver, pancreas, brain, esophagus, and the like. The cell type may be a hormone-secreting cell, such as a pituitary cell, a gut and respiratory tract cell, thyroid gland cell, adrenal gland cell, Leydig cell of testes, Theca interna cell of ovarian follicle, Juxtaglomerular cell, Macula densa cell, Peripolar cell, or Mesangial cell type. The cell may be a blood cell or a blood progenitor cell. The cell may be an immune system cell, e.g., monocytes, dendritic cell, neutrophile granulocyte, eosinophil granulocyte, basophil granulocyte, hybridoma cell, mast cell, helper T cell, suppressor T cell, cytotoxic T cell, Natural Killer T cell, B cell, or natural killer cell.

### Detection and Mapping of a Chromatin

A regulatory element may also be a chromatin. Provided herein is a method of detecting a chromatin, which may include contacting a cell sample with a detection agent, binding the detection agent to the chromatin, and analyzing a profile from the detection agent to determine the activity state of the chromatin. The activity level of a chromatin may be determined based on the presence or activity level of a nucleic acid of interest or the presence or absence of a chromatin associated protein. The activity level of a chromatin may be determined based on DHS locations. The one or more DHS locations on a chromatin may be used to map chromatin activity state. For example, one or more DHSs may be localized in a region and the surrounding chromatin may be decompacted and readily visualized relative to an inactive chromatin state when a DHS is not present. The one or more DHSs within a localized region may further form a localized DHS set and a plurality of localized DHS sets may further provide a global map or pattern of chromatin activity (e.g., an activity pattern).

Also included herein is a method of generating a chromatin map based on the pattern of DNaseI hypersensitive sites, RNA regulatory elements (e.g., eRNA), chromatin associated proteins or gene products, or a combination thereof. The method of generating a chromatin map may be based on the pattern of DNasel hypersensitive sites. The method may comprise generating a 3-dimensional map from a detection profile (or a 2-dimensional detection profile). A chromatin map may provide information on the compaction of chromatin, the spatial structure, spacing of regulatory elements, and localization of the regulatory elements to globally map chromatin structure and accessibility.

A chromatin map for a cell type may also be generated, in which each cell type comprises a different chromatin pattern. Each cell type may be associated with at least one unique marker. The at least one unique marker (or fiduciary marker) may be a genomic sequence. The at least one unique marker (or fiduciary marker) may be DHS. A cell type may comprise about 5, about 10, about 15, about 20, about 25, about 30, about 35, about 40, about 45, about 50, about 60, or more unique markers (or fiduciary markers). The cell type may be an epithelia cell, a connective tissue cell, a muscle cell, a nerve cell, a hormone-secreting cell, a blood cell, an immune system cell, or a stem cell type. The cell type may be a cancerous cell type.

A chromatin profile (e.g., based on DHSs) in the presence of an exogenous agent or condition may also be generated. The method may comprise incubating a cell sample with a set of fluorescently labeled probes specific to target sites (e.g., target DHSs) on a chromatin in the presence of an exogenous agent or condition; measuring a fluorescent signature of the set of fluorescently labeled probes; based on the fluorescent signature, generating a fluorescent profile of the chromatin; and comparing the fluorescent profile with a second fluorescent profile of a chromatin obtained from an equivalent sample incubated with an equivalent set of fluorescently labeled probes in the absence of the exogenous agent or condition, wherein a difference between the two sets of fluorescent profiles indicates a change in the chromatin density (e.g., changes in the presences or activation of DHSs) induced by the exogenous agent or condition. The exogenous agent or condition may comprise a small molecule or a drug. The exogenous agent may be a small molecule, such as a steroid. The exogenous agent or condition may comprise an environmental factor, such as a change in pH, temperature, nutrient, or a combination thereof.

### Methods of Determining the Localization of a Regulatory Element

Also described herein is a method for determining the localization of a regulatory element. The localization of a regulatory element may provide an activity state of the regulatory element. The localization of a regulatory element may also provide an interaction state with at least one additional regulatory element. For example, the localization of a first regulatory element with respect to a second regulatory element may provide spatial coordinate and distance information between the two regulatory elements, and v further provide information regarding whether the two regulatory elements may interact with each other. The activity state of a regulatory element may include, for example, a transcription or translation initiation event, a translocation event, or an interaction event with one or more additional regulatory elements. The regulatory element may comprise DNA, RNA, polypeptides, or a combination thereof. The regulatory element may be DNA. The regulatory element may be RNA. The regulatory element may be an enhancer RNA (eRNA). The regulatory element may be a DNasel hypersensitive site (DHS). The DHS may be an inactive DHS or an active DHS. The regulatory element may be a polypeptide. The regulatory element may be chromatin.

The localization of a regulatory element may include contacting a regulatory element with a first set of detection agents, photobleaching the first set of detection agents for a first time point at a first wavelength to generate a second set of detection agents capable of generating a light at a second wavelength, detecting at least one burst generated by the second set of detection agents to generate a detection profile of the second set of detection agents, and analyzing the detection profile to determine the localization of the regulatory element.

A detection agent may comprise a detectable moiety that is capable of generating a light, and a probe portion that is capable of hybridizing to a target site on a regulatory element. Each detection agent within the first set of detection agents may have the same or a different detectable moiety. Each detection agent within the first set of detection agents may have the same detectable moiety. A detectable moiety may comprise a small molecule (e.g., a fluorescent dye). A detectable moiety may comprise a fluorescently labeled polypeptide, a fluorescently labeled nucleic acid probe, and/or a fluorescently labeled polypeptide complex.

Upon photobleaching, a second set of detection agents may be generated from the first set of detection agents, in which the second set may include detection agents that are capable of generating a burst of light detectable at a second wavelength. For example, bleaching of the set of detection agents may lead to about 50%, about 60%, about 70%, about 80%, about 90%, or more detection agents within the set to enter into an “OFF-state”. An “OFF-state” may be a dark state in which the detectable moiety crosses from the singlet excited or ON state to the triplet state or OFF-state in which detection of light (e.g., fluorescence) may be low (e.g., less than 10%, less than 5%, less than 1%, or less than 0.5% of the light may be detected). The remainder of the detection agents that have not entered into the OFF-state may generate bursts of lights, or to cycle between a singlet excited state (or ON-state) and a singlet ground state. As such, bleaching of the set of detection agents may generate about 40%, about 30%, about 20%, about 10%, about 5%, or less detection agents within the set that may generate bursts of lights. The bursts of lights may be detected stochastically, at a single burst level in which each burst of light correlates to a single detection agent.

A single wavelength may be used for photobleaching a set of detection agents. At least two wavelengths may be used for photobleaching a set of detection agents. A wavelength at 491 nm may be used. A wavelength at 405 nm may be used in combination with the wavelength at 491 nm. The two wavelengths may be applied simultaneously to photobleach a set of detection agents. Alternatively, the two wavelengths may be applied sequentially to photobleach a set of detection agents.

The time for photobleaching a set of detection agents may be from about 10 seconds to about 4 hours. The time may be from about 30 seconds to about 3.5 hours, from about one minute to about 3 hours, from about 5 minutes to about 2 hours, from about 10 minutes to about 1 hours, from about one minutes to about 1 hour, from about 5 minutes to about 1 hour, or from about 30 minutes to about 2 hours. The time may be at least 10 seconds, at least 20 seconds, at least 30 seconds, at least 40 seconds, at least 50 seconds, at least 1 minute, at least 2 minutes, at least 3 minutes, at least 4 minutes, at least 5 minutes, at least 10 minutes, at least 15 minutes, at least 20 minutes, at least 30 minutes, at least 45 minutes, at least 1 hour, at least 1.5 hours, at least 2 hours, at least 2.5 hours, at least 3 hours, at least 3.5 hours, at least 4 hours, or more.

The concentration of the detection agents may be from about 5 nM to about 1 μM. The concentration of the detection agent may be from about 5 nM to about 900 nM, from about 10 nM to about 800 nM, from about 15 nM to about 700 nM, from about 20 nM to about 500 nM, from about 10 nM to about 500 nM, from about 10 nM to about 400 nM, from about 10 nM to about 300 nM, from about 10 nM to about 200 nM, from about 10 nM to about 100 nM, from about 50 nM to about 500 nM, from about 50 nM to about 400 nM, from about 50 nM to about 300 nM, from about 50 nM to about 200 nM, from about 100 nM to about 500 nM, from about 100 nM to about 300 nM, or from about 100 nM to about 200 nM. The concentration of the detection agents may be about 10 nM, about 15 nM, about 20 nM, about 30 nM, about 40 nM, about 50 nM, about 60 nM, about 70 nM, about 80 nM, about 90 nM, about 100 nM, about 150 nM, about 200 nM, about 250 nM, about 300 nM, about 400 nM, about 500 nM, about 600 nM, about 700 nM, about 800 nM, about 900 nM, or more.

The burst of lights from the set of detection agents may generate a detection profile. The detection profile may comprise about 100 image frames, about 500 frames, about 1000 frames, about 2000 frames, about 5000 frames, about 10,000 frames, about 20,000 frames, about 30,000 frames, about 40,000 frames, about 50,000 frames, or more. The detection profile may also include the signal intensity, signal location, or size of the signal. Analysis of the detection profile may determine the optical isolation and localization of the detection agents, which may correlate to the localization of the regulatory element.

The detection profile may comprise a chromatic aberration correction. The detection profile may comprise less than 5%, less than 4%, less than 3%, less than 2%, less than 1%, less than 0.5%, less than 0.1%, or 0% chromatic aberration. The detection profile may comprise less than 5% chromatic aberration. The detection profile may comprise less than 4% chromatic aberration. The detection profile may comprise less than 3% chromatic aberration. The detection profile may comprise less than 2% chromatic aberration. The detection profile may comprise less than 1% chromatic aberration. The detection profile may comprise less than 0.5% chromatic aberration. The detection profile may comprise less than 0.1% chromatic aberration. The detection profile may comprise 0% chromatic aberration.

More than one regulatory element may be detected at the same time. At least 2, at least 3, at least 4, at least 5, at least 6, at least 7, at least 8, at least 9, at least 10, at least 15, at least 20, or more regulatory elements may be detected at the same time. Each of the regulatory elements may be detected by a set of detection agents. The detectable moiety between the different set of detection agents may be the same. For example, two different sets of detection agents may be used to detect two different regulatory elements and the detectable moieties from the two sets of detection agents may be the same. As such, at least 2, at least 3, at least 4, at least 5, at least 6, at least 7, at least 8, at least 9, at least 10, at least 15, at least 20, or more regulatory elements may be detected at the same time at the same wavelength. Sometimes, the detectable moiety between the different set of detection agents may also be different. For example, two different sets of detection agents may be used to detect two different regulatory elements and the detectable moiety from one set of detection agents may be detected at a different wavelength from the detectable moiety of the second set of detection agents. As such, at least 2, at least 3, at least 4, at least 5, at least 6, at least 7, at least 8, at least 9, at least 10, at least 15, at least 20, or more regulatory elements may be detected at the same time in which each of the regulatory elements may be detected at a different wavelength. The regulatory element may comprise DNA, RNA, polypeptides, or a combination thereof.

### Methods of Measuring the Activity of a Regulatory Element

Also described herein is a method of measuring the activity of a target regulatory element. The method may include detection of a regulatory element and one or more products of the regulatory element. One or more products of the regulatory element may also include intermediate products or elements. The method may comprise contacting a cell sample with a first set and a second set of detection agents, in which the first set of detection agents interact with a target regulatory element within the cell and the second set of detection agents interact with at least one product of the target regulatory element, and analyzing a detection profile from the first set and the second set of detection agents, in which the presence or the absence of the at least one product indicates the activity of the target regulatory element.

As discussed herein, a detection agent may comprise a detectable moiety that is capable of generating a light, and a probe portion that is capable of hybridizing to a target site on a regulatory element. Each detection agent within the first set of detection agents may have the same or a different detectable moiety. Each detection agent within the first set of detection agents may have the same detectable moiety. A detectable moiety may comprise a small molecule (e.g., a fluorescent dye). A detectable moiety may comprise a fluorescently labeled polypeptide, a fluorescently labeled nucleic acid probe, and/or a fluorescently labeled polypeptide complex.

The method may also allow photobleaching of the first set and the second set of detection agents, thereby generating a subset of detection agents capable of generating a burst of light. A detection profile may be generated from the detection of a set of light bursts, in which the presence or the absence of the at least one product may indicate the activity of the target regulatory element.

The regulatory element may comprise DNA, RNA, polypeptides, or a combination thereof. The regulatory element may be DNA. The regulatory element may be RNA. The regulatory element may be an enhancer RNA (eRNA). The presence of an eRNA may correlate with target gene transcription that is downstream of eRNA. The regulatory element may be a DNasel hypersensitive site (DHS). The DHS may be an activated DHS. The pattern of the DHS on a chromatin may correlate to the activity of the chromatin. The regulatory element may be a polypeptide, e.g., a transcription factor, a DNA or RNA-binding protein or binding fragment thereof, or a polypeptide that is involved in chemical modification. The regulatory element may be chromatin.

### Target Nucleic Acid Sequence

A target nucleic acid sequence may be a nucleic acid sequence of interest or may encode a DNA, RNA, or protein of interest or a portion thereof. A DNA, RNA, or protein of interest may be a DNA, RNA, or protein produced by a cell or contained within a cell. A target nucleic acid sequence may be incorporated into a structure of a cell. A target nucleic acid sequence may also be associated with a cell. For example, a target nucleic acid sequence may be in contact with the exterior of a cell. A target nucleic acid sequence may be unassociated with a structure of a cell. For example, a target nucleic acid sequence may be a circulating nucleic acid sequence. A target nucleic acid sequence or a portion thereof may be artificially constructed or modified. A target nucleic acid sequence may be a natural biological product. A target nucleic acid sequence may be a short nucleic acid sequence. A target nucleic acid sequence may be a nucleic acid sequence that is from a source that is exogenous to a cell. A target nucleic acid sequence may be an endogenous nucleic acid sequence. A target nucleic acid sequence may be a nucleic acid sequence that comprises a combination of an endogenous nucleic acid sequence and a nucleic acid sequence from a source that is exogenous to a cell. A target nucleic acid sequence may be a chromosomal nucleic acid sequence or fragment thereof. A target nucleic acid sequence may be an episomal nucleic sequence or fragment thereof. A target nucleic acid sequence may be a sequence resulting from somatic rearrangement or somatic hypermutation, such as a nucleic acid sequence from a T cell recepto, B cell receptor, or fragment thereof.

A nucleic acid of a cell or sample, which may comprise the target nucleic acid sequence, may comprise a deoxyribonucleic acid (DNA) or a ribonucleic acid (RNA), or a combination thereof. A nucleic acid may be a chromosome, an oligonucleotide, a plasmid, an artificial chromosome, or a fragment or portion thereof. A nucleic acid may comprise genomic DNA, episomal DNA, complementary DNA, mitochondrial DNA, recombinant DNA, cell-free DNA (cfDNA), messenger RNA (mRNA), pre-mRNA, microRNA (miRNA), transfer RNA (tRNA), transfer messenger RNA (tmRNA), ribosomal RNA (rRNA), heterogeneous nuclear RNA (hnRNA), short interfering RNA (siRNA), anti-sense RNA, or short hairpin RNA (shRNA). A nucleic acid may be single-stranded, double-stranded, or a combination thereof.

A target nucleic acid sequence may comprise a naturally occurring nucleic acid sequence, an artificially constructed nucleic acid sequence (such as an artificially synthesized nucleic acid sequence), or a modified nucleic acid sequence (such as a naturally occurring nucleic acid sequence that has been altered or modified through a natural or artificial process).

A naturally occurring nucleic acid sequence may comprise a nucleic acid sequence present in a cellular sample. A naturally occurring nucleic acid sequence may comprise a nucleic acid sequence present in an unfixed cell. A naturally occurring nucleic acid sequence may comprise a nucleic acid sequence derived from a cellular sample.

A naturally occurring nucleic acid sequence may comprise a nucleic acid sequence present in an acellular sample. A naturally occurring nucleic acid sequence may comprise a nucleic acid sequence derived from an acellular sample. For example, a nucleic acid sequence may be a cell-free DNA sequence present in a bodily fluid (such as a sample of cerebrospinal fluid).

A nucleic acid may comprise a target nucleic acid sequence that is not endogenous to the source (exogenous) from which it was taken or in which it is analyzed.

A nucleic acid may be an artificially synthesized oligonucleotide.

A nucleic acid sequence may comprise one or more modifications. A modification to a nucleic acid sequence may be an insertion, a deletion, or a substitution. A nucleic acid sequence modification comprising an insertion may comprise transformation, transduction, or transfection of a sample. Modification of a nucleic acid sequence may be an artificial modification, resulting from, for instance, genetic engineering or intentional nucleic acid sequence modification during nucleic acid fabrication. A nucleic acid sequence may be the result of somatic rearrangement.

A modification to a nucleic acid sequence comprising an insertion, deletion or substitution may comprise a difference between the nucleic acid sequence and a reference sequence. A reference sequence may be a nucleic acid sequence in a database, an artificial nucleic acid, a nucleic acid sequence of the same cell, a nucleic acid sequence of a cell from the tissue, a nucleic acid sequence from a different tissue of the same subject, or a nucleic acid sequence from a subject of a different species.

A modification to a nucleic acid sequence may comprise a difference in 1 nucleotide (a single nucleotide polymorphism, SNP), at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 25 nucleotides, at least 50 nucleotides, at least 100 nucleotides, at least 250 nucleotides, at least 500 nucleotides, at least 1,000 nucleotides, from 1 to 1,000 nucleotides, from 1 to 500 nucleotides, from 1 to 250 nucleotides, from 1 to 100 nucleotides, from 1 to 50 nucleotides, from 1 to 25 nucleotides, from 1 to 10 nucleotides, from 1 to 9 nucleotides, from 1 to 8 nucleotides, from 1 to 7 nucleotides, from 1 to 6 nucleotides, from 1 to 5 nucleotides, from 1 to 4 nucleotides, from 1 to 3 nucleotides, or from 1 to 2 nucleotides. Modification to a nucleic acid sequence comprising a difference in a plurality of nucleotides may comprise differences in two or more adjacent nucleotides or nucleotide sequences relative to a reference nucleic acid sequence. Modifications to a nucleic acid sequence comprising a difference in a plurality of nucleotides may also comprise differences in two or more non-adjacent nucleotides or nucleotide sequences (such as two or more modifications to the nucleic acid sequence that are separated by at least one nucleotide) relative to a reference nucleic acid sequence.

A target sequence may be assayed in situ or it may be isolated and/or purified from a cellular or acellular sample. For example, a target sequence comprising a nucleic acid may comprise a portion (a region) of genomic DNA located in situ in the nucleus of a fixed (intact) cell. A target sequence may comprise a nucleic acid sequence that is isolated from a sample (such as an aliquot of cerebrospinal fluid).

### Detection Agents

Detection agents may be utilized to detect nucleic acid sequence of interest. A detection agent may comprise a probe portion. The probe portion may include a probe, or a combination of probes. The probe portion may comprise a nucleic acid molecule, a polypeptide, or a combination thereof. The detection agents may further comprise a detectable moiety. The detectable moiety may comprise a fluorophore. A fluorophore may be a molecule that may absorb light at a first wavelength and transmit or emit light at a second wavelength. The fluorophore may be a small molecule (such as a dye) or a fluorescent polypeptide. The detectable moiety may be a fluorescent small molecule (such as a dye). The detectable moiety may not contain a fluorescent polypeptide. The detection agent may further comprise a conjugating moiety. The conjugating moiety may allow attachment of the detection agent to a nucleic acid sequence of interest. The detection agent may comprise a probe that is synthesized with direct dye incorporation at 3′ end.

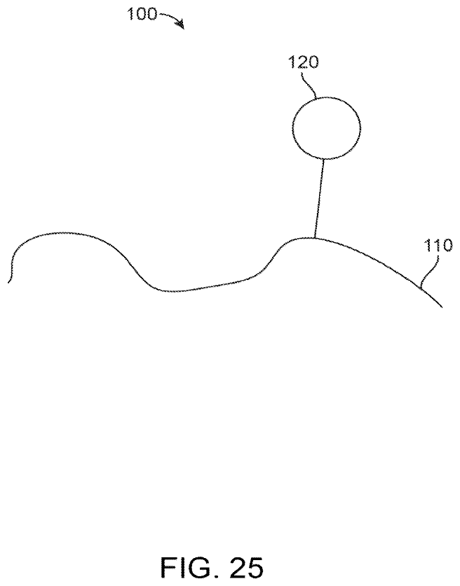

FIG. 24 shows a schematic of a detection agent 100 for use with a Nano-FISH procedure. The detection agent may comprise a probe portion 110, as described herein. The detection agent may comprise a detectable moiety 120, as described herein. The detection agent may comprise a conjugating moiety 130, as described herein.

### Probes

A detection agent may comprise a probe portion. A probe portion may comprise a probe or a combination of probes. A probe may be a nucleic acid probe, a polypeptide probe, or a combination thereof. A probe portion may be an unconjugated probe that does not contain a detectable moiety. A probe portion may be a conjugated probe which comprises a single probe with a detectable moiety, or two or more probes in which at least one probe may be an unconjugated probe bound to at least a second probe which comprises a detectable moiety.

A probe may be a nucleic acid probe. The nucleic acid probe may be a DNA probe, a RNA probe, or a combination thereof. The nucleic acid probe may be a DNA probe. The nucleic acid probe may be a RNA probe. The nucleic acid probe may be a double stranded nucleic acid probe, a single stranded nucleic acid probe, or may contain single-stranded and/or double stranded portions. The nucleic acid probe may further comprise overhangs on one or both termini, may further comprises blunt ends on one or both termini, or may further form a hairpin.

The nucleic acid probe may be at least 10, at least 15, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 65, at least 70, at least 80, at least 90, at least 100, or more nucleotides in length. The nucleic acid probe may be about 10, about 15, about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60, about 65, about 70, about 80, about 90, or about about 100 nucleotides in length. The nucleic acid probe may be about 20 nucleotides in length. The nucleic acid probe may be about 21 nucleotides in length. The nucleic acid probe may be about 22 nucleotides in length. The nucleic acid probe may be about 23 nucleotides in length. The nucleic acid probe may be about 24 nucleotides in length. The nucleic acid probe may be about 25 nucleotides in length. The nucleic acid probe may be about 26 nucleotides in length. The nucleic acid probe may be about 27 nucleotides in length. The nucleic acid probe may be about 28 nucleotides in length. The nucleic acid probe may be about 29 nucleotides in length. The nucleic acid probe may be about 30 nucleotides in length. The nucleic acid probe may be about 31 nucleotides in length. The nucleic acid probe may be about 32 nucleotides in length. The nucleic acid probe may be about 33 nucleotides in length. The nucleic acid probe may be about 34 nucleotides in length. The nucleic acid probe may be about 35 nucleotides in length. The nucleic acid probe may be about 36 nucleotides in length. The nucleic acid probe may be about 37 nucleotides in length. The nucleic acid probe may be about 38 nucleotides in length. The nucleic acid probe may be about 39 nucleotides in length. The nucleic acid probe may be about 40 nucleotides in length. The nucleic acid probe may be about 45 nucleotides in length. The nucleic acid probe may be about 50 nucleotides in length. The nucleic acid probe may be about 55 nucleotides in length. The nucleic acid probe may be about 60 nucleotides in length.

Table 1 lists exemplary probe nucleotide sequences according to the present disclosure.

TABLE 1
Exemplary Probe Nucleotide Sequences

| SEQ ID NO | Nucleotide Sequence |
|---|---|
| SEQ ID NO: 1 | TTTCCCTTGCTCTTCATGATTTTAACAACATGATGGATTT |
| SEQ ID NO: 2 | CCCTGCCCCCCATTAACTCACATCCTGAATTTTATGTTTA |
| SEQ ID NO: 3 | GCACTTCATCATCGTCTTTGAAGTCCCCTTCTTGTCCTCC |
| SEQ ID NO: 4 | TATGATGAACACCATGCACCACATGCAGGTTCTGGTGAAG |
| SEQ ID NO: 5 | GATACAAAAGAATATTGGTATGTATGTTGCACAGACTCAT |
| SEQ ID NO: 6 | CCTATTTCCCCCACACAGCCTTCCCACATTGGCCAACCCT |
| SEQ ID NO: 7 | TACAAAGGGCTTCTCTGGCCAGAGAGAGCCGGTGTCTGCT |
| SEQ ID NO: 8 | TGGGGGGGTTAATGGAGTTATGGACTGGGATGGGCAGCCT |
| SEQ ID NO: 9 | ACCTACCTAGGGAACTCTTTCTCCCTGGCACTAGGCTAGT |
| SEQ ID NO: 10 | ACTGACTGAGCTGACCTCCAGTACAGGGCCTGAGGCCACT |
| SEQ ID NO: 11 | CTGGGAGCTAAATAGAAGCAAATATCCCCAGGCCTGGGTG |
| SEQ ID NO: 12 | ATGCGTCAAGCAACTACACTCCCACAGTAAACTGGGAACC |
| SEQ ID NO: 13 | CAGCTCCTTGGCAGCCTAGGCTCTAGCTCAACATCTGCTT |
| SEQ ID NO: 14 | TGCTGGAGTCGCACCAACCTGGCTCTGCCTATCTCCAGCA |
| SEQ ID NO: 15 | CTCTGTAGGCTGCACAACGTGGAACAGATGAAAGGAACCA |
| SEQ ID NO: 16 | TGGGGTAAATTATAATCATGAAATTCCGTCAAGCTTGAAT |
| SEQ ID NO: 17 | AACATATTTAATATGGCATATTCAAATGACAGAAAGTACG |
| SEQ ID NO: 18 | CTTTATTCTTGCTAATGTTGACTCCTTAGCAAAGATAATT |
| SEQ ID NO: 19 | TGATCTTTGCTAAACTCTTCAGGAATAAATGAACATTTCC |
| SEQ ID NO: 20 | TTTTCAAGCAGTTAAGAAGCAAGAATTAATGACTCGAATA |
| SEQ ID NO: 21 | ATGAGAGTGTTGACTGATGAAGGGCTCCTATACGCGGGTT |
| SEQ ID NO: 22 | TCTTTCCCATCTGTTTCCCGGCCCCTACCAGAAATAAGTG |
| SEQ ID NO: 23 | ATGAACCTCCCTCGCTCCAAGACCAGAGCTCCTAGGAAGT |
| SEQ ID NO: 24 | TCTTTATTTTATTGGCCACAATTGAACATAGGTATAATTT |
| SEQ ID NO: 25 | CAGAAGCAAGCCCTGATCAAGGAAACCATTCACACTTGAT |
| SEQ ID NO: 26 | GTGGCTTTTGCTCAAAGTGAGGACGTTATCAGCTCTGCCC |
| SEQ ID NO: 27 | CTTTAAACAAAAACTAAAGGCGTAAGGAAAGATAACTACT |
| SEQ ID NO: 28 | CAGTTGCCACACTTTTTTTCACTGCTAAAGTTCGTAATGA |
| SEQ ID NO: 29 | GGCAATCAGAAGTATTTTGGTTGCTTCTAGGTCAGAATGA |
| SEQ ID NO: 30 | GGCAGCAAACTTGTTTAGGTATGATTCATCATTGTCTGCT |
| SEQ ID NO: 31 | CTACAAAACAATGAGTCTGATTACGACCCACAGAAATGAA |
| SEQ ID NO: 32 | CCTCCCACAGACCCAAACATGCTGCTGCAAATGTCTCACT |
| SEQ ID NO: 33 | GGACAAGCACACACATCGCTGGGAAGATCTGCAAGCCTCC |
| SEQ ID NO: 34 | TAAACCTGGATAACAAGAACACTGTTTCCACTGCGCTAGT |
| SEQ ID NO: 35 | TCATCACGATGACAATGGACAAGCCATATCCCTAACAGGG |
| SEQ ID NO: 36 | TTTCCATGACACCAGGACCGTAAAGCACCTTTTACACCGT |
| SEQ ID NO: 37 | AATTGGGATGTGCAAAACCTCTTAACTTGTAGCACCAAGT |
| SEQ ID NO: 38 | TCTTGTGTTATTCGCCTGCATTGAAATCCCATCCCAATCC |
| SEQ ID NO: 39 | TGAGTGATCTCTTTGCTGATCATAAACATATTCCTCCATC |
| SEQ ID NO: 40 | TGCATTCATTACTAAATACACAGGGCATAGCACATAGTAA |
| SEQ ID NO: 41 | CTTCAATGTTGCCAGGAAAATCCTTGCAGGAATCACACCC |
| SEQ ID NO: 42 | ATTTTTTTCTAAAGCTTTAGGAAATACACACGTTTCCCCT |
| SEQ ID NO: 43 | AGAGTAATCTTCAACAATCCTTGGTCTAAACACACACAAG |
| SEQ ID NO: 44 | CCCAGGGACCCACGCCAAGCTCACCGCACCTTCCACCAAA |
| SEQ ID NO: 45 | AGCTCCTGTACTAGCTGGTGGGGTGTGGAGCACACAGCCC |
| SEQ ID NO: 46 | TCACACAGGGAAAGTGAGGCTTGGTGGTTGATTTGAGCAA |
| SEQ ID NO: 47 | CCTTCCAACAGCCGTGTGAGACAAGAGGTCTTATCCTCTT |
| SEQ ID NO: 48 | ACAAGGGTCACTGAGCACATGCCATGTGTTGGGCACAGTG |
| SEQ ID NO: 49 | GTCTCCTAAGTCTCATTCTTTTCTTAGGATTCTTCAGATC |
| SEQ ID NO: 50 | TCCGCCTAAGTAAAACATAAAATTACTTAAGCTGCGTAAA |
| SEQ ID NO: 51 | CATTTTGACCTGATTATCTTTGTCTATAAGTCTTAAGCCA |
| SEQ ID NO: 52 | CCGGTTCCTCCACCCTCACTGCCCCAACAACTGAAAGAAG |
| SEQ ID NO: 53 | ACAGTGTGTTGAAAGAATCCATAACTCTTTCTTTCCAGCC |
| SEQ ID NO: 54 | GAAGTTTCATCTTTATCAAAATCTCCATTCCCAGGCGGAC |
| SEQ ID NO: 55 | AAGTCCATTTTTTTAAGCTTTGCGCTTCAGCTCCAGAACA |
| SEQ ID NO: 56 | TCTTCGTTATGAATACAAATAGGAAAACAATCAGACCCAA |
| SEQ ID NO: 57 | TCCTCGGGGCATTCTAGAACCGTAGCAGACCTGCTTACAT |
| SEQ ID NO: 58 | TCCTTATGTGGGAAAATAAAGAGGATAGACAGATTTGATT |
| SEQ ID NO: 59 | AGCTGCGAGTCCCTAACAGACTTCCAGGACAGCTGAAAAA |
| SEQ ID NO: 60 | AGGACAAGGGAGAGACGCCCACCCGCCTCTGTCAGGGATA |
| SEQ ID NO: 61 | AATCCATGAGGGTGACATACACATCCTTACTGTTCCCACA |
| SEQ ID NO: 62 | ACTTCCTTCCCTGAGATGCCCATCCTTTGATTCTGGGATT |
| SEQ ID NO: 63 | GCTCCCGGATAAATTAATTACCGTGACCCTGAGCTGCTTC |
| SEQ ID NO: 64 | TAGACTAAGAGAATCTAATTTGTGGCAAAGATCTTGAGTG |
| SEQ ID NO: 65 | TGAAGGATGACTAAGAGCTTCCCTATAAACCCCATACTGG |
| SEQ ID NO: 66 | AGCCAGGACTATAGAGTTTCAGAAAAGGGAGAAAATTCTA |
| SEQ ID NO: 67 | TGCTGCTAATTTAAGTTTCTGGCAAGTCAAAATAAATCTC |
| SEQ ID NO: 68 | CGAAAACCATCAATTAACTAGAATGATCAGGAAATTGCGT |
| SEQ ID NO: 69 | TTTATTTAGTCCCCAGGGTGTATGAAGTGCTCTTCCAGGC |
| SEQ ID NO: 70 | GGTCCTTCTTGGTACCGATATTGCCATATTGGCTGGACAT |
| SEQ ID NO: 71 | TGGCTTGGTAGGATGCACTCACATGGGCTGTAGTAATACT |
| SEQ ID NO: 72 | TATCACCAGCATAACTTGTGGTTCTTCAGCCAGTAATTTC |
| SEQ ID NO: 73 | GAACAACTGGGTATCTACAGGCAAAGAAATGAACCTTGAC |
| SEQ ID NO: 74 | TAGGTACTGTTGTGTCCCTATATATTTGACTTGGTAATAA |
| SEQ ID NO: 75 | TATGTGAACATCGGTGAATATCATAATTTATTATGCAAAC |
| SEQ ID NO: 76 | AGCTGAACACTCTTTGTGGTCCTCTTGAAGCCTAGAATTA |
| SEQ ID NO: 77 | CCCCACCTCACTGCCCCCCAGTTCTGACTCACGGTGTCCC |
| SEQ ID NO: 78 | ACTCCCATCACCTGGCCAGCTTGGCTGTCCCCTGACCCAC |
| SEQ ID NO: 79 | GGCTGCCCAGCTGCCCAGCAGCAAAACTGCATAGGAACTC |
| SEQ ID NO: 80 | GCCCAGGACGCCAAGTGTCACCACCCTCTCCCCAGGCAGG |
| SEQ ID NO: 81 | CACAAGGTCAGCTCCACCCGTGGGTCAGTGTGCCCCAGAT |
| SEQ ID NO: 82 | GGAGACAAAACGGGCACCCAGCCCAGTCATGCCCGTGCCT |
| SEQ ID NO: 83 | CTGAAATCAGTCAGCAGTTTCGGTGAGTCTGCAGCTGACA |
| SEQ ID NO: 84 | CGCCACATTTGGGGCTGGGAGAGATGTCACAGGGGCTGAC |
| SEQ ID NO: 85 | CACATGTTCTCTGCATAGGTTTTTAAGCAGCCAGCAGCTG |
| SEQ ID NO: 86 | TTTAAAATGAAAACCCACACTTCCAAAATAGCACTTGAGT |
| SEQ ID NO: 87 | AACATGTTTGTGTAATTAAGCATTTTAAAATCATAACCAT |
| SEQ ID NO: 88 | TGCTTATCTGTGCTTTTTATGTTCCACCCCCCCACCACCA |
| SEQ ID NO: 89 | ATTAATAATAATTCTGTGTTTATGGGGATTGCAGATACAT |
| SEQ ID NO: 90 | CCAGCTTTGTGTCTTCATGACCCAACTGGAGTAAGAATGG |
| SEQ ID NO: 91 | AAAGACCTCATTTGCAGCATGGTTAGCAGTGTCAAACATT |
| SEQ ID NO: 92 | TCTCGTAGCACTGGCTGCAGCCGGCCTGTGTGTGCCCACC |
| SEQ ID NO: 93 | GCCTTCATCCTGAACGGCTGACCAGCGGAAACAAAAGATC |
| SEQ ID NO: 94 | ATGGCCAGATAACAGTGTTTAGACATGTCTTTGATGTTTT |
| SEQ ID NO: 95 | CCCTGACTGTGTAAGGGGTCTCTCTCCATGGGGAATAGAG |
| SEQ ID NO: 96 | CTGAGCTTAGCTTCTACTGTGCTGTTAATTTCAGGCAAGA |
| SEQ ID NO: 97 | AGATCAATAATATTTGCATTAGCTACTTACATCAGTCTCT |
| SEQ ID NO: 98 | TAATTGCAGAAAACTTATAAAGCATGGAAGAATACAAAAC |
| SEQ ID NO: 99 | AAACAAATTCCTCTACCTGGACATGACTGTTGTTAGCATT |
| SEQ ID NO: 100 | GGGAGATTCTTCATATCCTTTTAATGTAGATATGCACATT |
| SEQ ID NO: 101 | ACAAAAAAGGCTATCATATTGTACATATAACTTTGCTGTA |
| SEQ ID NO: 102 | TCTGCTAGGAACCTGTACCCATGTCATTACTGTAAGCATT |
| SEQ ID NO: 103 | ACTACTCAAATTTTAGTATCTGCAGATATCAGATATCCTT |
| SEQ ID NO: 104 | TGAAATGGTATTGTTGCCCTTTCTGATTAGTAAAGTATAC |
| SEQ ID NO: 105 | TTATAATCTAGCAAGGTTAGAGATCATGGATCACTTTCAG |
| SEQ ID NO: 106 | ACAGCTTGCCTCCGATAAGCCAGAATTCCAGAGCTTCTGG |
| SEQ ID NO: 107 | TCAATCAACCTGATAGCTTAGGGGATAAACTAATTTGAAG |
| SEQ ID NO: 108 | GATCATGAAGGATGAAAGAATTTCACCAATATTATAATAA |
| SEQ ID NO: 109 | TTTAGCCATCTGTATCAATGAGCAGATATAAGCTTTACAC |
| SEQ ID NO: 110 | AGGGGTAGATTATTTATGCTGCCCATTTTTAGACCATAAA |
| SEQ ID NO: 111 | CACTACCATTTCACAATTCGCACTTTCTTTCTTTGTCCTT |
| SEQ ID NO: 112 | GCTCCATCAAATCATAAAGGACCCACTTCAAATGCCATCA |
| SEQ ID NO: 113 | TCCTACTTTCAGGAACTTCTTTCTCCAAACGTCTTCTGCC |
| SEQ ID NO: 114 | AATTCTATTTTTTCTTCAACGTACTTTAGGCTTGTAATGT |
| SEQ ID NO: 115 | TAAGATGCAAATAGTAAGCCTGAGCCCTTCTGTCTAACTT |
| SEQ ID NO: 116 | CTGTGTTTCAGAATAAAATACCAACTCTACTACTCTCATC |
| SEQ ID NO: 117 | GAAACCATGTTTATCTCAGGTTTACAAATCTCCACTTGTC |
| SEQ ID NO: 118 | CTTTGGAAAAGTAATCAGGTTTAGAGGAGCTCATGAGAGC |
| SEQ ID NO: 119 | GCTGAATCCCCAACTCCCAATTGGCTCCATTTGTGGGGGA |
| SEQ ID NO: 120 | GGTGTTATGAACTTAACGCTTGTGTCTCCAGAAAATTCAC |
| SEQ ID NO: 121 | AGTTAATGCACGTTAATAAGCAAGAGTTTAGTTTAATGTG |
| SEQ ID NO: 122 | TAATTGAGAAGGCAGATTCACTGGAGTTCTTATATAATTG |
| SEQ ID NO: 123 | CACGGTCAGATGAAAATATAGTGTGAAGAATTTGTATAAC |
| SEQ ID NO: 124 | CACAAGTCAGCATCAGCGTGTCATGTCTCAGCAGCAGAAC |
| SEQ ID NO: 125 | GGAGGTGGGGACTTAGGTGAAGGAAATGAGCCAGCAGAAG |
| SEQ ID NO: 126 | GTCACAGCATTTCAAGGAGGAGACCTCATTGTAAGCTTCT |
| SEQ ID NO: 127 | AAAGAGGTGAAATTAATCCCATACCCTTAAGTCTACAGAC |
| SEQ ID NO: 128 | CTTTACTAAGGAACTTTTCATTTTAAGTGTTGACGCATGC |
| SEQ ID NO: 129 | CAGGTTTTTCTTTCCACGGTAACTACAATGAAGTGATCCT |
| SEQ ID NO: 130 | GCTCTACAGGGAGGTTGAGGTGTTAGAGATCAGAGCAGGA |
| SEQ ID NO: 131 | TACTATTTCCAACGGCATCTGGCTTTTCTCAGCCCTTGTG |
| SEQ ID NO: 132 | AAGGTTTAGGCAGGGATAGCCATTCTATTTTATTAGGGGC |
| SEQ ID NO: 133 | AGGGGCTCAACGAAGAAAAAGTGTTCCAAGCTTTAGGAAG |
| SEQ ID NO: 134 | GGGCTGAACCCCCTTCCCTGGATTGCAGCACAGCAGCGAG |
| SEQ ID NO: 135 | CTGACGTCATAATCTACCAAGGTCATGGATCGAGTTCAGA |
| SEQ ID NO: 136 | GAAGGTAGAGCTCTCCTCCAATAAGCCAGATTTCCAGAGT |
| SEQ ID NO: 137 | CACCAATATTATTATAATTCCTATCAACCTGATAGGTTAG |
| SEQ ID NO: 138 | AGATATAAGCCTTACACAGGATTATGAAGTCTGAAAGGAT |
| SEQ ID NO: 139 | ACATGTATCTTTCTGGTCTTTTAGCCGCCTAACACTTTGA |
| SEQ ID NO: 140 | CAAAGAACAAGTGCAATATGTGCAGCTTTGTTGCGCAGGT |
| SEQ ID NO: 141 | TATTATTATGTGAGTAACTGGAAGATACTGATAAGTTGAC |
| SEQ ID NO: 142 | TAAAAATCTTTCTCACCCATCCTTAGATTGAGAGAAGTCA |
| SEQ ID NO: 143 | TTGGGTTCACCTCAGTCTCTATAATCTGTACCAGCATACC |
| SEQ ID NO: 144 | CACACCCATCTCACAGATCCCCTATCTTAAAGAGACCCTA |
| SEQ ID NO: 145 | ATGGAACCCAACCAGACTCTCAGATATGGCCAAAGATCTA |
| SEQ ID NO: 146 | GACACCAGTCTCTGACACATTCTTAAAGGTCAGGCTCTAC |
| SEQ ID NO: 147 | AGAGATTCAAAAGATTCACTTGTTTAGGCCTTAGCGGGCT |
| SEQ ID NO: 148 | TCCTTAGTCTGAGGAGGAGCAATTAAGATTCACTTGTTTA |
| SEQ ID NO: 149 | TAAATGGGGAAGTTGTTTGAAAACAGGAGGGATCCTAGAT |
| SEQ ID NO: 150 | GGGTTTATACATGACTTTTAGAACACTGCCTTGGTTTTTG |
| SEQ ID NO: 151 | AACTCTTAAAAGATATTGCCTCAAAAGCATAAGAGGAAAT |
| SEQ ID NO: 152 | AAATCGAGGAATAAGACAGTTATGGATAAGGAGAAATCAA |
| SEQ ID NO: 153 | TCAGTTAGGATTTAATCAATGTCAGAAGCAATGATATAGG |
| SEQ ID NO: 154 | CTTGAAAACACTTGAAATTGCTTGTGTAAAGAAACAGTTT |
| SEQ ID NO: 155 | ATAATCTTCAGAGGAAAGTTTTATTCTCTGACTTATTTAA |
| SEQ ID NO: 156 | AGATTCCTTCTGTCATTTTGCCTCTGTTCGAATACTTTCT |
| SEQ ID NO: 157 | ATTTCAGCTTCTAAACTTTATTTGGCAATGCCTTCCCATG |
| SEQ ID NO: 158 | GCAGGAGTTTGTTTTCTTCTGCTTCAGAGCTTTGAATTTA |
| SEQ ID NO: 159 | ACATATCAACGGCACTGGTTCTTTATCTAACTCTCTGGCA |
| SEQ ID NO: 160 | TTATGCTTCCCTGAAACAATACCACCTGCTATTCTCCACT |
| SEQ ID NO: 161 | TTCTCACTCCCTACCACTGAGGACAAGTTTATGTCCTTAG |
| SEQ ID NO: 162 | TTAGAGATTATGTCATTACCAGAGTTAAAATTCTATAATG |
| SEQ ID NO: 163 | GGTCATTCTTAGAATAGTAATCCAGCCAATAGTACAGGTT |
| SEQ ID NO: 164 | CAGGCAATAAGGGCTTTTTAAGCAAAACAGTTGTGATAAA |
| SEQ ID NO: 165 | ATGATGGGCACTGAAGGTTAAAACTTGAGTCTGTCAACTT |
| SEQ ID NO: 166 | AACTCATAAATATCCCATTTTCCGCTGAAATATAGCTTTA |
| SEQ ID NO: 167 | CCTGGTTTCTTTGACCTTTTGGGACCTTGAGTAAGTAAAG |
| SEQ ID NO: 168 | CTTCATTTATTTTCATGATTAAAATTCTAAGAAATTCTTG |
| SEQ ID NO: 169 | TTTTTAATTAAATTGCATTGCCTAATGTATTTATGAACTA |
| SEQ ID NO: 170 | CATAGAAATAAAACAATACTCTGAAGTAGTTCAGAATGTG |
| SEQ ID NO: 171 | CAATTTATATAAAGAGTTAATTCAAATGAGACTATTTTAA |
| SEQ ID NO: 172 | AGGGCTTTGAATCTTATGTCTAGAAATTTTGAAAAACCTC |
| SEQ ID NO: 173 | TATATGCTAAGATTCCACCTCTAGTGCTAGAACTGAGAAG |
| SEQ ID NO: 174 | TGACTTGGTGATCTTTTTTAAATTCTGAAACAACAGCAAC |
| SEQ ID NO: 175 | AGCTAAGGACTTTTTCTTGCCTATGCATGCTATCTTCAGT |
| SEQ ID NO: 176 | TGATTATTTAGTATTGAAACTATAACATAGTATGTTTCCT |
| SEQ ID NO: 177 | AAAAAATGTGTATTTCTCTGGAGAAGGTTAAAACTGAGGA |
| SEQ ID NO: 178 | CAAGTGAGCAAGGCTTAAATGGAAGAAGCAATGATCTCGT |
| SEQ ID NO: 179 | CCACCTTCATTAACGAGATCATCCATCATGAGGAAATATG |
| SEQ ID NO: 180 | ACCAGGCCCCCTCTGTTTTGTGTCACTAAGGGTGAGGATG |
| SEQ ID NO: 181 | ATGATTTTTCCCTCCCCCGGGCTTCTTTTAGCCATCAATA |
| SEQ ID NO: 182 | TAGCCCCACAGGAGTTTGTTCTGAAAGTAAACTTCCACAA |
| SEQ ID NO: 183 | AAGCTTATTGAGGCTAAGGCATCTGTGAAGGAAAGAAACA |
| SEQ ID NO: 184 | CTCTAAACCACTATGCTGCTAGAGCCTCTTTTCTGTACTC |
| SEQ ID NO: 185 | CTCATTCAGACACTAGTGTCACCAGTCTCCTCATATACCT |
| SEQ ID NO: 186 | TATTTTCTTCTTCTTGCTGGTTTAGTCATGTTTTCTGGGA |
| SEQ ID NO: 187 | GGCAAACCCATTATTTTTTTCTTTAGACTTGGGATGGTGA |
| SEQ ID NO: 188 | TGGGCAGCGTCAGAAACTGTGTGTGGATATAGATAAGAGC |
| SEQ ID NO: 189 | GACTATGCTGAGCTGTGATGAGGGAGGGGCCTAGCTAAAG |
| SEQ ID NO: 190 | TGAGAGTCAGAATGCTCCTGCTATTGCCTTCTCAGTCCCC |
| SEQ ID NO: 191 | TTGGTTTCTACACAAGTAGATACATAGAAAAGGCTATAGG |
| SEQ ID NO: 192 | TGTTTGAGAGTCCTGCATGATTAGTTGCTCAGAAATGCCC |
| SEQ ID NO: 193 | TTACAAATATGTGATTATCATCAAAACGTGAGGGCTAAAG |
| SEQ ID NO: 194 | CAGATAACTTGCAAGTCCTAGGATACCAGGAAAATAAATT |
| SEQ ID NO: 195 | AGCATTATGTCTGTCTGTCATTGTTTTTCATCCTCTTGTA |
| SEQ ID NO: 196 | TTCACAGTTACCCACACAGGTGAACCCTTTTAGCTCTCCT |
| SEQ ID NO: 197 | GAATGTTTCTTTCCTCTCAGGATCAGAGTTGCCTACATCT |
| SEQ ID NO: 198 | AATGCACCAAGACTGGCCTGAGATGTATCCTTAAGATGAG |
| SEQ ID NO: 199 | TCCCAGTAGCACCCCAAGTCAGATCTGACCCCGTATGTGA |
| SEQ ID NO: 200 | GTGTCCTCTAACAGCACAGGCCTTTTGCCACCTAGCTGTC |
| SEQ ID NO: 201 | GGCAAACAAGGTTTGTTTTCTTTTCCTGTTTTCATGCCTT |
| SEQ ID NO: 202 | TTCCATATCCTTGTTTCATATTAATACATGTGTATAGATC |
| SEQ ID NO: 203 | AAATCTATACACATGTATTAATAAAGCCTGATTCTGCCGC |
| SEQ ID NO: 204 | AGGTATAGAGGCCACCTGCAAGATAAATATTTGATTCACA |
| SEQ ID NO: 205 | CTAATCATTCTATGGCAATTGATAACAACAAATATATATA |
| SEQ ID NO: 206 | ATAATATATTCTAGAATATGTCACATTCTGTCTCAGGCAT |
| SEQ ID NO: 207 | TTTCTTTATGATGCCGTTTGAGGTGGAGTTTTAGTCAGGT |
| SEQ ID NO: 208 | AGCTTCTCCTTTTTTTTGCCATCTGCCCTGTAAGCATCCT |
| SEQ ID NO: 209 | GGGACCCAGATAGGAGTCATCACTCTAGGCTGAGAACATC |
| SEQ ID NO: 210 | CACACACCCTAAGCCTCAGCATGACTCATCATGACTCAGC |
| SEQ ID NO: 211 | CTGTGCTTGAGCCAGAAGGTTTGCTTAGAAGGTTACACAG |
| SEQ ID NO: 212 | AACTGCTCATGCTTGGACTATGGGAGGTCACTAATGGAGA |
| SEQ ID NO: 213 | CAGAAATGTAACAGGAACTAAGGAAAAACTGAAGCTTATT |
| SEQ ID NO: 214 | CAGAGATGAGGATGCTGGAAGGGATAGAGGGAGCTGAGCT |
| SEQ ID NO: 215 | AAAAGTATAGTAATCATTCAGCAAATGGTTTTGAAGCACC |
| SEQ ID NO: 216 | GTATCTTATTCCCCACAAGAGTCCAAGTAAAAAATAACAG |
| SEQ ID NO: 217 | GAAAAGAATGTTTCTCTCACTGTGGATTATTTTAGAGAGT |
| SEQ ID NO: 218 | AATGGTCAAGATTTTTTTAAAAATTAAGAAAACATAAGTT |
| SEQ ID NO: 219 | CTTGAGAAATGAAAATTTATTTTTTTGTTGGAGGATACCC |
| SEQ ID NO: 220 | TCTATCTCCCATCAGGGCAAGCTGTAAGGAACTGGCTAAG |
| SEQ ID NO: 221 | AGTGAGACAGAGTGACTTAGTCTTAGAGGCCCCACTGGTA |
| SEQ ID NO: 222 | GATGAGAAGGCACCTTCATCACTCATCACAGTCAGCTCTG |
| SEQ ID NO: 223 | TCTCCTCTCTCCTTTCTCATCAGAAATTTCATAAGTCTAC |
| SEQ ID NO: 224 | GTCAGGCAGATCACATAAGAAAAGAGGATGCCAGTTAAGG |
| SEQ ID NO: 225 | GTTGCTGTTAGACAATTTCATCTGTGCCCTGCTTAGGAGC |
| SEQ ID NO: 226 | TCTTTAATGAAAGCTAAGCTTTCATTAAAAAAAGTCTAAC |
| SEQ ID NO: 227 | TGCATTCGACTTTGACTGCAGCAGCTGGTTAGAAGGTTCT |
| SEQ ID NO: 228 | GAGGAGGGTCCCAGCCCATTGCTAAATTAACATCAGGCTC |
| SEQ ID NO: 229 | ACTGGCAGTATATCTCTAACAGTGGTTGATGCTATCTTCT |
| SEQ ID NO: 230 | CTTGCCTGCTACATTGAGACCACTGACCCATACATAGGAA |
| SEQ ID NO: 231 | ATAGCTCTGTCCTGAACTGTTAGGCCACTGGTCCAGAGAG |
| SEQ ID NO: 232 | CATCTCCTTTGATCCTCATAATAACCCTATGAGATAGACA |
| SEQ ID NO: 233 | TATTACTCTTACTTTATAGATGATGATCCTGAAAACATAG |
| SEQ ID NO: 234 | CAAGGCACTTGCCCCTAGCTGGGGGTATAGGGGAGCAGTC |
| SEQ ID NO: 235 | GTAGTAGTAGAATGAAAAATGCTGCTATGCTGTGCCTCCC |
| SEQ ID NO: 236 | CTTTCCCATGTCTGCCCTCTACTCATGGTCTATCTCTCCT |
| SEQ ID NO: 237 | CCTGGGAGTCATGGACTCCACCCAGCACCACCAACCTGAC |
| SEQ ID NO: 238 | CCACCTATCTGAGCCTGCCAGCCTATAACCCATCTGGGCC |
| SEQ ID NO: 239 | TAGCTGGTGGCCAGCCCTGACCCCACCCCACCCTCCCTGG |
| SEQ ID NO: 240 | TCTGATAGACACATCTGGCACACCAGCTCGCAAAGTCACC |
| SEQ ID NO: 241 | GGGTCTTGTGTTTGCTGAGTCAAAATTCCTTGAAATCCAA |
| SEQ ID NO: 242 | TTAGAGACTCCTGCTCCCAAATTTACAGTCATAGACTTCT |
| SEQ ID NO: 243 | GGCTGTCTCCTTTATCCACAGAATGATTCCTTTGCTTCAT |
| SEQ ID NO: 244 | CCATCCATCTGATCCTCCTCATCAGTGCAGCACAGGGCCC |
| SEQ ID NO: 245 | GCAGTAGCTGCAGAGTCTCACATAGGTCTGGCACTGCCTC |
| SEQ ID NO: 246 | ATGTCCGACCTTAGGCAAATGCTTGACTCTTCTGAGCTCA |
| SEQ ID NO: 247 | TGTCATGGCAAAATAAAGATAATAATAGTGTTTTTTTATG |
| SEQ ID NO: 248 | TAGCGTGAGGATGGAAAACAATAGCAAAATTGATTAGACT |
| SEQ ID NO: 249 | AAGGTCTCAACAAATAGTAGTAGATTTTATCGTCCATTAA |
| SEQ ID NO: 250 | TCCCTCTCCTCTCTTACTCATCCCATCACGTATGCCTCTT |
| SEQ ID NO: 251 | TTCCCTTACCTATAATAAGAGTTATTCCTCTTATTATATT |
| SEQ ID NO: 252 | TTATAGTGATTCTGGATATTAAAGTGGGAATGAGGGGCAG |
| SEQ ID NO: 253 | CTAACGAAGAAGATGTTTCTCAAAGAAGCCATTCTCCCCA |
| SEQ ID NO: 254 | GATCATCTCAGCAGGGTTCAGGAAGATAAAGGAGGATCAA |
| SEQ ID NO: 255 | TGTTGAGGTGGGAGGACCGCTTGAGCCTGGGAAGTGCAAG |
| SEQ ID NO: 256 | AGTGAGCCGAGATTTTGCCACTACACTCCCATTTGGGTGA |
| SEQ ID NO: 257 | GTGAGACCCTTTCTCAAAAACAAACTAATTAAAAAACCCT |
| SEQ ID NO: 258 | TTTACAGATGAAGAAACTGAGTCATACAACTACTAAGAGA |
| SEQ ID NO: 259 | GAGTCACTAATCACTCAGGTGGTCTGGCTCCAGCATCTGT |
| SEQ ID NO: 260 | TTAATCTCTGCTCTATACTGCCCAAGACTTTTATAAAGTC |
| SEQ ID NO: 261 | GTTGAGTCACTGAAATGAGTTATTGGGATGGCTGTGTGGG |
| SEQ ID NO: 262 | GTGCTAAGTTCTTTCCTAAAGGTATGTGAGAATACAAAGG |
| SEQ ID NO: 263 | AAGCATCCTCCTTTTTACACACGTGAACTAGTGCATGCAA |
| SEQ ID NO: 264 | GACACTCAGTGGGCCTGGGTGAAGGTGAGAATTTTATTGC |
| SEQ ID NO: 265 | TGAGAGCCTCTGGGGACATCTTGCCAGTCAATGAGTCTCA |
| SEQ ID NO: 266 | CAATTTCCTTCTCAGTCTTGGAGTAACAGAAGCTCATGCA |
| SEQ ID NO: 267 | ATAAACGGAAATTTTGTATTGAAATGAGAGCCATTGGAAA |
| SEQ ID NO: 268 | TTACTCCAGACTCCTACTTATAAAAAGAGAAACTGAGGCT |
| SEQ ID NO: 269 | GAAGGGTGGGGACTTTCTCAGTATGACATGGAAATGATCA |
| SEQ ID NO: 270 | TGGATTCAAAGCTCCTGACTTTCTGTCTAGTGTATGTGCA |
| SEQ ID NO: 271 | GCCCCTTTTCCTCTAACTGAAAGAAGGAAAAAAAAATGGA |
| SEQ ID NO: 272 | AAAATATTCTACATAGTTTCCATGTCACAGCCAGGGCTGG |
| SEQ ID NO: 273 | TCTCCTGTTATTTCTTTTAAAATAAATATATCATTTAAAT |
| SEQ ID NO: 274 | AAATAAGCAAACCCTGCTCGGGAATGGGAGGGAGAGTCTC |
| SEQ ID NO: 275 | GTCCACCCCTTCTCGGCCCTGGCTCTGCAGATAGTGCTAT |
| SEQ ID NO: 276 | GCCCTGACAGAGCCCTGCCCATTGCTGGGCCTTGGAGTGA |
| SEQ ID NO: 277 | GCCTAGTAGAGAGGCAGGGCAAGCCATCTCATAGCTGCTG |
| SEQ ID NO: 278 | GGAGAGAGAAAAGGGCTCATTGTCTATAAACTCAGGTCAT |
| SEQ ID NO: 279 | ATTCTTATTCTCACACTAAGAAAAAGAATGAGATGTCTAC |
| SEQ ID NO: 280 | ACCCTGCGTCCCCTCTTGTGTACTGGGGTCCCCAAGAGCT |
| SEQ ID NO: 281 | AAAAGTGATGGCAAAGTCATTGCGCTAGATGCCATCCCAT |
| SEQ ID NO: 282 | TATAAACCTGCATTTGTCTCCACACACCAGTCATGGACAA |
| SEQ ID NO: 283 | CCTCCTCCCAGGTCCACGTGCTTGTCTTTGTATAATACTC |
| SEQ ID NO: 284 | AATTTCGGAAAATGTATTCTTTCAATCTTGTTCTGTTATT |
| SEQ ID NO: 285 | TTTCAATGGCTTAGTAGAAAAAGTACATACTTGTTTTCCC |
| SEQ ID NO: 286 | ATTGACAATAGACAATTTCACATCAATGTCTATATGGGTC |
| SEQ ID NO: 287 | TGTTTGCTGTGTTTGCAAAAACTCACAATAACTTTATATT |
| SEQ ID NO: 288 | CTACTCTAAGAAAGTTACAACATGGTGAATACAAGAGAAA |
| SEQ ID NO: 289 | TTACAAGTCCAGAAAATAAAAGTTATCATCTTGAGGCCTC |
| SEQ ID NO: 290 | TTCTAGGAATAATATCAATATTACAAAATTAATCTAACAA |
| SEQ ID NO: 291 | GAACAGCAATGAGATAATGTGTACAAAGTACCCAGACCTA |
| SEQ ID NO: 292 | GTAGAGCATCAAGGAAGCGCATTGCGGAGCAGTTTTTTGT |
| SEQ ID NO: 293 | TTGTTTTTGTATTCTGTTTCGTGAGGCAAGGTTTCACTCT |
| SEQ ID NO: 294 | TCCAGGCTGGAGTGCAGTGGCAAGATCATGTCTCACTGCA |
| SEQ ID NO: 295 | TGACCTCCTGAGCTCAAGGGATCCTCCCATTTCGGCCTCC |
| SEQ ID NO: 296 | TAGCTGGGACTACAGGTGTACATCACATGCCTGGCTAATT |
| SEQ ID NO: 297 | TTTTTTTTTTAAGTAGAGACGAGGTCTTGCTATGTTGTCC |
| SEQ ID NO: 298 | TAATATCAAACTCTTGAGCTCAAGCAGTCCTCCCACTTCT |
| SEQ ID NO: 299 | TGGAGGTATCCAGTATGAAATTTAGATAATACCTGCCTTC |
| SEQ ID NO: 300 | GTTGAAATTAGAACTTAATGATATAATGCATCAATGAACT |
| SEQ ID NO: 301 | ATAGTTCCTAGCACAAAGTAAGAATCCTTTCAATGTGTGT |
| SEQ ID NO: 302 | GTGTATGTATTTATCTGTTATTAATAGGAATCTTATGGGC |
| SEQ ID NO: 303 | TCTCACTTAATCCTTATTAATAACTATGAAGCAGGTATTT |
| SEQ ID NO: 304 | GAGTTTTCCAAGTGAGTTAAGTATAGCTTGTAATACTTAA |
| SEQ ID NO: 305 | ATATCCACAGGTTACATAGCTAGTATATAACTGAGAAATA |
| SEQ ID NO: 306 | TATTTATATTATAAAACATTCTAACAATACAGATGTATAT |
| SEQ ID NO: 307 | TAAAAAACTGAAAGGGCTCATGCAACCCTACCTTCTCAAT |
| SEQ ID NO: 308 | CTTCTTCACTTAGAAAAAACCAGCCTTAGCTGTCTGCTAT |
| SEQ ID NO: 309 | CCTTTCAAAATATACTTCTGAGAAATGAGAGAGAGAAATG |
| SEQ ID NO: 310 | GGGTAGAAGGAAGGAAGATAGGGTAAGAGACAGGGAAGGA |
| SEQ ID NO: 311 | TGGGGAAAGAAATTAAATTATTCTTTTCTCTGTCTCTTGA |
| SEQ ID NO: 312 | GCTCTTTCCATTACATTGAATCAAAGGTAATGTTGCCATT |
| SEQ ID NO: 313 | GACTCTTGAAATAAAGAAAGACCGATGTATGAAATAATTT |
| SEQ ID NO: 314 | AGTCTATGGCATTTTCAAAATGCAAGGTGATGTCTTACTA |
| SEQ ID NO: 315 | GCCTTTGCTTTATTATTAGAAATGGGGAAGTGAGTATAGA |
| SEQ ID NO: 316 | TTATCAGGAGATATATTAGGAAAAAGGGAAACTGGAGAAA |
| SEQ ID NO: 317 | GAGGAGTATCCAGATGTCCTGTCCCTGTAAGGTGGGGGCA |
| SEQ ID NO: 318 | CCTTCAATCAAAAGGGCTCCTTAACAACTTCCTTGCTTGG |
| SEQ ID NO: 319 | CCACCATCTTGGACCATTAGCTCCACAGGTATCTTCTTCC |
| SEQ ID NO: 320 | AGTGGTCATAACAGCAGCTTCAGCTACCTCTCTAAAGAGT |
| SEQ ID NO: 321 | CCAGATATAGGTCAGGAAATATAATCCACTAATAAAAAGA |
| SEQ ID NO: 322 | CATTTTGACTGTAGTTGTTTGTTTTTTGTCATTGTGACTA |
| SEQ ID NO: 323 | TAACATTCTCACTCTTTCATCAGTAATCACTCAGGTTATT |
| SEQ ID NO: 324 | GACCAACAGACTGTGGGAAAAATCAGAGAAGGAGGCATCC |
| SEQ ID NO: 325 | GCTTACTAGCCTAAACTGAAATTGCTATAGCAGAGTGAAC |
| SEQ ID NO: 326 | AGGTTTACAGATATTTTCCACAAAGAGTAAAAGGATTGAA |
| SEQ ID NO: 327 | TCTCCAGATCAATGCATAGGAAATAATAATGGACCATAAA |
| SEQ ID NO: 328 | ATATTATGACGAACAACATTAGGATAAGTCCATATCAATT |
| SEQ ID NO: 329 | ATCCAGTCATAAGCACAGACTACGTGAAGCACGTCCAAGT |
| SEQ ID NO: 330 | GCAGGAGAAATGAGAGGAGCAAGAAAGAGGAGCCATTTGA |
| SEQ ID NO: 331 | GAATAGCAGAAAAAGGAAAGGCAAGTCATATTAACAAATG |
| SEQ ID NO: 332 | TCATGCCAACAGTACAGATAACTCTGCTAATAAAGGTAGA |
| SEQ ID NO: 333 | TAATACAGGTAGTAGCAGATATCTACATAGTAGTTAAAGG |
| SEQ ID NO: 334 | GGCCATCAGTACAGAAGATTCCATAAAGGAGAACCTAAAG |
| SEQ ID NO: 335 | AGAATAATTTGTCAGAAGCTTAAAAGCTGAACTCTGAGGC |
| SEQ ID NO: 336 | AACTACAATATCCTTTTGACTGTGGAAAGGGTGGTGAAAG |
| SEQ ID NO: 337 | GTTCAAGGACATTTGAGCCAACATAGAGAGGAACATTGGC |
| SEQ ID NO: 338 | TGAGGGATATCTGTCCTGATGTTGTCCAGGATGGTGATGA |
| SEQ ID NO: 339 | CATATAAATAACGTAGAGAAAACAGGAGGGGATAGAGATC |
| SEQ ID NO: 340 | CAAAGAGGCATCAAAGATAGGGATGTTTGTAAGGATGAAA |
| SEQ ID NO: 341 | CTGTTCTTCTCTGAGTAGCCAAGCTCAGCTTGGTTCAAGC |
| SEQ ID NO: 342 | CATACTGTGGATCTGTAGCAAATTCCCCCTGAAAACCCAG |
| SEQ ID NO: 343 | TCTGACCCTCACATTCAAGTTCTGAGGAAGGGCCACTGCC |
| SEQ ID NO: 344 | GCCTTGAGATACCTGGTCCTTATTCCTTGGACTTTGGCAA |
| SEQ ID NO: 345 | ATAGGGCTTGTTTTAGGGAGAAACCTGTTCTCCAAACTCT |
| SEQ ID NO: 346 | CTGGTGTCCATACTCTGAATGGGAAGAATGATGGGATTAC |
| SEQ ID NO: 347 | AGCAGGAGAGGATCAACCCCATACTCTGAATCTAAGAGAA |
| SEQ ID NO: 348 | TCAGATCCCTGGATGCAAGCCAGGTCTGGAACCATAGGCA |
| SEQ ID NO: 349 | CTCCTCCCTACCACCTTTAGCCATAAGGAAACATGGAATG |
| SEQ ID NO: 350 | GACACAAACCTGGGCCTTTCAATGCTATAACCTTTCTTGA |
| SEQ ID NO: 351 | CTACCTGACTTCTGAGTCAGGATTTATAAGCCTTGTTACT |
| SEQ ID NO: 352 | TGAACCAACAAGCATCGAAGCAATAATGAGACTGCCCGCA |
| SEQ ID NO: 353 | GAAAAGCAATAATCCATTTTTCATGGTATCTCATATGATA |
| SEQ ID NO: 354 | TAACACTTATCTCTCTGAACTTTGGGCTTTTAATATAGGA |
| SEQ ID NO: 355 | TTTTCTGACTGTCTAATCTTTCTGATCTATCCTGGATGGC |
| SEQ ID NO: 356 | ATCTTCATCGAATTTGGGTGTTTCTTTCTAAAAGTCCTTT |
| SEQ ID NO: 357 | GAAATTACAAATGCTAAAGCAAACCCAAACAGGCAGGAAT |
| SEQ ID NO: 358 | ATTAGGCATCTTACAGTTTTTAGAATCCTGCATAGAACTT |
| SEQ ID NO: 359 | TACAATATTTGACTCTTCAGGTTAAACATATGTCATAAAT |
| SEQ ID NO: 360 | AACATTCAGTGAAGTGAAGGGCCTACTTTACTTAACAAGA |
| SEQ ID NO: 361 | TCTTTTCCTATCAGTGGTTTACAAGCCTTGTTTATATTTT |
| SEQ ID NO: 362 | TATTTTTGTTCTGAGAATATAGATTTAGATACATAATGGA |
| SEQ ID NO: 363 | CAAAATCTAACACAAAATCTAGTAGAATCATTTGCTTACA |
| SEQ ID NO: 364 | AGAATTTATGACTTGTGATATCCAAGTCATTCCTGGATAA |
| SEQ ID NO: 365 | TTACACTAGAAAATAGCCACAGGCTTCCTGCAAGGCAGCC |
| SEQ ID NO: 366 | AGTTTGAACACTTGTTATGGTCTATTCTCTCATTCTTTAC |
| SEQ ID NO: 367 | ACTTCGTGAGAGATGAGGCAGAGGTACACTACGAAAGCAA |
| SEQ ID NO: 368 | TCTTGAGAATGAGCCTCAGCCCTGGCTCAAACTCACCTGC |
| SEQ ID NO: 369 | AATAGGATGTCTGTGCTCCAAGTTGCCAGAGAGAGAGATT |
| SEQ ID NO: 370 | ATTAAAGATCCCTCCTGCTTAATTAACATTCACAAGTAAC |
| SEQ ID NO: 371 | ACTTAAAGTAGCGATACCCTTTCACCCTGTCCTAATCACA |
| SEQ ID NO: 372 | TCTCAGGTGTTAACTTTATAGTGAGGACTTTCCTGCCATA |
| SEQ ID NO: 373 | ATAGTTTCATATAAATGGGTTCCTCATCATCTATGGGTAC |
| SEQ ID NO: 374 | GGTATTTACATTTGCCATTCCCTATGCCCTAAATATTTAA |
| SEQ ID NO: 375 | TATTGATATTCCTTGAAAATTCTAAGCATCTTACATCTTT |
| SEQ ID NO: 376 | CTTTTATTCTCCCCTTCACCGAATCTCATCCTACATTGGC |
| SEQ ID NO: 377 | TAGTGTCCCAAATTTTATAATTTAGGACTTCTATGATCTC |
| SEQ ID NO: 378 | ATATGGTCACCTCTTTGTTCAAAGTCTTCTGATAGTTTCC |
| SEQ ID NO: 379 | ACAATCTTCCTGCTTCTACCACTGCCCCACTACAATTTCT |
| SEQ ID NO: 380 | AGTCACTGTCACCACCACCTAAATTATAGCTGTTGACTCA |
| SEQ ID NO: 381 | CTGACCCCTTGCCTTCACCTCCAATGCTACCACTCTGGTC |
| SEQ ID NO: 382 | AGAAAATCCTGTTGGTTTTTCGTGAAAGGATGTTTTCAGA |
| SEQ ID NO: 383 | ACATATACTCACAGCCAGAAATTAGCATGCACTAGAGTGT |
| SEQ ID NO: 384 | ACCCAAAGACTCACTTTGCCTAGCTTCAAAATCCTTACTC |
| SEQ ID NO: 385 | TGAGGTAGAGACTGTGATGAACAAACACCTTGACAAAATT |
| SEQ ID NO: 386 | TCCATATCCACCCACCCAGCTTTCCAATTTTAAAGCCAAT |
| SEQ ID NO: 387 | AAGGTATGATGTGTAGACAAGCTCCAGAGATGGTTTCTCA |
| SEQ ID NO: 388 | CTCTGGTCAGCATCCAAGAAATACTTGATGTCACTTTGGC |
| SEQ ID NO: 389 | AACTGTGAACTTCCTTCAGCTAGAGGGGCCTGGCTCAGAA |
| SEQ ID NO: 390 | TGATTGTTCTCTGACTTATCTACCATTTTCCCTCCTTAAA |
| SEQ ID NO: 391 | AAACAAAACCCATCAAATTCCCTGACCGAACAGAATTCTG |
| SEQ ID NO: 392 | CAGAGGTCACAGCCTAAACATCAAATTCCTTGAGGTGCGG |
| SEQ ID NO: 393 | GAAGGCAGGTGTGGCTCTGCAGTGTGATTGGGTACTTGCA |
| SEQ ID NO: 394 | CATGGAGGAAAAACTCATCAGGGATGGAGGCACGCCTCTA |
| SEQ ID NO: 395 | AGCTTGTTAAATTGAATTCTATCCTTCTTATTCAATTCTA |
| SEQ ID NO: 396 | CATAGTTGTCAGCACAATGCCTAGGCTATAGGAAGTACTC |
| SEQ ID NO: 397 | GCAGATATAGCTTGATGGCCCCATGCTTGGTTTAACATCC |
| SEQ ID NO: 398 | CTAAATAACTAGAATACTCTTTATTTTTTCGTATCATGAA |
| SEQ ID NO: 399 | AGTGTTTAAAGGGTGATATCAGACTAAACTTGAAATATGT |
| SEQ ID NO: 400 | GGATGGGTCTAGAAAGACTAGCATTGTTTTAGGTTGAGTG |
| SEQ ID NO: 401 | TGCTGCCAACATTAACAGTCAAGAAATACCTCCGAATAAC |
| SEQ ID NO: 402 | TATTGTGAGAGGTCTGAATAGTGTTGTAAAATAAGCTGAA |
| SEQ ID NO: 403 | TTACAACATGATGGCTTGTTGTCTAAATATCTCCTAGGGA |
| SEQ ID NO: 404 | CTAAGTAGAAGGGTACTTTCACAGGAACAGAGAGCAAAAG |
| SEQ ID NO: 405 | GTCTTGTATTGCCCAGTGACATGCACACTGGTCAAAAGTA |
| SEQ ID NO: 406 | CCCTATGTCTTCCCTGATGGGCTAGAGTTCCTCTTTCTCA |
| SEQ ID NO: 407 | AAAGTTTCCCCAAATTTTACCAATGCAAGCCATTTCTCCA |
| SEQ ID NO: 408 | AACTGCAGATTCTCTGCATCTCCCTTTGCCGGGTCTGACA |
| SEQ ID NO: 409 | TAGTGCTGTGGTGCTGTGATAGGTACACAAGAAATGAGAA |
| SEQ ID NO: 410 | TAACTAGCGTCAAGAACTGAGGGCCCTAAACTATGCTAGG |
| SEQ ID NO: 411 | CATTGGCTCCGTCTTCATCCTGCAGTGACCTCAGTGCCTC |
| SEQ ID NO: 412 | TGTTTATGTGTTATAGTGTTCATTTACTCTTCTGGTCTAA |
| SEQ ID NO: 413 | CCTTTGACCCCTTGGTCAAGCTGCAACTTTGGTTAAAGGG |
| SEQ ID NO: 414 | TTCTCTTGGGTTACAGAGATTGTCATATGACAAATTATAA |
| SEQ ID NO: 415 | TGGAAGTTGTGGTCCAAGCCACAGTTGCAGACCATACTTC |
| SEQ ID NO: 416 | CTGCCCTGTGGCCCTTGCTTCTTACTTTTACTTCTTGTCG |
| SEQ ID NO: 417 | AACTCAGATATTGTGGATGCGAGAAATTAGAAGTAGATAT |
| SEQ ID NO: 418 | TACAGAACCACCAAGTAGTAAGGCTAGGATGTAGACCCAG |
| SEQ ID NO: 419 | TGAGCTCTCCTACTGTCTACATTACATGAGCTCTTATTAA |
| SEQ ID NO: 420 | AAGCTAATAAGTAGACAATTAGTAATTAGAAGTCAGATGG |
| SEQ ID NO: 421 | AGCCCAATGTACTTGTAGTGTAGATCAACTTATTGAAAGC |
| SEQ ID NO: 422 | CCAATACTCAGAAGTAGATTATTACCTCATTTATTGATGA |
| SEQ ID NO: 423 | GCTAGAATCAAATTTAAGTTTATCATATGAGGCCGGGCAC |
| SEQ ID NO: 424 | TAATACTAATGATAAGTAACACCTCTTGAGTACTTAGTAT |
| SEQ ID NO: 425 | ATGGTAATTCTGTGAGATATGTATTATTGAACATACTATA |
| SEQ ID NO: 426 | TGAAAGAGAAGTGGGAATTAATACTTACTGAAATCTTTCT |
| SEQ ID NO: 427 | GAGAGACACGAGGAAATAGTGTAGATTTAGGCTGGAGGTA |
| SEQ ID NO: 428 | GTTGAGAGGGAAACAAGATGGTGAAGGGACTAGAAACCAC |
| SEQ ID NO: 429 | CAAGGTTCTGAACATGAGAAATTTTTAGGAATCTGCACAG |
| SEQ ID NO: 430 | TGCCATCTAAAAAAATCTGACTTCACTGGAAACATGGAAG |
| SEQ ID NO: 431 | GGGATCCTCTCTTAAGTGTTTCCTGCTGGAATCTCCTCAC |
| SEQ ID NO: 432 | GTTTCCTTCATGTGACAGGGAGCCTCCTGCCCCGAACTTC |
| SEQ ID NO: 433 | TTGGATAAGAGTAGGGAAGAACCTAGAGCCTACGCTGAGC |
| SEQ ID NO: 434 | ATCTGGGGCTTTGTGAAGACTGGCTTAAAATCAGAAGCCC |
| SEQ ID NO: 435 | ACCGCAATGCTTCCTGCCCATTCAGGGCTCCAGCATGTAG |
| SEQ ID NO: 436 | TATGGGGAAGCAGGGTATGAAAGAGCTCTGAATGAAATGG |
| SEQ ID NO: 437 | GGTTGCATGAATCAGATTATCAACAGAAATGTTGAGACAA |
| SEQ ID NO: 438 | AATGCAGGCCTAGGCATGACTGAAGGCTCTCTCATAATTC |
| SEQ ID NO: 439 | TAACGTTTTCTTGTCTGCTACCCCATCATATGCACAACAA |
| SEQ ID NO: 440 | TTAATTCCCAAACTCATATAGCTCTGAGAAAGTCTATGCT |
| SEQ ID NO: 441 | CCCTATAGGGGATTTCTACCCTGAGCAAAAGGCTGGTCTT |
| SEQ ID NO: 442 | TCCTCACCATATAGAAAGCTTTTAACCCATCATTGAATAA |
| SEQ ID NO: 443 | TAAGCTGTCTAGCAAAAGCAAGGGCTTGGAAAATCTGTGA |
| SEQ ID NO: 444 | AGGATTAGAAGATTCTTCTGTGTGTAAGAATTTCATAAAC |
| SEQ ID NO: 445 | ATTATCTTCTGGAATAGGGAATCAAGTTATATTATGTAAC |
| SEQ ID NO: 446 | CTCTCTGGTTGACTGTTAGAGTTCTGGCACTTGTCACTAT |
| SEQ ID NO: 447 | TCTTCAGTTAGATGGTTAACTTTGTGAAGTTGAAAACTGT |
| SEQ ID NO: 448 | CTACACCATGTGGAGAAGGGGTGGTGGTTTTGATTGCTGC |
| SEQ ID NO: 449 | ACTTTCCTAACCTGAGCCTAACATCCCTGACATCAGGAAA |
| SEQ ID NO: 450 | TACACTTTATTCGTCTGTGTCCTGCTCTGGGATGATAGTC |
| SEQ ID NO: 451 | TACTCTTTGCATTCCACTGTTTTTCCTAAGTGACTAAAAA |
| SEQ ID NO: 452 | AAAGGCCTCCCAGGCCAAGTTATCCATTCAGAAAGCATTT |
| SEQ ID NO: 453 | TATTGACATGTACTTCTTGGCAGTCTGTATGCTGGATGCT |
| SEQ ID NO: 454 | TTTGGTCCTAATTATGTCTTTGCTCACTATCCAATAAATA |
| SEQ ID NO: 455 | GTTAAAAAAACTACCTCTCAACTTGCTCAAGCATACACTC |
| SEQ ID NO: 456 | TAATTAGTGCTTTGCATAATTAATCATATTTAATACTCTT |
| SEQ ID NO: 457 | ACTAGTGTTCTGTACTTTATGCCCATTCATCTTTAACTGT |
| SEQ ID NO: 458 | GTATTTTTTGTTTAACTGCAATCATTCTTGCTGCAGGTGA |
| SEQ ID NO: 459 | GCAGTGACTTATAAATGCTAACTACTCTAGAAATGTTTGC |
| SEQ ID NO: 460 | TTATAAGCATGATTACAGGAGTTTTAACAGGCTCATAAGA |
| SEQ ID NO: 461 | AGTATCCCTCAAGTAGTGTCAGGAATTAGTCATTTAAATA |
| SEQ ID NO: 462 | AGTCACCCATTTGGTATATTAAAGATGTGTTGTCTACTGT |
| SEQ ID NO: 463 | TGGTCATAAAACATTGAATTCTAATCTCCCTCTCAACCCT |
| SEQ ID NO: 464 | ACAGTTGAAAAGACCTAAGCTTGTGCCTGATTTAAGCCTT |
| SEQ ID NO: 465 | CAACTACAGGGCCTTGAACTGCACACTTTCAGTCCGGTCC |
| SEQ ID NO: 466 | GTGGTTCTTTGAAGAGACTTCCACCTGGGAACAGTTAAAC |
| SEQ ID NO: 467 | TGGAGGAAATATTTATCCCCAGGTAGTTCCCTTTTTGCAC |
| SEQ ID NO: 468 | GCCTGGTGCTTTTGGTAGGGGAGCTTGCACTTTCCCCCTT |
| SEQ ID NO: 469 | TCTCATTTCTTTGAGAACTTCAGGGAAAATAGACAAGGAC |
| SEQ ID NO: 470 | CAAACTTTTCAAGCCTTCTCTAATCTTAAAGGTAAACAAG |
| SEQ ID NO: 471 | TCAACAAAGGAGAAAAGTTTGTTGGCCTCCAAAGGCACAG |
| SEQ ID NO: 472 | GATGCAACAGACCTTGGAAGCATACAGGAGAGCTGAACTT |
| SEQ ID NO: 473 | CATCTGAGATCCCAGCTTCTAAGACCTTCAATTCTCACTC |
| SEQ ID NO: 474 | TATCTTAACAGTGAGTGAACAGGAAATCTCCTCTTTTCCC |
| SEQ ID NO: 475 | AACTCATGCTTTGTAGATGACTAGATCAAAAAATTTCAGC |
| SEQ ID NO: 476 | TCAAAGGAAGTCAAAAGATGTGAAAAACAATTTCTGACCC |
| SEQ ID NO: 477 | TGCCTTCACTTAAGTAATCAATTCCTAGGTTATATTCTGA |
| SEQ ID NO: 478 | CCCTACCTTGTTCAAAATGTTCCTGTCCAGACCAAAGTAC |
| SEQ ID NO: 479 | GCACTTACAAATTATACTACGCTCTATACTTTTTGTTTAA |
| SEQ ID NO: 480 | CTTTAGTTTCATTTCAAACAATCCATACACACACAGCCCT |
| SEQ ID NO: 481 | TAGGGACCACAGGGTTAAGGGGGCAGTAGAATTATACTCC |
| SEQ ID NO: 482 | CTCACAATTAAGCTAAGCAGCTAAGAGTCTTGCAGGGTAG |
| SEQ ID NO: 483 | GTTGAAAGACAGAGAGGATGGGGTGCTATGCCCCAAATCA |
| SEQ ID NO: 484 | GCTTGTCTAATTTTATATATCACCCTACTGAACATGACCC |
| SEQ ID NO: 485 | AATATTGTACACGTACACCAAAGCATCATGTTGTACCCCA |
| SEQ ID NO: 486 | TGTGAAGTGGTGGATTTGTTAATTAGCCTTATTTAACCAT |
| SEQ ID NO: 487 | TGACACATATGACATTTTAACTATGTTCCAGATTTTTGAA |
| SEQ ID NO: 488 | GCAAGGAATCATTCAATGTTTTCTAAATCTATTACTGCAT |
| SEQ ID NO: 489 | CATTTTCATAGGTTTTCCTCGATTGATCATTATTCATGAT |
| SEQ ID NO: 490 | AAAGTGATCAAGATATTTTTAGTTCAGGCTCCAAAATTTT |
| SEQ ID NO: 491 | CTTTACAGGCCGAGAAAAATGAATCTGAATTCCTGACCTC |
| SEQ ID NO: 492 | TCCACTCAAGGCCTACATTCTGCTATAATGCAATTTCAAG |
| SEQ ID NO: 493 | AACTGCTTAAAATTAATGGCACAAGTCATGTTTTTGATGT |
| SEQ ID NO: 494 | CTGACTGTGACGTAGCAATAAAGAAACCCACGTTTCATAT |
| SEQ ID NO: 495 | CTGGCCCACTGCTTGGAGGAGAGCACTCAGGACCATGAAC |
| SEQ ID NO: 496 | TTCTGAAATGATAAAGTCAATCACAGGAAGGCACCTGGAC |
| SEQ ID NO: 497 | ATCATTCTCTTTCCCTTCCTCTATGTGGCAGAAAGTAAAA |
| SEQ ID NO: 498 | GGAGATAATAATGTGTTACTCCCTAAGGCAGAGTGCCCTT |
| SEQ ID NO: 499 | CAATTAACTTGGCCATGTGACTGGTTGTGACTAAAATAAT |
| SEQ ID NO: 500 | CACTAAATCAATATACTTCTCAACAATTTCCAACAGCCCT |
| SEQ ID NO: 501 | CTAGGCTCCTGAGTTTGCTGGGGATGCGAAGAACCCTTAT |
| SEQ ID NO: 502 | CCGAGGACCCCGCACTCGGAGCCGCCAGCCGGCCCCACCG |
| SEQ ID NO: 503 | TTGGAAGCACAGGGTGTGGGATAATGCTAATTACTAGTGA |
| SEQ ID NO: 504 | GTTCAGTATGCCTTTGATTTTACAATAATATTCCTGTTAT |
| SEQ ID NO: 505 | AGATTCCATGAAGTATTACAGCATTTGGTAGTCTTTTTGC |
| SEQ ID NO: 506 | TATTTGCTCTGAAATAAGACATAATTTGGGGTGAGAAAGC |
| SEQ ID NO: 507 | ACTCATGATATTTGGCTCTAGAATACATGCTCTGAATCAT |
| SEQ ID NO: 508 | TCCAAGATGAAGTGGCTACTAACTGACAGAGGGCATAATT |
| SEQ ID NO: 509 | TATTCACAGTAACTCTGTGCCTCAAGTACTATTGTAATAC |
| SEQ ID NO: 510 | ACATCCTCAATCTACACACTAGGATAGTATAAAAGTAATA |
| SEQ ID NO: 511 | GTCTACCCATATGTGACCTTCATGTCTTTGCTCTAAGCCC |
| SEQ ID NO: 512 | CGTGTAATCCTTGACAATGTCATCTCATCTATTTATTCCC |
| SEQ ID NO: 513 | TCTGAAAGAGACTAACCTTCCCTCGCTTTGCAGAGAAAGA |
| SEQ ID NO: 514 | ATGCATGGATTCTCTTGAAAAAATGTTTCTGCCATGATGT |
| SEQ ID NO: 515 | TAGTTGAAGACCTACTGTGTTCAGGGCCGTGAGCCAGGGC |
| SEQ ID NO: 516 | CAACGTGGAGAGCTGTCCTGGCACCATTTCTTCCTGCTGT |
| SEQ ID NO: 517 | ATCCTCAAAGGAGCCTGGCTTGGGCTAACAAGGAAGAACT |
| SEQ ID NO: 518 | TGCCTGGGACCCTGCCCCAAGCAAAGTAATAATCTGAATG |
| SEQ ID NO: 519 | CTGGTGTGTCCAGTGTGATCCCTGCACCCATGCCCGGAGC |
| SEQ ID NO: 520 | CTGCCCCCTGCAGCAGGGAAGGGGCTCTGGAAGGGTCTGA |
| SEQ ID NO: 521 | TAGCTGCTGCCCCACTATGCACCATCGCTTATCTGTTCTT |
| SEQ ID NO: 522 | GAAACCCGAAAAATGTCCTGGTCCTCTTCTTAAGTCTGGG |
| SEQ ID NO: 523 | GCTGAGAACATGACTCTGCTTGGCGTTCCATTTAATTGAC |
| SEQ ID NO: 524 | GAGAGGGTGTGCATTTGAAGTATAGATTTGTTAAACATAG |
| SEQ ID NO: 525 | CATCAGGCAAAAATACTTCGATGGGACTGTGTTCTTTCAG |
| SEQ ID NO: 526 | TCTAAAGTGATGTAATGTTGCCACGGAAATTCTAATCCCT |
| SEQ ID NO: 527 | CGTGCAGAACCAGCTCTGTCTTCCCAGACACTGTCGCTTT |
| SEQ ID NO: 528 | ACCCCTGAGCACCTCAGTGTCCGTGACTGTGGAGCGGAGG |
| SEQ ID NO: 529 | CTGCCTGGGACACGTACGGCTGCCCAGTGATCCTGAGCGC |
| SEQ ID NO: 530 | CACAGCCGGATGGTGTGGGAGCTGGCACTGCCGGGGCTCC |
| SEQ ID NO: 531 | CGTCTTGGCAGAGGCTCCCTGTCATCAAGGACCTGAGGTT |
| SEQ ID NO: 532 | GACCCCACAAAGATGAGCGGGTCCCCTTCCCAATTTTCGG |
| SEQ ID NO: 533 | TCAGGAAGCCGGTGCTCAGCAAACTTATCTGAAGCTCTTG |
| SEQ ID NO: 534 | GAGGCTGCAGAGGAACATCGTTTGGTCAAATGTGAAATGT |
| SEQ ID NO: 535 | CTAGCTTCTAGAAAGTGCTGCCAATTTGGGGACCAAGGGA |
| SEQ ID NO: 536 | GGAAACACTTCTTTTTCCCTTGACAAAGGACATCCTCTGC |
| SEQ ID NO: 537 | GCATGTGCATAAACACTCGTGTGTGTGTCCTTTTATCCCA |
| SEQ ID NO: 538 | CCAAATCTCTATACATGTCCATAGAGAGAGGCAGACGTAT |
| SEQ ID NO: 539 | GGGTTGAAGACAAGGGGCTCAGAGCTTGCTTTTTATACAC |
| SEQ ID NO: 540 | AGATTCATCTTCATGGCAGGACTTCAGGCAAGAGAGGCCC |
| SEQ ID NO: 541 | CTCACCCCTTAGCAGGACCCTGACGGAACTGGGTACAGGC |
| SEQ ID NO: 542 | GGTTGGGAGACAATGGGTGGCCCCTCGGTGTGGTGTCCTC |
| SEQ ID NO: 543 | AGAGTCTAGAGGGCCCGTGGGGACGGGAGTCCTGGGAACC |
| SEQ ID NO: 544 | GCGGCATGTCCGGCTTCACCCTGCCCAGAATCACAGCCTC |
| SEQ ID NO: 545 | ATGGTTAAAAAATTCTCCTACTTAAGACTCCCAGACCCCT |
| SEQ ID NO: 546 | GGGTTGAAGACAAGGGGCTCAGAGCTTGCTTTTTATACAC |
| SEQ ID NO: 547 | AGATTCATCTTCATGGCAGGACTTCAGGCAAGAGAGGCCC |
| SEQ ID NO: 548 | CTCACCCCTTAGCAGGACCCTGACGGAACTGGGTACAGGC |
| SEQ ID NO: 549 | GGTTGGGAGACAATGGGTGGCCCCTCGGTGTGGTGTCCTC |
| SEQ ID NO: 550 | AGAGTCTAGAGGGCCCGTGGGGACGGGAGTCCTGGGAACC |
| SEQ ID NO: 551 | GCGGCATGTCCGGCTTCACCCTGCCCAGAATCACAGCCTC |
| SEQ ID NO: 552 | TGAGATTCCAGGGCTGGTTCCACAACGGCCGGCATCGGCC |
| SEQ ID NO: 553 | CTGAGTCACTAACAAAGCTCAGGCCTGACCACAGGACATT |
| SEQ ID NO: 554 | GGCTGGCCTACCTGCCACGGGGCCAGGGCTGGGTGCTTTC |
| SEQ ID NO: 555 | GGGCTCTGGACGCTGGAGGCCTGAGGCTGCACCCCAGGTT |
| SEQ ID NO: 556 | ACAGTGGCCACTCACCCACTGGGCCCACATCCCCACAGGC |
| SEQ ID NO: 557 | ACTCTGCCAGCCTTTGATGCCTCGCTGAGACAGAGGGTCT |
| SEQ ID NO: 558 | AGCCGGGGCTCTGGCCCCATCCAGGGGCTCCCCCAGCAGC |
| SEQ ID NO: 559 | CCTTGGAAGTCAGTCAGCAGGTCAGGACACAGTTCAGCCC |
| SEQ ID NO: 560 | TTACATGCAGTTGGTCTTCTCCTGTGAATGGGGAAACTGA |
| SEQ ID NO: 561 | CTGCATCACAGAACAGCTGCATTTCTAATGTCAGGCTTCT |
| SEQ ID NO: 562 | CAGCCTGGGAGGCTTGTCAACCTCCTTTGACAAGCACGCC |
| SEQ ID NO: 563 | AGAAACTGGGGCTCCAGGGCATGGAGGCTGCCTGTGGCCA |
| SEQ ID NO: 564 | TCCCGGCCTGGAGGAAGTCTTATTAGCCTCATTTCATGGA |
| SEQ ID NO: 565 | TCCTGCCAGCCCCCTCACGCTCACGAATTCAGTCCCAGGG |
| SEQ ID NO: 566 | AATTCTAAAGGTGAAGGGACGTCTACACCCCCAACAAAAC |
| SEQ ID NO: 567 | GGAAATATTAGTCCCCTCTGCCTGGGACAAGACCACCGAA |
| SEQ ID NO: 568 | AAACACACCTCTGAATGGAAAGCTGAGAAACAGTGATCTC |
| SEQ ID NO: 569 | ACTGCACCCCCTCCCTTCCCGTGCCGGCAATTTAACCGGG |
| SEQ ID NO: 570 | TGCCTTCCTACCTTGACCAGTCGGTCCTTGCGGGGGTCCC |
| SEQ ID NO: 571 | ATTTCCTTCATCTTGTCCTTCTAGCCTGGAGACTCTTCGG |
| SEQ ID NO: 572 | AATGCCCGAAAATTCCAGCAGCAGCCCAAGATGGTGGCCA |
| SEQ ID NO: 573 | CGTTGCAAATGCCCAAGGGGGTAACCCTAAAAGTTAAAGG |
| SEQ ID NO: 574 | ACACAACCCCTGTGCAAGTTTCATTCCGGCGCACAGGGGC |
| SEQ ID NO: 575 | TGCAAGAACTAATTTAGCATGCAAGGACGGGGAGGACCGG |
| SEQ ID NO: 576 | GCCACGAGGGCACCCACGGGCGGACAGACGGCCAAAGAAT |
| SEQ ID NO: 577 | ACCCCATATCCAAGCCGGCAGAATGGGCGCATTTCCAAGA |
| SEQ ID NO: 578 | GCCTGGGGAGACCACGAGAAGGGGTGACTGGGGCGCGGCG |
| SEQ ID NO: 579 | CTGCAGTAGGGGACAACTAGGAAGGCCGGCAGGCCACACG |
| SEQ ID NO: 580 | GAGTGGGTCCCCCGGGATTTAGGGGGTGAGGTGGAGGTGG |
| SEQ ID NO: 581 | TCCCCGCCAGGGAAGAGGGGTGCAGGGGGCCCCGTCCGCC |
| SEQ ID NO: 582 | TGAGGCGCCGCGCCTGCCCTGCGGCGGAGTTGCCCCTGTA |
| SEQ ID NO: 583 | AAACGCCGGGAGCAGCGAGGGGCAGAGCCCAAAAGCCATC |
| SEQ ID NO: 584 | TTGTTAAGCAAAGATCAAAGCCCGGCAGAGAATGGGAGCG |
| SEQ ID NO: 585 | CAACTTCAACAAAACTCCCCTGTAGTCCGTGTGACGTTAC |
| SEQ ID NO: 586 | CTGCTACTGCGCCGACAGCCCTCTGGAGGCTCCAGGACTT |
| SEQ ID NO: 587 | GCTCTTCTGCCCCTCGCCGGAGCGTGCGGACTCTGCTGCT |
| SEQ ID NO: 588 | TCCGCGCTCGGCTCTCGCTTCTGCTGCCCCGCGCTCCCTC |
| SEQ ID NO: 589 | TTTCCACTTCGCAGCACAGGAGCTGGTGTTCCATGGCTGG |
| SEQ ID NO: 590 | GGTCGTTGAGGAGGTTGGCATCGGGGTACGCGCGGCGGAT |
| SEQ ID NO: 591 | TGTCCTACTTCAAATGTGTGCAGAAGGAGGTCCTGCCGTC |
| SEQ ID NO: 592 | TCGGGCGGCTCTCTTAAGACTTCCCTGCAACTTGTTGCCC |
| SEQ ID NO: 593 | ACCCACGTTTCTTTGCTACTCACCCCCCTCCCTTCTCTCC |
| SEQ ID NO: 594 | CTAGAACTTTGAAGTTTGCCGTGGTGTTTCTAGGGATCCG |
| SEQ ID NO: 595 | AGAAGGGGGTCCGGGAGGGGTGCCTTCGGGAGAAGCCAGT |
| SEQ ID NO: 596 | CAGGGGCACCCCAATGGGCCCGAGGGTGCGGGCTGGCAGG |
| SEQ ID NO: 597 | GGGTGCGCTTTGTGTCCCCCGCCTGCGCCCCAGCCCGGCT |
| SEQ ID NO: 598 | GCCTCAGCGGCCGGGAGCCGCCAACTCCGGGGGGAGGGGG |
| SEQ ID NO: 599 | AAAGTGCAGTAATACCCTTGATCAGAGTTGATGACTTGAA |
| SEQ ID NO: 600 | GAGAGAAATAAAGTAGTTGCTCTATTTGTAAATTGAAAAG |
| SEQ ID NO: 601 | GGTAGCAGTGATTGCTGTATATTTGTGAAAAGGAGGCAAG |
| SEQ ID NO: 602 | TGCTGATAATGGAAGTGCAGTGGGTTAGCTTTGTTTCCAT |
| SEQ ID NO: 603 | CCGTTCTACCGTGACTAGTATGGAATTGTGGGAACCAGAA |
| SEQ ID NO: 604 | TTAACATCAGTGTCAACTGCAGTGTTGTTTCTGAGTAATA |
| SEQ ID NO: 605 | CATAACTCCATGCTCTCAAACCAATCACTCCTTCATTCAT |
| SEQ ID NO: 606 | TTCTCCTATGCTGCACCAGAAAGGGTTTTGTGGGTTATCA |
| SEQ ID NO: 607 | ATCGTTCAGCATCTTTAGGAAATATCCAGAGACTGCATTG |
| SEQ ID NO: 608 | TTTATTAAGAGCAAAAAAAGCCTGTTTCGTTAGCCAGTCA |
| SEQ ID NO: 609 | TTGTTCATATGCCTAACTTAATAAATTCTTCATACAGAAA |
| SEQ ID NO: 610 | ATAACTTTTAAACCCAAACACCTAGAGATTTCATTATGTA |
| SEQ ID NO: 611 | TTCTTACCATTAAGTCTTCCAAATGATAATTTATTATAAA |
| SEQ ID NO: 612 | TATGTAAGGACAACTTCATTATATGCTTGAAGAAATTGTT |
| SEQ ID NO: 613 | AATCTTAAAAGTGACACTAGTCACATTCCACACGGTTAAA |
| SEQ ID NO: 614 | ATTTTGAAAACTATTCCTTTATCTGGAATGAATGTAAACC |
| SEQ ID NO: 615 | TTGCATTAAGGGCACCAGAAACTTATAGAAAACCAAAAAG |
| SEQ ID NO: 616 | TAAAAGACAGTGAACTGAACAGTAATTAACATTACATCCA |
| SEQ ID NO: 617 | CAAAAAACTGTGTTTATCATATACCAAACATTTTCAAGTT |
| SEQ ID NO: 618 | TCTCAGGATATTTTGTTCTCTGACACAAATACACCAGTCA |
| SEQ ID NO: 619 | TAGCTTTACATCTCAGAATGAATCAATGTGGGGGCAGAAA |
| SEQ ID NO: 620 | AGACCTATATACCTATAGTGCCTAATAGACAATAAGCCAC |
| SEQ ID NO: 621 | TCTCTCCCCTGCCTAGACTAAGGTAAGTGGGTCTTACCTT |
| SEQ ID NO: 622 | CATCCTGCTTTTAAAACCCTTAGTGCTCAGCGGCTTGTCT |
| SEQ ID NO: 623 | AGCTTATAAACTTCAGAGTAATGTAGCACAAATGTCTGTC |
| SEQ ID NO: 624 | AACTTGAAATAAAACTTTAAACGTTGATTGATTCTTTCCC |
| SEQ ID NO: 625 | GACAGGCTTAGAGTCCATAACAAACAATCTTAGCTGGAAA |
| SEQ ID NO: 626 | TGCTCAACAACACTTGTGGAAGAGCAGGGCAAGCTATTTC |
| SEQ ID NO: 627 | TTACAACATCACTGTAGACATTACTTTTACCCACAGTGCC |
| SEQ ID NO: 628 | ATCCTAGTTGTATATACTTCTTGGATAAAGTATCTTCGTA |
| SEQ ID NO: 629 | ATTTTTGGGGAGTGCCATTCCTGCAGGTCTTGAAGACAGG |
| SEQ ID NO: 630 | CACACAGCCAATGAAACTGACAGAGCCAATGCAACCAAAA |
| SEQ ID NO: 631 | ACGACTTCAATCAAGAGAAACAGGCAGGTCAGAGTGTGAA |
| SEQ ID NO: 632 | CTGGTTATCAGGGTTCATAGCACATAGGTTTGACAACCAC |
| SEQ ID NO: 633 | TTTATTATTCAGCTGGGTAAGCCAAGTGACAGTCTTCCCC |
| SEQ ID NO: 634 | GTTTTATTCTAGGAATCAACTGCTTTCTAAAAATGTCTAA |
| SEQ ID NO: 635 | TTTACTGATGGTACTTATTCCCCCAATTATTGATTATTGA |
| SEQ ID NO: 636 | GCATTTAGGAATATTCAATATTGATACTAAGGTCATCTTT |
| SEQ ID NO: 637 | TACTCTGTAATGTAGTAATCTTTATGAAGAAATAAATTTG |
| SEQ ID NO: 638 | ATTTTGAAAAAATGTTTCACTGCATTTTACTATACAAGCT |
| SEQ ID NO: 639 | ACCACACATTCATCAAAAAATACCTCAAAGAAAATTCTGC |
| SEQ ID NO: 640 | GTTGTCACAATAAACTCAGTACTGAGTAAAATATCACAAA |
| SEQ ID NO: 641 | GAGTATATATTGTATTACTTACCTGATGCGCAAAGACCCA |
| SEQ ID NO: 642 | AAAATGACAGCAACATAGGTGCCACCTGAGGTCCACATCT |
| SEQ ID NO: 643 | TGGAGAGAGTGGGGTTAATCTGTTACTACACTTTGCTACT |
| SEQ ID NO: 644 | ATTTCCATCATTTTGTCTTTCAGTAAGCATGTACGAAGTA |
| SEQ ID NO: 645 | GAGATGAAGATGGTACATCAGTAGGGAGCCCCTCTACTGG |
| SEQ ID NO: 646 | TCTAATTCATCAAAGTATTCTGGGTTGATTCCAGGTACGT |
| SEQ ID NO: 647 | ACAAACTCGTTTTGTACAGAGAGGAAAATATTAAAACACC |
| SEQ ID NO: 648 | ATGTTAATTATAAACACTGTTATAAGTTTTACAAATGTAA |
| SEQ ID NO: 649 | TCCACTGGCAGAGAGAATATATGTTTCCATTACGGTCCCA |
| SEQ ID NO: 650 | TCAAAGGTTTTCTATCACGTTTTCTATTATTTACTCACAT |
| SEQ ID NO: 651 | AAAAACAAGAGTCACACAACCTATGCTCCACAATATCTGC |
| SEQ ID NO: 652 | ATAGGTTATTCTACAATCGACACCAACTATCAGCGGCTTT |
| SEQ ID NO: 653 | ATTGAATTAAATGATGGCTTGATTATCCAGGAATCAGCCA |
| SEQ ID NO: 654 | CTTACCATAACAGAGTAATCTCTAGCTTATTCCAAGGATA |
| SEQ ID NO: 655 | ACCTAAAATTTAACTAGAATCACTTTTCAATGAAGCTGCT |
| SEQ ID NO: 656 | TAAACTAAGAGCCTTTGATCTTGCCTTATTCTGATAAAAT |
| SEQ ID NO: 657 | AAATAATAATTCACAAGGAAATCCTTATTGTTTATTTAAA |
| SEQ ID NO: 658 | GTAATATGTAGGTTAAACAGAAATGTTGGTTGAATCATGT |
| SEQ ID NO: 659 | TGCAGACACTAATCAAACCAAACAGGGCCAATTAAAATTG |
| SEQ ID NO: 660 | TAAAGTGCAATGGGACAGAGCAACTTCATTTTCACAAACA |
| SEQ ID NO: 661 | TAATCTAATTGCCAGAAATGCTTGCCCATTGCAATGGGAG |
| SEQ ID NO: 662 | AGTTGACAATGACTGCTTAGTTTAGGGTTTTGAAGTAAAC |
| SEQ ID NO: 663 | CAGATGGCAGGTATTCTGTGAATTAACACTGATGCTTCTG |
| SEQ ID NO: 664 | AGTCAAGTTCAGAAATGATCTGTTATGACCCCATGAAACG |
| SEQ ID NO: 665 | GGGATGCTCTGATACATCATTCAGTAAAATGATAGAAAAA |
| SEQ ID NO: 666 | TAGCTGTATTGCTTGATAGCTTCATAGCTTGATAACCATT |
| SEQ ID NO: 667 | TTTTAGCAGGGAATTAACACAGGTATATAAATGAAGAAAA |
| SEQ ID NO: 668 | TTGATTGTTTATGAAGCTGAGATTGTTTACTGGTTTCGAG |
| SEQ ID NO: 669 | TCTGTGTTTTTATGTTTGGGAACATGAGGGAATCAGTTCT |
| SEQ ID NO: 670 | TTCTTAAGCTTTCATTTTTCCAGTGGTGAATGTAGAGAGA |
| SEQ ID NO: 671 | ACGGTAACTGAATAAACTTAAGAACTGAGGTAAAGTTTTC |
| SEQ ID NO: 672 | TCAATATGTAAAATTGATCAATTCAGACACCTTTATATGG |
| SEQ ID NO: 673 | TGTCTCTTTCATGCTGTAAATAGAGCATTGCATGAAAGAT |
| SEQ ID NO: 674 | TTCATAGCACAGTTTATAAACCTAAGAAAGCAAAGATGAA |
| SEQ ID NO: 675 | AACCAAGCAGGATTCTATGACTAAAAAAGTGTATTTGTAT |
| SEQ ID NO: 676 | AGATAGAGAATTTCAAAGAAACCATCTTTATCAGCTGCAC |
| SEQ ID NO: 677 | CCAAGAATGAAAAGATGCACTAATTCGACTGAAAGCCAAG |
| SEQ ID NO: 678 | TCATAGTTGAGACATATAACAACCATAAAGGTCCGCATAT |
| SEQ ID NO: 679 | AGGAAAGGGTGGAAAGGCAAGCAGCGGGGAGTGTTGGCTG |
| SEQ ID NO: 680 | CTATAAATTGACCTATCCTGTAAAAAAGGATGTCACAGCA |
| SEQ ID NO: 681 | ACAATTGACCTAAGACTGTAAATTGTAAATTGACTATAAA |
| SEQ ID NO: 682 | GCAAGACTGGGTATACTATTAATAGGAAAAAATGAACTTC |
| SEQ ID NO: 683 | ATTGCTTTGATATTGATTGAATCACAGAGAAAATCCTAAG |
| SEQ ID NO: 684 | TAGATTATGCTGGCAAATCTCAGTGATCAGAGAATTATAT |
| SEQ ID NO: 685 | ATTCAGAAATGGAATAGGAAGATATTTATGTGCCATCCTG |
| SEQ ID NO: 686 | GTTTGAATTATTATTCAAACAGTGTATGTTTGTTTGTACT |
| SEQ ID NO: 687 | AATGCAACAGAGACAGGTATTTATAGCATCTGTTTTCCAT |
| SEQ ID NO: 688 | TTTAATATCCAAATATGTATGGACACATACAATTGTACAT |
| SEQ ID NO: 689 | ACGTCTACCGTCATTTTCGTAATTATTCGGTTTCCCTGTC |
| SEQ ID NO: 690 | GGAGCGCTCCTGCGCGCCTTGTTCGTTAGGATTTATTTTT |
| SEQ ID NO: 691 | GGTGGCTCCCTAATGCCTGCTCGTTTCAGGTCTCAGCTCT |
| SEQ ID NO: 692 | CCTTAGTGTGTTGAGGACGCTGCAGAAGGTACAGAGGAGA |
| SEQ ID NO: 693 | GACCAGATGGTAGGACAGTCATTCTCCTCTGCGTCTCCGC |
| SEQ ID NO: 694 | CGTGAGGCATGGAGTTTTTGTCCTGCCCCTGCCTGGTTAG |
| SEQ ID NO: 695 | TTTAAGTCTCTGGCACCGTGCATAGCAGAATTGGTTGGGA |
| SEQ ID NO: 696 | TCTTTCTCCAAGTGCCTCTATGTTGGCACATCTCTGAAAT |
| SEQ ID NO: 697 | TGCGTCCCGGCCAGGTAAGCAGCTTCCCTCTCAGCTGCCT |
| SEQ ID NO: 698 | GGGTGTATGTAGCTGGCAGAAGTGGGACTTGGTCGCAACC |
| SEQ ID NO: 699 | CGTGGCGAGTGGGCGGTAGCTGCTCGTAGAGCGTGTGAAA |
| SEQ ID NO: 700 | GTTGGCCCTAAAAGTTATCATTCATGCTAGTTTGACCAAT |
| SEQ ID NO: 701 | AAGTGGGAGGAGCTGGGCAAGAAAGTCCACCCCTTTTTCT |
| SEQ ID NO: 702 | GCCGAGCCGAAGTCATCTGCCAATCAAAACAGCCACAGGG |
| SEQ ID NO: 703 | CGCGTACCTAATGGGAGACAGACAGGTGCCTTTAAAGCGG |
| SEQ ID NO: 704 | TGGGGAAAGCGGAGGAAGGCATGGAGTGTGGGCGTTAGGG |
| SEQ ID NO: 705 | GCATATTCTGCCTTGAAGTCATTGGTTGGTCCTGGAAGTG |
| SEQ ID NO: 706 | AATTGGTCTGGGGGAGGAGCTACGACAGTCCAGGGGCGGG |
| SEQ ID NO: 707 | GTGTCGTGCTGATTGGATGTATCCGCCCCCCTCTCTTAAA |
| SEQ ID NO: 708 | CAACACGCCAGCGCGAGGACCCGAACGTCAATCAAGAGAC |
| SEQ ID NO: 709 | GCGTTCGATTGGCCTCCCGCGCAGGCTGCTAGGATTGGCT |
| SEQ ID NO: 710 | CCCTGCCCCCTTTCGCGGATTGGGTGATCGCTCCAAGGCG |
| SEQ ID NO: 711 | CTGACCCTTGGAGGCTTTCTATTGGTTCCTGGCAGGGATG |
| SEQ ID NO: 712 | TCCCGAATATAGGCCAGTCATTGCTCCTGCTGAACGTCGC |
| SEQ ID NO: 713 | CCCCTCCTCTCTTCTCGTCTCTGGCGCCGACCCGCCCCCG |
| SEQ ID NO: 714 | GCTCAAGGGAGGCCGCGGCGTCTGCCGATGGCTCCGCGGA |
| SEQ ID NO: 715 | TGGGGGAGTGGGCCCGGGGTTGTTCTGACGACGGGGGTCG |
| SEQ ID NO: 716 | CCCGGGCGCTATCGCGATAGCGGCGCGAAGCGGAAGTGGG |
| SEQ ID NO: 717 | CGGGGGAGGCGAGCGCCCGCCGCCTTTTTCTCGCGCCCCG |
| SEQ ID NO: 718 | CACAGGAGCTGGCGCCGCCGCTGAGGAGCGTATCGCGACA |
| SEQ ID NO: 719 | GTTGCCGACTCGCGCTCTCGGCTTCTGCTCCGGGGCTTCT |
| SEQ ID NO: 720 | ACTCGGAGCTCGGATCCCAGTGTGGACCTGGACTCGAATC |
| SEQ ID NO: 721 | GGCTCCTCCTTGTTCCGAGCCCGAAGGCCCGCCCCTTCAC |
| SEQ ID NO: 722 | CTTTCCGGAGCCCGTCTGTTCCCCTTCGGGTCCAAAGCTT |
| SEQ ID NO: 723 | GACCCCGCCTCATTCCTCACGGCGAGCTCCAGACCCCGCC |
| SEQ ID NO: 724 | AGAACTCAAGCTCCCGATTGTGCCCGAAGGAACCCGAAGG |
| SEQ ID NO: 725 | ACTATTGCCGAAGTGAGCCGAAGTTTGTGGCCCCGCTTCC |
| SEQ ID NO: 726 | ACATGTGGCTCCGCCCACACTGGCCTCAGCTCTCCGTTCT |
| SEQ ID NO: 727 | ACAGTGACCCTAAGGACTCGACTACCTCCGAAGAAAGCCG |
| SEQ ID NO: 728 | CTTGTACCCAACTATCTACGAAGTAAACCGAAGCTTGTGG |
| SEQ ID NO: 729 | TATCTGGCGAACCTGTTGACTCCGCCTATCATCCTAGCGT |
| SEQ ID NO: 730 | GGCAAGTCGCTTTCGCCCCGCCCCCTTGTAAATACTCATG |
| SEQ ID NO: 731 | CTCCTCTACTTGGGAACTTGAGGATCGTCACCCTGGCCCG |
| SEQ ID NO: 732 | TTGGCTCCGCCCCACTGAGCGCACCTCCCTCTGCCGCTTC |
| SEQ ID NO: 733 | TCCTTGCTCCACCCCCTCATGCCGACACCCTCGTCAACTT |
| SEQ ID NO: 734 | TCCACCGATAGAACCAGCGAGTCACCTCATAAACAGTAAT |
| SEQ ID NO: 735 | CGCTCAGTCCGCCTCCTTGCCTCCCTTCAGAATGTCCCAC |
| SEQ ID NO: 736 | GCCGTCCACTCTCCGCTCGGGCGGGCTCACCCCAATTGGG |
| SEQ ID NO: 737 | CGACCGAACCCCACAGCCGAAAGCCCCGCCCCCTGGACAC |
| SEQ ID NO: 738 | CTCCGAGCGCCAGCGCACCCCAGTTGGGGAGTTCCCGCCC |
| SEQ ID NO: 739 | AGCCCCGCCTCCTCCCGGACGCAATAGGTTCGGCGTTCGG |
| SEQ ID NO: 740 | AGCAATTTGACGTTCGGGTGTTCTCGGCTCGGCCGAATCC |
| SEQ ID NO: 741 | TGCCCCCTCCCGAGCACAGGAAGTTCGGCGTTCGGGCGTC |
| SEQ ID NO: 742 | TTTCGGACCTCCTCGCTCTCAGACTCCCACAGTACAAAAC |
| SEQ ID NO: 743 | CGAGCCTTCGCTCCTCCTCTTTCCGAACGACTGTGATTCG |
| SEQ ID NO: 744 | GAGGCTAAGGCACCGCCGAGGCCACACCCTCTTCCGGACG |
| SEQ ID NO: 745 | GCGTCCCCCTTCGGGTGTTCCCGTCAGCGGTCAGAAGCTC |
| SEQ ID NO: 746 | CCTTACAAAGGTCCATTTTGGCACCACCCTCTTGCAAAGT |
| SEQ ID NO: 747 | GGAGCGTGAAAAACAAACCTCCGCAAGCGCGGCGACACGC |
| SEQ ID NO: 748 | ACCCGCTCTGTGCCCGCACTGCCGTACCTACCATTGCGCC |
| SEQ ID NO: 749 | GGTCCTCAGCATCTGCATATGTAGCCCCTCCCGCTGGTCA |
| SEQ ID NO: 750 | CCCAACCCCTACCCCCAATCCATCTTAGAGCTGATTCTCT |
| SEQ ID NO: 751 | ACTCCAGTGATTCTTCCTTATGCTAGGGACTCGAGGACCC |
| SEQ ID NO: 752 | GAGAATTGAGAAGTCAGTGTGGGAGGGGATGTCCCAGTAC |
| SEQ ID NO: 753 | TTTCTGGTTCGCGTTGGCTGCATTGTGGAGCTGAGGGATG |
| SEQ ID NO: 754 | TAGCTTCTTAATCTCCTTCTTTAGGTCAGCCTCATACTTT |
| SEQ ID NO: 755 | TTCTCCCTGGGACCCAGCAGTCCACTCTCCCAGTTCCCTC |
| SEQ ID NO: 756 | AAAGTCAGACCTCAGGACCCAGGAACTGGGGCCCACAGCT |
| SEQ ID NO: 757 | TCTTGATTTGGTCCCTCAGCCGCTGCAGATGGGAAAAGCA |
| SEQ ID NO: 758 | TAAGCTGCCTCTTGTCCTTGATCTCGTTGGACGCTACCCA |
| SEQ ID NO: 759 | GGCTCTGGGCTCCTACCGTCTCAATGAGCTTGCGGTTGTC |
| SEQ ID NO: 760 | TGAGGACCTCTGGGGTCTGGCCGCTCTGCCTCCGCCCCTT |
| SEQ ID NO: 761 | CTGCCTCTTCACTTCCCTTAGGTGCAGAAACCTTACTTCT |
| SEQ ID NO: 762 | CGACCTGAGCCTCGTGACCCTACTTTCTGAGCTCTGAGTC |
| SEQ ID NO: 763 | TCAAAGGTGGGAAAGGAGCTGACTAAGGGCCAGCAGACAC |
| SEQ ID NO: 764 | CCGTTCCATTTGCTGTAGAGAGTGCAGTTGGCAGGGGGGC |
| SEQ ID NO: 765 | GCTGTAAGCTTTGGTTTTGGTCTCTCGTTCCACAACTTTG |
| SEQ ID NO: 766 | CCAACTCACCGTGAGCCACTGGCCAACCTCTTCCTTCTCC |
| SEQ ID NO: 767 | CCAGGGCTCAGGATCCTCAGAGTTCACCTCCTCTTCTCTA |
| SEQ ID NO: 768 | GTCCACCTGCATGTTGAGCGTGTCGATGGTATTCTAGGGG |
| SEQ ID NO: 769 | GCGTGTCTGCACTGACAGTGACTCCACTTCACTCTCAAAC |
| SEQ ID NO: 770 | TGTCGGGTCTCCCTCACTCACATCCTTGTCGCCCTTCTTC |
| SEQ ID NO: 771 | CTGCTGGCCAGCCCATTCCCATGCCCATCCCCATCCCAAA |
| SEQ ID NO: 772 | GAATCCAGGCCCCAACTCCCAGGAGCATAAATGACTGGCC |
| SEQ ID NO: 773 | TCTCAAATCCCTAATCCCGGCTGTTGGCCCTGTCCGCCTG |
| SEQ ID NO: 774 | CCTGCCCCACGCGTGCAGCTGCTAAGCCCTCCCAATCCTG |
| SEQ ID NO: 775 | CCCAGACACCCAGGGGACCCTGAGATTCTGTCTGACCTCC |
| SEQ ID NO: 776 | CTTCCCCCAAGTCGCTCCTCTTCACAAAGGCCCCACGGTC |
| SEQ ID NO: 777 | CCTCTGGGTGCCAGGAGGCCTCTTGCCATGGGTGTCCTTC |
| SEQ ID NO: 778 | CTGCCTTGTCTCTACCCACTGTGCTCTCCCTAGGACCAGG |
| SEQ ID NO: 779 | GGCGAGGGGGAGGTCCTGCAGCTGCTCGCGTGGGCTGCCC |
| SEQ ID NO: 780 | TGCGCTCGATCTCATCCTTCAGTTCGTAGCCCACCTGGGG |
| SEQ ID NO: 781 | TCACCTGCTTCACAGGCGGCGGCTCCTGCCACTTGTCGAA |
| SEQ ID NO: 782 | CTCGCTTCTTCCGCTGTCCATCCAGGGGCGCAGGCAGCGG |
| SEQ ID NO: 783 | CCCATGCCTACCGGACCCCCAGGGCCCCTCACCTGCGGCC |
| SEQ ID NO: 784 | AGTCGGCTGGGAGGAGGACGCCGGCTTCTCCCCTCCATGA |
| SEQ ID NO: 785 | ATCTTGCGGTACCTGGGGACGGGTGGGTGGGCGGCGCCAG |
| SEQ ID NO: 786 | TTGGCCTGCTTCCGGATCTCCGTCAGCCCCAGCCGCTCCT |
| SEQ ID NO: 787 | GGAGGGCGCTCTGGGAGTCTGACCTCTCCGAAGCTCATAC |
| SEQ ID NO: 788 | AGGAGGCAGAGGGCGGTGGCGGCTGGCTGGCTGTGGGGTT |
| SEQ ID NO: 789 | AGACATGAGCCAGGGCCACAGGACGAGAGGAGGGGCGGTG |
| SEQ ID NO: 790 | CCAAGGGCCGCGAGGGTCGCTTTGGGGCTGAATGGATGGA |
| SEQ ID NO: 791 | GATGGGAAGCCGCGGGGGCTCTAAGCAGCGGAGACACAGG |
| SEQ ID NO: 792 | GGAGCCTCTGGGCAGGGAGGAACCGGCCAAGGAGCCCGGG |
| SEQ ID NO: 793 | GGCGGGGCCCAGGGACGGGGCGGCCGTGCAGCAGGGCACT |
| SEQ ID NO: 794 | CTGCAGGACCAAGGGGATGACGCTGGGATAACAGAGGAGA |
| SEQ ID NO: 795 | CAGAACAGGTTTAATAGGATGAGGTGGCCTCTGAGTTCGG |
| SEQ ID NO: 796 | CCATTCCTTCCTTACTCGTGTGGGTCGGGGGATGTCAGGA |
| SEQ ID NO: 797 | GGCCCGGTCCCAGCACTGCTCTGTGAGCTCAGAGTTGGGA |
| SEQ ID NO: 798 | TGGGGGCCCACACACGCGGGGGATGCCGGGGAGCCTGAGA |
| SEQ ID NO: 799 | CACGGGCACCTGCTCCGGTACCCACTCGGCCCGGCTGAGG |
| SEQ ID NO: 800 | CTCCACCAGCCGGAAGCCCAGCGGTCACCAGCCGGCCGGT |
| SEQ ID NO: 801 | AGGCGTCCTCCTCGATCTAGGGGGAAGAGGAGGCGCCCTG |
| SEQ ID NO: 802 | ACTTGCCCAGGTGGCCCAGGCTGAATCCCAGGTCCTCCTG |
| SEQ ID NO: 803 | TGGCCTCGTTTACCTGTGTCTGCCGCACACGCCCACTGCC |
| SEQ ID NO: 804 | GTCTGGCCCATACCTGCAGCGTCTTGGAGATCCTGGCCTT |
| SEQ ID NO: 805 | GCTCCCCCCACCTTGTGTCCCTCGGTCCCCAGCCCCACCT |
| SEQ ID NO: 806 | TGCAGGGTCCGCTGTGGGGAGGACAGGGAGGCTGCGATCT |
| SEQ ID NO: 807 | TCGCGGATGGTGGACTTCCCGCCATATACGACGCTCTGCT |
| SEQ ID NO: 808 | AGTGGGGTGAAGGCCACGCTGGAGGCCGTGCCCGAGGAGC |
| SEQ ID NO: 809 | CGGCTGCTGAGCCTAACCACCTCCTGGGCTTCTTTCCAGC |
| SEQ ID NO: 810 | GCTCATGGTATCCCTACCGCAGGCAATCTGTGGACAGCAC |
| SEQ ID NO: 811 | CTGAATGTCACCTGAAGGGTCACAGAAGCTACTCACAGGG |
| SEQ ID NO: 812 | TTAAGTGTTCTCAATATGAGATTAGCTGGAGCCGCCTAAT |
| SEQ ID NO: 813 | GAAGATCCATCTGTTGGAAGCCAGAGGACTAGTGGGAAAC |
| SEQ ID NO: 814 | CCCCCACAGGGATCTGACACACAACTTAGGTTGTCAGCCA |
| SEQ ID NO: 815 | GCCCAGCTTCCCAAGTCCTGCCTGGACACCGCCCCATGGA |
| SEQ ID NO: 816 | AATCACCTTCATGCTTAAAACACTCACACTGATTTCCAGC |
| SEQ ID NO: 817 | CCTCTTGGGGACCTGGGTGACCTTACTCACCCTCATGGCT |
| SEQ ID NO: 818 | GTTGCTGTGGACAGGCTTGGAGCCGTTTTTGGCTGGAGAC |
| SEQ ID NO: 819 | GGAGGGGTAGGTGGGCGGCACAGCTGGGGACTGAGGGTGC |
| SEQ ID NO: 820 | GCCAGGAGTGGTGCTCAAGGCAGAGGCAGCAGGCGGGGGG |
| SEQ ID NO: 821 | CAGGGCACTTGGGGGTGCTGCGGGGGCGGGGACCCCATTG |
| SEQ ID NO: 822 | GGTGCCCGAGTTGTGGCTGGGAGCTGGACTGGCCTTGGGG |
| SEQ ID NO: 823 | CTGCTTGCCAGCCCCTCCACCGGCACTGCTGTTACTACTG |
| SEQ ID NO: 824 | GCCCCCCACCCCGCTGCCTCCTCACTCACTGGTGGCGCCA |
| SEQ ID NO: 825 | CGGGCTGTCTGCCACAACTGAGCTGTAACCTGGGAACAAA |
| SEQ ID NO: 826 | GCTGGCATTGTTGCCCCCACTGCTGCTCAAAGCCACCTCT |
| SEQ ID NO: 827 | AGGTGGGTTGTGGGGGCCGGAAGGGGGGCCCAAGGCCTGG |
| SEQ ID NO: 828 | TCCCAACCCTGCCGATGGCCGAGACACTCACGAGGTGCTG |
| SEQ ID NO: 829 | GGGGGTGAGGCGCCTGCGCCTCTCTGTTTCAAAAGGCTGC |
| SEQ ID NO: 830 | ATTCCCAGCAGCAAGGGCGGGGGGTTCAGAACCCACCGAT |
| SEQ ID NO: 831 | GGGGGTGTAACACCCGAGGGAGATGGAGGATAGCGCTTGG |
| SEQ ID NO: 832 | CAAAGCAGGGAGGCTGATGTAGTTTCCTTGCTGGAAAGAA |
| SEQ ID NO: 833 | CTTCCACTTAGATGAGAACGTATTTTAGAATGTTCTGAAG |
| SEQ ID NO: 834 | TAACAGAAATGGGGAGGAAAGGGTATGGGGCTCTTGAGAA |
| SEQ ID NO: 835 | AAACAGTGACCCTCCGGTGGCAGTCAATTGGCCTCAGGCA |
| SEQ ID NO: 836 | GCAGAGGAATAAGGACTTCGGGACAATTCACTTTGAAAAG |
| SEQ ID NO: 837 | GACCCAGTGGAATGGTCTGAGCTAAGATTTGAAGGAGTGG |
| SEQ ID NO: 838 | TGCACACTGATCTTTCTTAGGGCATTCTTCGGGAAACAGG |
| SEQ ID NO: 839 | GGCTCAGGATGAACAGCAACAGGGGTTGGGATGATCACTG |
| SEQ ID NO: 840 | GATCATGGAGATGTGATCTAGGGAACAAAGCCAGAGAAGG |
| SEQ ID NO: 841 | AGGCATTCCCACGGTGTGAGGTCAGATTGGGCAGGGCCTA |
| SEQ ID NO: 842 | AGAGCCAGCACTTGCTGTTCCACACATACTAGATCAGTCT |
| SEQ ID NO: 843 | TGGACAACCCCCTCCCACACCCAGAGCTGTGGAAGGGGAG |
| SEQ ID NO: 844 | CACCTAGATGCTGACCAAGGCCCTCCCCATGCTGCTGGAG |
| SEQ ID NO: 845 | ATAAAGCCTTCATTCTCCAGGACCCCGCCCTTGCCCTGTT |
| SEQ ID NO: 846 | AGGTGGTGAGTTTGGGGCTGGGGGGCCTCCCTGAGGAGCC |
| SEQ ID NO: 847 | GAGAGAACCAGGTCCCACATGCTGACACAGGTGTCCACGG |
| SEQ ID NO: 848 | ATCCCCCCAATCTCACCAGTGCACCCCACAGACAAGGCGA |
| SEQ ID NO: 849 | AAGGGCTTCAGCATAAGAGTCAGAACCCGCCCCCCTTCCT |
| SEQ ID NO: 850 | TGTGGGCTGAAGGGACGAGGCTGGGGCACTGGGTGGGAGG |
| SEQ ID NO: 851 | TTGCAATGTGGAAGAGTCAGGGGCACATTGTCTGGGCTGA |
| SEQ ID NO: 852 | TAAGTGGGAGGGAGCGGGGACCTAGTGTGGGCATGAGGAC |
| SEQ ID NO: 853 | GGAGCAGGGATTTGGCTGGGCAATGGAGAGAAAGGTCTGA |
| SEQ ID NO: 854 | ACACAGAGATGCCCAGGAACTTGCTCTTTAGTAAAGCAGC |
| SEQ ID NO: 855 | TGGAGAGAGGTCCTTGAAAGGTTTTGAACCCCATAAAGAG |
| SEQ ID NO: 856 | TCAGGAGGCAGCCCAGTGATAGGGTCCAAGGAACCAGTGG |
| SEQ ID NO: 857 | ACAGTCTACTGACTTTTCCTATTCAGCTGTGAGCATTCAA |
| SEQ ID NO: 858 | CTGTCCCCTGGACCTTGACACCTGGCTCCCCAACCCTGTC |
| SEQ ID NO: 859 | AGGAAACCCAGATTCCACCAGACACTTCCTTCTTCCCCCC |
| SEQ ID NO: 860 | GGCTATCTGGCCTGAGACAACAAATGCTGCCTCCCACCCT |
| SEQ ID NO: 861 | GTCTGGCACTGGGACTTTCAGAACTCCTCCTTCCCTGACT |
| SEQ ID NO: 862 | TTGCCCCAGACCCGTCATTCAATGGCTAGCTTTTTCCATG |
| SEQ ID NO: 863 | AAAAACACGAGCACCCCCAACCACAACGGCCAGTTCTCTG |
| SEQ ID NO: 864 | TTAACCTTGGACATGGTAAACCATCCAAAACCTTCCTCTC |
| SEQ ID NO: 865 | AGCAACTAAACCTCTCCACTGGGCACTTATCCTTGGTTTC |
| SEQ ID NO: 866 | GAACCTCTTATTCTCTTAGAACCCACAGCTGCCACCACAG |
| SEQ ID NO: 867 | TCCCTTCTCCCAGTGTAAGACCCCAAATCACTCCAAATGA |
| SEQ ID NO: 868 | CAACCCCCAACCCGATGCCTGCTTCAGATGTTTCCCATGT |
| SEQ ID NO: 869 | CATAAACCTGGCTCCTAAAGGCTAAATATTTTGTTGGAGA |
| SEQ ID NO: 870 | CTGCTGACCTGCCCTCCCAGGTCAGAATCATCCTCATGCA |
| SEQ ID NO: 871 | TGTTCTCCAGACCTGTGCACTCTATCTGTGCAACAGAGAT |
| SEQ ID NO: 872 | CGTGCAGCAAACAATGTGGAATTCCAATAACCCCCCACTC |
| SEQ ID NO: 873 | AAATATGAGTCTCCCAAAGTTCCCTAGCATTTCAAAATCC |
| SEQ ID NO: 874 | CATCATAAAAAGATCTTGTGGTCCACAGATCCTCTAGCCC |
| SEQ ID NO: 875 | CTCCCAACCCAGAATCCAGCTCCACAGATACATTGCTACT |
| SEQ ID NO: 876 | CACTCTGAGACCAGAAACTAGAACTTTTATTCCTCATGCT |
| SEQ ID NO: 877 | CACCAGCACTCAGGAGATTGTGAGACTCCCTGATCCCTGC |
| SEQ ID NO: 878 | TGCCTAGATCCTTTGCACTCCAAGACCCAGTGTGCCCTAA |
| SEQ ID NO: 879 | GGGGGTGGGTACGATCCCCGATTCTTCATACAAAGCCTCA |
| SEQ ID NO: 880 | GGACAAAGGCAGAGGAGACACGCCCAGGATGAAACAGAAA |
| SEQ ID NO: 881 | TGGATGCACCAGGCCCTGTAGCTCATGGAGACTTCATCTA |
| SEQ ID NO: 882 | GGGAGAGCTAGCACTTGCTGTTCTGCAATTACTAGATCAC |
| SEQ ID NO: 883 | GGCTGGACAACCCCCTCCCACACCCAGAGCTGTGGAAGGG |
| SEQ ID NO: 884 | TGGCACCCAGAGGCTGACCAAGGCCCTCCCCATGCTGCTG |
| SEQ ID NO: 885 | CCTATAAAACCTTCATTCCCCAGGACTCCGCCCCTGCCCT |
| SEQ ID NO: 886 | TGCAGGTGGTAAGCTTGGGGCTGGGGAGCCTCCCCCAGGA |
| SEQ ID NO: 887 | AGGAAGACAACCGGGACCCACATGGTGACACAGCTCTCCG |
| SEQ ID NO: 888 | CAACCATGGCCCCTCTCACCAATCCACGTCACGGACAGGG |
| SEQ ID NO: 889 | TCAGCTTGACAGTCAGGGCTGGCTCCCTCTCCTGCATCCC |
| SEQ ID NO: 890 | TCCCTGTCTGGGCTGGGGTGCTGGGTTGGGGGGGAAAGAG |
| SEQ ID NO: 891 | TGTGGGAGTGAGGACTGTTGCAATATGGAGGGGCTGGGGG |
| SEQ ID NO: 892 | GGGAGAAAGTTCTGGGGTAAGTGGGAGGGAGCGGGGACCT |
| SEQ ID NO: 893 | TTGTGGGGCTCAAAACCTCCAAGGACCTCTCTCAATGCCA |
| SEQ ID NO: 894 | TGCCCAACCCTATCCCAGAGACCTTGATGCTTGGCCTCCC |
| SEQ ID NO: 895 | TCTTGCCCTAGGATACCCAGATGCCAACCAGACACCTCCT |
| SEQ ID NO: 896 | TTCCTAGCCAGGCTATCTGGCCTGAGACAACAAATGGGTC |
| SEQ ID NO: 897 | TCTTAGCCCCAGACTCTTCATTCAGTGGCCCACATTTTCC |
| SEQ ID NO: 898 | AGGAAAAACATGAGCATCCCCAGCCACAACTGCCAGCTCT |
| SEQ ID NO: 899 | CCCCTTCAGAGTTACTGACAAACAGGTGGGCACTGAGACT |
| SEQ ID NO: 900 | TGGAAAGTTAGCTTATTTGTTTGCAAGTCAGTAAAATGTC |
| SEQ ID NO: 901 | GACTCAGGAGTCTCATGGACTCTGCCAGCATTCACAAAAC |
| SEQ ID NO: 902 | ATGCTGTCTGCTAAGCTGTGAGCAGTAAAAGCCTTTGCCT |
| SEQ ID NO: 903 | GATTTGGGGGGGGCAAGGTGTACTAATGTGAACATGAACC |
| SEQ ID NO: 904 | GTGTGCACAGCATCCACCTAGACTGCTCTGGTCACCCTAC |
| SEQ ID NO: 905 | AGGATTCCTAATCTCAGGTTTCTCACCAGTGGCACAAACC |
| SEQ ID NO: 906 | CAAAGGCTGAGCAGGTTTGCAAGTTGTCCCAGTATAAGAT |
| SEQ ID NO: 907 | GTCAAGGACAATCGATACAATATGTTCCTCCAGAGTAGGT |
| SEQ ID NO: 908 | GCAAGATGATATCTCTCTCAGATCCAGGCTTGCTTACTGT |
| SEQ ID NO: 909 | TCTGTGTGTCTTCTGAGCAAAGACAGCAACACCTTTTTTT |
| SEQ ID NO: 910 | AACGTTGAGACTGTCCTGCAGACAAGGGTGGAAGGCTCTG |
| SEQ ID NO: 911 | CATAAATAAGCAGGATGTGACAGAAGAAGTATTTAATGGT |
| SEQ ID NO: 912 | GCTGCCAGACACAGTCGATCGGGACCTAGAACCTTGGTTA |
| SEQ ID NO: 913 | GGGATCCTGAGCGCTGCCTTATTCTGGGTTTGGCAGTGGA |
| SEQ ID NO: 914 | TCACTCAAACCCAGAAGTTCTGATCCCCAGCCATGCCCCT |
| SEQ ID NO: 915 | AGCCTCTTCCTCCTTTGAAATTCAAGAGGGTGGACCCACT |
| SEQ ID NO: 916 | GGAGCTGGGACCTTACCAGTCTCCTCCCTCATTGACCTAA |
| SEQ ID NO: 917 | GAGGATATGAGATTCTTAGGCCATTCCCACATCAGTACCT |
| SEQ ID NO: 918 | TACCCAGAACTCTACCCCTCAGGATTCCAGCACCTTCTTC |
| SEQ ID NO: 919 | GCCTCTGCCCTTCAGGGGCCAAAGAGCCTTAAGCCACAAA |
| SEQ ID NO: 920 | ATCCCATTACTATCACCCCAAACCCTGGACCTAATGGTTC |
| SEQ ID NO: 921 | AATGGGCAACCCTCGATCCTCAGACTCTTGAGGAATCAAG |
| SEQ ID NO: 922 | GATACCCTCAAGTGGAGTAAGGATTAGGTGGCAAGATGGA |
| SEQ ID NO: 923 | GTGCTTGCCCAGGGGCACCTTCATGGAGCTAGAAGGGCTG |
| SEQ ID NO: 924 | GATGACACCCAAGGCCTCTGGGGCATCTTTCATGCTCAGA |
| SEQ ID NO: 925 | TGCTGGCCACACCCTCAGAGTGTGGATGCTGGATGATGAG |
| SEQ ID NO: 926 | GAGGCACGCTGCAGGGATAGTCACAGCAACATGACGTCAT |
| SEQ ID NO: 927 | AGAGGAGGATGTCGGCAGCTCTACGGTTGGCAGGTGGCTG |
| SEQ ID NO: 928 | GACACTAGGCCTCAGCCTGGCACCATGCAGGCCACTCCCA |
| SEQ ID NO: 929 | ACTTTTGAGTCCTGGATCCCTATGATTCCAGGCTCCCTGT |
| SEQ ID NO: 930 | CCTTGAGATTTCATGGATGGTGACATATGGCCATTCTCTA |
| SEQ ID NO: 931 | AAAACCCATAAGTTCAGGTCCCTGTGCCCTCCACCCAGAA |
| SEQ ID NO: 932 | TCGTATCTGGGAGACTCACTTGGGAGAGCAATAGACTTGG |
| SEQ ID NO: 933 | TACAAGATGTGGTGGAGATAAGGCTGATGCTGGCACAGTG |
| SEQ ID NO: 934 | GTACACACCATGGTGTTCATCAGGGCCCTGGGTAGTCCCT |
| SEQ ID NO: 935 | GCTGTGACCTCACAGGAGTCCGTGCCTCCACCCCCTACTC |

A nucleic acid probe may be a non-labeled probe, or a probe that does not contain a detectable moiety. A non-labeled probe may further interact with a labeled probe (e.g., a labeled nucleic acid probe). A non-labeled probe may hybridize with a labeled nucleic acid probe. A non-labeled probe may also interact with a labeled polypeptide probe. The labeled polypeptide probe may be a protein that recognizes a sequence within the non-labeled probe. A labeled probe may include a nucleic acid portion and a polypeptide tag portion and the polypeptide tag portion may further interact with a molecule comprising a detectable moiety. For example, a non-labeled probe may be a nucleic acid probe comprising a streptavidin which may interact with a biotinylated molecule comprising a detectable moiety.

A nucleic acid probe may have about 95%, about 96%, about 97%, about 98%, about 99%, or about 100% sequence specificity or sequence complementarity to a target site of a regulatory element. The hybridization may be a high stringent hybridization condition.

A nucleic acid probe may hybridize with a genomic sequence that is present in low or single copy numbers (e.g., genomic sequences that are not repetitive elements). As used herein, repetitive element refers to a DNA sequence that is present in many identical or similar copies in the genome. Repetitive elements are not intended to refer to a DNA sequence that is present on each copy of the same chromosome (e.g., a DNA sequence that is present only once, but is found on both copies of chromosome 11, would not be considered a repetitive element, and would be considered a sequence that is present in the genome as one copy). The genome may consist of three broad sequence components: single copy or at least very low copy number DNA (approximately 60% of the human genome); moderately repetitive elements (approximately 30% of the human genome); and highly repetitive elements (approximately 10% of the human genome). For a review, see Human Molecular Genetics, Chapter 7 (1999), John Wiley & Sons, Inc.

A nucleic acid probe may have reduced off-target interaction. For example, “off-target” or “off-target interaction” may refer to an instance in which a nucleic acid probe against a given target hybridizes or interact with another target site (e.g., a different DNA sequence, RNA sequence, or a cellular protein or other moiety).

A nucleic acid probe may further be cross-linked to a target site of a regulatory element. For example, the nucleic acid probe may be cross-linked by a photo-crosslinking means such as UV or by a chemical cross-linking means such as by formaldehyde, or through a reactive group within the nucleic acid probe. Reactive group may include sulfhydryl-reactive linkers such as bismaleimidohexane (BMH), and the like.

A nucleic acid probe may include natural or unnatural nucleotide analogues or bases or a combination thereof. The unnatural nucleotide analogues or bases may comprise modifications at one or more of ribose moiety, phosphate moiety, nucleoside moiety, or a combination thereof. The unnatural nucleotide analogues or bases may comprise 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O-N-methylacetamido (2′-O-NMA) modified, locked nucleic acid (LNA), ethylene nucleic acid (ENA), peptide nucleic acid (PNA), 1′, 5′-anhydrohexitol nucleic acids (HNA), morpholino, methylphosphonate nucleotides, thiolphosphonate nucleotides, or 2′-fluoro N3-P5′-phosphoramidites. The nucleic acid probes may further comprise one or more abasic sites. The abasic site may further be functionalized with a detectable moiety.

A nucleic acid probe may comprise a Transcription Activator-Like Effector (TALE) sequence. A TALE may comprise a DNA-binding module which includes a variable number of about 33-35 amino acid residue repeats. Each amino acid repeat recognizes one base pair through two adjacent amino acids (such as at amino acid positions 12 and 13 of the repeat). As such, the amino acid repeat may also be referred to as repeat-variable diresidue (RVD).

A TALE probe described herein may comprise between about 1 to about 50 TALE repeat modules. A TALE probe described herein may comprise between about 5 and about 45, between about 8 and about 45, between about 10 and about 40, between about 12 and about 35, between about 15 and about 30, between about 20 and about 30, between about 8 and about 40, between about 8 and about 35, between about 8 and about 30, between about 10 and about 35, between about 10 and about 30, between about 10 and about 25, between about 10 and about 20, or between about 15 and about 25 TAL effector repeat modules.

A TALE probe described herein may comprise about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 45, or about 50 TALE repeat modules. A TALE probe described herein may comprise about 5 TALE repeat modules. A TALE probe described herein may comprise about 10 TALE repeat modules. A TALE probe described herein may comprise about 11 TALE repeat modules. A TALE probe described herein may comprise about 12 TALE repeat modules. A TALE probe described herein may comprise about 13 TALE repeat modules. A TALE probe described herein may comprise about 14 TALE repeat modules. A TALE probe described herein may comprise about 15 TALE repeat modules. A TALE probe described herein may comprise about 16 TALE repeat modules. A TALE probe described herein may comprise about 17 TALE repeat modules. A TALE probe described herein may comprise about 18 TALE repeat modules. A TALE probe described herein may comprise about 19 TALE repeat modules. A TALE probe described herein may comprise about 20 TALE repeat modules. A TALE probe described herein may comprise about 21 TALE repeat modules. A TALE probe described herein may comprise about 22 TALE repeat modules. A TALE probe described herein may comprise about 23 TALE repeat modules. A TALE probe described herein may comprise about 24 TALE repeat modules. A TALE probe described herein may comprise about 25 TALE repeat modules. A TALE probe described herein may comprise about 26 TALE repeat modules. A TALE probe described herein may comprise about 27 TALE repeat modules. A TALE probe described herein may comprise about 28 TALE repeat modules. A TALE probe described herein may comprise about 29 TALE repeat modules. A TALE probe described herein may comprise about 30 TALE repeat modules. A TALE probe described herein may comprise about 35 TALE repeat modules. A TALE probe described herein may comprise about 40 TALE repeat modules. A TALE probe described herein may comprise about 45 TALE repeat modules. A TALE probe described herein may comprise about 50 TALE repeat modules.

A TAL effector repeat module may be a wild-type TALE DNA-binding module or a modified TALE DNA-binding repeat module enhanced for specific recognition of a nucleotide. A TALE probe described herein may comprise one or more wild-type TALE DNA-binding module. A TALE probe described herein may comprise one or more modified TAL effector DNA-binding repeat module enhanced for specific recognition of a nucleotide. A modified TALE DNA-binding repeat module may comprise 1, 2, 3, 4, 5, 6, 7, 8, 9, 10, 15, 20, 25 or more mutations that may enhance the repeat module for specific recognition of a nucleic acid sequence (e.g., a target sequence). In some cases, a modified TALE DNA-binding repeat module is modified at amino acid position 2, 3, 4, 11, 12, 13, 21, 23, 24, 25, 26, 27, 28, 30, 31, 32, 33, 34, or 35. In some cases, a modified TALE DNA-binding repeat module is modified at amino acid positions 12 or 13.

A TALE repeat module may be a repeat module-like domain or RVD-like domain. A RVD-like domain has a sequence different from naturally occurring polynucleotidic repeat module comprising RVD (RVD domain) but have a similar function and/or global structure. Non-limiting examples of RVD-like domains include protein domains selected from Puf RNA binding protein or Ankyrin super-family.

A TALE repeat module may comprise a RVD domain of Table 1. A TALE probe described herein may comprise one or more RVD domains selected from Table 1. Sometimes, A TALE probe described herein may comprise up to 1, up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, up to 30, up to 31, up to 32, up to 33, up to 34, up to 35, up to 36, up to 37, up to 38, up to 39, up to 40, up to 45, up to 50, up to 60, up to 70, up to 80, up to 90, or up to 100 RVD domains selected from TABLE 2.

TABLE 2

| RVD | Nucleotide |
|---|---|
| HD | C |
| NG | T |
| NI | A |
| NN | G > A |
| NS | G, A > C > T |
| NH | G |
| N* | T > C >> G, A |
| NP | T > A, C |
| HG | T |
| H* | T |
| IG | T |
| HA | C |
| ND | C |
| NK | G |
| HI | C |
| HN | G > A |
| NT | G > A |
| NA | G |
| SN | G or A |
| SH | G |
| YG | T |
| IS | — |

*Denotes a gap in the repeat sequence corresponding to a lack of an amino acid residue at the second position of the RVD.

An RVD domain may recognize or interact with one nucleotide. An RVD domain may recognize or interact with more than one nucleotides. The efficiency of a RVD domain at recognizing a nucleotide is ranked as “strong”, “intermediate” or “weak”. The ranking may be according to a ranking described in Streubel et al., “TAL effector RVD specificities and efficiencies,” Nature Biotechnology 30 (7): 593-595 (2012). The ranking of RVD may be as illustrated in TABLE 3, based on the ranking provided in Streubel et al. Nature Biotechnology 30 (7): 593-595 (2012).

TABLE 3

| RVD | Nucleotide | Efficiency |
|---|---|---|
| HD | C | strong |
| NG | T | weak |
| NI | A | weak |
| NN | G > A | Strong (G), intermediate (A) |
| NS | G, A > C > T | intermediate |
| NH | G | intermediate |
| N* | T > C >> G, A | weak |
| NP | T > A, C | intermediate |
| NK | G | weak |
| HN | G > A | intermediate |
| NT | G > A | intermediate |
| SN | G or A | Weak |
| SH | G | Weak |
| IS | — | weak |

*Denotes a gap in the repeat sequence corresponding to a lack of an amino acid residue at the second position of the RVD.

A TALE DNA-binding domain may further comprise a C-terminal truncated TALE DNA-binding repeat module. A C-terminal truncated TALE DNA-binding repeat module may be between about 18 and about 40 residues in length. A C-terminal truncated TALE DNA-binding repeat module may be between about 20 and about 40, between about 22 and about 38, between about 24 and about 35, between about 28 and about 32, between about 25 and about 40, between about 25 and about 38, between about 25 and about 30, between about 28 and about 40, or between about 28 and about 35 residues in length. A C-terminal truncated TALE DNA-binding repeat module may be at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or more residues in length. A C-terminal truncated TALE DNA-binding repeat module may be about 18, about 19, about 20, about 21, about 22, about 23, about 24, about 25, about 26, about 27, about 28, about 29, about 30, about 31, about 32, about 33, about 34, about 35, about 36 about 37, about 38, about 39, or about 40 residues in length. A C-terminal truncated TALE DNA-binding repeat module may be about 18 residues in length. A C-terminal truncated TALE DNA-binding repeat module may be about 19 residues in length. A C-terminal truncated TALE DNA-binding repeat module may be about 20 residues in length. A C-terminal truncated TALE DNA-binding repeat module may be about 21 residues in length. A C-terminal truncated TALE DNA-binding repeat module may be about 22 residues in length. A C-terminal truncated TALE DNA-binding repeat module may be about 23 residues in length. A C-terminal truncated TALE DNA-binding repeat module may be about 24 residues in length. A C-terminal truncated TALE DNA-binding repeat module may be about 25 residues in length. A C-terminal truncated TALE DNA-binding repeat module may be about 26 residues in length. A C-terminal truncated TALE DNA-binding repeat module may be about 27 residues in length. A C-terminal truncated TALE DNA-binding repeat module may be about 28 residues in length. A C-terminal truncated TALE DNA-binding repeat module may be about 29 residues in length. A C-terminal truncated TALE DNA-binding repeat module may be about 30 residues in length. A C-terminal truncated TALE DNA-binding repeat module may be about 31 residues in length. A C-terminal truncated TALE DNA-binding repeat module may be about 32 residues in length. A C-terminal truncated TALE DNA-binding repeat module may be about 33 residues in length. A C-terminal truncated TALE DNA-binding repeat module may be about 34 residues in length. A C-terminal truncated TALE DNA-binding repeat module may be about 35 residues in length. A C-terminal truncated TALE DNA-binding repeat module may be about 36 residues in length. A C-terminal truncated TALE DNA-binding repeat module may be about 37 residues in length. A C-terminal truncated TALE DNA-binding repeat module may be about 38 residues in length. A C-terminal truncated TALE DNA-binding repeat module may be about 39 residues in length. A C-terminal truncated TALE DNA-binding repeat module may be about 40 residues in length. A C-terminal truncated TALE DNA-binding repeat module may be a RVD domain of TABLE 2.

A TALE DNA-binding domain may further comprise an N-terminal cap. An N-terminal cap may be a polypeptide portion flanking the DNA-binding repeat module. An N-terminal cap may be any length and may comprise from about 0 to about 136 amino acid residues in length. An N-terminal cap may be about 5, about 10, about 15, about 20, about 25, about 30, about 35, about 40, about 45, about 50, about 60, about 70, about 80, about 90, about 100, about 110, about 120, or about 130 amino acid residues in length. An N-terminal cap may modulate structural stability of the DNA-binding repeat modules. An N-terminal cap may modulate nonspecific interactions. An N-terminal cap may decrease nonspecific interaction. An N-terminal cap may reduce off-target effect. As used here, off-target effect refers to the interaction of a TALE protein with a sequence that is not the target sequence of interest. An N-terminal cap may further comprise a wild-type N-terminal cap sequence of a TALE protein or may comprise a modified N-terminal cap sequence.

A TALE DNA-binding domain may further comprise a C-terminal cap sequence. A C-terminal cap sequence may be a polypeptide portion flanking the C-terminal truncated TALE DNA-binding repeat module. A C-terminal cap may be any length and may comprise from about 0 to about 278 amino acid residues in length. A C-terminal cap may be about 5, about 10, about 15, about 20, about 25, about 30, about 35, about 40, about 45, about 50, about 60, about 80, about 100, about 150, about 200, or about 250 amino acid residues in length. A C-terminal cap may further comprise a wild-type C-terminal cap sequence of a TALE protein, or may comprise a modified C-terminal cap sequence.

A nuclease domain may be linked to a TALE DNA-binding domain either directly or through a linker. A linker may be between about 1 and about 50 amino acid residues in length. A linker may be from about 5 to about 45, from about 5 to about 40, from about 5 to about 35, from about 5 to about 30, from about 5 to about 25, from about 5 to about 20, from about 5 to about 15, from about 10 to about 40, from about 10 to about 35, from about 10 to about 30, from about 10 to about 25, from about 10 to about 20, from about 12 to about 40, from about 12 to about 35, from about 12 to about 30, from about 12 to about 25, from about 12 to about 20, from about 14 to about 40, from about 14 to about 35, from about 14 to about 30, from about 14 to about 25, from about 14 to about 20, from about 14 to about 16, from about 15 to about 40, from about 15 to about 35, from about 15 to about 30, from about 15 to about 25, from about 15 to about 20, from about 15 to about 18, from about 18 to about 40, from about 18 to about 35, from about 18 to about 30, from about 18 to about 25, from about 18 to about 24, from about 20 to about 40, from about 20 to about 35, from about 20 to about 30, or from about 25 to about 30 amino acid residues in length.

A linker for linking a nuclease domain to a TALE DNA-binding domain may be about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, or about 50 amino acid residues in length. A linker may be about 10 amino acid residues in length. A linker may be about 11 amino acid residues in length. A linker may be about 12 amino acid residues in length. A linker may be about 13 amino acid residues in length. A linker may be about 14 amino acid residues in length. A linker may be about 15 amino acid residues in length. A linker may be about 16 amino acid residues in length. A linker may be about 17 amino acid residues in length. A linker may be about 18 amino acid residues in length. A linker may be about 19 amino acid residues in length. A linker may be about 20 amino acid residues in length. A linker may be about 21 amino acid residues in length. A linker may be about 22 amino acid residues in length. A linker may be about 23 amino acid residues in length. A linker may be about 24 amino acid residues in length. A linker may be about 25 amino acid residues in length. A linker may be about 26 amino acid residues in length. A linker may be about 27 amino acid residues in length. A linker may be about 28 amino acid residues in length. A linker may be about 29 amino acid residues in length. A linker may be about 30 amino acid residues in length.

A TALE probe may be designed to recognize each strand of a double-stranded segment of DNA by engineering the TALE to include a sequence of repeat-variable diresidue subunits that may comprise about 22, about 23, about 24, about 25, about 26, about 27, about 28, about 29, about 30, about 31, about 32, about 33, about 34, about 35, about 36, about 37, about 38, about 39, or about 40 amino acid repeats capable of associating with specific DNA sequences, such that the detectable label of the TALE probe is located at the target nucleic acid sequence.

A nucleic acid probe may be a locked nucleic acid probe (such as a labeled locked nucleic acid probe), a labeled or unlabeled peptide nucleic acid (PNA) probe, a labeled or unlabeled oligonucleotide, an oligopaint, an ECHO probe, a molecular beacon probe, a padlock (or molecular inversion probe), a labeled or unlabeled toe-hold probe, a labeled TALE probe, a labeled ZFN probe, or a labeled CRISPR probe.

A nucleic acid probe may be a labeled or unlabeled locked nucleic acid probe or a labeled or unlabeled peptide nucleic acid probe. Locked nucleic acid probes and peptide nucleic acid probes are known to those of skill in the art and are described in Briones et al., Anal Bioanal Chem (2012) 402:3071-3089.

A nucleic acid probe may be a padlock (or molecular inversion probe). A padlock probe may be hybridized to a target regulatory element sequence in which the two ends may correspond to the target sequence. A padlock probe may be ligated together by a ligase (such as T4 ligase) when bound to the target sequence. An amplification (such as a rolling circle amplification or RCA) may be performed utilizing for example @29 polymerase, which may result in a single stranded DNA comprising multiple tandem copies of the target sequence.

A nucleic acid probe may be an oligopaint as described in U.S. Publication No. 2010/0304994; and in Beliveau, et al., “Versatile design and synthesis platform for visualizing genomes with oligopaint FISH probes,” PNAS 109 (52): 21301-21306 (2012). Oligopaint may refer to detectably labeled polynucleotides that have sequences complementary to an oligonucleotide sequence (such as a portion of a DNA sequence, like a particular chromosome or sub-chromosomal region of a particular chromosome). Oligopaints may be generated from synthetic probes and arrays that are, optionally, computationally patterned (rather than using natural DNA sequences and/or chromosomes as a template).

A nucleic acid probe may be a labeled or unlabeled toe-hold probe. Toe-hold probes are known to those of skill in the art as described in Zhang et al., Optimizing the Specificity of Nucleic Acid Hybridization, Nature Chemistry 4:208-214 (2012).

A nucleic acid probe may be a molecular beacon. Molecular beacons may be hairpin shaped molecules with an internally quenched fluorophore whose fluorescence is restored when they bind to a target nucleic acid sequence. Molecular beacons are known to those of skill in the art as described in Guo et al., Anal. Bioanal. Chem. (2012) 402:3115-3125.

A nucleic acid probe may be an ECHO probe. ECHO probes may be sequence-specific, hybridization-sensitive, quencher-free fluorescent probes for RNA detection, which may be designed using the concept of fluorescence quenching caused by intramolecular excitonic interaction of fluorescent dyes. ECHO probes are known to those of skill in the art as described in Kubota et al., PLOS ONE, Vol. 5, Issue 9, e13003 (2010); or Okamoto, Chem. Soc. Rev., 2011, 40, 5815-5828, Wang et al., RNA (2012), 18:166-175.

A probe may be a clustered regularly interspaced palindromic repeat (CRISPR) probe. The CRISPR system may use a Cas9 protein to recognize DNA sequences, in which the target specificity may be solely determined by a small guide (sg) RNA and a protospacer adjacent motif (PAM). Upon binding to target DNA, the Cas9-sgRNA complex may generate a DNA double-stranded break. For imaging applications, a Cas9 protein may be replaced with an endonuclease-deactivated Cas9 (dCas9) protein. For example, imaging a cell, such as by fluorescence in situ hybridization (FISH), may be achieved by synthesizing a dCas9 within the cell, synthesizing RNA within the cell to bind genomic DNA and to complex with the dCas9 forming a dCas9/RNA complex, labeling the dCas9/RNA complex, and imaging the labeled dCas9/RNA complex within the live cell bound to genomic DNA. The endonuclease-deactivated Cas9 may be synthesized in vivo by using an integrated construct, a transiently transfected construct, by injection into the cell of a syncitia of nuclei or via electroporation into cells and/or nuclei.

A probe may comprise an endonuclease-deactivated Cas9 (dCas9) protein as described in Chen et al., “Dynamic imaging of genomic loci in living human cells by an optimized CRISPR/Cas system,” Cell 155 (7): 1479-1491 (2013); or Ma et al., “Multicolor CRISPR labeling of chromosomal loci in human cells,” PNAS 112 (10): 3002-3007 (2015). The dCas9 protein may be further labeled with a detectable moiety.

The RNA of the Cas9/RNA complex may be synthesized in vivo by using an integrated construct, a transiently transfected construct, by injection into the cell of a syncitia of nuclei or via electroporation into cells and/or nuclei. The Cas9/RNA complex may be labeled by making a fusion protein that includes Cas9 and a reporter, by injection of RNA that has been attached to a reporter into the cell or by a syncitia of nuclei including RNA that has been attached to a reporter, by electroporation into cells or nuclei or by indirect labeling of the RNA by hybridization with a labeled secondary oligonucleotide. The label may be a conditional reporter, based on the binding of Cas9/RNA to the target nucleic acid. The label may be quenched and may then be activated upon the Cas9/RNA complex binding to the target nucleic acid.

A probe may be a transcription activator-like effector nuclease (TALEN) probe. TALENs are engineered restriction enzymes generated by fusing the TALE DNA binding domain to a FokI DNA cleavage domain. A FokI DNA cleavage domain may comprise an endonuclease-deactivated FokI domain. A nucleic acid probe may be a TALEN probe comprising an endonuclease-deactivated FokI domain.

A probe may be a zinc-finger nuclease (ZFN) probe. Similar to TALEN, a zinc-finger nuclease is an engineered restriction enzyme generated by fusing a zinc finger DNA-binding domain to a zinc finger nuclease. A zinc finger nuclease may comprise an endonuclease-deactivated zinc finger nuclease. A nucleic acid probe may be a ZFN probe comprising an endonuclease-deactivated zinc finger nuclease.

A probe disclosed herein may be a polypeptide probe. A polypeptide probe may include a protein or a binding fragment thereof that interacts with a target site (such as a nucleic acid target site or a protein target) of interest. A polypeptide probe may comprise a DNA-binding protein, a RNA-binding protein, a protein involved in the transcription/translation process or detects the transcription/translation process, a protein that may detect an open or relaxed portion of a chromatin, or a protein interacting partner of a product of a regulatory element.

A polypeptide probe may be a DNA-binding protein. The DNA-binding protein may be a transcription factor that modulates the transcription process, polymerases, or histones. A DNA-binding protein may comprise a zinc finger domain, a helix-turn-helix domain, a leucine zipper domain (such as a basic leucine zipper domain), a high mobility group box (HMG-box) domain, and the like. The DNA-binding protein may interact with a nucleic acid region in a sequence specific manner. The DNA-binding protein may interact with a nucleic acid region in a sequence non-specific manner. The DNA-binding protein may interact with single-stranded DNA. The DNA-binding protein may interact with double-stranded DNA. The DNA-binding protein probe may further comprise a detectable moiety.

A polypeptide probe may be a RNA-binding protein. The RNA-binding protein may participate in forming ribunucleoprotein complexes. The RNA-binding protein may modulate post-transcription such as in splicing, polyadenylation, mRNA stabilization, mRNA localization, or in translation. A RNA-binding protein may comprise a RNA recognition motif (RRM), dsRNA binding domain, zinc finger domain, K-Homology domain (KH domain), and the like. The RNA-binding protein may interact with single-stranded RNA. The RNA-binding protein may interact with double-stranded RNA. The RNA-binding protein probe may further comprise a detectable moiety.

A polypeptide probe may be a protein that may detect an open or relaxed portion of a chromatin. The polypeptide probe may be a modified enzyme that lacks cleavage activity. The modified enzyme may be an enzyme that recognizes DNA or RNA (double-stranded or single-stranded). Examples of modified enzymes may be obtained from oxidoreductases, transferases, hydrolases, lyases, isomerases, or ligases. A modified enzyme may be an endonuclease (such as a deactivated restriction endonuclease such as the TALEN or CRISPR probes described herein).

A polypeptide probe may be an antibody or binding fragment thereof. The antibody or binding fragment thereof may be a protein interacting partner of a product of a regulatory element. The antibody or binding fragment thereof may comprise a humanized antibody or binding fragment thereof, murine antibody or binding fragment thereof, chimeric antibody or binding fragment thereof, monoclonal antibody or binding fragment thereof, monovalent Fab′, divalent Fab2, F (ab)′3 fragments, single-chain variable fragment (scFv), bis-scFv, (scFv) 2, diabody, minibody, nanobody, triabody, tetrabody, disulfide stabilized Fv protein (dsFv), single-domain antibody (sdAb), Ig NAR, camelid antibody or binding fragment thereof, or a chemically modified derivative thereof. The antibody or binding fragment thereof may further comprise a detectable moiety.

Multiple probes may be used together in a probe set to detect a nucleic acid sequence using Nano-FISH. The probe set may be designed for the detection of the target nucleic acid sequence. For example, the probe set may be optimized for probes based on GC content, 16mer base matches (for determining binding specificity of the probe), and their predicted melting temperature when hybrized. The 16mer base matches may have a total of 24 matches to the 16mer database. In some embodiments, probe sets with greater than 100 16-mer database matches may be discarded.

Exemplary probe nucleotide sequences are shown in TABLE 1 for probe sets for different target sequences. Some exemplary probe sequences may be target sequences located in the GREB 1 promoter of chromosome 2, ER iDHS 1 of chromosome 2, ER iDHS2 of chromosome 2, HBG1up of chromosome 11, HBG2 up of chromosome 11, HS 1 of chromosome 11, HS2 of chromosome 11, HS3 of chromosome 11, HS4 of chromosome 11, HS5 of chromosome 11, HS1 Lflank of chromosome 11, HS1 2 flank of chromosome 11, HS2 3 flank of chromosome 11, HS3 4flank of chromosome 11, HS4 5 flank of chromosome 11, HS5 Rflank of chromosome 11, CCNDl SNP of chromosome 11, CCNDl CTL of chromosome 11, the CCNDl promoter of chromosome 11, Chromosome 18 dead1 of chromosome 18, Chromosome 18 dead2 of chromosome 18, Chromosome dead3 of chromosome 18, CNOT promoter of chromosome 19, CNOT inter1 of chromosome 19, CNOT inter2 of chromosome 19, CNOT inter3 of chromosome 19, TSEN promoter of chromosome 19, KLK2 promoter of chromosome 19, KLK3 promoter of chromosome 19, or KLK eRNA of chromosome 19. GREB 1 is gene that may be induced by estrogen stimulation of MCF-7 breast cancer cells. ER iDHS1 and ER iDHS2 are DHS that may be induced by estrogen stimulation of MCF-7 breast cancer cells. HBG1up and HBG2up are hemoglobin genes expressed in K562 erthyro leukemia cells. HS1, HS2, HS3, HS4, and HS5 are hypersensitive sits in the beta-globin locus control region, and HS1 Lflank, HS2 3flank, HS3 4flank, HS4 5flank, HS5 Rflank are sequences in the intervening regions between HS 1-HS5. CCND SNP is an enhancer for the CCND1 gene, CCND1 CTL is a control region adjacent to the CCND1 SNP, and the CCND1 promoter is the promoter region of the CCND1 gene. Chromosome 18 dead1, Chromosome 18 dead 2, and Chromosome 18 dead3 are non-hypersensitive regions of chromosome 18. The CNOT promoter is the promoter (active region) of CNOT. The TSEN promoter is the promoter (active region) of TSEN. The KLK2 promoter is the promoter KLK2. The KLK3 promoter is the promoter of KLK3. KLK eRNA is an enhancer for the KLK2 gene and/or the KLK3 gene, and which may also enhance RNA. For example, a probe set comprising at least nine different Q570 labeled probes selected from the group consisting of SEQ ID NO: 1-SEQ ID NO: 39 may be used to detect the GREB 1 promoter in chromosome 2. A Q570 labeled probe set comprising probes with SEQ ID NO: 7-SEQ ID NO: 35 may be used to detect the GREB 1 promoter in chromosome 2. A probe set comprising at least nine different Q670 labeled probes selected from the group consisting of SEQ ID NO: 40-SEQ ID NO: 72 may be used to detect the ER iDHS 1 in chromosome 2. A probe set comprising at least nine different Q670 labeled probes selected from the group consisting of SEQ ID NO: 73-SEQ ID NO: 104 may be used to detect the ER iDHS 2 in chromosome 2. A probe set comprising at least nine different Q570 labeled probes selected from the group consisting of SEQ ID NO: 105-SEQ ID NO: 134 may be used to detect the HBGlup in chromosome 11. A probe set comprising at least nine different Q570 labeled probes selected from the group consisting of SEQ ID NO: 135-SEQ ID NO: 164 may be used to detect the HBG2up in chromosome 11. A probe set comprising at least nine different Q570/670 labeled probes selected from the group consisting of SEQ ID NO: 165-SEQ ID NO: 194 may be used to detect HS 1 in chromosome 11. A probe set comprising at least nine different Q570/670 labeled probes selected from the group consisting of SEQ ID NO: 195-SEQ ID NO: 224 may be used to detect HS2 in chromosome 11. A probe set comprising at least nine different Q570/670 labeled probes selected from the group consisting of SEQ ID NO: 225-SEQ ID NO: 254 may be used to detect HS3 in chromosome 11. A probe set comprising at least nine different Q670 labeled probes selected from the group consisting of SEQ ID NO: 255-SEQ ID NO: 298 may be used to detect HS4 in chromosome 11. A probe set comprising at least nine different Q570/670 labeled probes selected from the group consisting of SEQ ID NO: 299-SEQ ID NO: 340 may be used to detect HS5 in chromosome 11. A probe set comprising at least nine different Q670 labeled probes selected from the group consisting of SEQ ID NO: 341-SEQ ID NO: 370 may be used to detect HS 1 Lflank in chromosome 11. A probe set comprising at least nine different Q570 labeled probes selected from the group consisting of SEQ ID NO: 371-SEQ ID NO: 400 may be used to detect HS 1 2flank in chromosome 11. A probe set comprising at least nine different Q670 labeled probes selected from the group consisting of SEQ ID NO: 401-SEQ ID NO: 430 may be used to detect HS2 3flank in chromosome 11. A probe set comprising at least nine different Q570 labeled probes selected from the group consisting of SEQ ID NO: 431-SEQ ID NO: 460 may be used to detect HS3 4flank in chromosome 11. A probe set comprising at least nine different Q670 labeled probes selected from the group consisting of SEQ ID NO: 461-SEQ ID NO: 484 may be used to detect HS4 5flank in chromosome 11. A probe set comprising at least nine different Q570 labeled probes selected from the group consisting of SEQ ID NO: 485-SEQ ID NO: 514 may be used to detect HS5 Rflank in chromosome 11. A probe set comprising at least nine different Q570 labeled probes selected from the group consisting of SEQ ID NO: 515-SEQ ID NO: 544 may be used to detect CCND1 SNP in chromosome 11. A probe set comprising at least nine different Q670 labeled probes selected from the group consisting of SEQ ID NO: 545-SEQ ID NO: 564 may be used to detect CCND1 CTL in chromosome 11. A probe set comprising at least nine different Q670 labeled probes selected from the group consisting of SEQ ID NO: 565-SEQ ID NO: 598 may be used to detect the CCND1 promoter in chromosome 11. A probe set comprising at least nine different Q670 labeled probes selected from the group consisting of SEQ ID NO: 599-SEQ ID NO: 628 may be used to detect Chromosome 18 dead1 in chromosome 18. A probe set comprising at least nine different Q670 labeled probes selected from the group consisting of SEQ ID NO: 629-SEQ ID NO: 658 may be used to detect Chromosome 18 dead2 in chromosome 18. A probe set comprising at least nine different Q670 labeled probes selected from the group consisting of SEQ ID NO: 659-SEQ ID NO: 688 may be used to detect Chromosome 18 dead3 in chromosome 18. A probe set comprising at least nine different Q670 labeled probes selected from the group consisting of SEQ ID NO: 689-SEQ ID NO: 718 may be used to detect the CNOT3 promoter in chromosome 19. A probe set comprising at least nine different Q670 labeled probes selected from the group consisting of SEQ ID NO: 719-SEQ ID NO: 748 may be used to detect the TSEN34 promoter in chromosome 19. A probe set comprising at least nine different Q670 labeled probes selected from the group consisting of SEQ ID NO: 749-SEQ ID NO: 778 may be used to detect CNOT3 inter1 in chromosome 19. A probe set comprising at least nine different Q670 labeled probes selected from the group consisting of SEQ ID NO: 779-SEQ ID NO: 808 may be used to detect CNOT3 inter2 in chromosome 19. A probe set comprising at least nine different Q670 labeled probes selected from the group consisting of SEQ ID NO: 809 SEQ ID NO: 838 may be used to detect CNOT3 inter3 in chromosome 19. A probe set comprising at least nine different Q570 labeled probes selected from the group consisting of SEQ ID NO: 839-SEQ ID NO: 868 may be used to detect the KLK2 promoter in chromosome 19. A probe set comprising at least nine different Q570 labeled probes selected from the group consisting of SEQ ID NO: 869-SEQ ID NO: 898 may be used to detect the KLK3 promoter in chromosome 19. A probe set comprising at least nine different Q670 labeled probes selected from the group consisting of SEQ ID NO: 899-SEQ ID NO: 935 may be used to detect KLK eRNA in chromosome 19.

### Detectable Moieties

A detecting agent may comprise a detectable moiety. A detectable moiety may be a small molecule (such as a dye) or a macromolecule. A macromolecule may include polypeptides (such as proteins and/or protein fragments), nucleic acids, carbohydrates, lipids, macrocyles, polyphenols, and/or endogenous macromolecule complexes. A detectable moiety may be a small molecule. A detectable moiety may be a macromolecule.

A detectable moiety may include a moiety that is detectable by a colorimetric method or a fluorescent method. For example, a colorimetric method may be an assay which utilizes reagents that undergo a measurable color change in the presence of an analyte (such as an enzyme, an antibody, a compound, a hormone). Exemplary colorimetric method may include enzyme-mediated detection method such as tyramide signal amplification (TSA) which utilizes horseradish peroxidase (HRP) to generate a signal when digested by tyramide substrate and 3,3′,5,5′-Tetramethylbenzidine (TMB) which generates a blue color upon oxidation to 3,3′5,5′-tetramethylbenzidine diamine in the presence of a peroxidase enzyme such as HRP. A detectable moiety described herein may include a moiety that is detectable by a colorimetric method.

A detectable moiety may also include a moiety that is detectable by a fluorescent method. Sometimes, the detectable moiety may be a fluorescent moiety. A fluorescent moiety may be a small molecule (such as a dye) or a fluorescently labeled macromolecule. A fluorescently labeled macromolecule may include a fluorescently labeled polypeptide (such as a labeled protein and/or a protein fragment), a fluorescently labeled nucleic acid molecule, a fluorescently labeled carbohydrate, a fluorescently labeled lipid, a fluorescently labeled macrocyle, a fluorescently labeled polyphenol, and/or a fluorescently labeled endogenous macromolecule complex (such as a primary antibody-secondary antibody complex).

A fluorescent small molecule may comprise rhodamine, rhodol, fluorescein, thiofluorescein, aminofluorescein, carboxyfluorescein, chlorofluorescein, methylfluorescein, sulfofluorescein, aminorhodol, carboxyrhodol, chlororhodol, methylrhodol, sulforhodol; aminorhodamine, carboxyrhodamine, chlororhodamine, methylrhodamine, sulforhodamine, thiorhodamine, cyanine, indocarbocyanine, oxacarbocyanine, thiacarbocyanine, merocyanine, cyanine 2, cyanine 3, cyanine 3.5, cyanine 5, cyanine 5.5, cyanine 7, oxadiazole derivatives, pyridyloxazole, nitrobenzoxadiazole, benzoxadiazole, pyren derivatives, cascade blue, oxazine derivatives, Nile red, Nile blue, cresyl violet, oxazine 170, acridine derivatives, proflavin, acridine orange, acridine yellow, arylmethine derivatives, auramine, crystal violet, malachite green, tetrapyrrole derivatives, porphin, phtalocyanine, bilirubin 1-dimethylaminonaphthyl-5-sulfonate, 1-anilino-8-naphthalene sulfonate, 2-p-touidinyl-6-naphthalene sulfonate, 3-phenyl-7-isocyanatocoumarin, N-(p-(2-benzoxazolyl)phenyl) maleimide, stilbenes, pyrenes, 6-FAM (Fluorescein), 6-FAM (NHS Ester), 5 (6)-FAM, 5-FAM, Fluorescein dT, 5-TAMRA-cadavarine, 2-aminoacridone, HEX, JOE (NHS Ester), MAX, TET, ROX, TAMRA, TARMA™ (NHS Ester), TEX 615, ATTO™ 488, ATTO™ 532, ATTO™ 550, ATTO™ 565, ATTO™ Rho101, ATTO™ 590, ATTO™ 633, ATTO™ 647N, TYE™ 563, TYE™ 665, or TYE™ 705.

A fluorescent moiety may comprise Cy3, Cy5, Cy5.5, Cy7, Q570, Alexa488, Alexa555, Alexa594, Alexa647, Alexa680, Alexa 750, Alexa 790, Atto488, Atto532, Atto647N, TexasRed, CF610, Propidium iodide, Q670, IRDye700, IRDye800, Indocyanine green, Pacific Blue dye, Pacific Green dye, or Pacific Orange dye.

A fluorescent moiety may comprise a quantum dot (QD). Quantum dots may be a nanoscale seminconducting photoluminescent material, for example, as described in Alivisatos A. P., “Semiconductor clusters, nanocrystals, and quantum dots,” Science 271 (5251): 933-937 (1996).

Exemplary QDs may include, but are not limited to, CdS quantum dots, CdSe quantum dots, CdSe/CdS core/shell quantum dots, CdSe/ZnS core/shell quantum dots, CdTe quantum dots, PbS quantum dots, and/or PbSe quantum dots. As used herein, CdSe/ZnS may mean that a ZnS shell is coated on a CdSe core surface (a “core-shell” quantum dot). The shell materials of core-shell QDs may have a higher bandgap and passivate the core QDs surfaces, resulting in higher quantum yield and higher stability and wider applications than core QDs.

QDs may absorb a wide spectrum of light, and may be physically tuned with emission bandwidths in various wavelengths. See, e.g., Badolato, et al., Science 208:1158-61 (2005). For example, the emission bandwidth may be in the visible spectrum (from about 350 to about 750 nm), the ultraviolet-visible spectrum (from about 100 nm to about 750 nm), or in the near-infrared spectrum (from about 750 nm to about 2500 nm). QDs that emit energy in the visible range may include, but are not limited to, CdS, CdSe, CdTe, ZnSe, ZnTe, GaP, and GaAs. QDs that emit energy in the blue to near-ultraviolet range include, but are not limited to, ZnS and GaN. QDs that emit energy in the near-infrared range include, but are not limited to, InP, InAs, InSb, PbS, and PbSe.

The radius of a QD may be modulated to manipulate the emission bandwidth. For example, a radius of between about 5 and about 6 nm QD may emit wavelengths resulting in emission colors such as orange or red. A radius of between about 2 and about 3 nm may emit wavelengths resulting in emission colors such as blue or green.

A QD may further form a QD microstructure, which encompasses one or more layers of QD. For example, each quantum dot containing layer may comprise a single type of quantum dot of a specific emission color. For example, each layer may be made of any material suitable for use that (a) allows excitation light to reach the quantum dot and allows fluorescence generated from the quantum dot to pass through the layer(s) for detection and (b) may be combined with a quantum dot to form a layer. Examples of materials that may be used to form layers containing quantum dots include, but are not limited to, inorganic, organic, or polymeric material, each with or without biodegradable properties, and combinations thereof. The layers may comprise silica-based compounds or polymers. Exemplary silica-based layers may include, but are not limited to, those comprising tetramethoxy silane or tetraethylorthosilicate. Exemplary polymer layers may include, but are not limited to, those comprising polystyrene, poly (methyl methacrylate), polyhydroxyalkanoate, polylactide, or co-polymers thereof.

The quantum dot further may comprise a spacer layer which serves as a barrier to prevent interactions between different QD layers, and may be made of any material suitable for use that (a) allows excitation light to reach the quantum dots in the quantum dot containing layer(s) below it and allows fluorescence generated from those quantum dots to pass through it and (b) may segregate the quantum dots in one layer from those in other layers. Examples of materials that may be used to form spacer layers are the same as for the quantum dot containing layers.

The materials used for the quantum dot containing and spacer layers may be the same or different. The same material may be used in the quantum dot containing layers and the spacer layers.

The quantum dot containing layers and the spacer layers within a given QD molecule may be any thickness and may be varied. For example, thicker QD-containing layers may allow for the loading of increased QDs in the shell, resulting in greater fluorescence intensity for that layer than for a thinner layer containing the same concentration of QDs. Thus, varying layer thickness may facilitate preparing QD-containing layer of various intensities, thereby generating spectrally distinct QD bar codes. In various instances, the QD-containing layers may be between 5 nm and 500 nm; 10 nm and 500 nm; 5 nm and 100 nm, and 10 nm and 100 nm. Those of skill in the art will understand that other methods for varying intensity also exist, for example, modifying concentrations of the same QD in one microstructure with a first unique barcode compared to a second QD microstructure with a different fluorescent barcode. The ability to vary the intensities for the same QD color allows for an increased number of distinct and distinguishable microstructures (e.g., spectrally distinct barcodes). The spacer layers may be greater than 10 nm, up to approximately 5 μm thick; the spacer layers may be greater than 10 nm, up to approximately 500 nm thick; the space layers may be greater than 10 nm, up to approximately 100 nm thick.

The quantum dot-containing and spacer layers may be arranged in any order. Examples include, but are not limited to, alternating QD-containing layers and spacer layers, or quantum dot containing layers separated by more than one spacer layer. Thus, a “spacer layer” may comprise a single layer, or may comprise two or more such spacer layers.

The QD microstructure may comprise any number of quantum dot containing layers suitable for use with the microstructure. For example, a microstructure described herein may comprise 2, 3, 4, 5, 6, 7, 8, 9, 10, or more quantum dot-containing layers and an appropriate number of spacer layers based on the number of quantum dot-containing layers. Further, the number of quantum dot containing layers in a given microstructure may range from 1 to “m,” where “m” is the number of quantum dots that may be used.

A defined intensity level may refer to a known amount of quantum dots in each quantum dot containing layer, resulting in a known amount of fluorescent intensity generated from the QD containing layer upon appropriate stimulation. Since each QD containing layer has a defined intensity level, each microstructure may possess a defined ratio of fluorescence intensities generated from the various QD-containing layers upon stimulation. This defined ratio is referred to herein as a barcode. Thus, each type of microstructure with the same QD layers possesses a similar barcode that may be distinguished from microstructures with different QD layers.

Thus, each quantum dot containing layer may comprise a single type of quantum dot of a specific emission color and the layer is produced to possess a defined intensity level, based on the concentration of the QD in the layer. By varying the intensity levels of QDs (“n”) in different microstructures and using a variety of different quantum dots (“m”), the number of different unique barcodes (and thus the number of different unique microstructure populations that may be produced) is approximated by the equation, (n^{m}−1) unique codes. This may provide the ability to generate a large number of different populations of microstructures each with its own unique barcode.

A set of QD-labeled probes may further generate a spectrally distinct barcode. For example, each probe with the set of QD-labeled probes may comprise a QD with a distinct excitation wavelength and the combination of the set may generate a distinct barcode. A set of spectrally distinct QD-labeled probes may be utilized to detect a regulatory element. As such, when detecting two or more regulatory elements, each regulatory element may be spectrally barcoded.

A quantum dot provided herein may include QDot525, QDot 545, QDot 565, QDot 585, QDot 605, or QDot 655. A probe described herein may comprise a quantum dot. A probe described herein may comprise QDot525, QDot 545, QDot 565, QDot 585, QDot 605, or QDot 655. A probe described herein may comprise QDot525. A probe described herein may comprise QDot 545. A probe described herein may comprise QDot 565. A probe described herein may comprise QDot 585. A probe described herein may comprise QDot 605. A probe described herein may comprise QDot 655.

A quantum dot may comprise a quantum dot as described in Han et al., “Quantum-dot-tagged microbeads for multiplexed optical coding of biomolecules,” Nat. Biotechnol. 19:631-635 (2001); Gao X., “QD barcodes for biosensing and detection,” Conf Proc IEEE Eng Med Biol Soc 2009:6372-6373 (2009); and Zrazhevskiy, et al., “Multicolor multicycle molecular profiling with quantum dots for single-cell analysis,” Nat Protoc 8:1852-1869 (2013).

A QD may further comprise a functional group or attachment moiety. One example of such a QD that has a functional group or attachment moiety is a QD with a carboxylic acid terminated surface, such as those commercially available though, for example, Quantum Dot, Inc., Hayward, CA.

### Conjugating Moiety

The probe may include a conjugating moiety. The conjugation moiety may be attached at the 5′ terminus, the 3′ terminus, or at an internal site. The conjugating moiety may be a nucleotide analog (such as bromodeoxyuridine). The conjugating moiety may be a conjugating functional group. The conjugating functional group may be an azido group or an alkyne group. The probe may further be derivatized through a chemical reaction such as click chemistry. The click chemistry may be a copper (I)-catalyzed [3+2]-Huisgen 1,3-dipolar cyclo-addition of alkynes and azides leading to 1,2,3-triazoles. The click chemistry may be a copper free variant of the above reaction.

The conjugating moiety may comprise a hapten group. A hapten group may include digoxigenin, 2,4-dinitrophenyl, biotin, avidin, or are selected from azoles, nitroaryl compounds, benzofurazans, triterpenes, ureas, thioureas, rotenones, oxazoles, thiazoles, coumarins, cyclolignans, heterobiaryl compounds, azoaryl compounds or benzodiazepines. A hapten group may include biotin.

The probe comprising the conjugating moiety may further be linked to a second probe (such as a nucleic acid probe or a polypeptide probe), a fluorescent moiety (such as a dye such as a quantum dot), a target nucleic acid, or a conjugating partner such as a polymer (such as PEG), a macromolecule (such as a carbohydrate, a lipid, a polypeptide), and the like.

### Samples

A sample described herein may be a fresh sample or a fixed sample. The sample may be a fresh sample. The sample may be a fixed sample. The sample may be a live sample. The sample may be subjected to a denaturing condition. The sample may be cryopreserved.

The sample may be a cell sample. The cell sample may be obtained from the cells or tissue of an animal. The animal cell may comprise a cell from an invertebrate, fish, amphibian, reptile, or mammal. The mammalian cell may be obtained from a primate, ape, equine, bovine, porcine, canine, feline, or rodent. The mammal may be a primate, ape, dog, cat, rabbit, ferret, or the like. The rodent may be a mouse, rat, hamster, gerbil, hamster, chinchilla, or guinea pig. The bird cell may be from a canary, parakeet, or parrot. The reptile cell may be from a turtle, lizard, or snake. The fish cell may be from a tropical fish. For example, the fish cell may be from a zebrafish (such as Danio rerio). The amphibian cell may be from a frog. An invertebrate cell may be from an insect, arthropod, marine invertebrate, or worm. The worm cell may be from a nematode (such as Caenorhabditis elegans). The arthropod cell may be from a tarantula or hermit crab.

The cell sample may be obtained from a mammalian cell. For example, the mammalian cell may be an epithelial cell, connective tissue cell, hormone secreting cell, a nerve cell, a skeletal muscle cell, a blood cell, an immune system cell, or a stem cell. A cell may be a fresh cell, live cell, fixed cell, intact cell, or cell lysate.

Cell samples may be cells derived from a cell line. Exemplary cell lines include, but are not limited to, 293A cell line, 293FT cell line, 293F cell line, 293 H cell line, HEK 293 cell line, CHO DG44 cell line, CHO-S cell line, CHO-K1 cell line, Expi293F™ cell line, Flp-In™ T-REX™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cell line, FreeStyle™ CHO-S cell line, GripTite™ 293 MSR cell line, GS-CHO cell line, HepaRG™ cell line, T-REX™ Jurkat cell line, Per.C6 cell line, T-REX™-293 cell line, T-REx™-CHO cell line, T-REX™-HeLa cell line, NC-HIMT cell line, and PC12 cell line.

The cell sample may be obtained from cells of a primate. The primate may be a human, or a non-human primate. The cell sample may be obtained from a human. For example, the cell sample may comprise cells obtained from blood, urine, stool, saliva, lymph fluid, cerebrospinal fluid, synovial fluid, cystic fluid, ascites, pleural effusion, amniotic fluid, chorionic villus sample, vaginal fluid, interstitial fluid, buccal swab sample, sputum, bronchial lavage, Pap smear sample, or ocular fluid. The cell sample may comprise cells obtained from a blood sample, an aspirate sample, or a smear sample.

The cell sample may be a circulating tumor cell sample. A circulating tumor cell sample may comprise lymphoma cells, fetal cells, apoptotic cells, epithelia cells, endothelial cells, stem cells, progenitor cells, mesenchymal cells, osteoblast cells, osteocytes, hematopoietic stem cells, foam cells, adipose cells, transcervical cells, circulating cardiocytes, circulating fibrocytes, circulating cancer stem cells, circulating myocytes, circulating cells from a kidney, circulating cells from a gastrointestinal tract, circulating cells from a lung, circulating cells from reproductive organs, circulating cells from a central nervous system, circulating hepatic cells, circulating cells from a spleen, circulating cells from a thymus, circulating cells from a thyroid, circulating cells from an endocrine gland, circulating cells from a parathyroid, circulating cells from a pituitary, circulating cells from an adrenal gland, circulating cells from islets of Langerhans, circulating cells from a pancreas, circulating cells from a hypothalamus, circulating cells from prostate tissues, circulating cells from breast tissues, circulating cells from circulating retinal cells, circulating ophthalmic cells, circulating auditory cells, circulating epidermal cells, circulating cells from the urinary tract, or combinations thereof.

A cell sample may be a peripheral blood mononuclear cell sample.

A cell sample may comprise cancerous cells. The cancerous cells may form a cancer which may be a solid tumor or a hematologic malignancy. The cancerous cell sample may comprise cells obtained from a solid tumor. The solid tumor may include a sarcoma or a carcinoma. Exemplary sarcoma cell sample may include, but are not limited to, cell sample obtained from alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, or telangiectatic osteosarcoma.

Exemplary carcinoma cell samples may include, but are not limited to, cell samples obtained from an anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

The cancerous cell sample may comprise cells obtained from a hematologic malignancy. Hematologic malignancy may comprise a leukemia, a lymphoma, a myeloma, a non-Hodgkin's lymphoma, or a Hodgkin's lymphoma. The hematologic malignancy may be a T-cell based hematologic malignancy. The hematologic malignancy may be a B-cell based hematologic malignancy. Exemplary B-cell based hematologic malignancy may include, but are not limited to, chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. Exemplary T-cell based hematologic malignancy may include, but are not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

A cell sample described herein may comprise a tumor cell line sample. Exemplary tumor cell line sample may include, but are not limited to, cell samples from tumor cell lines such as 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

A cell sample may comprise cells obtained from a biopsy sample, necropsy sample, or autopsy sample.

The cell samples (such as a biopsy sample) may be obtained from an individual by any suitable means of obtaining the sample using well-known and routine clinical methods. Procedures for obtaining tissue samples from an individual are well known. For example, procedures for drawing and processing tissue sample such as from a needle aspiration biopsy are well-known and may be employed to obtain a sample for use in the methods provided.

Typically, for collection of such a tissue sample, a thin hollow needle is inserted into a mass such as a tumor mass for sampling of cells that, after being stained, will be examined under a microscope.

A cell may be a eukaryotic cell. A cell may be a yeast cell. A cell may be a plant cell. A cell may be obtained from an agricultural plant.

### Detection of a Target Nucleic Acid Sequence

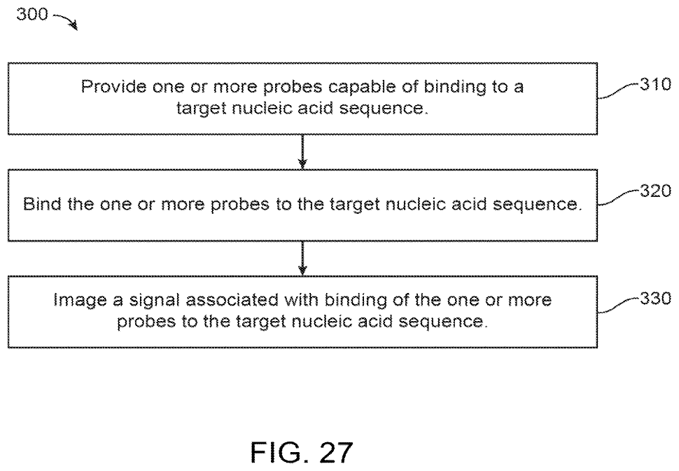

FIG. 26 shows a flowchart for a method 200 of detecting a target nucleic acid sequence. The method may comprise an operation 210 of providing one or more probes capable of binding to a target nucleic acid sequence, as described herein. The method may comprise an operation 220 of binding the one or more probes to the target nucleic acid sequence, as described herein. The method may comprise an operation 230 of detecting a signal associated with binding of the one or more probes to the target nucleic acid sequence, as described herein.

The target nucleic acid sequence may be detected in an intact cell. The target nucleic acid sequence may be detected in a fixed cell. The target nucleic sequence may be detected in a lysate or chromatin spread.

A probe may be used to detect a nucleic acid sequence in a sample. For example, a probe comprising a probe sequence capable of binding a nucleic acid sequence (such as a target nucleic acid sequence) and a detectable label (such as a detectable agent) may be used to detect the nucleic acid sequence. A method for detecting a nucleic acid sequence may comprise contacting a nucleic acid sequence with a probe comprising a probe sequence configured to bind at least a portion of the nucleic acid sequence and detecting the probe (such as detecting the detectable label of the probe). The detection of a nucleic acid sequence may comprise binding the probe to the nucleic acid sequence. For example, the detection of a nucleic acid sequence may comprise binding the probe sequence, such as the sequence of an oligonucleotide probe, to a target nucleic acid sequence. In some cases, the detection of a nucleic acid sequence may comprise hybridizing the probe sequence (such as the nucleic acid binding region) of a nucleic acid probe to a target nucleic acid sequence.

A nucleic acid sequence may be contacted with a plurality of probes. A nucleic acid sequence may be contacted with a number of probes ranging from about 1 to about 10⁸ probes, from about 2 to about 10⁷ probes, from about 10 to about 10⁶ probes, from about 100 to about 10⁵ probes, from about 1,000 to about 10⁴ probes, from about 1,000 to about 5,000 probes, from about 1,000 probes to about 50,000 probes, from about 1,000 to about 10⁵ probes, from about 1,000 to about 500,000 probes, from about 1,000 probes to about 10⁶ probes, from about 1,000 probes to about 50 million probes, or from about 1,000 probes to about 10⁸ probes. The probes of the plurality of probes may be the same. A plurality of probes may have sequences such that the probes are tiled across the nucleic acid sequence. Each probe can bind to a target nucleic acid sequence along the nucleic acid sequence. The probes of a plurality may be different. A first probe of the plurality of probes may be different than a second probe of the plurality of probes. The plurality of probes may bind to the nucleic acid sequence with from 0 to 10 nucleotides separating each probe.

A nucleic acid sequence may be washed after it has been contacted with a probe. Washing a nucleic acid sequence after it has been contacted with a probe may reduce background signal for detection of the detectable label of the probe.

A nucleic acid sequence (such as a target nucleic acid sequence) may be contacted by a plurality of probes. A nucleic acid sequence may be contacted with a plurality of types of probes. That is, a method of detection of a nucleic acid sequence (such as a target nucleic acid sequence) may comprise contacting the target nucleic acid sequence with a plurality of sets of probes (such as a plurality of types of probes). A first probe set (such as a first type of probe) may be different from a second probe set (such a second type of probe) in that the first probe type comprises a first probe sequence which is different than the probe sequence of the second probe type. The probe sequence of a first type of probe may be the same as the probe sequence of a second type of probe. A first probe set may comprise a first detectable label and a first probe sequence and a second probe set may comprise a second detectable label and a second probe sequence, wherein the first and second probe sequences are the same and the first and second detectable labels are different. The first and second probe sequences may be different and the first and second detectable labels of a first and second probe set may be the same. The first and second probe sequences of a first and second probe set may be different and the first and second detectable labels of a first and second probe set may be different. A method of detecting a nucleic acid sequence may comprise contacting a nucleic acid sequence with 1, 2, 3, 4, 5, 6, 7, 8, 9, 10, 11, 12, 13, 14, 15, 16, 17, 18, 19, or 20 types of probes.

A first probe sequence may be configured to specifically recognize (such as to bind to or to hybridize with) a first nucleic acid sequence (such as a first target nucleic acid sequence). A second probe sequence may be configured to specifically recognize (such as to bind to or to hybridize with) a second nucleic acid sequence (such as a second target nucleic acid sequence).

A detectable label may be detected with a detector. A detector may detect the signal intensity of the detectable label. A detector may spatially distinguish between two detectable labels. A detector may also distinguish between a first and second detectable label based on the spectral pattern produced by the first and second detectable labels, wherein the first and second detectable label do not produce an identical spectral intensity pattern. For example, a detector may distinguish between a first and second detectable signal, wherein the wavelength of the signal produced by the first detectable label is not the same as the wavelength of the signal produced by the second detectable label. A detector may resolve (such as by spatially distinguishing or spectrally distinguishing) a first and second detectable label that are less than 1 kb apart, less than kb apart, less than kb apart, less than 2 kb apart, less than 2.5 kb apart, less than 3 kb apart, less than 3.5 kb apart, less than 4 kb apart, less than 4.5 kb apart, less than 5 kb apart, less than 5.5 kb apart, less than 6 kb apart, less than 6.5 kb apart, less than 7 kb apart, less than 7.5 kb apart, less than 8 kb apart, less than 8.5 kb apart, less than 9 kb apart, less than 9.5 kb apart, less than 10 kb apart, less than 10.5 kb apart, less than 11 kb apart, less than 11.5 kb apart, less than 12 kb apart, less than 20 kb apart, less than 50 kb apart, or less than 100 kb apart. The detectable label of the probe may be detected optically. For example, a detectable label of a probe may be detected by light microscopy, fluorescence microscopy, or chromatography. Detection of the detectable label of a probe may comprise stimulating the probe or a portion thereof (such as the detectable label) with a source of radiation (such as a light source, such as a laser). Detection of the detectable label of a probe may also comprise an enzymatic reaction.

Detection of the target nucleic acid sequence may be within a period of not more than 48 hours, not more than 36 hours, not more than 24 hours, not more than 23 hours, not more than 22 hours, not more than 21 hours, not more than 20 hours, not more than 19 hours, not more than 18 hours, not more than 17 hours, 1 not more than 6 hours, not more than 15 hours, not more than 14 hours, not more than 13 hours, or not more than 12 hours.

Determining the presence of a genetic modification in a cell using the Nano-FISH method described herein may be useful is assessing the phenotype of the cell resulting from the genetic modification. A method for assessing a phenotype of an intact genetically modified cell may comprise: a) providing the intact genetically modified cell comprising a target nucleic acid sequence less than 2.5 kilobases in length; b) contacting the intact genetically modified cell with a first plurality of probes, wherein each probe comprises a first detectable label and a probe sequence that binds to a portion of the target nucleic acid sequence; c) detecting a presence of the first detectable label in the intact cell, wherein the presence of the first detectable label indicates the presence of the target nucleic acid sequence; d) determining a phenotype of the intact genetically modified cell; and e) correlating the phenotype of the intact genetically modified cell with the presence of the target nucleic acid sequence. The method may further comprise determining a number or location of genetic modifications in the intact genetically modified cell. The method may further comprise f) selecting a first intact genetically modified cell comprising a phenotype of interest; g) determining a set of conditions used for a genetic modification of the first intact genetically modified cell; and h) preparing a second genetically modified cell using the set of conditions for genetic modification. The intact genetically modified cell may be a eukaryotic cell that was genetically modified. The intact genetically modified cell may be a bacteria cell that was genetically modified. The intact genetically modified cell may be a mammalian cell that was genetically modified. The intact genetically modified cell may be any cell as described herein that was genetically modified. The phenotype may be a product expressed as a result of the genetic modification of the cell. The phenotype may be an increased level or decreased level of the product expressed as a result of the genetic modification of the cell. The phenotype may be an increased quality of the product expressed as a result of the genetic modification of the cell. The expressed product may be protein, such as an enzyme. The expressed product may be a transgene protein, RNA, or a secondary product of the genetic modification. For example, if an enzyme is produced as a result of the genetic modification of the cell, a secondary product of the genetic modification is a product of the enzyme.

Determining the number of target nucleic acid sequences in a cell may be useful in determining the phenotype of the cell. Cells with a specific number of target nucleic acid sequences may be tested for increased cellular activity, decreased cellular activity, or toxicity. Increased cellular activity may be increased expression of a protein or a cellular product. Decreased cellular activity may be decreased expression of a protein or a cellular product. Toxicity may be a result of cellular activity that may be too high or too low, resulting in cell death. For example, the contacting a sample of cells with a probe configured to bind to a particular target nucleic acid sequence and then determining the number of target nucleic acid sequences in the cell may be an expedient means of determining whether the number of target nucleic acid sequences may be affecting the cell phenotype or function.

A method of detecting or determining the presence of a nucleic acid sequence may comprise determining the number of probes associated with the nucleic acid sequence. A method of detecting or determining the presence of a nucleic acid sequence may comprise determining the number of probes hybridized to the nucleic acid sequence.

It may also be possible to determine the quantity of target nucleic acid sequences in this manner. Quantification of target nucleic acid sequences in a sample using the methods, compositions, and systems described herein may be useful in determining the number of repeated sequences in a nucleic acid of a sample.

Determining the presence, absence, identity, spatial position or sequence position of a target nucleic acid sequence in a sample may be useful in determining a condition of a patient. For example, contacting a patient sample with a probe configured to bind to a particular nucleic acid sequence may be an expedient means of determining whether the patient has the nucleic acid sequence. Similarly, contacting a patient sample with a plurality of types of probes, each configured to bind to a different nucleic acid sequence, may be an expedient means of screening patients for various genetic or acquired conditions, such as inherited mutations.

### Determination of the Spatial Position of a Nucleic Acid Sequence

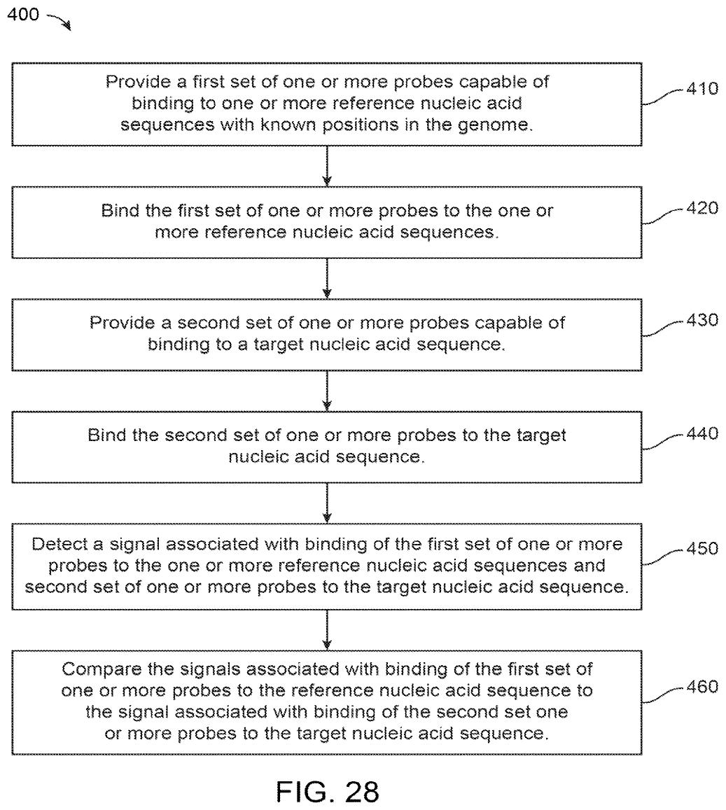

FIG. 27 shows a flowchart for a method 300 of determining the spatial position of a nucleic acid sequence. The method may comprise an operation 310 of providing one or more probes capable of binding to a target nucleic acid sequence, as described herein. The method may comprise an operation 320 of binding the one or more probes to the target nucleic acid sequence, as described herein. The method may comprise an operation 330 of imaging a signal associated with binding of the one or more probes to the target nucleic acid sequence, as described herein.

A method of detecting or determining the presence of a nucleic acid sequence may comprise determining the spatial position of a nucleic acid sequence (such as a target nucleic acid sequence). Determining the spatial position of a nucleic acid sequence may comprise contacting a nucleic acid sequence with a probe, which may comprise a detectable label and a probe sequence configured to bind to the nucleic acid sequence, and detecting the detectable label of the probe.

The spatial position of the nucleic acid sequence may be determined relative to features of the sample (such as features of a cell), structures of the sample (such structures or organelles of the cell), or other nucleic acids by using the same or a different imaging modality to detect the reference features, structures, or nucleic acids. For instance, the spatial position of a nucleic acid sequence in a cell relative to the nucleus of a cell by using a plurality of antibodies with a detectable label to counter-label structures of the cell, such as the cell membrane. A cell line expressing a detectable label (such as a fusion protein with a structural protein expressed by the cell) may be used to determine spatial position of a nucleic acid sequence in a cell.

Data collected from detection of all or a portion of the detectable labels in a sample may be used to form one or more two-dimensional images or a three-dimensional rendering or to make calculations determining or estimating the spatial position of the target nucleic acid sequence.

A first probe comprising a first detectable label and a first probe sequence configured to bind to a nucleic acid sequence (such as a target nucleic acid sequence) may be used as a reference position for a second probe comprising a second detectable label and a second probe sequence configured to bind to a second nucleic acid sequence (such as a second target nucleic acid sequence). For example, a first probe specific to a first target nucleic acid sequence of a nucleic acid with a known or anchored position on the nucleic acid may be used as a reference to determine the spatial position of a second target nucleic acid sequence bound by a second probe prior to or during imaging.

### Detection of the Sequence Position of a Nucleic Acid Sequence

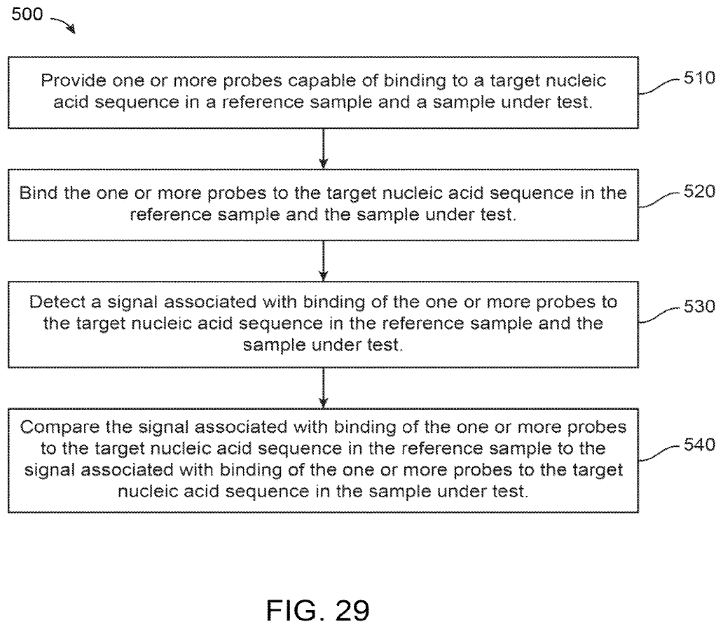

FIG. 28 shows a flowchart for a method 400 of detecting the sequence position of a nucleic acid sequence. The method may comprise an operation 410 of providing a first set of one or more probes capable of binding to one or more reference nucleic acid sequences with known positions in the genome, as described herein. The method may comprise an operation 420 of binding the first set of one or more probes to the one or more reference nucleic acid sequences, as described herein. The method may comprise an operation 430 of providing a second set of one or more probes capable of binding to a target nucleic acid sequence, as described herein. The method may comprise an operation 440 of binding the second set of one or more probes to the target nucleic acid sequence, as described herein. The method may comprise an operation 450 of detecting a signal associated with binding of the first set of one or more probes to the one or more reference nucleic acid sequences and of the second set of one or more probes to the target nucleic acid sequence, as described herein. The method may comprise an operation 460 of comparing the signals associated with binding of the first set of one or more probes to the reference nucleic acid sequences to the signal associated with binding of the second set of one or more probes to the target nucleic acid sequence.

A method of detecting or determining the presence of a nucleic acid sequence may comprise determining the sequence position of a nucleic acid sequence (such as a target nucleic acid sequence). For example, a probe with a probe sequence configured to recognize a first target sequence with a known position in the sequence of a nucleic acid may be used as reference for calculations or estimations of the sequence position of a second target nucleic acid sequence on the nucleic acid. For example, a first probe having a probe sequence configured to recognize a first target sequence with a first known position in the sequence of a nucleic acid and a second probe having a probe sequence configured to recognize a second target nucleic acid sequence with a second known position in the sequence of the nucleic acid may be used as reference points for a third probe configured to recognize a third target nucleic acid sequence with an unknown position in the nucleic acid. The relative sequence position of the third target nucleic acid sequence may be determined or estimated by comparing it to the positions of the first and second target nucleic acid sequences, as indicated by the signals from the first and second probes.

### Detection of Nucleic Acid Sequences in a Sample Relative to a Control

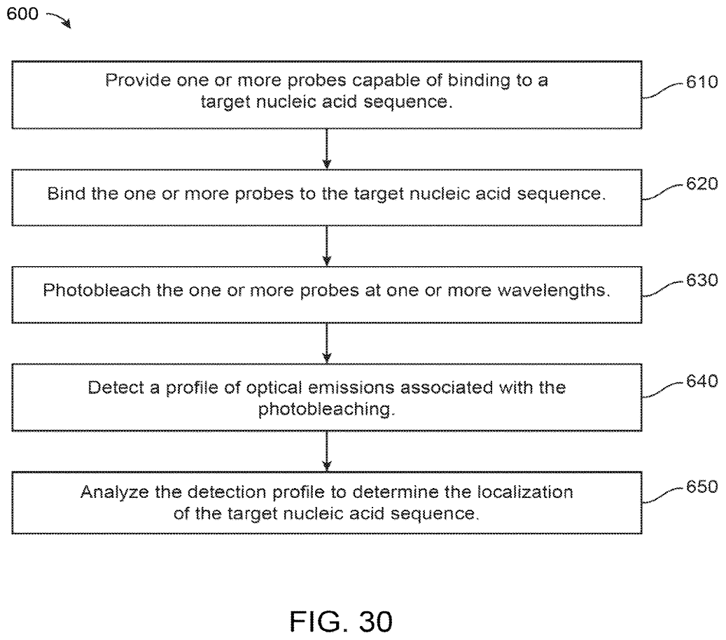

FIG. 29 shows a flowchart for a method 500 of detecting a nucleic acid in a sample relative to a control. The method may comprise an operation 510 of providing a one or more probes capable of binding to a target nucleic acid sequence in a reference sample and a target nucleic acid sequence in a sample under test, as described herein. The method may comprise an operation 520 of binding the one or more probes to the target nucleic acid sequence in the reference sample and the target nucleic acid sequence in the sample under test, as described herein. The method may comprise an operation 530 of detecting a signal associated with binding of the set of one or more probes to the target nucleic acid sequence in the reference sample and the target nucleic acid sequence in the sample being tested, as described herein. The method may comprise an operation 540 of comparing the signal associated with binding of the one or more probes to the target nucleic acid sequence in the reference sample to the signal associated with binding of the one or more probes to the target nucleic acid sequence in the sample under test, as described herein.

Correlation of the Detection of a Target Nucleic Acid Sequence in a Sample with a Target Protein Expression

The detection of a target nucleic acid sequence in a cell may be correlated with a target protein expression in the same cell. The method may comprise providing a one or more probes capable of binding to a target nucleic acid sequence in a sample and a target nucleic acid sequence in a sample being tested, as described herein, and further comprise providing one or more detectable labels to detect the target protein expression. The presence, absence, or quantity of the detected target nucleic acid sequence may be correlated to the presence, absence, or quantity of the target protein expression. This information may be used to further investigate the relationship between the target nucleic acid sequence and the target protein, and/or how different treatments may perturb this correlation.

### Optical Detection of Nucleic Acid Sequences

Described herein is a method of detecting a nucleic acid sequence. The detection may encompass identification of the nucleic acid sequence, determining the presence or absence of the nucleic acid sequence, and/or determining the activity of the nucleic acid sequence. A method of detecting a nucleic acid sequence may include contacting a cell sample with a detection agent, binding the detection agent to the nucleic acid sequence, and analyzing a detection profile from the detection agent to determine the presence, absence, or activity of the nucleic acid sequence.

The method may involve utilizing one or more intrinsic properties associated with a detection agent to aid in detection of the nucleic acid sequence. The intrinsic properties may encompass the size of the detection agent, the intensity of the signal, and the location of the detection agent. The size of the detection agent may include the length of the probe and/or the size of the detectable moiety (such as the size of a fluorescent dye molecule) may modulate the specificity of interaction with a regulatory element. The intensity of the signal from the detection agent may correlate to the sensitivity of detection. For example, a detection agent with a molar extinction coefficient of about 0.5-5×10⁶ M⁻¹ cm⁻¹ may have a higher intensity signal relative to a detection agent with a molar extinction coefficient outside of the 0.5-5×10⁶ M⁻¹ cm⁻¹ range and may have lower attenuation due to scattering and absorption. Further, a detection agent with a longer excited state lifetime and a large Stoke shift (measured by the distance between the excitation and emission peaks) may further improve the sensitivity of detection. The location of the detection agent may, for example, provide the activity state of a nucleic acid sequence. A combination of intrinsic properties of the detection agent may be used to detect a regulatory element of interest.

A detection agent may comprise a detectable moiety that is capable of generating a light, and a probe portion that is capable of hybridizing to a target site on a nucleic acid sequence. As described herein, a detection agent may include a DNA probe portion, an RNA probe portion, a polypeptide probe portion, or a combination thereof. A DNA or RNA probe portion may be between about 10 and about 100 nucleotides in length, between about 15 and about 100 nucleotides in length, between about 20 and about 100 nucleotides in length, between about 20 and about 80 nucleotides in length, between about 20 and about 60 nucleotides in length, between about 25 and about 55 nucleotides in length, between about 30 and about 50 nucleotides in length, between about 15 and about 80 nucleotides in length, between about 15 and about 60 nucleotides in length, between about 20 and about 40 nucleotides in length, or between about 20 and about 30 nucleotides in length. A DNA or RNA probe portion may be about 10, about 15, about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60, about 65, about 70, about 80, about 90, or about 100 nucleotides in length. A DNA or RNA probe portion may be a TALEN probe, ZFN probe, or a CRISPR probe. A DNA or RNA probe portion may be a padlock probe. A polypeptide probe may comprise a DNA-binding protein, a RNA-binding protein, a protein involved in the transcription/translation process or detects the transcription/translation process, a protein that may detect an open or relaxed portion of a chromatin, or a protein interacting partner of a product of a regulatory element (such as an antibody or binding fragment thereof).

In some instances, a detection agent may comprise a DNA or RNA probe portion which may be between about 10 and about 100 nucleotides in length, between about 15 and about 100 nucleotides in length, between about 20 and about 100 nucleotides in length, between about 20 and about 80 nucleotides in length, between about 20 and about 60 nucleotides in length, between about 25 and about 55 nucleotides in length, between about 30 and about 50 nucleotides in length, between about 15 and about 80 nucleotides in length, between about 15 and about 60 nucleotides in length, between about 20 and about 40 nucleotides in length, or between about 20 and about 30 nucleotides in length. A detection agent may comprise a DNA or RNA probe portion which may be about 10, about 15, about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60, about 65, about 70, about 80, about 90, or about 100 nucleotides in length.

A detection agent may comprise a DNA or RNA probe selected from a TALEN probe, a ZFN probe, or a CRISPR probe.

A set of detection agents may be used to detect a nucleic acid sequence. The set of detection agents may comprise about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, or more detection agents. Each of the detection agents within the set of detection agents may recognize and interact with a distinct region of a nucleic acid sequence. About 1, about 2, about 3, about 4, about 5, about 6, about 7, about 8, about 9, about 10, about 11, about 12, about 13, about 14, about 15, about 16, about 17, about 18, about 19, about 20, or more detection agents may be used for detection of a nucleic acid sequence. About 1 or more detection agents may be used for detection of a nucleic acid sequence. About 2 or more detection agents may be used for detection of a nucleic acid sequence. About 3 or more detection agents may be used for detection of a nucleic acid sequence. About 4 or more detection agents may be used for detection of a nucleic acid sequence. About 5 or more detection agents may be used for detection of a nucleic acid sequence. About 6 or more detection agents may be used for detection of a nucleic acid sequence. About 7 or more detection agents may be used for detection of a nucleic acid sequence. About 8 or more detection agents may be used for detection of a nucleic acid sequence. About 9 or more detection agents may be used for detection of a nucleic acid sequence. About 10 or more detection agents may be used for detection of a nucleic acid sequence. About 11 or more detection agents may be used for detection of a nucleic acid sequence. About 12 or more detection agents may be used for detection of a nucleic acid sequence. About 13 or more detection agents may be used for detection of a nucleic acid sequence. About 14 or more detection agents may be used for detection of a nucleic acid sequence. About 15 or more detection agents may be used for detection of a nucleic acid sequence. About 20 or more detection agents may be used for detection of a nucleic acid sequence.

A detection agent may comprise a polypeptide probe selected from a DNA-binding protein, a RNA-binding protein, a protein involved in the transcription/translation process or detects the transcription/translation process, a protein that may detect an open or relaxed portion of a chromatin, or a protein interacting partner of a product of a regulatory element (such as an antibody or binding fragment thereof).

A detectable moiety that is capable of generating a light may be directly conjugated or bound to a probe portion. A detectable moiety may indirectly conjugated or bound to a probe portion by a conjugating moiety. As described herein, a detectable moiety may be a small molecule (such as a dye) which may be directly conjugated or bound to a probe portion. A detectable moiety may be a fluorescently labeled protein or molecule which may be attached to a conjugating moiety (such asa hapten group, an azido group, an alkyne group) of a probe.

A profile or a detection profile or signature may include the signal intensity, signal location, and/or size of the signal of the detection agent. The profile or the detection profile may comprise about 100 image frames, about 500 frames, about 1000 frames, about 2000 frames, about 5000 frames, about 10,000 frames, about 20,000 frames, about 30,000 frames, about 40,000 frames, about 50,000 frames, or more image frames. Analysis of the profile or the detection profile may determine the activity of the regulatory element. The degree of activation may also be determined from the analysis of the profile or detection profile. Analysis of the profile or the detection profile may further determine the optical isolation and localization of the detection agents, which may correlate to the localization of the nucleic acid sequence.

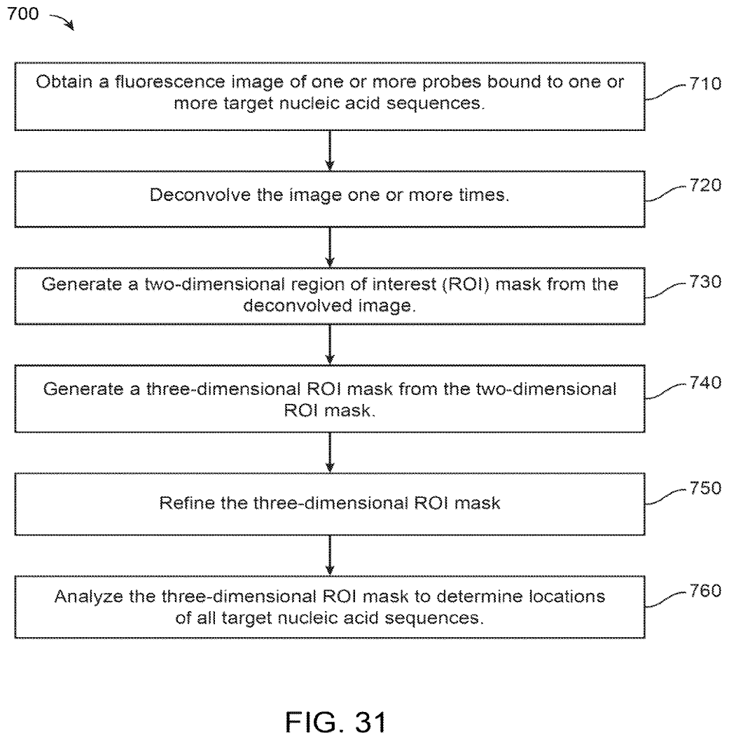

FIG. 30 shows a flowchart for a method 600 of fluorescently detecting a target nucleic acid sequence. The method may comprise an operation 610 of providing a one or more probes capable of binding to a target nucleic acid sequence, as described herein. The method may comprise an operation 620 of binding the one or more probes to the target nucleic acid sequence, as described herein. The method may comprise an operation 630 of photobleaching the one or more probes at one or more wavelengths, as described herein. The method may comprise an operation 640 of detecting a profile of optical emissions associated with the photobleaching, as described herein. The method may comprise an operation 650 of analyzing the detection profile to determine the localization of the target nucleic acid sequence, as described herein.

The localization of a nucleic acid sequence may include contacting a nucleic acid sequence with a first set of detection agents, photobleaching the first set of detection agents for a first time point at a first wavelength to generate a second set of detection agents capable of generating a light at a second wavelength, detecting at least one burst generated by the second set of detection agents to generate a detection profile of the second set of detection agents, and analyzing the detection profile to determine the localization of the nucleic acid sequence.

A detection agent may comprise a detectable moiety that is capable of generating a light, and a probe portion that is capable of hybridizing to a target site on a nucleic acid sequence. Each detection agent within the first set of detection agents may have the same or a different detectable moiety. Each detection agent within the first set of detection agents may have the same detectable moiety. A detectable moiety may comprise a small molecule (such as a fluorescent dye). A detectable moiety may comprise a fluorescently labeled polypeptide, a fluorescently labeled nucleic acid probe, and/or a fluorescently labeled polypeptide complex.

Upon photobleaching, a second set of detection agents may be generated from the first set of detection agents, in which the second set may include detection agents that are capable of generating a burst of light detectable at a second wavelength. For example, bleaching of the set of detection agents may lead to about 50%, about 60%, about 70%, about 80%, about 90%, or more detection agents within the set to enter into an “OFF-state”. An “OFF-state” may be a dark state in which the detectable moiety crosses from the singlet excited electronic or ON state to the triplet electronic state or OFF-state in which detection of light (such as fluorescence) may be low (for instance, less than 10%, less than 5%, less than 1%, or less than 0.5% of light may be detected). The remainder of the detection agents that have not entered into the OFF-state may generate bursts of lights, or to cycle between a singlet excited electronic state (or ON-state) and a singlet ground electronic state. As such, bleaching of the set of detection agents may generate about 40%, about 30%, about 20%, about 10%, about 5%, or less detection agents within the set that may generate bursts of lights. The bursts of lights may be detected stochastically, at a single burst level in which each burst of light correlates to a single detection agent.

A single wavelength may be used for photobleaching a set of detection agents. At least two wavelengths may be used for photobleaching a set of detection agents. A wavelength at 491 nm may be used. A wavelength at 405 nm may be used in combination with the wavelength at 491 nm. The two wavelengths may be applied simultaneously to photobleach a set of detection agents. The two wavelengths may be applied sequentially to photobleach a set of detection agents.

The time for photobleaching a set of detection agents may be from about 10 seconds to about 4 hours. The time may be from about 30 seconds to about 3.5 hours, from about one minute to about 3 hours, from about 5 minutes to about 2 hours, from about 10 minutes to about 1 hours, from about one minutes to about 1 hour, from about 5 minutes to about 1 hour, or from about 30 minutes to about 2 hours. The time may be at least 10 seconds, 20 seconds, 30 seconds, 40 seconds, 50 seconds, 1 minute, 2 minutes, 3 minutes, 4 minutes, 5 minutes, 10 minutes, 15 minutes, 20 minutes, 30 minutes, 45 minutes, 1 hour, 1.5 hours, 2 hours, 2.5 hours, 3 hours, 3.5 hours, 4 hours, or more.

The concentration of the detection agents may be from about 5 nM to about 1 μM. The concentration of the detection agent may be from about 5 nM to about 900 nM, from about 10 nM to about 800 nM, from about 15 nM to about 700 nM, from about 20 nM to about 50 0 nM, from about 10 nM to about 500 nM, from about 10 nM to about 400 nM, from about 10 nM to about 300 nM, from about 10 nM to about 200 nM, from about 10 nM to about 100 nM, from about 50 nM to about 500 nM, from about 50 nM to about 400 nM, from about 50 nM to about 300 nM, from about 50 nM to about 200 nM, from about 100 nM to about 500 nM, from about 100 nM to about 300 nM, or from about 100 nM to about 200 nM. The concentration of the detection agents may be about 10 nM, 15 nM, 20 nM, 30 nM, 40 nM, 50 nM, 60 nM, 70 nM, 80 nM, 90 nM, 100 nM, 150 nM, 200 nM, 250 nM, 300 nM, 400 nM, 500 nM, 600 nM, 700 nM, 800 nM, 900 nM, or more.

The burst of lights from the set of detection agents may generate a detection profile. The detection profile may comprise about 100 image frames, about 500 frames, about 1000 frames, about 2000 frames, about 5000 frames, about 10,000 frames, about 20,000 frames, about 30,000 frames, about 40,000 frames, about 50,000 frames, or more image frames. The detection profile may also include the signal intensity, signal location, or size of the signal. Analysis of the detection profile may determine the optical isolation and localization of the detection agents, which may correlate to the localization of the nucleic acid sequence.

The detection profile may comprise a chromatic aberration correction. The detection profile may comprise less than 5%, 4%, 3%, 2%, 1%, 0.5%, 0.1%, or 0% chromatic aberration. The detection profile may comprise less than 5% chromatic aberration. The detection profile may comprise less than 4% chromatic aberration. The detection profile may comprise less than 3% chromatic aberration. The detection profile may comprise less than 2% chromatic aberration. The detection profile may comprise less than 1% chromatic aberration. The detection profile may comprise less than 0.5% chromatic aberration. The detection profile may comprise less than 0.1% chromatic aberration. The detection profile may comprise 0% chromatic aberration.

More than one nucleic acid sequence may be detected at the same time. Sometimes, at least 2, at least 3, at least 4, at least 5, at least 6, at least 7, at least 8, at least 9, at least 10, at least 15, at least 20, or more nucleic acid sequence may be detected at the same time. Each of the nucleic acid sequences may be detected by a set of detection agents. The detectable moiety between the different set of detection agents may be the same. For example, two different sets of detection agents may be used to detect two different nucleic acid sequences and the detectable moieties from the two sets of detection agents may be the same. As such, at least 2, at least 3, at least 4, at least 5, at least 6, at least 7, at least 8, at least 9, at least 10, at least 15, at least 20, or more nucleic acid sequences may be detected at the same time at the same wavelength. The detectable moiety between the different set of detection agents may also be different. For example, two different sets of detection agents may be used to detect two different nucleic acid sequences and the detectable moiety from one set of detection agents may be detected at a different wavelength from the detectable moiety of the second set of detection agents. As such, at least 2, at least 3, at least 4, at least 5, at least 6, at least 7, at least 8, at least 9, at least 10, at least 15, at least 20, or more nucleic acid sequences may be detected at the same time in which each of the nucleic acid sequences may be detected at a different wavelength. The nucleic acid sequence may comprise DNA, RNA, polypeptides, or a combination thereof.

The activity of a target nucleic acid sequence may be measuring utilizing the methods described herein. The methods may include detection of a nucleic acid sequence and one or more products of the nucleic acid sequence. One or more products of the nucleic acid sequence may also include intermediate products or elements. The method may comprise contacting a cell sample with a first set and a second set of detection agents, in which the first set of detection agents interact with a target nucleic acid sequence within the cell and the second set of detection agents interact with at least one product of the target nucleic acid sequence, and analyze a detection profile from the first set and the second set of detection agents, in which the presence or the absence of the at least one product indicates the activity of the target nucleic acid sequence.

As described herein, a detection agent may comprise a detectable moiety that is capable of generating a light, and a probe portion that is capable of hybridizing to a target site on a nucleic acid sequence. Each detection agent within the first set of detection agents may have the same or a different detectable moiety. Each detection agent within the first set of detection agents may have the same detectable moiety. A detectable moiety may comprise a small molecule (such as a fluorescent dye). A detectable moiety may comprise a fluorescently labeled polypeptide, a fluorescently labeled nucleic acid probe, and/or a fluorescently labeled polypeptide complex.

The method may also allow photobleaching of the first set and the second set of detection agents, whereby generating a subset of detection agents capable of generating a burst of light. A detection profile may be generated from the detection of a set of light bursts, in which the presence or the absence of the at least one product may indicate the activity of the target nucleic acid sequence.

The nucleic acid sequence may comprise DNA, RNA, polypeptides, or a combination thereof. The nucleic acid sequence may be DNA. The nucleic acid sequence may be RNA. The nucleic acid sequence may be an enhancer RNA (eRNA). The presence of an eRNA may correlate with target gene transcription that is downstream of eRNA. The nucleic acid sequence may be a DNasel hypersensitive site (DHS). The DHS may be an activated DHS. The pattern of the DHS on a chromatin may correlate to the activity of the chromatin. The nucleic acid sequence may be a polypeptide, such as a transcription factor, a DNA or RNA-binding protein or binding fragment thereof, or a polypeptide that is involved in chemical modification. The nucleic acid sequence may be chromatin.

### Epifluorescence Imaging

One or more far-field or near-field fluorescence techniques may be utilized for the detection, localization, activity determination, and mapping of one or more nucleic acid sequences described herein. A microscopy method may be a high magnification oil immersion microscopy method. In such a method, wide-field and/or confocal fluorescent microscopes may achieve sub-cellular resolution.

### Super-Resolution Imaging

A microscopy method may utilize a super-resolution microscopy, which allows images to be taken with a higher resolution than the diffraction limit. A super-resolution microscopy method may utilize a deterministic super-resolution microscopy method, which utilizes a fluorophore's nonlinear response to excitation to enhance resolution. Exemplary deterministic super-resolution methods may include stimulated emission depletion (STED), ground state depletion (GSD), reversible saturable optical linear fluorescence transitions (RESOLFT), and/or saturated structured illumination microscopy (SSIM). A super-resolution microscopy method may also include a stochastic super-resolution microscopy method, which utilizes a complex temporal behavior of a fluorophore, to enhance resolution. Exemplary stochastic super-resolution method may include super-resolution optical fluctuation imaging (SOFI), all single-molecular localization method (SMLM) such as spectral precision determination microscopy (SPDM), SPDMphymod, photo-activated localization microscopy (PALM), fluorescence photo-activated localization microscopy (FPALM), stochastic optical reconstruction microscopy (STORM), and dSTROM.

A microscopy method may be a single-molecular localization method (SMLM). A microscopy method may be a spectral precision determination microscopy (SPDM) method. A SPDM method may rely on stochastic burst or blinking of fluorophores and subsequent temporal integration of signals to achieve lateral resolution at, for example, between about 10 nm and about 100 nm.

A microscopy method may be a spatially modulated illumination (SMI) method. A SMI method may utilize phased lasers and interference patterns to illuminate specimens and increase resolution by measuring the signal in fringes of the resulting Moire patterns.

A microscopy method may be a synthetic aperture optics (SAO) method. A SAO method may utilize a low magnification, low numerical aperture (NA) lens to achieve large field of view and depth of field, without sacrificing spatial resolution. For example, an SAO method may comprise illuminating the detection agent-labeled target (such as a target nucleic acid sequence) with a predetermined number (N) of selective excitation patterns, where the number (N) of selective excitation patterns is determined based upon the detection agent's physical characteristics corresponding to spatial frequency content (such as the size, shape, and/or spacing of the detection agents on the imaging target) from the illuminated target, optically imaging the illuminated target at a resolution insufficient to resolve the objects on the target, and processing optical images of the illuminated target using information on the selective excitation patterns to obtain a final image of the illuminated target at a resolution sufficient to resolve the objects on the target. The number (N) of selective excitation patterns may correspond to the number of k-space sampling points in a k-space sampling space in a frequency domain, with the extent of the k-space sampling space being substantially proportional to an inverse of a minimum distance (Δx) between the objects that is to be resolved by SAO, and with the inverse of the k-space sampling interval between the k-space sampling points being less than a width (w) of a detected area captured by a pixel of a system for said optical imaging. The number (N) may include a function of various parameters of the imaging system (such as a magnification of the objective lens, numerical aperture of the objective lens, wavelength of the light emitted from the imaging target, and/or effective pixel size of the pixel sensitive area of the image detector, etc.).

A SAO method may analyze a set of detection agent profiles from at least 100, at least 200, at least 250, at least 500, at least 1000, or more cells imaged simultaneously within one field of view utilizing an imaging instrument. The one field of view may be a single wide field of view allowing image capture of at least 100, at least 200, at least 250, at least 500, at least 1000, or more cells. The single wide field of view may be about 0.70 mm by about 0.70 mm field of view. The SAO imaging instrument may enable a resolution of about 0.25 μm with a 20×/0.45 NA lens. The SAO imaging instrument may enable a depth of field of about 2.72 μm with a 20×/0.45 NA lens. The imaging instrument may enable a working distance of about 7 mm with a 20×/0.45 NA lens. The imaging instrument may enable a z-stack of 1 with a 20×/0.45 NA lens. The SAO method may further integrate and interpolate 3-dimensional images from 2-dimensional images.

The SAO imaging instrument may be an SAO instrument as described in U.S. Publication No. 2011/0228073 (Lightspeed Genomics, Inc).

### Analysis of Fluorescence Images

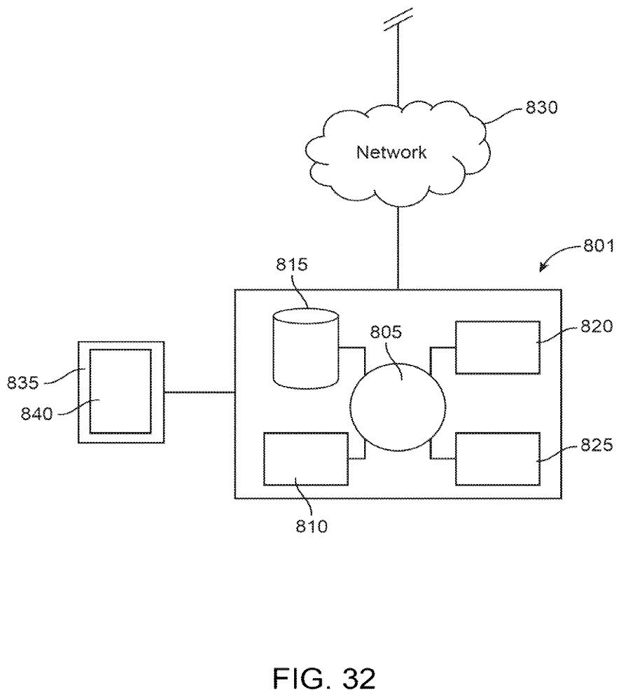

FIG. 31 shows a flowchart for a method 700 of analyzing a fluorescence image of one or more target nucleic acid sequences. The method may comprise an operation 710 of obtaining a fluorescence image of one or more probes bound to one or more target nucleic acid sequences, as described herein. The method may comprise an operation 720 of deconvolving the image one or more times, as described herein. The method may comprise an operation 730 of generating a two-dimensional region of interest (ROI) mask from the deconvolved image, as described herein. The method may comprise an operation 740 of generating a three-dimensional ROI mask from the two-dimensional ROI mask, as described herein. The method may comprise an operation 750 of refining the three-dimensional ROI mask, as described herein. The method may comprise an operation 760 of analyzing the three-dimensional ROI mask to determine the locations of all target nucleic acid sequences, as described herein.

Images obtained using the systems and methods described herein may be subjected to an image analysis method. The images may be obtained using the epifluorescence imaging systems and methods described herein. The image may be obtained using the super-resolution imaging systems and methods described herein. The image analysis method may allow a quantitative morphometric analysis to be conducted on regions of interest (ROIs) within the images.

The image analysis method may comprise a deconvolution of the image. The image analysis method may comprise an iterative deconvolution of the image. The image analysis method may comprise 1, 2, 3, 4, 5, 6, 7, 8, 9, or 10 iterations of deconvolving the image. The image analysis method may comprise more than 1, more than 2, more than 3, more than 4, more than 5, more than 6, more than 7, more than 8, more than 9, or more than 10 iterations of deconvolving the image. The deconvolution procedure may reduce out-of-focus blur in the epifluorescence images or super-resolution images, enhancing the signal-to-noise ratio (SNR) within ROIs.

The image analysis method may further comprise an identification of the ROIs. The ROIs may be identified using an automated detection method.

The image analysis method may further comprise segmentation of the ROIs. This may allow the rapid delineation of ROIs within the epifluorescence or super-resolution images. The segmentation of ROIs may comprise utilizing a maximum intensity projection image to generate a two-dimensional ROI mask. The two-dimensional ROI mask may act as a template for an initial three-dimensional mask. For instance, the initial three-dimensional mask may be generated by projecting the two-dimensional ROI mask into a third spatial dimension. The projection may be a weighted projection. The initial three-dimensional mask may be further refined to obtain a refined three-dimensional ROI mask. Refinement of the initial three-dimensional mask may be achieved utilizing adaptive thresholding and/or region growing methods. Refinement of the initial three-dimensional mask may be achieved by iteratively applying adaptive thresholding and/or region growing methods. The iterative procedure may result in a final three-dimensional ROI mask. The final three-dimensional ROI mask may comprise information regarding the locations of all FISH-labeled nucleic acid sequences within each cell in a sample.

The image analysis method may be implemented in an automated manner, such as using the digital processing devices described herein.

### Digital Processing Device

The systems, apparatus, and methods described herein may include a digital processing device, or use of the same. The digital processing device may include one or more hardware central processing units (CPU) that carry out the device's functions. The digital processing device may further comprise an operating system configured to perform executable instructions. In some instances, the digital processing device is optionally connected to a computer network, is optionally connected to the Internet such that it accesses the World Wide Web, or is optionally connected to a cloud computing infrastructure. In other instances, the digital processing device is optionally connected to an intranet. In other instances, the digital processing device is optionally connected to a data storage device.

In accordance with the description herein, suitable digital processing devices may include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers may include those with booklet, slate, and convertible configurations, known to those of skill in the art.

The digital processing device may include an operating system configured to perform executable instructions. The operating system may be, for example, software, including programs and data, which may manage the device's hardware and provides services for execution of applications. Those of skill in the art will recognize that suitable server operating systems may include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some cases, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360® Microsoft Xbox One, Nintendo® Wii®, Nintendo® Wii U®, and Ouya®.

In some instances, the device may include a storage and/or memory device. The storage and/or memory device may be one or more physical apparatuses used to store data or programs on a temporary or permanent basis. In some instances, the device is volatile memory and requires power to maintain stored information. In other instances, the device is non-volatile memory and retains stored information when the digital processing device is not powered. In still other instances, the non-volatile memory comprises flash memory. The non-volatile memory may comprise dynamic random-access memory (DRAM). The non-volatile memory may comprise ferroelectric random access memory (FRAM). The non-volatile memory may comprise phase-change random access memory (PRAM). The device may be a storage device including, by way of non-limiting examples, CD-ROMs, DVDs, flash memory devices, magnetic disk drives, magnetic tapes drives, optical disk drives, and cloud computing based storage. The storage and/or memory device may also be a combination of devices such as those disclosed herein.

The digital processing device may include a display to send visual information to a user. The display may be a cathode ray tube (CRT). The display may be a liquid crystal display (LCD). Alternatively, the display may be a thin film transistor liquid crystal display (TFT-LCD). The display may further be an organic light emitting diode (OLED) display. In various cases, on OLED display is a passive-matrix OLED (PMOLED) or active-matrix OLED (AMOLED) display. The display may be a plasma display. The display may be a video projector. The display may be a combination of devices such as those disclosed herein.

The digital processing device may also include an input device to receive information from a user. For example, the input device may be a keyboard. The input device may be a pointing device including, by way of non-limiting examples, a mouse, trackball, track pad, joystick, game controller, or stylus. The input device may be a touch screen or a multi-touch screen. The input device may be a microphone to capture voice or other sound input. The input device may be a video camera or other sensor to capture motion or visual input. Alternatively, the input device may be a Kinect™, Leap Motion™, or the like. In further aspects, the input device may be a combination of devices such as those disclosed herein.

### Non-Transitory Computer Readable Storage Medium

In some instances, the systems, apparatus, and methods disclosed herein may include one or more non-transitory computer readable storage media encoded with a program including instructions executable by the operating system of an optionally networked digital processing device. In further instances, a computer readable storage medium is a tangible component of a digital processing device. In still further instances, a computer readable storage medium is optionally removable from a digital processing device. A computer readable storage medium may include, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

The systems, apparatus, and methods disclosed herein may include at least one computer program, or use of the same. A computer program includes a sequence of instructions, executable in the digital processing device's CPU, written to perform a specified task. In some embodiments, computer readable instructions are implemented as program modules, such as functions, objects, Application Programming Interfaces (APIs), data structures, and the like, that perform particular tasks or implement particular abstract data types. In light of the disclosure provided herein, those of skill in the art will recognize that a computer program, in certain embodiments, is written in various versions of various languages.

The functionality of the computer readable instructions may be combined or distributed as desired in various environments. A computer program may comprise one sequence of instructions. A computer program may comprise a plurality of sequences of instructions. In some instances, a computer program is provided from one location. In other instances, a computer program is provided from a plurality of locations. In additional cases, a computer program includes one or more software modules. Sometimes, a computer program may include, in part or in whole, one or more web applications, one or more mobile applications, one or more standalone applications, one or more web browser plug-ins, extensions, add-ins, or add-ons, or combinations thereof.

### Web Application

A computer program may include a web application. In light of the disclosure provided herein, those of skill in the art will recognize that a web application, in various aspects, utilizes one or more software frameworks and one or more database systems. In some cases, a web application is created upon a software framework such as Microsoft®.NET or Ruby on Rails (RoR). In some cases, a web application utilizes one or more database systems including, by way of non-limiting examples, relational, non-relational, object oriented, associative, and XML database systems. Sometimes, suitable relational database systems may include, by way of non-limiting examples, Microsoft® SQL Server, mySQL™, and Oracle®. Those of skill in the art will also recognize that a web application, in various instances, is written in one or more versions of one or more languages. A web application may be written in one or more markup languages, presentation definition languages, client-side scripting languages, server-side coding languages, database query languages, or combinations thereof. A web application may be written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). Aweb application may be written to some extent in a client-side scripting language such as Asynchronous Javascript and XML (AJAX), Flash® Actionscript, Javascript, or Silverlight®. A web application may be written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. Sometimes, a web application may be written to some extent in a database query language such as Structured Query Language (SQL). Other times, a web application may integrate enterprise server products such as IBM® Lotus Domino®. In some instances, a web application includes a media player element. In various further instances, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

A computer program may include a mobile application provided to a mobile digital processing device. In some cases, the mobile application is provided to a mobile digital processing device at the time it is manufactured. In other cases, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

A computer program may include a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. A computer program may include one or more executable complied applications.

### Web Browser Plug-In

The computer program may include a web browser plug-in. In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

Web browsers (also called Internet browsers) may be software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The systems and methods disclosed herein may include software, server, and/or database modules, or use of the same. In view of the disclosure provided herein, software modules may be created by techniques known to those of skill in the art using machines, software, and languages known to the art. The software modules disclosed herein may be implemented in a multitude of ways. A software module may comprise a file, a section of code, a programming object, a programming structure, or combinations thereof. A software module may comprise a plurality of files, a plurality of sections of code, a plurality of programming objects, a plurality of programming structures, or combinations thereof. In various aspects, the one or more software modules comprise, by way of non-limiting examples, a web application, a mobile application, and a standalone application. In some instances, software modules are in one computer program or application. In other instances, software modules are in more than one computer program or application. In some cases, software modules are hosted on one machine. In other cases, software modules are hosted on more than one machine. Sometimes, software modules may be hosted on cloud computing platforms. Other times, software modules may be hosted on one or more machines in one location. In additional cases, software modules are hosted on one or more machines in more than one location.

### Databases

The methods, apparatus, and systems disclosed herein may include one or more databases, or use of the same. In view of the disclosure provided herein, those of skill in the art will recognize that many databases are suitable for storage and retrieval of analytical information described elsewhere herein. In various aspects described herein, suitable databases may include, by way of non-limiting examples, relational databases, non-relational databases, object oriented databases, object databases, entity-relationship model databases, associative databases, and XML databases. A database may be internet-based. A database may be web-based. A database may be cloud computing-based. Alternatively, a database may be based on one or more local computer storage devices.

### Services

Methods and systems described herein may further be performed as a service. For example, a service provider may obtain a sample that a customer wishes to analyze. The service provider may then encodes the sample to be analyzed by any of the methods described herein, performs the analysis and provides a report to the customer. The customer may also perform the analysis and provides the results to the service provider for decoding. In some instances, the service provider then provides the decoded results to the customer. In other instances, the customer may receive encoded analysis of the samples from the provider and decodes the results by interacting with softwares installed locally (at the customer's location) or remotely (e.g. on a server reachable through a network). Sometimes, the softwares may generate a report and transmit the report to the costumer. Exemplary customers include clinical laboratories, hospitals, industrial manufacturers and the like. Sometimes, a customer or party may be any suitable customer or party with a need or desire to use the methods provided herein.

### Server

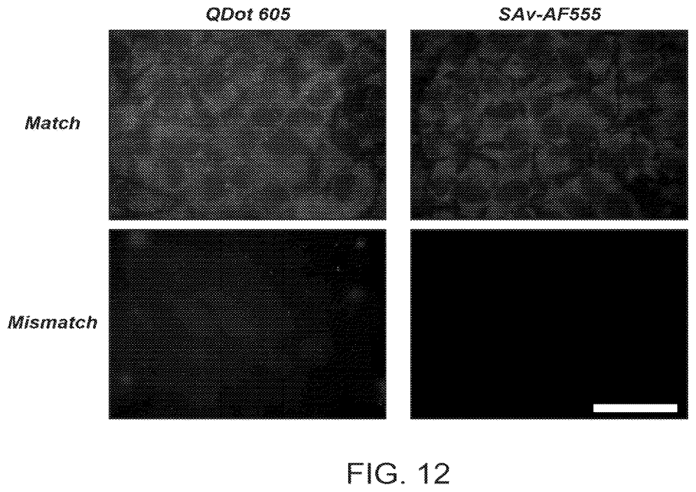

The methods provided herein may be processed on a server or a computer server, as shown in FIG. 13). The server 801 may include a central processing unit (CPU, also “processor”) 805 which may be a single core processor, a multi core processor, or plurality of processors for parallel processing. A processor used as part of a control assembly may be a microprocessor. The server 801 may also include memory 810 (e.g. random access memory, read-only memory, flash memory); electronic storage unit 815 (e.g. hard disk); communications interface 820 (e.g. network adaptor) for communicating with one or more other systems; and peripheral devices 825 which includes cache, other memory, data storage, and/or electronic display adaptors. The memory 810, storage unit 815, interface 820, and peripheral devices 825 may be in communication with the processor 805 through a communications bus (solid lines), such as a motherboard. The storage unit 815 may be a data storage unit for storing data. The server 801 may be operatively coupled to a computer network (“network”) 830 with the aid of the communications interface 820. A processor with the aid of additional hardware may also be operatively coupled to a network. The network 830 may be the Internet, an intranet and/or an extranet, an intranet and/or extranet that is in communication with the Internet, a telecommunication or data network. The network 830 with the aid of the server 801, may implement a peer-to-peer network, which may enable devices coupled to the server 801 to behave as a client or a server. The server may be capable of transmitting and receiving computer-readable instructions (e.g., device/system operation protocols or parameters) or data (e.g., sensor measurements, raw data obtained from detecting metabolites, analysis of raw data obtained from detecting metabolites, interpretation of raw data obtained from detecting metabolites, etc.) via electronic signals transported through the network 830. Moreover, a network may be used, for example, to transmit or receive data across an international border.

The server 801 may be in communication with one or more output devices 835 such as a display or printer, and/or with one or more input devices 840 such as, for example, a keyboard, mouse, or joystick. The display may be a touch screen display, in which case it functions as both a display device and an input device. Different and/or additional input devices may be present such an enunciator, a speaker, or a microphone. The server may use any one of a variety of operating systems, such as for example, any one of several versions of Windows®, or of MacOS®, or of Unix®, or of Linux®.

The storage unit 815 may store files or data associated with the operation of a device, systems or methods described herein.

The server may communicate with one or more remote computer systems through the network 830. The one or more remote computer systems may include, for example, personal computers, laptops, tablets, telephones, Smart phones, or personal digital assistants.

A control assembly may include a single server 801. In other situations, the system may include multiple servers in communication with one another through an intranet, extranet and/or the Internet.

The server 801 may be adapted to store device operation parameters, protocols, methods described herein, and other information of potential relevance. Such information may be stored on the storage unit 815 or the server 801 and such data is transmitted through a network.

### Kits

A composition described herein may be supplied in the form of a kit. A composition may be a probe set designed for a target nucleic acid sequence. The kits of the present disclosure may further comprise instructions regarding the method of using the probe set to detect the target nucleic acid sequence.

In some embodiments, a kit comprises the compositions and methods for detecting a target nucleic acid sequence (to perform a Nano-FISH assay). The compostions and methods may be for fast detection of the target nucleic acid sequence, e.g., in about 24 hours or less, or in about 48 hours or less. The compostions and methods may be for detection of the target nucleic acid sequence, wherein the target nucleic acid sequence is a short nucleic acid sequence, e.g., less than 2 kb, less than 1.5 kb, or less than 0.5 kb. The compostions and methods may be for detecting and quantifying the target nucleic acid sequence in a cell or in a population of cells. In some embodiments, a kit may further comprise components useful in using the kit components and instructions on how to prepare the components for detection of a target nucleic acid sequence. In some embodiments, the kit may further comprise software needed for detection of the target nucleic acid sequence.

The components of the kit may be in dry or liquid form. If they are in dry form, the kit may include a solution to solubilize the dried material. The kit may also include transfer factor in liquid or dry form. In some embodiments, if the transfer factor is in dry form, the kit includes a solution to solubilize the transfer factor. The kit may also include containers for mixing and preparing the components. The kits as described herein also may include a means for containing compositions of the present disclosure in close confinement for commercial sale and distribution.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts may be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

### EXAMPLES

These examples are provided for illustrative purposes only and not to limit the scope of the claims provided herein.

### Example 1

### DNase Treatment and TUNEL Assay

A TUNEL assay as described below may be used to label DNasel cut sites on a global cell. For example, all of the DNasel cut sites within a call's nucleus may be labeled.

Cells were prepared for a 2-color SPDM for DNA density and DNase I sensitivity (TUNEL) assay.

An adherent cell line, A549 (lung adenocarcinoma), was used for these experiments. They were plated overnight on uncoated 18 mm (#1 thickness) coverslips. Cells were deliberately plated sparsely to be ˜20% confluent on the day of the assay.

For all coverslips, cells were fixed with 4% formaldehyde in PBS for 10 minutes at room temperature, and then equilibrated in buffer A at room temperature for 15 minutes. The cells were permeabilized with 0.1% NP-40 in buffer A for 10 minutes at room temperature.

The DNasel assay was performed with 80 U/ml DNasel for 3 minutes at 37° C. Cells were then post fixed in 4% formaldehyde in buffer A for 10 minutes at room temperature. The coverslips were permeabilized for 20 minutes with buffer A with 0.25% TX-100, and washed twice with distilled water and were equilibrated with 100 μl of TdT reaction buffer for 10 minutes at room temperature. The terminal deoxynucleotide transferase (TdT) reaction with EdUTP-alkyne (100 μl per coverslip) was performed for 1 hour at 37° C. At the end of the TdT reaction, the coverslips were washed twice with 3% BSA/PBS. The ClickIT reaction was then performed for 2 coverslips to add Alexafluor647 to incorporated EdUTP-alkyne. This reaction was performed for 30 minutes at room temperature, in the dark. The other coverslips were kept in 3% BSA/PBS at room temperature. The coverslips were washed once with 3% BSA/PBS before being stained with Vybrant Violet staining and imaged by a SMLM method.

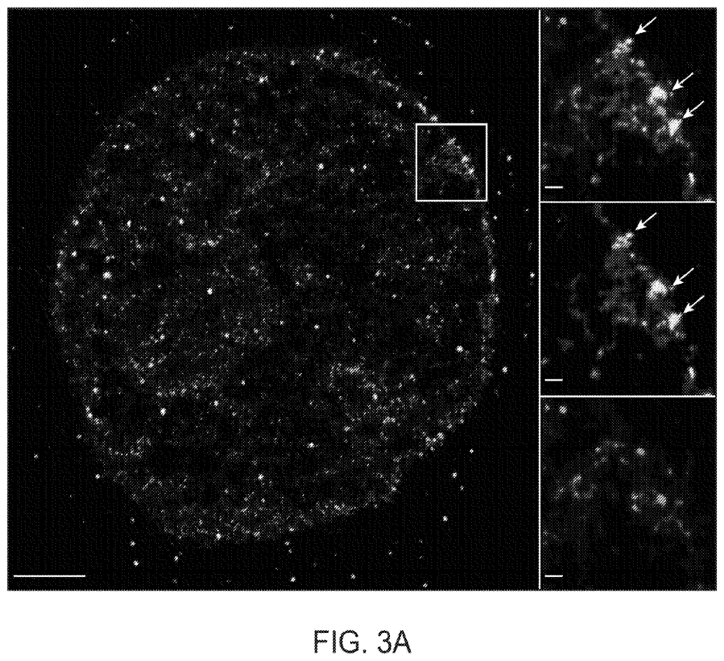

FIG. 3A shows a two color SPDM image (experimental) of chromatin (blue) with a DNA sensitive element (red), showing anti-colocalisation of the DNA sensitive element with chromatin. Scale bars: 1000 nm, inserts: 100 nm. FIG. 3B is the inset of FIG. 3A.

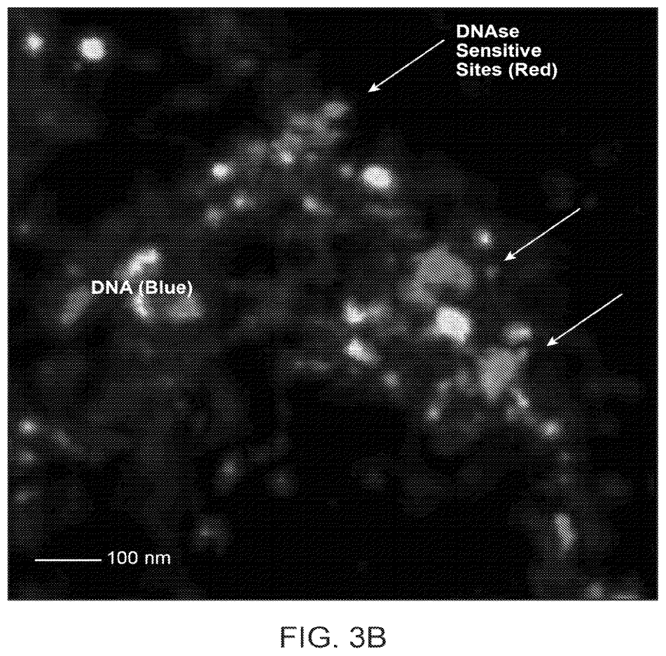

FIG. 4 illustrates the localization precision and nearest neighbor distances for DNA and DNase sensitive elements.

### Example 2

### DNA Encoding of Molecular Targets on a Multi-Omics Imaging Platform

Integration of imaging data across different molecular target types may provide in-depth insights into cell physiology and pathology. A multi-omics imaging platform may be utilized which enables simultaneous visualization of multiple molecular targets irrespective of target type and imaging probes used. The multi-omics imaging platform comprise (i) decoupling of target binding and labeling steps, (ii) translation of heterogeneous molecular information into an intermediate standardized molecular code amenable to read-out via imaging probes, and (iii) employing encoding capacity and self-assembly capabilities of DNA bonding. Specifically, molecular targets of interest are first encoded with unique ssDNA tags via binding by ssDNA-conjugated target-recognition moieties under optimized conditions favoring specific target binding. Individual ssDNA tags are then converted into detectable signals via sequence-specific hybridization with complementary ssDNA′-conjugated imaging probes under probe-optimized conditions. As such, molecular target uniqueness, localization, abundance, and specimen morphology information are preserved through all steps of labeling procedure, producing comprehensive molecular signatures of a physiological or pathological process.

### Methods

Oligonucleotide probe design. Sequences for 6 ssDNA/ssDNA′ encoding pairs were selected from a random pool. Selection criteria were: continuous 16 bp complementarity, balanced nucleotide composition, lack of stable secondary structures at room temperature, lack of substantial cross-hybridization between mismatch pairs. See Table 1 for a complete list of ssDNA/ssDNA′ encoding pairs.

Sequences for human GAPDH mRNA (NM_002046.5) and HSP90-alpha mRNA (NM_001271969.1) were obtained from NCBI. Sets of mRNA ISH probes were designed using Stellaris RNA FISH Probe Designer (Biosearch Technologies). Probe sets contained 36 unique probes for GAPDH mRNA and 48 probes for HSP90-alpha mRNA. Each probe featured 5′ terminal 20 nt-long region complementary to mRNA, a spacer (either AAAAA for smaller 41 nt probes or AAAA-dsSpacer-AAAA for longer 60 nt probes), and a 16 nt-long QDot binding tag. ISH probe strand of the dsSpacer was 5′-TTCCCAAGCGTCATCT-3′ (SEQ ID NO:), pre-hybridized with a complementary 5′-AGATGACGCTTGGGAA-3′ ssDNA at 1:1 molar ratio to form a 16 bp dsDNA spacer prior to specimen labeling. See Supplementary Tables 2-3 for a complete list of ISH probes. All oligonucleotides were purchased from IDT DNA.

Antibody-ssDNA conjugation. Purified primary and secondary antibodies in PBS were purchased from Sigma-Aldrich. Amine-terminated HPLC purified ssDNA tags were purchased from IDT DNA (see Table 1, sequences 1B-6B). Covalent antibody-ssDNA bioconjugation was achieved either a) via maleimide-mediated amine-sulfhydryl crosslinking or b) using Thunder-Link oligo conjugation system (Innova Biosciences).

For maleimide-mediated crosslinking, IgG was partially reduced by TCEP to expose free sulfhydryl groups, while 5′ amine-terminated ssDNA oligonucleotides were activated by sulfo-SMCC (Thermo Scientific). IgG was diluted to 1 mg/mL in 100 μL PBS with 10 mM EDTA, mixed with 0.5 mM TCEP, and incubated for 30 min at 37° C. At the same time, ssDNA was diluted to 40 μM in 100 μL PBS, mixed with 10 mM sulfo-SMCC, and incubated for 30 min at RT. Reduced IgG and activated ssDNA were then purified by 3 rounds of desalting in Zeba desalting spin columns (Thermo Scientific) pre-washed with PBS/10 mM EDTA, mixed, and reacted for 4 Hrs at RT. Finally, unreacted sulfhydryl groups were capped by addition of 1 mM sulfo-SMCC pre-quenched by excess glycine. Antibody-ssDNA bioconjugates were purified by ultrafiltration for at least 6 times with Amicon Ultra 50 KDa MWCO centrifugal filter (Millipore) and stored in PBS solution at 4° C.

For antibody-ssDNA conjugation with Thunder-Link oligo conjugation system, IgG was diluted to 1 mg/mL in 100 μL PBS, activated by the Antibody Activation Reagent for 1 Hr at RT, and purified using desalting column. At the same time, 5′ amine-terminated ssDNA oligonucleotides were diluted to 80 μM in 100 μL PBS, activated by the Oligo Activation Reagent for 1 Hr at RT, and desalted. Activated IgG and ssDNA were mixed at a volume ratio of 2:1 (200 μL IgG+100 μL ssDNA+100 μL wash buffer), reacted overnight at RT, and stored at 4° C. For optimization studies, following IgG: ssDNA volume ratios were tested: 50+50, 50+30, 50+20, and 50+10.

QDot-ssDNA conjugation. Amine-functionalized PEG-coated QDots (Qdot ITK amino (PEG) quantum dots, Invitrogen) with emission peaks centered at 525, 545, 565, 585, 605, and 655 nm were used for the preparation of QDot-ssDNA probes. Amine-terminated HPLC purified 16 nt-long ssDNA tags were purchased from IDT DNA (see Table 1, sequences 1A-6A). Oligonucleotides were activated with bifunctional cross-linker BS3 (Bis[sulfosuccinimidyl] suberate, Thermo Scientific), followed by covalent conjugation with QDots. 100 μL 40 μM ssDNA solution in PBS was mixed with 500 molar excess of BS3 and incubated for 30 minutes at room temperature. Excess crosslinker was removed by 3 rounds of desalting in Zeba desalting spin columns (Thermo Scientific) pre-washed with PBS. Activated ssDNA was then mixed with 25 μL 8 μM stock QDot solution. The reaction was incubated overnight at room temperature and purified by ultrafiltration for at least 6 times with Amicon Ultra 100 KDa MWCO centrifugal filter (Millipore). Purified QDot-ssDNA probes were stored in PBS solution at 4° C.

Agarose gel electrophoresis was used for characterization of QDot-ssDNA probes. Procedure was performed on a 2% agarose gel in 1×TBE at 90V for 2 Hrs.

Cell culture and processing. Human cervical cancer cell line HeLa (ATCC) was used as a model specimen for evaluation of the multi-omics imaging via DNA encoding. Cells were grown in glass-bottom 24-well plates (Greiner Bio-One) in a humidified atmosphere at 37° C. with 5% CO₂ to a density of 80-90% using MEM culture medium with L-glutamine (Gibco) supplemented with 10% fetal bovine serum (Gibco). Prior to labeling, cells were rinsed with PBS, fixed with 4% formaldehyde in PBS for 5 min at room temperature followed by 15 min at 4° C., permeabilized with ice-cold 0.5% detergent Triton™ X-100 (Thermo Scientific) in PBS for 15 min at 4° C., and washed with PBS. For mRNA imaging, cells were immediately processed for in situ hybridization to minimize degradation of mRNA prior to labeling. For protein imaging only, fixed cells could be stored in PBS with 0.03% sodium azide at 4° C. for several days.

Encoding via immunorecognition. Encoding of protein targets in formalin-fixed cells was performed via incubation with antibody-ssDNA bioconjugates. Prior to labeling, cells were blocked by 2% BSA (from 10% BSA/PBS solution, Thermo Scientific), 0.5% Western blot blocking reagent (from 10% solution, Roche), 0.1% low MW dextran sulfate (9-20 kDa MW, Sigma-Aldrich), 0.1 mg/mL shredded salmon sperm DNA (Invitrogen), and 1×PBS for 30 min at RT. Antibodies were used at a final concentration of 5 μg/mL diluted in 2% BSA, 0.1% dextran sulfate, 0.1 mg/mL shredded salmon sperm DNA, and 1×PBS and incubated with cells for 1-2 Hrs at RT. Following labeling, cells were washed with PBS.

For reference studies, cell labeling with unmodified antibodies was performed in a similar fashion.

Encoding via in situ hybridization (ISH). Encoding of mRNA targets was performed via hybridization with ssDNA-tagged mRNA ISH probes. Cells were equilibrated with 10% formamide (Thermo Scientific), 2 mM RVC (New England BioLabs), 2×SSC (Invitrogen) buffer for 30 min at RT and then incubated with 400 μL/well 250 nM mix of mRNA ISH probes in 1% dextran sulfate (>500 kDa MW, Sigma-Aldrich), 1 mg/mL tRNA (from E. coli, Roche), 10% formamide, 2 mM RVC, 2×SSC hybridization buffer for 4 Hrs (or overnight) at 37° C. Following hybridization, cells were washed with warm 10% formamide, 2×SSC buffer for 30 min at 37° C., two changes of 1×PBS for 10 min at RT, and blocked by 2% BSA, 0.5% Western blot blocking reagent, 0.1% low MW dextran sulfate, 0.1 mg/mL shredded salmon sperm DNA, 1×PBS for 30 min at RT.

Encoding for multi-omics studies. Encoding of protein and mRNA targets on the same specimen was performed by combining immunorecognition and in situ hybridization procedures. First, cells were hybridized with ssDNA-tagged mRNA ISH probes as described above. Following hybridization and washing, cells were blocked, incubated with antibody-ssDNA bioconjugates for 1-2 Hrs at RT, and washed with PBS.

Specimen labeling with QDot probes. Following encoding of targets with ssDNA tags, cells were simultaneously labeled with complementary QDot-ssDNA′ probes. QDots were used at a final concentration of 5 nM in 2% BSA, 0.1% low MW dextran sulfate, 0.1 mg/mL shredded salmon sperm DNA, 1×PBS and incubated with cells for 2-4 Hrs at RT. Following staining cells were washed with PBS. Optionally, nuclei could be counter-stained by a 5-min incubation with DAPI.

For reference immunofluorescence studies, cell staining with QDots functionalized with secondary Ab fragments (Qdot goat F(ab′)2 anti-mouse or anti-rabbit IgG conjugates (H+L), Invitrogen) was performed in a similar fashion.

RNAi. Knock-down of GAPDH expression was done via cell transfection with GAPDH siRNA (Ambion). For forward transfection, cells were grown in a glass-bottom 24-well plate overnight and then treated with 500 μl/well culture medium containing 25 nM GAPDH siRNA and 0.5 μl/well DharmaFECT-2 transfection reagent (Dharmacon) for 24 Hrs. For reverse transfection, cells were grown in a 10 cm TC-treated dish, trypsinized, mixed in suspension with culture medium containing 25 nM GAPDH siRNA and 0.5 μl/well DharmaFECT-2 transfection reagent, seeded into a glass-bottom 24-well plate at 500 μl/well cell suspension, and incubated for 24 or 48 Hrs. Following transfection, cells were processed for staining. Triplicate samples were also prepared for RT-PCR analysis.

RT-PCR analysis. Total RNA was isolated from cell pellets using TRIzol reagent (Invitrogen) according to the manufacturer's protocol. Two hundred nanograms of RNA was converted to cDNA using random hexamer primer and MultiScribe Reverse Transcriptase Reagent (Applied Biosystems). One hundred nanograms of cDNA was amplified by the Real-Time PCR using SensiFAST™ Real-Time PCR Kits (Bioline, UK) on Chromo4 Real-Time PCR detection system (Bio-Rad). The primers used for GAPDH amplification were 5′-TCGCTCTCTGCTCCTCCTGTTC-3′ (forward primer) and 5′-CGCCCAATACGACCAAATCC-3′ (reverse primer). Cyclophilin A (PPIA) was used as an internal control, and the primers were 5′-GTCAACCCCACCGTGTTCTTC-3′ (forward primer) and 5′-TTTCTGCTGTCTTTGGGACCTTG-3′ (reverse primer). To confirm the PCR specificity, PCR products were subjected to a melting-curve analysis. The comparative threshold (Ct) method was used to calculate the relative mRNA amount of the treated sample in comparison to control samples. Mean value from triplicate samples was reported.

Imaging and signal analysis. IX-71 inverted fluorescence microscope (Olympus) equipped with a true-color CCD (QColor5, Olympus) and a hyperspectral imaging camera (Nuance, 420-720 nm spectral range, CRI, now PerkinElmer) was used for cell imaging. Low-magnification images were obtained with ×20 dry objective (NA 0.75, Olympus) and high-magnification with ×40 (NA 1.30, Olympus) and ×100 (NA 1.40, Olympus) oil-immersion objectives. Wide UV filter cube (330-385 nm band-pass excitation, 420 nm long-pass emission, Olympus) was used for imaging of all QDot probes, while Rhodamine LP cube (530-560 nm band-pass excitation, 572 nm long-pass emission, Chroma) was used for Alexa Fluor 555 detection. All images were acquired with cells attached to the coverslip bottom of the well and immersed in PBS without use of anti-fading reagents.

Nuance image analysis software was used to unmix the obtained multispectral images based on the reference spectra of each QDot component along with an extra channel for background fluorescence. In a false-color composite image, brightness and contrast of each channel was automatically adjusted for best visual representation and clear depiction of relative target distribution, unless noted otherwise. For direct comparison of QDot staining intensity individual QDot channels were normalized.

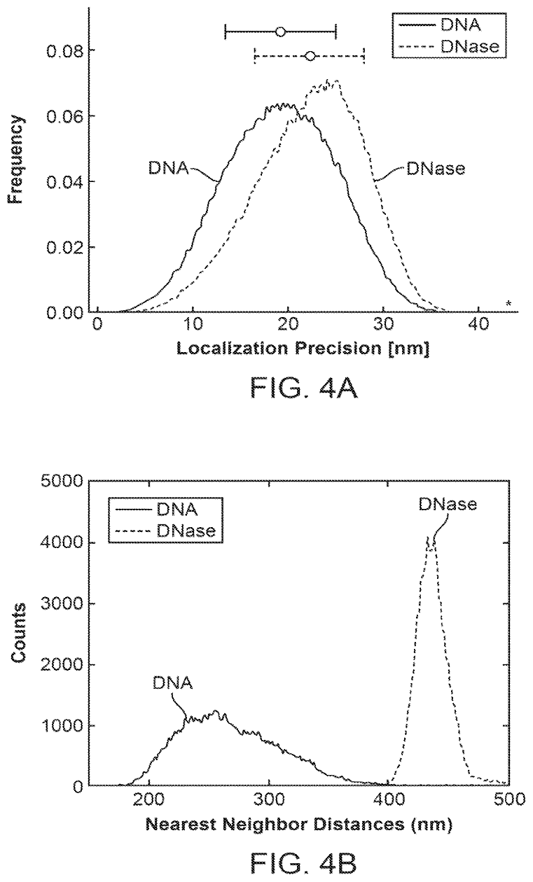

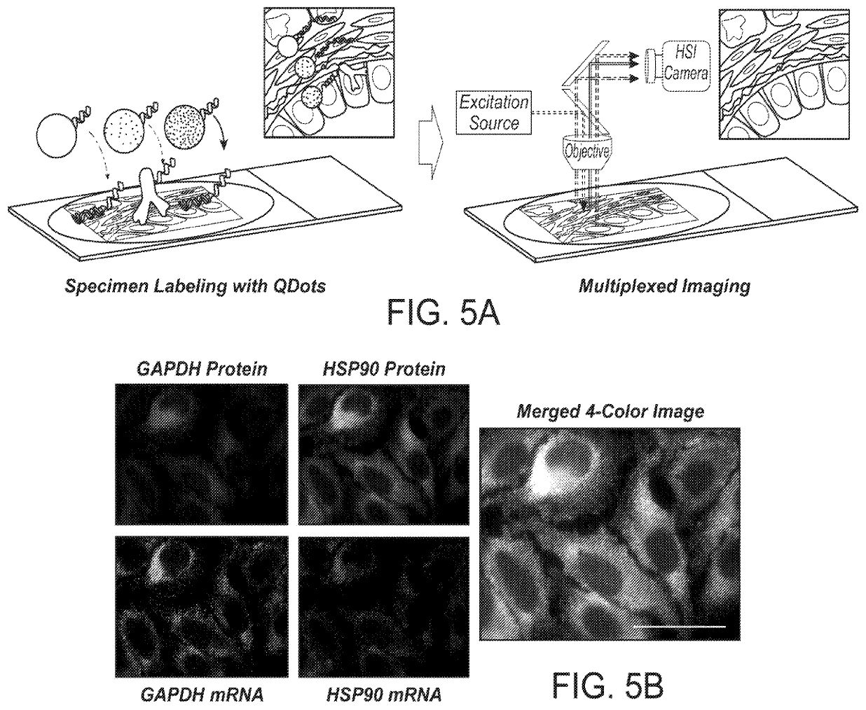

DNA Encoding for Multi-Omic Imaging Studies. To demonstrate the DNA encoding for multi-omics imaging studies concurrent analysis of single-cell molecular expression profiles at mRNA and protein levels were performed. Fluorescent quantum dot probes (QDots) in combination with fluorescence microscopy and hyperspectral imaging (HSI) were employed for simultaneous visualization of all ssDNA tags following separate encoding of mRNA and protein targets (FIG. 5A). For example, GAPDH and HSP90-alpha mRNA molecules and their respective product proteins can be readily labeled by 4-color QDots to highlight relative intracellular distribution and abundance of the two target types at a single-cell level (FIG. 5B). Unlike direct labeling procedures performed at a single incubation condition fixed for all targets and probes, DNA encoding enables tuning of conditions to favor recognition of individual target types and hybridization with detection probes in separate steps, offering great flexibility in choice of specimens, targets, and imaging systems (FIG. 6).

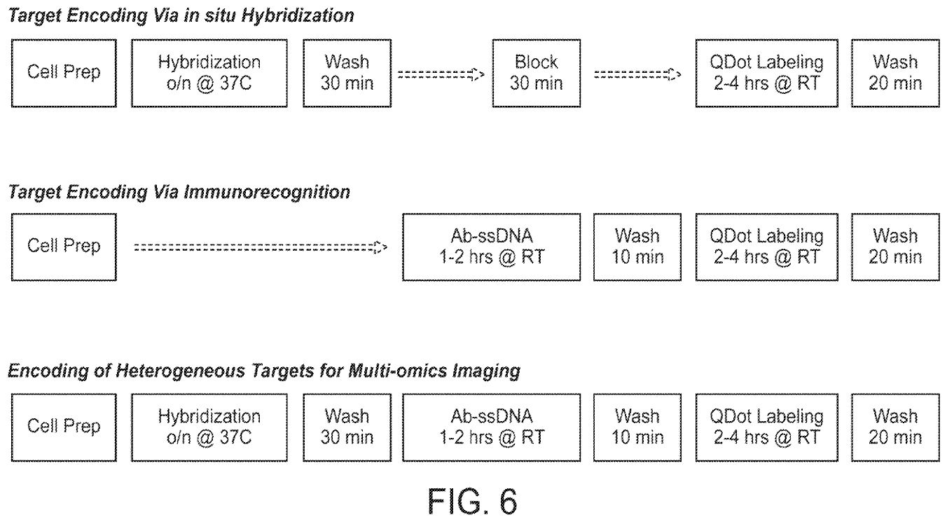

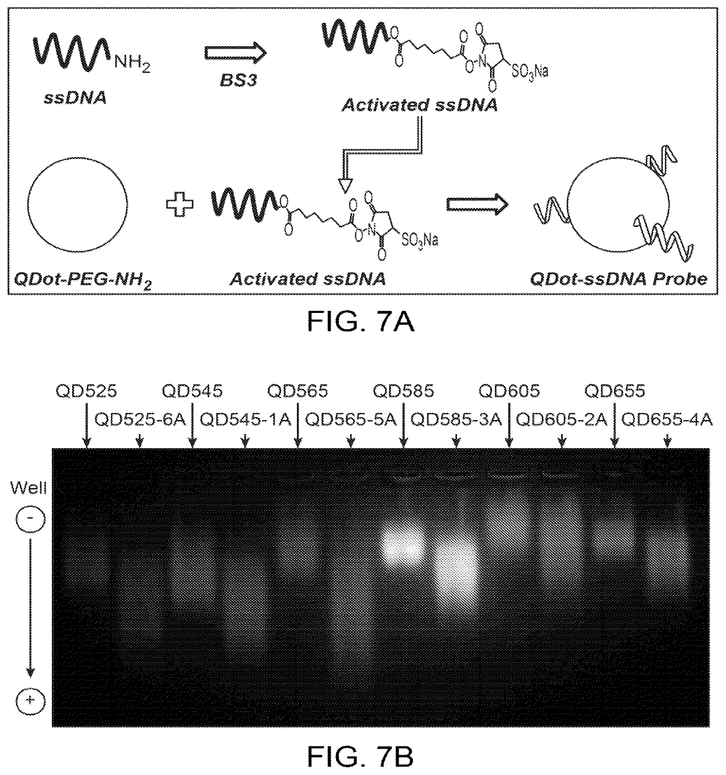

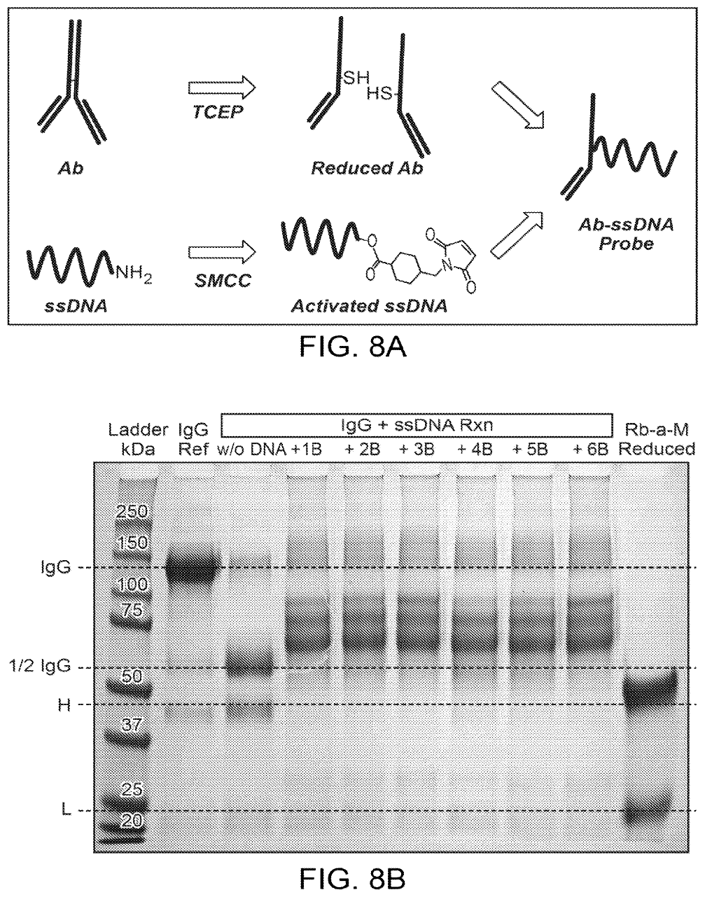

QDot-based Multi-Omics Imaging Platform. To implement and systematically characterize the model QDot-based multi-omics imaging platform, a set of 6 unique 16 bp ssDNA/ssDNA′ linkers was developed for encoding of up to 6 different molecular targets (Table 1) along with a library of complementary 6-color QDot-ssDNA probes (FIG. 7A and FIG. 7B) and a control set of 6 secondary antibody-ssDNA (2′Ab-ssDNA) bioconjugates (FIG. 8A and FIG. 8B). Indirect labeling of β-tubulin in HeLa cells via a 3-step procedure involving incubation with unmodified primary antibodies, 2′Ab-ssDNA bioconjugates, and complementary QDot-ssDNA′ probes demonstrated preserved antigen-recognition functionality of ssDNA-modified antibodies and high specificity of QDot staining via DNA hybridization (FIG. 9).

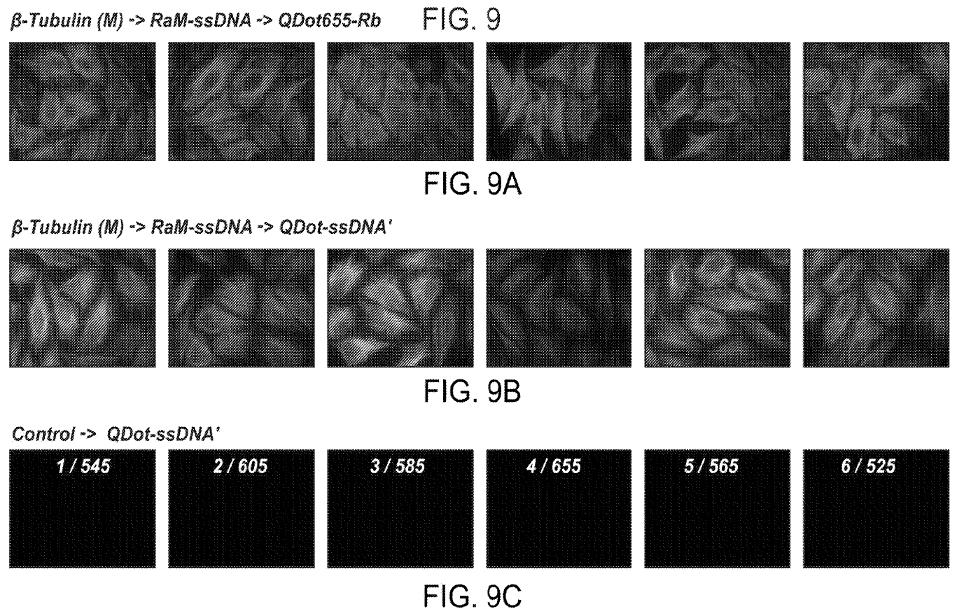

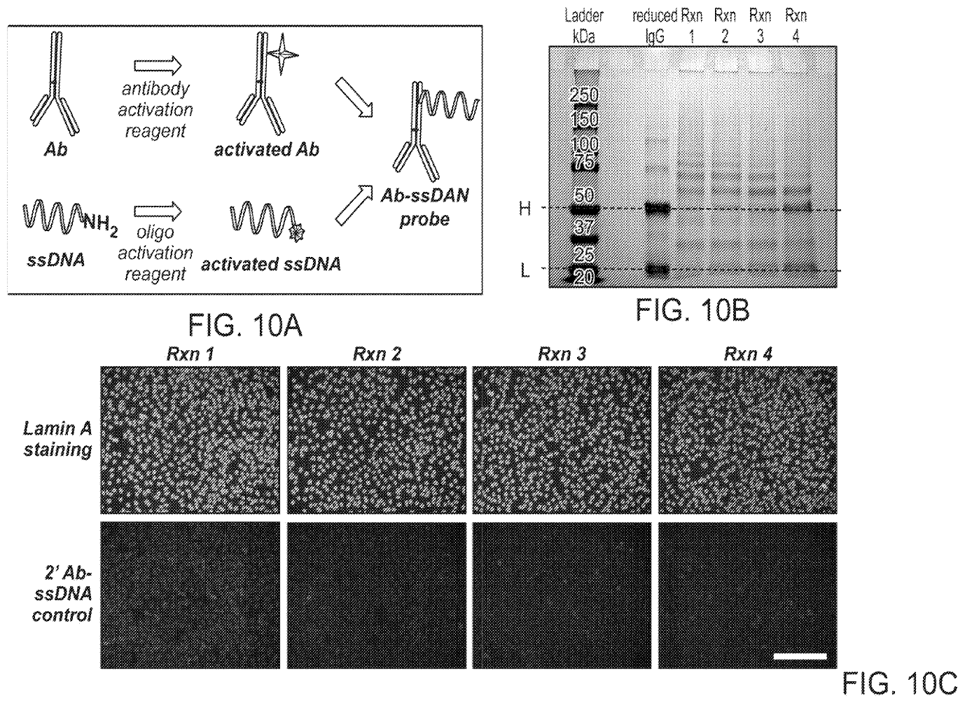

Multiplex Protein Immuno-labeling. Multiplexed protein immuno-labeling was realized through preparation of a library of primary antibody-ssDNA (1′Ab-ssDNA) bioconjugates (FIG. 10A, FIG. 10B, and FIG. 10C; and FIG. 11A, FIG. 11B, FIG. 11C, FIG. 11D, and FIG. 11E). Characterization of such bioconjugates with PAGE and cell staining confirmed preserved stability and antigen-binding functionality of antibodies, specificity of target staining with QDots in a 2-step procedure, and consistent target identification with different QDot colors in a multiplexed imaging format (FIG. 11A, FIG. 11B, FIG. 11C, FIG. 11D, and FIG. 11E). Nuclear envelope protein Lamin A, microtubule β-tubulin, and cytoplasmic proteins HSP90-alpha and GAPDH were labeled as model target molecules with distinct characteristic intracellular localization.

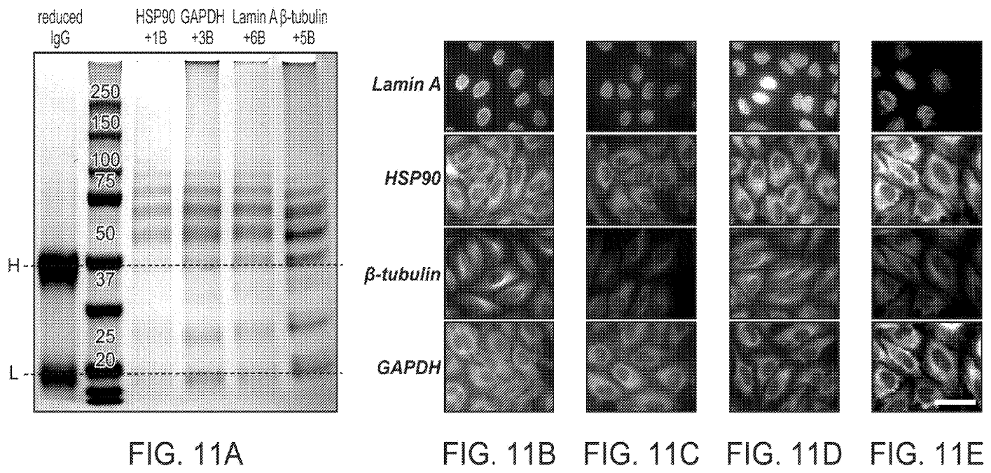

Labeling of model GAPDH and HSP90-alpha mRNA molecules via an indirect in situ hybridization (ISH) procedure was done with modified mRNA ISH oligonucleotide probes featuring 5′ 20 nt mRNA-recognition portion and a 3′ 16 nt QDot-binding tag separated by a single-stranded AAAAA spacer (Tables 2 and 3). Hybridization of oligonucleotide probes under optimized ISH conditions yielded labeling of each mRNA molecule with multiple ssDNA tags (up to 36 for GAPDH and 48 for HSP90-alpha), producing distinct spots upon staining with complementary QDot-ssDNA probes consistent with results achieved with conventional mRNA ISH protocols (FIG. 12). In some instances, non-complementary QDot-ssDNA probes failed to hybridize to exposed ssDNA tags, producing minimal non-specific staining background. To explore effects of potential secondary structure formation in 41 nt ssDNA oligonucleotides as well as steric hindrance experienced by QDots approaching tightly spaced ssDNA tags, an alternative mRNA ISH probe set was designed with each probe containing a 16 bp dsDNA spacer between 5′ mRNA-recognition and 3′ QDot-binding portions. Indeed, physical separation of functional ssDNA portions improved mRNA staining intensity in comparison to linear 41 nt ssDNA oligonucleotides (FIG. 13A, FIG. 13B, FIG. 13C, and FIG. 13D), offering one strategy for enhancing per-spot signal intensity and improving signal-to-noise ratio.

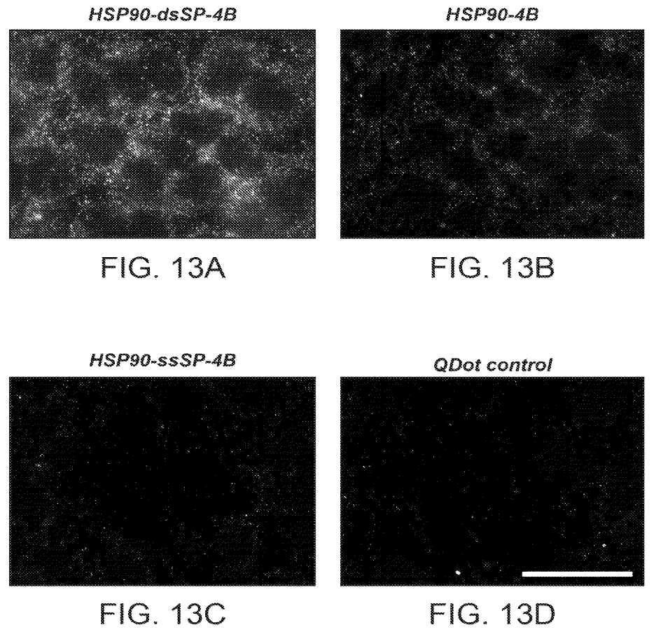

Separation of target-recognition and QDot-labeling events via an intermediate DNA encoding enabled straightforward implementation of a model multi-omics imaging protocol, with both mRNA and protein targets being robustly labeled by respective QDot probes and accurately identified through hyperspectral imaging and analysis (FIG. 14), corroborating broad applicability of the DNA encoding strategy for simultaneous detection and imaging of various types of targets within the same specimen.

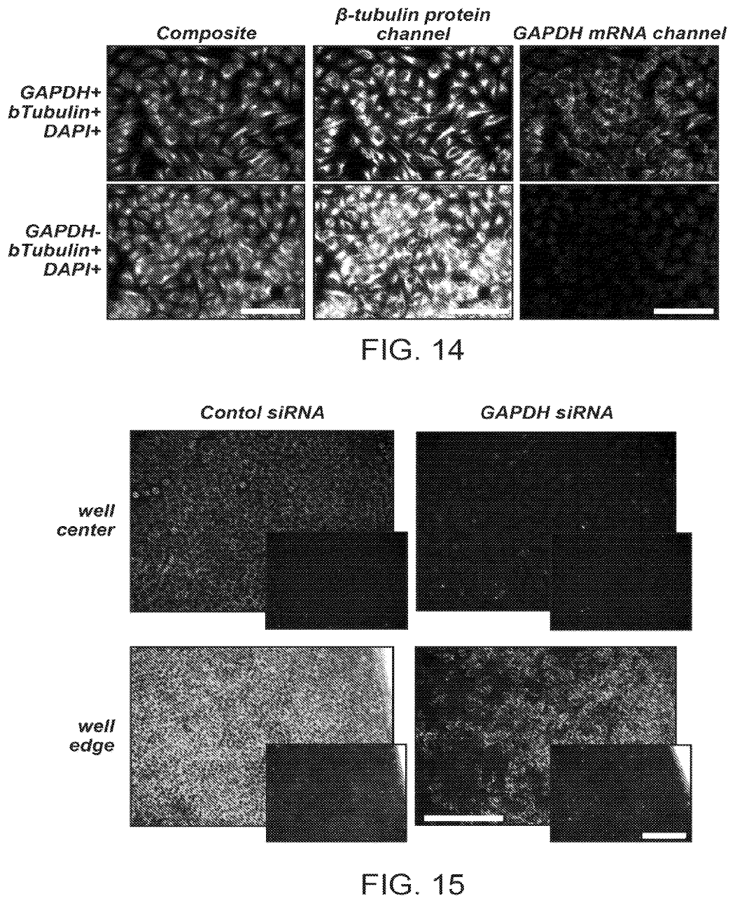

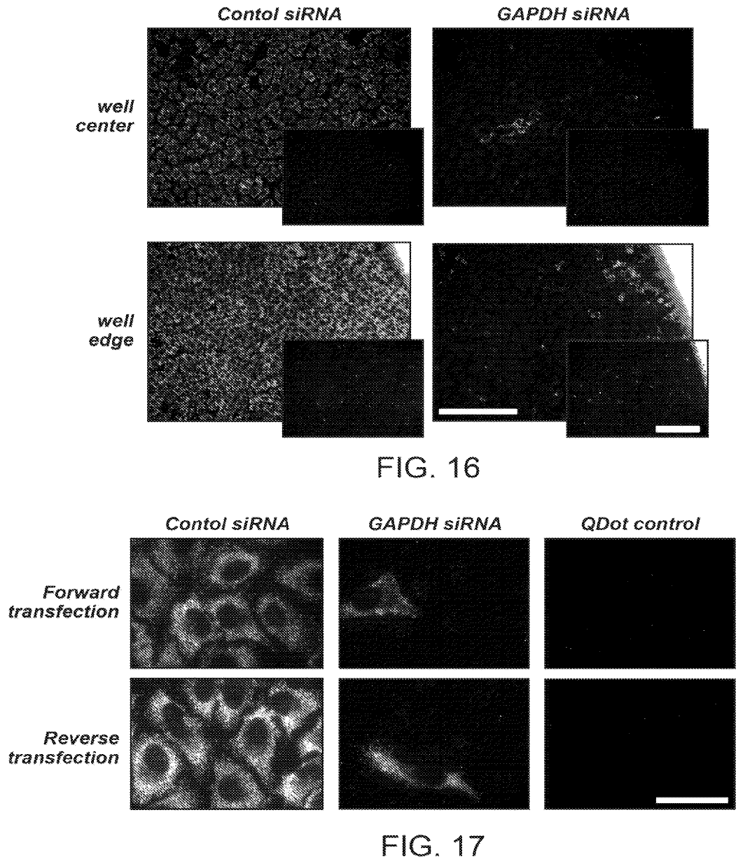

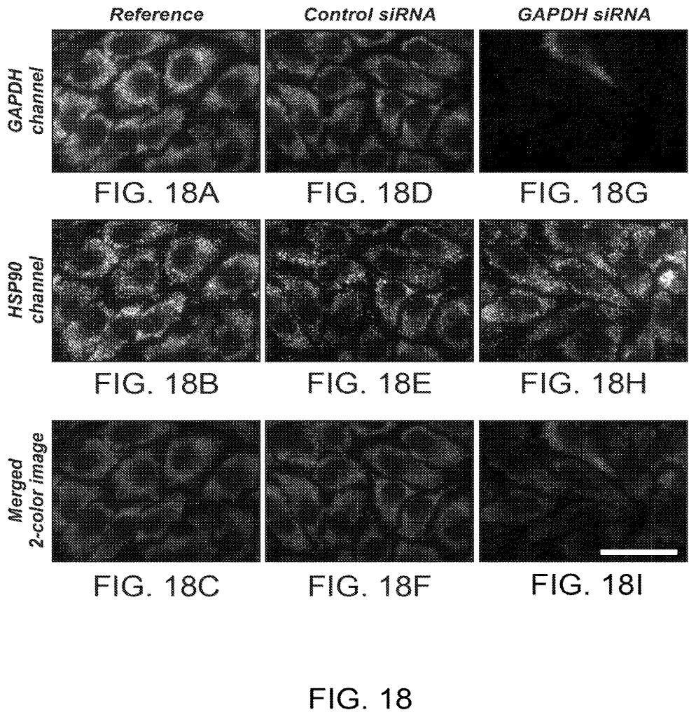

Multi-omics imaging platform was then applied to study gene knock-down via RNAi at a single-cell level. HeLa cells were transfected with GAPDH-targeting siRNA (as well as non-targeting siRNA for control) for 24 Hrs, and GAPDH mRNA abundance was assessed with RT-PCR and QDot-based imaging. In some cases, bulk GAPDH mRNA measurement by RT-PCR indicated silencing efficiency of 78% with forward transfection and 95% with reverse transfection. At the same time, imaging revealed heterogeneity in RNAi, likely resulting from heterogeneous cell transfection with siRNA throughout different regions of cell culture. For example, forward transfection failed to achieve efficient GAPDH mRNA degradation in dense cell populations, yielding areas of completely silenced cells along with patches of cells with normal GAPDH mRNA expression levels (FIG. 15). In contrast, reverse transfection achieved a more uniform cell transfection in suspension, producing a greater proportion of silenced cells with only a few wild-type clones (FIG. 16). Direct comparison of mRNA imaging results obtained from forward vs. reverse transfection further corroborated complete mRNA degradation upon successful transfection with either method along with unperturbed GAPDH mRNA levels in non-transfected cells (FIG. 17), suggesting an all-on/all-off mode of GAPDH RNAi and attributing incomplete silencing observed with bulk RT-PCR analysis to heterogeneity in siRNA transfection.

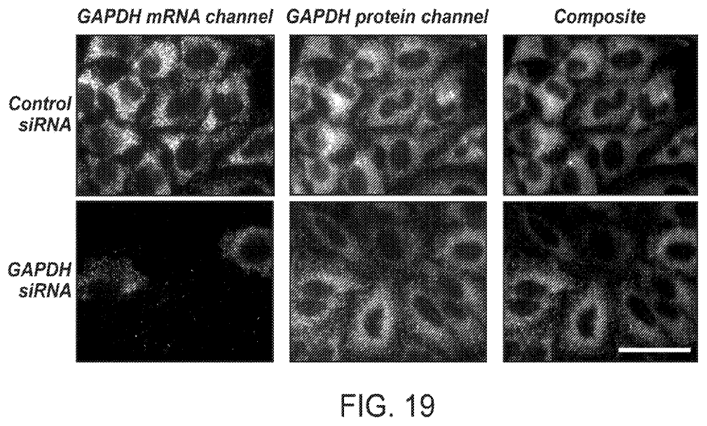

Selectivity of GAPDH RNAi was confirmed by performing dual-target imaging of GAPDH mRNA and HSP90-alpha mRNA. Target-selective siRNA should trigger degradation of only its complementary target mRNA, having no immediate effect on non-targeted mRNA molecules. This was indeed observed with GAPDH RNAi studies (FIG. 18). Indirect dual-target ISH produced robust staining of both mRNA species in reference HeLa cells grown in culture medium. Similarly, cell transfection with non-targeting control siRNA failed to produce any effect on mRNA expression. Transfection with GAPDH-targeting siRNA, however, triggered rapid degradation of GAPDH mRNA within 24 Hrs post-transfection, while leaving non-targeted HSP90-alpha mRNA intact. A single non-transfected cell within the field of view features intact expression of both GAPDH and HSP90 mRNA, consistent with discussion above.

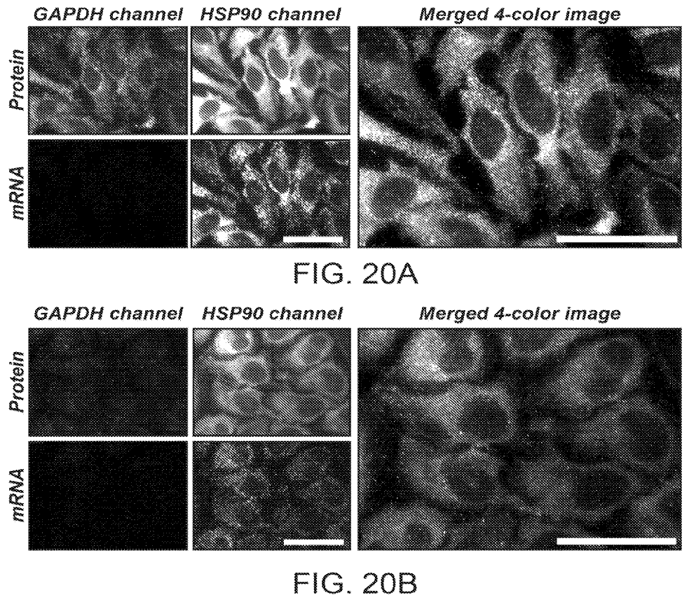

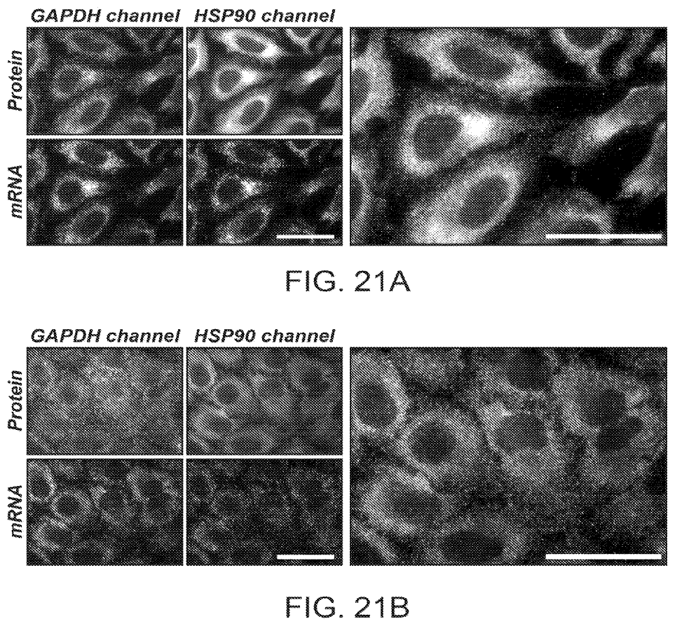

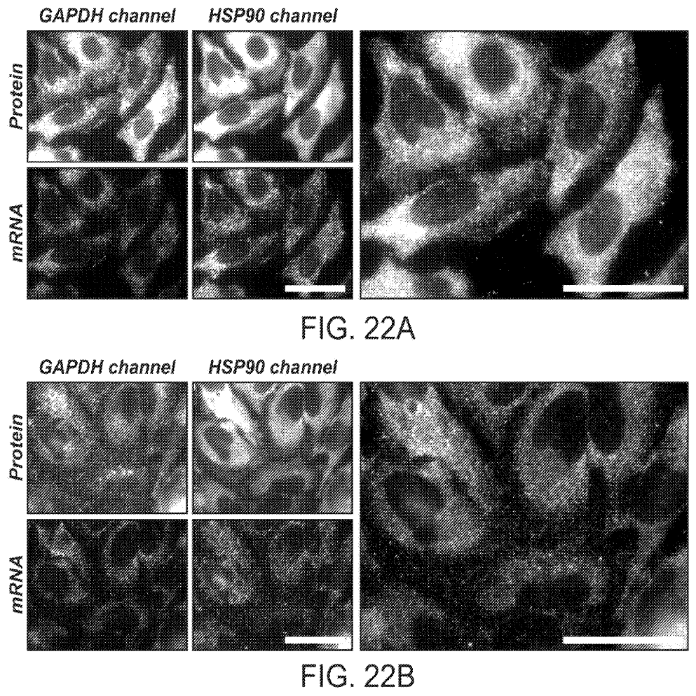

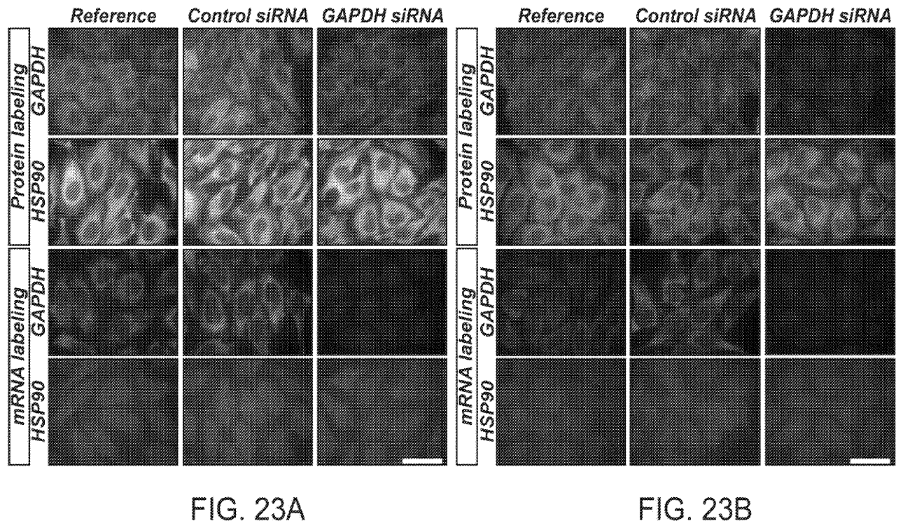

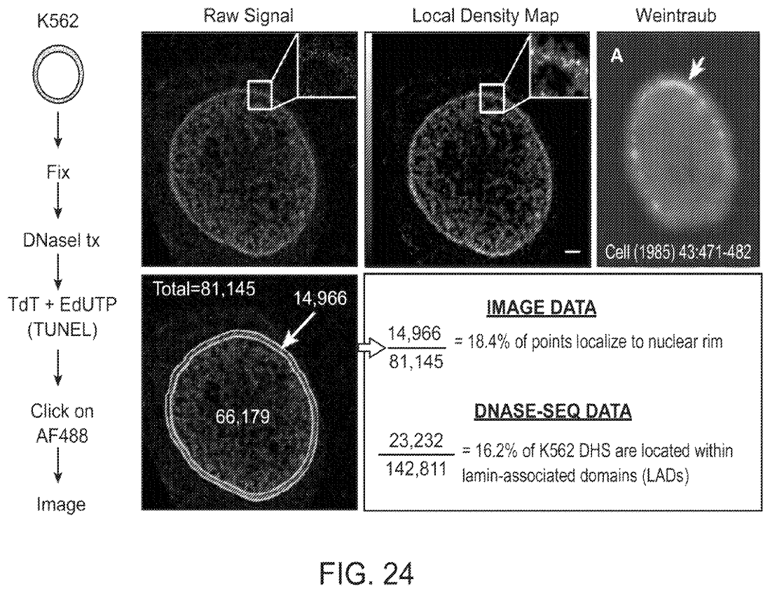

Imaging of mRNA unambiguously demonstrated heterogeneity in RNAi stemming from incomplete cell transfection with siRNA. However, such heterogeneity could not be detected at the protein level, as GAPDH protein remained unperturbed 24 Hrs post-transfection in both transfected and non-transfected cells, as was evident from dual labeling of GAPDH mRNA and protein (FIG. 19). To further investigate the disparity between RNAi effect at mRNA and protein levels, HeLa cells were reverse transfected with GAPDH-targeting siRNA for 24 and 48 Hrs and processed for multiplexed imaging of GAPDH and HSP90-alpha mRNA and their respective protein products. Consistent with studies discussed earlier, 24 Hrs post-transfection a complete degradation of GAPDH mRNA was observed, whereas GAPDH protein level remained unperturbed (FIG. 20A). In contrast, 48 Hrs post-transfection a substantial reduction of GAPDH protein level could be observed, with GAPDH mRNA remaining below the detection limit (FIG. 20B). HSP90 mRNA and protein levels remained unperturbed through 48 hours, confirming selectivity of GAPDH silencing. Further, all molecular targets exhibited consistent unperturbed levels in reference non-transfected cells (FIG. 21A and FIG. 21B) and cells transfected with non-targeting siRNA (FIG. 22A and FIG. 22B) throughout the study, corroborating that the observed GAPDH knock-down indeed resulted from RNAi mechanism. Multiplexed analysis was fully confirmed by a series of single-plex studies to mitigate any artifacts that could potentially be introduced from the multi-omics labeling methodology, HSI, and image analysis (FIG. 23A and FIG. 23B).

In some cases, delay in RNAi effect at the protein level is present, as proteins are typically degraded and cleared slower in comparison to siRNA-mediated mRNA degradation. In other cases, heterogeneity in cell transfection can modulate assessing RNAi efficiency with bulk RT-PCR measurement and downstream phenotypic and molecular signaling analysis. Non-transfected cells might gain growth advantage and achieve substantial clonal expansion during the time it takes for higher-level manifestations of RNAi to occur, thus distorting observed RNAi effect at a population level. Imaging-based analysis at a single-cell level can by-pass this ambiguity and can offer a more accurate insight into molecular processes.

TABLE 4
List of ssDNA/ssDNA′ tag pairs for encoding
of molecular targets

| Tag ID | | Sequence* | SEQ |
|---|---|---|---|
| | | | ID |
| | | | NO: |
| QDot- | 1A | 5′-/5AmMC6/iSp18/CGTCGCACCAAG | 936 |
| | | AAAT-3′ | |
| | 2A | 5′-/5AmMC6/iSp18/TAGACTTGCCAT | 937 |
| | | ACGT-3′ | |
| | 3A | 5′-/5AmMC6/iSp18/AATTCTTGAGAC | 938 |
| | | CAGG-3′ | |
| | 4A | 5′-/5AmMC6/iSp18/ATCTGCCCAAAC | 939 |
| | | TCCA-3′ | |
| | 5A | 5′-/5AmMC6/iSp18/TTCCCAAGCGTC | 940 |
| | | ATCT-3′ | |
| | 6A | 5′-/5AmMC6/iSp18/TCTATCGGACGC | 941 |
| | | TGTA-3′ | |
| IgG- | 1B | 5′-/5AmMC6/AAAAAAAAAAATTTCTTG | 942 |
| compound | | GTGCGACG-3′ | |
| | 2B | 5′-/5AmMC6/AAAAAAAAAAACGTATGG | 943 |
| | | CAAGTCTA-3′ | |
| | 3B | 5′-/5AmMC6/AAAAAAAAAACCTGGTCT | 944 |
| | | CAAGAATT-3′ | |
| | 4B | 5′-/5AmMC6/AAAAAAAAAATGGAGTTT | 945 |
| | | GGGCAGAT-3′ | |
| | 5B | 5′-/5AmMC6/AAAAAAAAAAAGATGACG | 946 |
| | | CTTGGGAA-3′ | |
| | 6B | 5′-/5AmMC6/AAAAAAAAAATACAGCGT | 947 |
| | | CCGATAGA-3′ | |

*all ssDNA tags have 5′ terminal amine group /5AmMC6/ for bioconjugation separated from the pairing sequence by either a hexa-ethyleneglycol spacer /iSp18/ for QDot-coupled tags or 10A oligonucleotide spacer for IgG-coupled tags.

TABLE 5
Sequences of GAPDH mRNA ISH probes
(with 2B encoding tag)

| | | | |
|---|---|---|---|
| | | | SEQ |
| | | | ID |
| # | mRNA-recognition region | encoding tag 2B | NO: |

| | | |
|---|---|---|
| 1 | 5′-ATTTATAGAAACCGGGGGCG-AAAAA- | 947 |
| | ACGTATGGCAAGTCTA-3′ | |
| 2 | 5′-CGAACAGGAGGAGCAGAGAG-AAAAA- | 949 |
| | ACGTATGGCAAGTCTA-3′ | |
| 3 | 5′-GCTGGCGACGCAAAAGAAGA-AAAAA- | 950 |
| | ACGTATGGCAAGTCTA-3′ | |
| 4 | 5′-CATGGTGTCTGAGCGATGTG-AAAAA- | 951 |
| | ACGTATGGCAAGTCTA-3′ | |
| 5 | 5′-TACGACCAAATCCGTTGACT-AAAAA- | 952 |
| | ACGTATGGCAAGTCTA-3′ | |
| 6 | 5′-CAGAGTTAAAAGCAGCCCTG-AAAAA- | 953 |
| | ACGTATGGCAAGTCTA-3′ | |
| 7 | 5′-GGGTCATTGATGGCAACAAT-AAAAA- | 954 |
| | ACGTATGGCAAGTCTA-3′ | |
| 8 | 5′-AACCATGTAGTTGAGGTCAA-AAAAA- | 955 |
| | ACGTATGGCAAGTCTA-3′ | |
| 9 | 5′-GGGTGGAATCATATTGGAAC-AAAAA- | 956 |
| | ACGTATGGCAAGTCTA-3′ | |
| 10 | 5′-TTGACGGTGCCATGGAATTT-AAAAA- | 957 |
| | ACGTATGGCAAGTCTA-3′ | |
| 11 | 5′-CATTGATGACAAGCTTCCCG-AAAAA- | 958 |
| | ACGTATGGCAAGTCTA-3′ | |
| 12 | 5′-TCCTGGAAGATGGTGATGGG-AAAAA- | 959 |
| | ACGTATGGCAAGTCTA-3′ | |
| 13 | 5′-CCACTTGATTTTGGAGGGAT-AAAAA- | 960 |
| | ACGTATGGCAAGTCTA-3′ | |
| 14 | 5′-GGACTCCACGACGTACTCAG-AAAAA- | 961 |
| | ACGTATGGCAAGTCTA-3′ | |
| 15 | 5′-TTCTCCATGGTGGTGAAGAC-AAAAA- | 962 |
| | ACGTATGGCAAGTCTA-3′ | |
| 16 | 5′-AGAGATGATGACCCTTTTGG-AAAAA- | 963 |
| | ACGTATGGCAAGTCTA-3′ | |
| 17 | 5′-GACGAACATGGGGGCATCAG-AAAAA- | 964 |
| | ACGTATGGCAAGTCTA-3′ | |
| 18 | 5′-CATACTTCTCATGGTTCACA-AAAAA- | 965 |
| | ACGTATGGCAAGTCTA-3′ | |
| 19 | 5′-ATTGCTGATGATCTTGAGGC-AAAAA- | 966 |
| | ACGTATGGCAAGTCTA-3′ | |
| 20 | 5′-CTAAGCAGTTGGTGGTGCAG-AAAAA- | 967 |
| | ACGTATGGCAAGTCTA-3′ | |
| 21 | 5′-CCACGATACCAAAGTTGTCA-AAAAA- | 968 |
| | ACGTATGGCAAGTCTA-3′ | |
| 22 | 5′-TCTTCTGGGTGGCAGTGATG-AAAAA- | 969 |
| | ACGTATGGCAAGTCTA-3′ | |
| 23 | 5′-TAGAGGCAGGGATGATGTTC-AAAAA- | 970 |
| | ACGTATGGCAAGTCTA-3′ | |
| 24 | 5′-TCAGCTCAGGGATGACCTTG-AAAAA- | 971 |
| | ACGTATGGCAAGTCTA-3′ | |
| 25 | 5′-CACTGACACGTTGGCAGTGG-AAAAA- | 972 |
| | ACGTATGGCAAGTCTA-3′ | |
| 26 | 5′-CAGGTTTTTCTAGACGGCAG-AAAAA- | 973 |
| | ACGTATGGCAAGTCTA-3′ | |
| 27 | 5′-CACCTTCTTGATGTCATCAT-AAAAA- | 974 |
| | ACGTATGGCAAGTCTA-3′ | |
| 28 | 5′-GCTGTTGAAGTCAGAGGAGA-AAAAA- | 975 |
| | ACGTATGGCAAGTCTA-3′ | |
| 29 | 5′-CGTCAAAGGTGGAGGAGTGG-AAAAA- | 976 |
| | ACGTATGGCAAGTCTA-3′ | |
| 30 | 5′-AGTGGTCGTTGAGGGCAATG-AAAAA- | 977 |
| | ACGTATGGCAAGTCTA-3′ | |
| 31 | 5′-TCATACCAGGAAATGAGCTT-AAAAA- | 978 |
| | ACGTATGGCAAGTCTA-3′ | |
| 32 | 5′-CCTGTTGCTGTAGCCAAATT-AAAAA- | 979 |
| | ACGTATGGCAAGTCTA-3′ | |
| 33 | 5′-TGAGGAGGGGAGATTCAGTG-AAAAA- | 980 |
| | ACGTATGGCAAGTCTA-3′ | |
| 34 | 5′-CTCTTCAAGGGGTCTACATG-AAAAA- | 981 |
| | ACGTATGGCAAGTCTA-3′ | |
| 35 | 5′-TACATGACAAGGTGCGGCTC-AAAAA- | 982 |
| | ACGTATGGCAAGTCTA-3′ | |
| 36 | 5′-TGAGCACAGGGTACTTTATT-AAAAA- | 983 |
| | ACGTATGGCAAGTCTA-3′ | |

Note:
mRNA-recognition region and encoding tag are separated by a spacer SP. Shorter 41 nt mRNA ISH probes contain -AAAAA- single-stranded spacer. Longer 60 nt mRNA ISH probes contain pre-hybridized 16 bp double-stranded spacer flanked by -AAAA- single-stranded linkers.

TABLE 6
Sequences of HSP90-alpha mRNA ISH probes
(with 4B encoding tag)

| | | | |
|---|---|---|---|
| | | | SEQ |
| | | | ID |
| # | mRNA-recognition region | encoding tag 4B | NO: |

| | | |
|---|---|---|
| 1 | 5′-AGGAGTATGATTGTCAACCC-AAAAA- | 984 |
| | TGGAGTTTGGGCAGAT-3′ | |
| 2 | 5′-CCTATATAAGGCGAAGCAC-AAAAAA- | 985 |
| | TGGAGTTTGGGCAGAT-3′ | |
| 3 | 5′-GAGTGACTCGAGAGAGCT-ACAAAAA- | 986 |
| | TGGAGTTTGGGCAGAT-3′ | |
| 4 | 5′-ATAGTGAGCAACGTAGGCTT-AAAAA- | 987 |
| | TGGAGTTTGGGCAGAT-3′ | |
| 5 | 5′-GGACATGAGTTGGGCAATTT-AAAAA- | 988 |
| | TGGAGTTTGGGCAGAT-3′ | |
| 6 | 5′-GAGATCAACTCCCGAAGGAA-AAAAA- | 989 |
| | TGGAGTTTGGGCAGAT-3′ | |
| 7 | 5′-AATCTTGTCCAAGGCATCAG-AAAAA- | 990 |
| | TGGAGTTTGGGCAGAT-3′ | |
| 8 | 5′-AACTTCGAAGGGTCTGTCAG-AAAAA- | 991 |
| | TGGAGTTTGGGCAGAT-3′ | |
| 9 | 5′-GGTTGGGGATGATGTCAATT-AAAAA- | 992 |
| | TGGAGTTTGGGCAGAT-3′ | |
| 10 | 5′-TACCAAAGTCAGGGTACGTT-AAAAA- | 993 |
| | TGGAGTTTGGGCAGAT-3′ | |
| 11 | 5′-TGAGATCAGCTTTGGTCATG-AAAAA- | 994 |
| | TGGAGTTTGGGCAGAT-3′ | |
| 12 | 5′-TTGGCAATGGTTCCCAAATT-AAAAA- | 995 |
| | TGGAGTTTGGGCAGAT-3′ | |
| 13 | 5′-CTGAAGAGCCTCCATGAATG-AAAAA- | 996 |
| | TGGAGTTTGGGCAGAT-3′ | |
| 14 | 5′-CCACCAAGTAGGCAGAATAA-AAAAA- | 997 |
| | TGGAGTTTGGGCAGAT-3′ | |
| 15 | 5′-TGCTTTGTGATCACAACCAC-AAAAA- | 998 |
| | TGGAGTTTGGGCAGAT-3′ | |
| 16 | 5′-CAGAAGACTCCCAAGCATAC-AAAAA- | 999 |
| | TGGAGTTTGGGCAGAT-3′ | |
| 17 | 5′-AGCACGCACAGTGAAGGAAC-AAAAA- | 1000 |
| | TGGAGTTTGGGCAGAT-3′ | |
| 18 | 5′-TCTAGGTACTCTGTCTGATC-AAAAA- | 1001 |
| | TGGAGTTTGGGCAGAT-3′ | |
| 19 | 5′-TAAAGGGTGATGGGATAGCC-AAAAA- | 1002 |
| | TGGAGTTTGGGCAGAT-3′ | |
| 20 | 5′-TGTTTAGTTCTTCCTGATCA-AAAAA- | 1003 |
| | TGGAGTTTGGGCAGAT-3′ | |
| 21 | 5′-AGGGTTTCTGGTCCAAATAG-AAAAA- | 1004 |
| | TGGAGTTTGGGCAGAT-3′ | |
| 22 | 5′-TCATTAGTGAGGCTCTTGTA-AAAAA- | 1005 |
| | TGGAGTTTGGGCAGAT-3′ | |
| 23 | 5′-AAAGTGCTTGACTGCCAAGT-AAAAA- | 1006 |
| | TGGAGTTTGGGCAGAT-3′ | |
| 24 | 5′-TGAATTCCAACTGACCTTCT-AAAAA- | 1007 |
| | TGGAGTTTGGGCAGAT-3′ | |
| 25 | 5′-GAGCCCGACGAGGAATAAAT-AAAAA- | 1008 |
| | TGGAGTTTGGGCAGAT-3′ | |
| 26 | 5′-TGAACACACGGCGGACATAG-AAAAA- | 1009 |
| | TGGAGTTTGGGCAGAT-3′ | |
| 27 | 5′-ATCAACTCATCACAGCTGTC-AAAAA- | 1010 |
| | TGGAGTTTGGGCAGAT-3′ | |
| 28 | 5′-AAGATTTTGCTCTGCTGGAG-AAAAA- | 1011 |
| | TGGAGTTTGGGCAGAT-3′ | |
| 29 | 5′-AGAGAAGAGCTCAAGGCACT-AAAAA- | 1012 |
| | TGGAGTTTGGGCAGAT-3′ | |
| 30 | 5′-GTGGATTCCAAGCTTGAGAT-AAAAA- | 1013 |
| | TGGAGTTTGGGCAGAT-3′ | |
| 31 | 5′-AGACTGGGAGGTATGATAGC-AAAAA- | 1014 |
| | TGGAGTTTGGGCAGAT-3′ | |
| 32 | 5′-CTCTGACAGAGATGTCATCT-AAAAA- | 1015 |
| | TGGAGTTTGGGCAGAT-3′ | |
| 33 | 5′-TAGATGGACTTCTGTGTCTC-AAAAA- | 1016 |
| | TGGAGTTTGGGCAGAT-3′ | |
| 34 | 5′-GCTCCACAAAAGCTGAGTTG-AAAAA- | 1017 |
| | TGGAGTTTGGGCAGAT-3′ | |
| 35 | 5′-CATATATACCACCTCGAAGC-AAAAA- | 1018 |
| | TGGAGTTTGGGCAGAT-3′ | |
| 36 | 5′-ACACAGTACTCGTCAATGGG-AAAAA- | 1019 |
| | TGGAGTTTGGGCAGAT-3′ | |
| 37 | 5′-TTCCCATCAAATTCCTTGAG-AAAAA- | 1020 |
| | TGGAGTTTGGGCAGAT-3′ | |
| 38 | 5′-GAGATTGTCACCTTCTCAAC-AAAAA- | 1021 |
| | TGGAGTTTGGGCAGAT-3′ | |
| 39 | 5′-TGCAGCAAGGTGAAGACACA-AAAAA- | 1022 |
| | TGGAGTTTGGGCAGAT-3′ | |
| 40 | 5′-GCTTTTTGGCCATCATATAG-AAAAA- | 1023 |
| | TGGAGTTTGGGCAGAT-3′ | |
| 41 | 5′-AACTGCCTTATCATTCTTGT-AAAAA- | 1024 |
| | TGGAGTTTGGGCAGAT-3′ | |
| 42 | 5′-ATCCTCAAGGGAAAAGCCAG-AAAAA- | 1025 |
| | TGGAGTTTGGGCAGAT-3′ | |
| 43 | 5′-TGATCATGCGATAGATGCGG-AAAAA- | 1026 |
| | TGGAGTTTGGGCAGAT-3′ | |
| 44 | 5′-CATCAGGAACTGCAGCATTG-AAAAA- | 1027 |
| | TGGAGTTTGGGCAGAT-3′ | |
| 45 | 5′-CAAGGGCACAAGTTTTCCAA-AAAAA- | 1028 |
| | TGGAGTTTGGGCAGAT-3′ | |
| 46 | 5′-TACTGCCTTCAACACAAGGA-AAAAA- | 1029 |
| | TGGAGTTTGGGCAGAT-3′ | |
| 47 | 5′-AGAGTAGAGAGGGAATGGGG-AAAAA- | 1030 |
| | TGGAGTTTGGGCAGAT-3′ | |
| 48 | 5′-TACACAACATCCAATCCTGC-AAAAA- | 1031 |
| | TGGAGTTTGGGCAGAT-3′ | |

Note:
mRNA-recognition portion and encoding tag are separated by a spacer. Shorter 41 nt mRNA ISH probes contain -AAAAA- single-stranded spacer. Longer 60 nt mRNA ISH probes contain pre-hybridized 16 bp double-stranded spacer flanked by -AAAA- single-stranded linkers.

### Example 3

### Global In Situ Visualization of the DNasel Hypersensitivity Site (DHS) Compartment of a Cell

This example shows the global in situ visualization of the DNasel Hypersensitivity Site (DHS) compartment of a cell, which allows for identification of nuclear compartments where regulatory DNA activation occurs. As shown in the graphic on the left side of FIG. 24, K562 cells were fixed with Paxgene reagent, treated with DNasel, DNasel-induced nicks were labeled using terminal transferase (TdT) and ethynyl-dUTP (EdUTP) (TUNEL assay), Alexafluor-488 (AF488) was conjugated to the EdUTP via copper click chemistry, and then SPDM imaging was performed. FIG. 24 shows multiple images of this. The top left image is of the raw signal data. The local density map image (top middle) shows a ring of condensation at the nuclear lamina, which is similar to findings reported by the Weintraub lab 30 years ago (Weintraub, Cell (1985) 43:471-482); see FIG. 24 top right reproduced image). Approximately 18.4% of the localized points are within the ring density at the nuclear lamina, as shown the calculations in the lower right box, in which the image data calculation was based off the image on the lower left of FIG. 24. The image data calculation is similar to the proportion of K562 DHS within lamina-associated domains (LADS). These findings indicate labeling of DNasel cut sites in a cell's nucleus using a TUNEL assay may be used for better understanding of the nuclear localization of regulatory DNA activation.

### Example 4

### Nano-FISH Methods

This example shows how nano-FISH was used to detect the presence or absence of the locus control region in human erythroleukemia K562 cells.

### Cells and Tissue Culture

Human erythroleukemia K562 cells and their derivative lacking the locus control region (ALCR) were maintained in RPMI 1640 media supplemented with 2 mM L-glutamine (0.3 g/L), 10% Fetal Bovine Serum, penicillin, and streptomycin at 37° C. in 5% CO₂.

### In Silico Design of Nano-FISH Probe Pools

Tiled 40 bp probe pools with a minimum of 2 bp spacing between consecutive probes were designed using Primer3 with default parameters. The resulting tiled probe sets were compared to a 16-mer database of genomic sequences in each register to model partial matches of probes to genomic sequences that could result in inappropriate background staining. A uniquely mapping oligonucleotide would therefore have a total of 24 matches to the 16-mer database. Individual probe sets with >100 16-mer database matches were empirically discarded from consideration. For the genomic target regions examined in this study, a pool of at least 30 oligonucleotides that satisfied these design criteria was used.

### Nano-FISH Protocol

Cells were harvested, washed once in phosphate buffered saline (PBS), re-suspended in a small volume of PBS and subsequently seeded on 18 mm×18 mm coverslips in a 6-well plate that had been coated with poly-L-lysine (Sigma P1399). After allowing cells to adhere for 5 to 10 minutes at room temperature they were fixed by the addition of 4% formaldehyde (Polysciences 18814-10) in PBS for 10 minutes, washed with PBS, and then permeabilized for 15 minutes with 0.5% detergent Triton™ X-100 in PBS. Following two washes in PBS, the cells were subjected to a 5 minute treatment of 0.1 M HCl and subsequently washed twice in saline sodium citrate (2×SSC) before incubation with RNase A (25 ug/mL in 2×SSC) at 37° C. for 30 minutes. The cover slips were washed in 2×SSC again and then pre-equilibrated for at least one hour in 50% formamide (Amresco 0606), 2×SSC (pH 7.0) at room temperature. To denature the cellular DNA the cover slips were incubated for 4.5 minutes in 70% formamide, 2×SSC (pH 7.0) preheated to 78° C. in a 6-well plate on a heat block equipped with an aluminum block designed for tissue culture plates. For consistency, only the center two wells were used for denaturations and the temperature allowed to re-equilibrate before the next batch. Cover slips were then inverted onto 80 μl of hybridization solution (50% formamide, 10% dextran sulfate, 2×SSC, 250 pM oligonucleotide pool) on parafilm in a humid chamber and incubated overnight at 37° C. Post-hybridization washes included two 15 minute incubations in 2×SSC followed by two 7 minute washes in 0.2×SSC/0.2% Tween-20 at 56° C. on a heat block and one wash in 4×SSC/0.2% Tween-20 at room temperature. Cellular DNA was counterstained with DAPI (100 ng/mL in 2×SSC), followed by two more washes in 2×SSC. Cover slips were then mounted on slides for imaging with Prolong Gold (Molecular Probes P36930).

### Imaging

For standard widefield microscopy, slides were imaged on an inverted Nikon Eclipse Ti widefield microscope with a 60× Nikon Plan Apo lambda NA 1.40 oil objective and an Andor Zyla 4.2CL10 CMOS camera.

### Example 5

### Nano-FISH Detection of a 1.8 kb Nucleic Acid Sequence

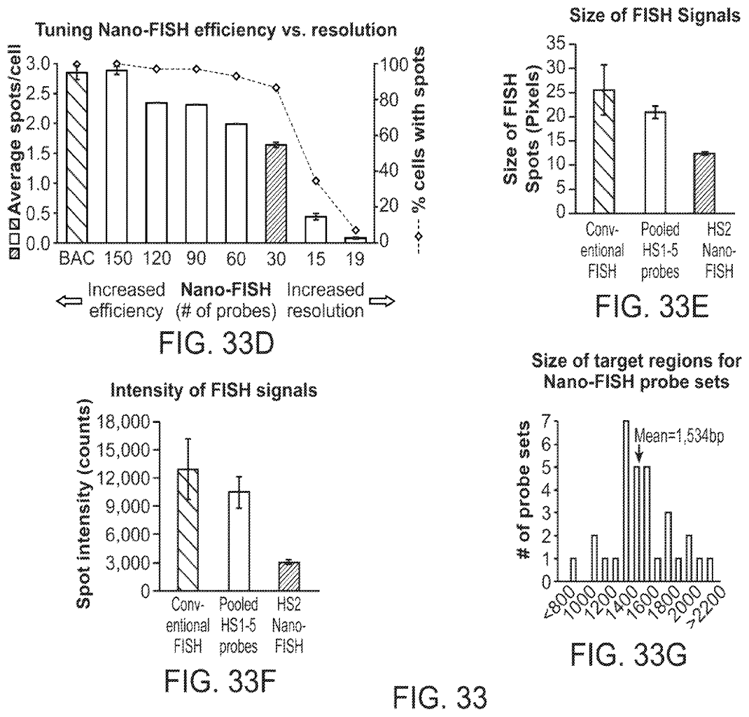

This example and FIG. 33 shows the use of Nano-FISH to detect a 1.8 kb nucleic acid sequence. FIG. 33A shows a schematic of a Nano-FISH experiment. FIG. 33B shows the application of the Nano-FISH strategy to detect a 1.8 kb region encompassing the HS2 hypersensitive site of the β-globin locus control region (LCR) in triploid K562 erythroleukemia cells. FIG. 33C shows colocalization of the Nano-FISH signals (−1.8 kb target region) with those from standard BAC-derived probes (conventional DNA-FISH; −170 kb target region), confirming the specificity of the detected Nano-FISH signal. Compared to the large size of BAC probes used to detect the β-globin LCR, Nano-FISH probes targeting HS2 covered a target region that was approximately 2 orders of magnitude smaller in size. Although most, but not all spots corresponding to the alleles in the triploid cell, were consistently detected using 30 tiled 40 bp oligonucleotide probes targeting the HS2 hypersensitive site, increasing the number of probes and expanding the corresponding labeled genomic DNA target region, Nano-FISH was shown to be tunable. A modest increase in the number of probes (−90), the frequency of allele detecting by Nano-FISH matches the perform of the BAC-probe based gold performance benchmark of Nano-FISH versus standard BAC probes. Conversely, decreasing the number probes below 30 drastically reduced allele detection sensitivity. Therefore, as shown in FIG. 33D, the sensitivity of efficiency and resolution of detection using Nano-FISH may be tuned according to the number of probes being used.

Using an efficient and robust automated image processing pipeline, results from hundreds of cells across multiple replicates were quantified. These studies showed that the diffraction-limited signals produced by Nano-FISH were smaller and dimmer than those generated by BAC-based probes (FIG. 33E and FIG. 33E). Despite this, Nano-FISH still showed robust detection of genomic regions with varying size, such as genomic region size ranging from about 800 bp to 2.1 kb, as shown if FIG. 33G. Thus, Nano-FISH is able to successfully label endogenous non-repetitive DNA loci that are much smaller than the current limit of resolution of BAC- and fosmid-based DNA-FISH approaches.

### Example 6

### Fine Structural Analysis Using Nano-FISH

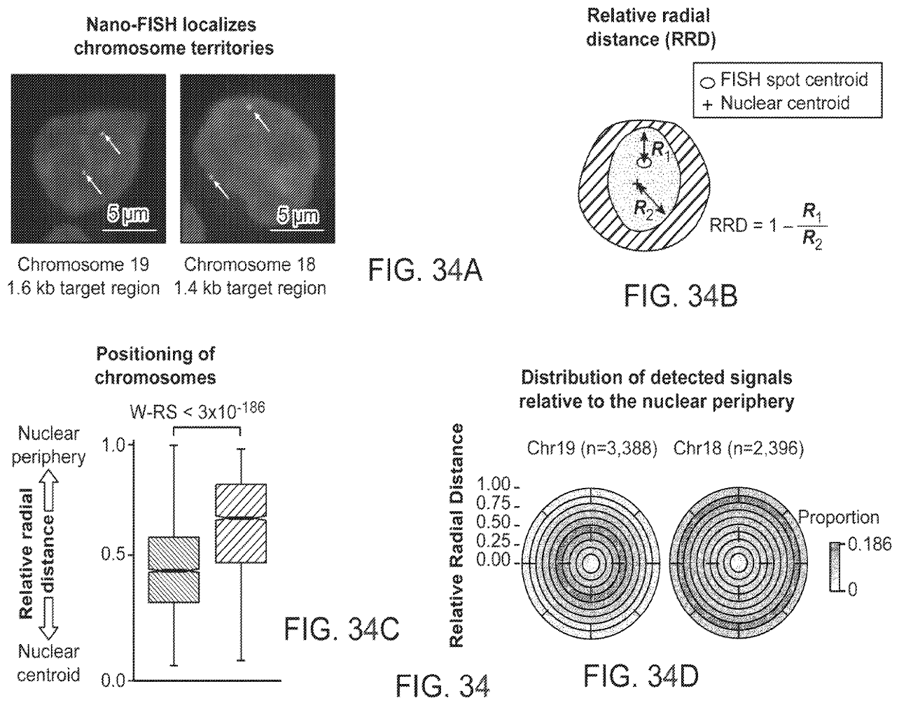

This example and FIG. 34 show the use of Nano-FISH to perform fine structural analysis of specific genomic loci within the nucleus. Probe pools were designed to target a 1.6 kb region of chromosome 19 and a 1.4 kb region of chromosome 18. These chromosomes were chosen since chromosome 19 is known to occupy a central position within the nucleus while chromosome 18 is more marginally located. FIG. 34A shows the distinct spots produced by Nano-FISH probes targeting specific loci on these chromosomes. To measure the relative localization of the detected loci, the relative radial distance (RRD), a normalized measure of the position of the detected spot with respect to the nuclear centroid, was calculated. FIG. 34B shows a schematic of the relative radial distance. FIG. 34C shows that the chromosome 18 Nano-FISH signals are closer to the nuclear periphery. The distributions were obtained across 2,396 chromosome 18 signals and 3,388 chromosome 19 signals. FIG. 34D shows radial histograms of the two target loci. The differences in the distribution of signals with respect to the nuclear centroid are readily apparent in the histograms.

### Example 7

### Examination of Enhancer-Promoter Interactions Using Nano-FISH

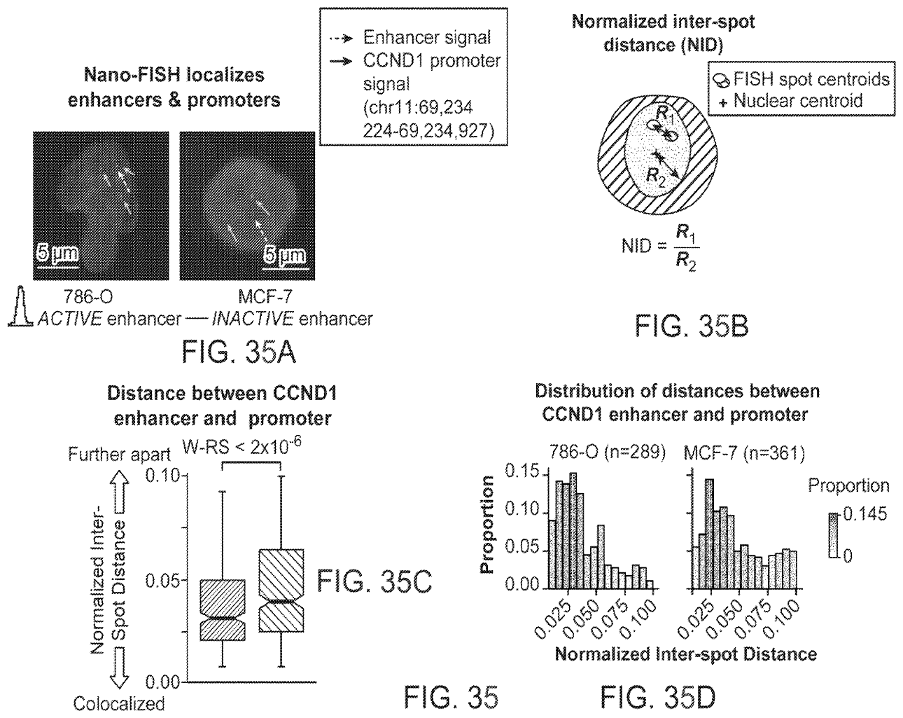

This example and FIG. 35 show the use of Nano-FISH for examining the interaction of a gene enhancer with its target gene promoter. The positioning of a known enhancer of the CCND1 gene in 786-O and MCF-7 cells was examined. Based on DNasel hypersensitivity mapping, this enhancer is active in 786-O cells, but is inactive in MCF-7 cells. Using large (˜225 kb) probes, others have demonstrated that this enhancer is located in proximity to the CCND1 gene promoter in 786-O cells, but not in MCF-7 cells. Nano-FISH probes targeting the enhancer and promoter were designed and synthesized. FIG. 35A shows two-color Nano-FISH in 786-O and MCF-7 cells. The normalized inter-spot distance (NID) between these two genomic loci were compared. FIG. 35B shows a schematic of the normalized inter-spot distance. FIG. 34C shows that, on average, the spots are situated closer together in 786-O cells compared to MCF-7 cells. FIG. 35D shows that, in spite of this, absolute colocalization (NID=0) was actually a rare event in both cell types. Thus, the small size of genomic regions targeted by Nano-FISH permits fine scale localization of regulatory DNA regions and provides a granular view of their spatial localizations within nuclei.

### Example 8

### Detection of Small Genomic Structural Variations Using Nano-FISH

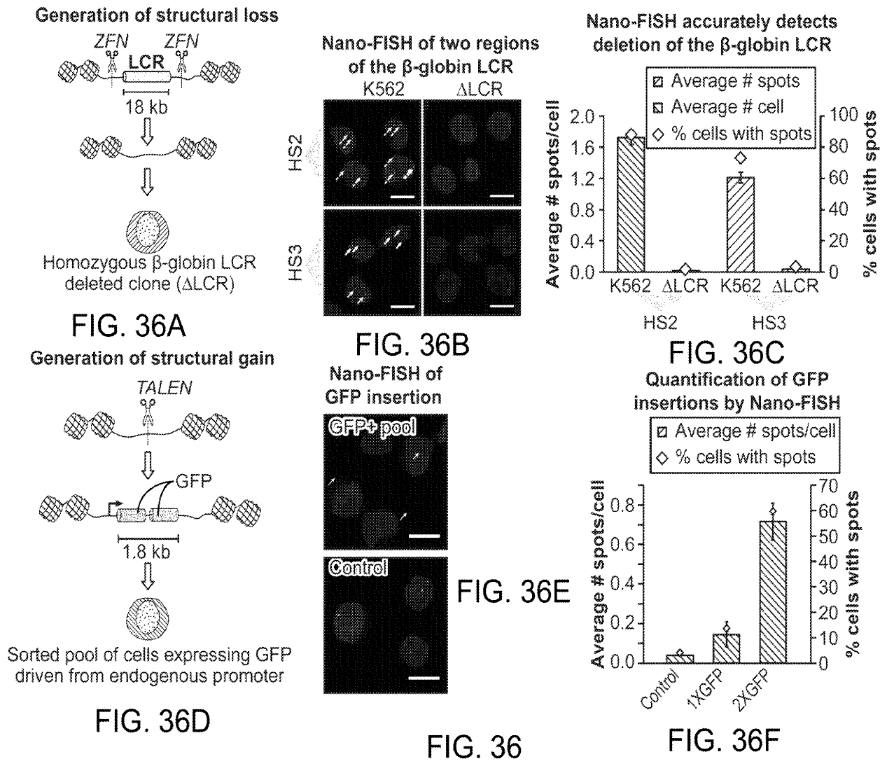

This example and FIG. 36 show the use of Nano-FISH to detect small genomic structural variations such as small losses or gains of DNA. ZFN-mediated genome editing was used to generate a triploid homozygous deletion of the β-globin locus control region (LCR, ˜18 kb) in K562 cells, as shown in FIG. 36A. Cells imbued with this deletion are referred to as ALCR. Probes targeting either the HS2 or HS3 hypersensitive sites within the deleted region were utilized to detect loss of LCR in the genome edited cells, as shown in FIG. 36B and FIG. 36C. For the converse scenario, using TALEN-mediated homology directed repair, a sequence encoding for eGFP was inserted into the AAVS1 safe harbor locus on chromosome 19, as shown in FIG. 36D. This exogenously-derived sequenced was readily identified by Nano-FISH, as shown in FIG. 36E and FIG. 36F.

### Example 9

### Fine Scale Genome Localization Using Nano-FISH and Super-Resolution Microscopy

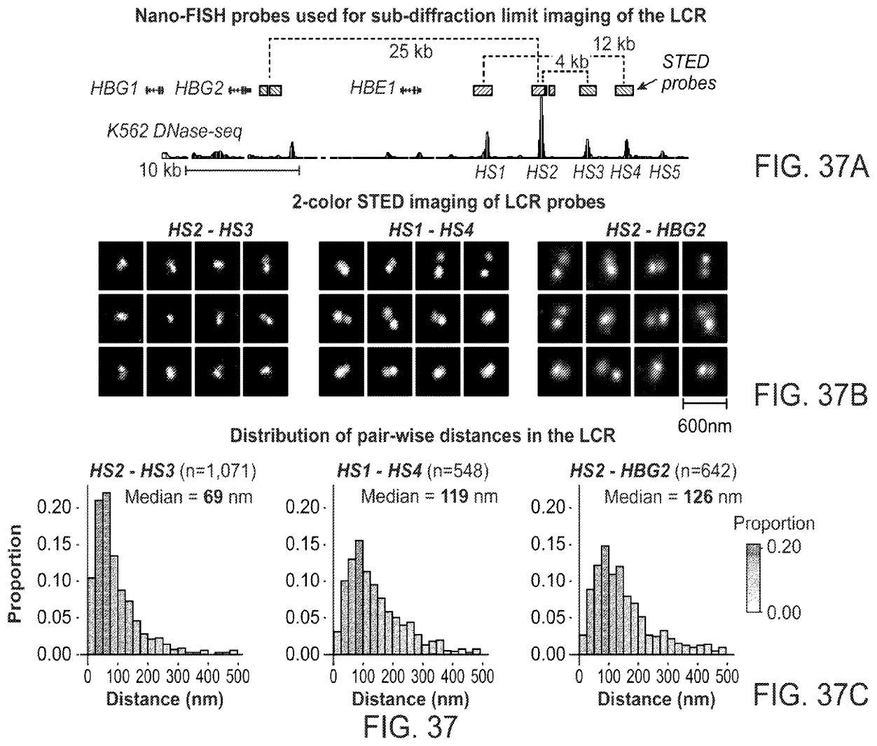

This example and FIG. 37 show the combination of Nano-FISH and super-resolution microscopy to obtain very fine-scale genome localization. A custom automated stimulated emission and depletion (STED) microscope was utilized to efficiently acquire multiple measurements of the physical distance between the HS2 and HS3 genomic loci, which are separated by 4.1 kb of linear genomic distance. FIG. 37A shows that these closely apposed loci are readily discernible as distinct spots by STED microscopy. Pair-wise measurements of other closely situated genomic segments such as HS1-HS4 (˜12 kb) and HS2-HGB2 (˜25 kb) were also readily obtained and revealed non-linear compaction of the β-globin locus control region and the surrounding genome which contains its target genes, as shown in FIG. 37B. Importantly, the high-throughput STED microscopy approach enables calculation of the distribution of actual distances between these various loci, as shown in FIG. 37C. These results demonstrated the suitability of Nano-FISH for super-resolution STED microscopy experiments.

### Example 10

### Optimal Nano-FISH Parameters

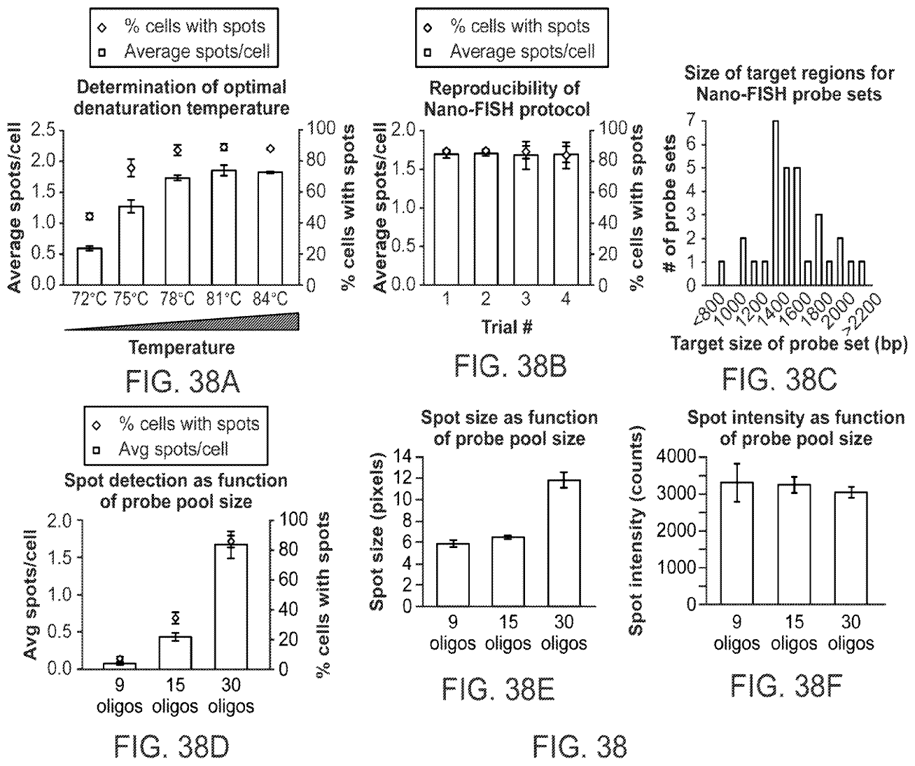

This example and FIG. 38 show a series of experiments to determine the optimal operating parameters for a Nano-FISH experiment. FIG. 38A shows how the labeling efficiency of the Nano-FISH procedure depends on denaturation temperature. With increasing temperature, the efficiency of Nano-FISH labeling increases, until a plateau is reached at a temperature of 78° C. FIG. 38B shows that the Nano-FISH labeling procedure is repeatable across experiments. FIG. 38C shows Nano-FISH detected for genomic regions with varying size, such as genomic region size ranging from about 800 bp to 2.1 kb. FIG. 38D shows how the labeling efficiency of the Nano-FISH experiment depends on the number of oligo probes used. The labeling efficiency increases with the number of oligo probes used, attaining a maximum efficiency when 30 oligo probes are utilized. FIG. 38E shows how the detected fluorescence spot size depends on the number of oligo probes. FIG. 38F shows how the intensity of the fluorescence spot size depends on the number of oligo probes.

### Example 11

### Comparison of Nano-FISH and Conventional FISH

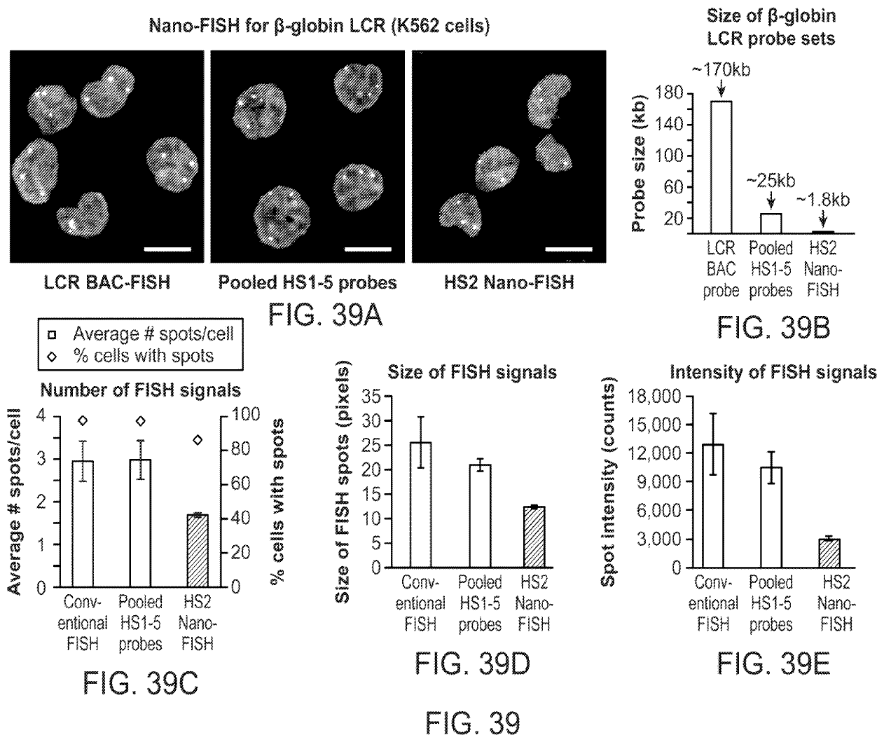

This example and FIG. 39 show a comparison of Nano-FISH and conventional FISH. FIG. 39A shows fluorescence images of β-globin lacking the LCR using conventional BAC probes (left panel), a pool of HS1-5 probes (middle panel), and the HS2 Nano-FISH technique (right panel). FIG. 39B shows the size of the probe sets used for the BAC, HS1-5, and HS2 experiments. As can be seen, the HS2 Nano-FISH experiment utilizes a significantly smaller nucleic acid sequence than conventional FISH techniques. FIG. 39C shows the labeling efficiency of the BAC, HS1-5, and HS2 experiments. FIG. 39D shows the size of the FISH spots for the BAC, HS1-5, and HS2 experiments. FIG. 39E shows the intensity of the FISH signals for the BAC, HS1-5, and HS2 experiments. As can be seen, the Nano-FISH experiment produces a lower signal-to-noise ratio (SNR) than conventional methods, with the Nano-FISH experiment producing a SNR smaller than the BAC method by a factor of approximately 2. The loss in SNR comes with a reduction in the size of the nucleic acid sequence by a factor of approximately 100. Thus, the tradeoff in SNR is well worth the significant reduction in size of nucleic acid sequence.

### Example 12

### Discovery of Novel Biomarkers Using Nano-FISH

This example shows the discovery of the expression of novel biomarkers that correlate the number of the target nucleic acid sequence in a cell. A sample with a population of cells that is heterogenous for the number of target nucleic acid sequences in a cell is obtained. Individual cells from the sample are distributed into a single well of a plate and are allowed to clonally expand. Samples of cells from each clone is then characterized for the number of target nucleic acid sequences in a cell using Nano-FISH and is characterized by RNA-Seq to determine novel biomarkers that correlate with the number of target nucleic acid sequences. If a novel biomarker is found to be a surface protein, then the surface protein is used as a selectable marker/sortable marker to isolate cells with the desired number of target nucleic acid sequences from the sample.

The examples and embodiments described herein are for illustrative purposes only and various modifications or changes suggested to persons skilled in the art are to be included within the spirit and purview of this application and scope of the appended claims.

1.-68. (canceled)

69. A method for detecting presence of a genomic structural variation in a genome of cells, the method comprising:
contacting the cells with a plurality of probes comprising a detectable label and a probe sequence that binds to a portion of the genomic structural variation,
wherein:
the probe sequence is between 30 and 50 nucleotides in length,
the plurality of probes comprises 30 probes to 50 probes, and
wherein:
(i) the genomic structural variation comprises an insertion that is less than 2.5 kb long and the method comprises detecting a presence or absence of the detectable label in the genome of the cells, wherein the presence of the detectable label indicates the presence of the insertion and wherein the absence of the detectable label indicates the absence of the insertion; or
(ii) the genomic structural variation comprises a deletion that is less than 2.5 kb long and the method comprises detecting a presence or absence of the detectable label in the genome of the cells, wherein the presence of the detectable label indicates the absence of the deletion and wherein the absence of the detectable label indicates the presence of the deletion.

70. The method of claim 69, wherein the genomic structural variation comprises an insertion.

71. The method of claim 70, wherein the insertion is an endogenous insertion.

72. The method of claim 71, wherein the endogenous insertion is a translocation.

73. The method of claim 72, wherein the translocation is a somatic chromosomal rearrangement.

74. The method of claim 73, wherein the somatic chromosomal rearrangement comprises a nucleic acid sequence from a T cell receptor, B cell receptor or fragment thereof.

75. The method of claim 70, wherein the insertion is an exogenous insertion.

76. The method of claim 75, wherein the exogenous insertion is inserted by a genetic engineering technique.

77. The method of claim 75, wherein the exogenous insertion comprises a viral sequence.

78. The method of claim 69, wherein the probe sequence is about 40 nucleotides in length.

79. The method of claim 69, wherein the plurality of probes comprises about 40 probes.

80. The method of claim 69, wherein the genomic structural variation comprises a sequence that is 800 nucleotides to 2.1 kb nucleotides long.

81. The method of claim 69, wherein the cells are mammalian cells.

82. The method of claim 69, wherein the detectable label is a fluorescent label.

83. The method of claim 69, wherein the genome of the cells is denatured at a temperature of no higher than 78° C.


SEQUENCE LISTING (1037 sequences)

SEQ ID NO: 1 - DNA, 40 bp, synthetic construct
  source 1..40: mol_type = other DNA
tttcccttgctcttcatgattttaacaacatgatggattt

SEQ ID NO: 2 - DNA, 40 bp, synthetic construct
  source 1..40: mol_type = other DNA
ccctgccccccattaactcacatcctgaattttatgttta

SEQ ID NO: 3 - DNA, 40 bp, synthetic construct
  source 1..40: mol_type = other DNA
gcacttcatcatcgtctttgaagtccccttcttgtcctcc

SEQ ID NO: 4 - DNA, 40 bp, synthetic construct
  source 1..40: mol_type = other DNA
tatgatgaacaccatgcaccacatgcaggttctggtgaag

SEQ ID NO: 5 - DNA, 40 bp, synthetic construct
  source 1..40: mol_type = other DNA
gatacaaaagaatattggtatgtatgttgcacagactcat

SEQ ID NO: 6 - DNA, 40 bp, synthetic construct
  source 1..40: mol_type = other DNA
cctatttcccccacacagccttcccacattggccaaccct

SEQ ID NO: 7 - DNA, 40 bp, synthetic construct
  source 1..40: mol_type = other DNA
tacaaagggcttctctggccagagagagccggtgtctgct

SEQ ID NO: 8 - DNA, 40 bp, synthetic construct
  source 1..40: mol_type = other DNA
tgggggggttaatggagttatggactgggatgggcagcct

SEQ ID NO: 9 - DNA, 40 bp, synthetic construct
  source 1..40: mol_type = other DNA
acctacctagggaactctttctccctggcactaggctagt

SEQ ID NO: 10 - DNA, 40 bp, synthetic construct
  source 1..40: mol_type = other DNA
actgactgagctgacctccagtacagggcctgaggccact

SEQ ID NO: 11 - DNA, 40 bp, synthetic construct
  source 1..40: mol_type = other DNA
ctgggagctaaatagaagcaaatatccccaggcctgggtg

SEQ ID NO: 12 - DNA, 40 bp, synthetic construct
  source 1..40: mol_type = other DNA
atgcgtcaagcaactacactcccacagtaaactgggaacc

SEQ ID NO: 13 - DNA, 40 bp, synthetic construct
  source 1..40: mol_type = other DNA
cagctccttggcagcctaggctctagctcaacatctgctt

SEQ ID NO: 14 - DNA, 40 bp, synthetic construct
  source 1..40: mol_type = other DNA
tgctggagtcgcaccaacctggctctgcctatctccagca

SEQ ID NO: 15 - DNA, 40 bp, synthetic construct
  source 1..40: mol_type = other DNA
ctctgtaggctgcacaacgtggaacagatgaaaggaacca

SEQ ID NO: 16 - DNA, 40 bp, synthetic construct
  source 1..40: mol_type = other DNA
tggggtaaattataatcatgaaattccgtcaagcttgaat

SEQ ID NO: 17 - DNA, 40 bp, synthetic construct
  source 1..40: mol_type = other DNA
aacatatttaatatggcatattcaaatgacagaaagtacg

SEQ ID NO: 18 - DNA, 40 bp, synthetic construct
  source 1..40: mol_type = other DNA
ctttattcttgctaatgttgactccttagcaaagataatt

SEQ ID NO: 19 - DNA, 40 bp, synthetic construct
  source 1..40: mol_type = other DNA
tgatctttgctaaactcttcaggaataaatgaacatttcc

SEQ ID NO: 20 - DNA, 40 bp, synthetic construct
  source 1..40: mol_type = other DNA
ttttcaagcagttaagaagcaagaattaatgactcgaata

SEQ ID NO: 21 - DNA, 40 bp, synthetic construct
  source 1..40: mol_type = other DNA
atgagagtgttgactgatgaagggctcctatacgcgggtt

SEQ ID NO: 22 - DNA, 40 bp, synthetic construct
  source 1..40: mol_type = other DNA
tctttcccatctgtttcccggcccctaccagaaataagtg

SEQ ID NO: 23 - DNA, 40 bp, synthetic construct
  source 1..40: mol_type = other DNA
atgaacctccctcgctccaagaccagagctcctaggaagt

SEQ ID NO: 24 - DNA, 40 bp, synthetic construct
  source 1..40: mol_type = other DNA
tctttattttattggccacaattgaacataggtataattt

SEQ ID NO: 25 - DNA, 40 bp, synthetic construct
  source 1..40: mol_type = other DNA
cagaagcaagccctgatcaaggaaaccattcacacttgat

SEQ ID NO: 26 - DNA, 40 bp, synthetic construct
  source 1..40: mol_type = other DNA
gtggcttttgctcaaagtgaggacgttatcagctctgccc

SEQ ID NO: 27 - DNA, 40 bp, synthetic construct
  source 1..40: mol_type = other DNA
ctttaaacaaaaactaaaggcgtaaggaaagataactact

SEQ ID NO: 28 - DNA, 40 bp, synthetic construct
  source 1..40: mol_type = other DNA
cagttgccacactttttttcactgctaaagttcgtaatga

SEQ ID NO: 29 - DNA, 40 bp, synthetic construct
  source 1..40: mol_type = other DNA
ggcaatcagaagtattttggttgcttctaggtcagaatga

SEQ ID NO: 30 - DNA, 40 bp, synthetic construct
  source 1..40: mol_type = other DNA
ggcagcaaacttgtttaggtatgattcatcattgtctgct

SEQ ID NO: 31 - DNA, 40 bp, synthetic construct
  source 1..40: mol_type = other DNA
ctacaaaacaatgagtctgattacgacccacagaaatgaa

SEQ ID NO: 32 - DNA, 40 bp, synthetic construct
  source 1..40: mol_type = other DNA
cctcccacagacccaaacatgctgctgcaaatgtctcact

SEQ ID NO: 33 - DNA, 40 bp, synthetic construct
  source 1..40: mol_type = other DNA
ggacaagcacacacatcgctgggaagatctgcaagcctcc

SEQ ID NO: 34 - DNA, 40 bp, synthetic construct
  source 1..40: mol_type = other DNA
taaacctggataacaagaacactgtttccactgcgctagt

SEQ ID NO: 35 - DNA, 40 bp, synthetic construct
  source 1..40: mol_type = other DNA
tcatcacgatgacaatggacaagccatatccctaacaggg

SEQ ID NO: 36 - DNA, 40 bp, synthetic construct
  source 1..40: mol_type = other DNA
tttccatgacaccaggaccgtaaagcaccttttacaccgt

SEQ ID NO: 37 - DNA, 40 bp, synthetic construct
  source 1..40: mol_type = other DNA
aattgggatgtgcaaaacctcttaacttgtagcaccaagt

SEQ ID NO: 38 - DNA, 40 bp, synthetic construct
  source 1..40: mol_type = other DNA
tcttgtgttattcgcctgcattgaaatcccatcccaatcc

SEQ ID NO: 39 - DNA, 40 bp, synthetic construct
  source 1..40: mol_type = other DNA
tgagtgatctctttgctgatcataaacatattcctccatc

SEQ ID NO: 40 - DNA, 40 bp, synthetic construct
  source 1..40: mol_type = other DNA
tgcattcattactaaatacacagggcatagcacatagtaa

SEQ ID NO: 41 - DNA, 40 bp, synthetic construct
  source 1..40: mol_type = other DNA
cttcaatgttgccaggaaaatccttgcaggaatcacaccc

SEQ ID NO: 42 - DNA, 40 bp, synthetic construct
  source 1..40: mol_type = other DNA
atttttttctaaagctttaggaaatacacacgtttcccct

SEQ ID NO: 43 - DNA, 40 bp, synthetic construct
  source 1..40: mol_type = other DNA
agagtaatcttcaacaatccttggtctaaacacacacaag

SEQ ID NO: 44 - DNA, 40 bp, synthetic construct
  source 1..40: mol_type = other DNA
cccagggacccacgccaagctcaccgcaccttccaccaaa

SEQ ID NO: 45 - DNA, 40 bp, synthetic construct
  source 1..40: mol_type = other DNA
agctcctgtactagctggtggggtgtggagcacacagccc

SEQ ID NO: 46 - DNA, 40 bp, synthetic construct
  source 1..40: mol_type = other DNA
tcacacagggaaagtgaggcttggtggttgatttgagcaa

SEQ ID NO: 47 - DNA, 40 bp, synthetic construct
  source 1..40: mol_type = other DNA
ccttccaacagccgtgtgagacaagaggtcttatcctctt

SEQ ID NO: 48 - DNA, 40 bp, synthetic construct
  source 1..40: mol_type = other DNA
acaagggtcactgagcacatgccatgtgttgggcacagtg

SEQ ID NO: 49 - DNA, 40 bp, synthetic construct
  source 1..40: mol_type = other DNA
gtctcctaagtctcattcttttcttaggattcttcagatc

SEQ ID NO: 50 - DNA, 40 bp, synthetic construct
  source 1..40: mol_type = other DNA
tccgcctaagtaaaacataaaattacttaagctgcgtaaa

SEQ ID NO: 51 - DNA, 40 bp, synthetic construct
  source 1..40: mol_type = other DNA
cattttgacctgattatctttgtctataagtcttaagcca

SEQ ID NO: 52 - DNA, 40 bp, synthetic construct
  source 1..40: mol_type = other DNA
ccggttcctccaccctcactgccccaacaactgaaagaag

SEQ ID NO: 53 - DNA, 40 bp, synthetic construct
  source 1..40: mol_type = other DNA
acagtgtgttgaaagaatccataactctttctttccagcc

SEQ ID NO: 54 - DNA, 40 bp, synthetic construct
  source 1..40: mol_type = other DNA
gaagtttcatctttatcaaaatctccattcccaggcggac

SEQ ID NO: 55 - DNA, 40 bp, synthetic construct
  source 1..40: mol_type = other DNA
aagtccatttttttaagctttgcgcttcagctccagaaca

SEQ ID NO: 56 - DNA, 40 bp, synthetic construct
  source 1..40: mol_type = other DNA
tcttcgttatgaatacaaataggaaaacaatcagacccaa

SEQ ID NO: 57 - DNA, 40 bp, synthetic construct
  source 1..40: mol_type = other DNA
tcctcggggcattctagaaccgtagcagacctgcttacat

SEQ ID NO: 58 - DNA, 40 bp, synthetic construct
  source 1..40: mol_type = other DNA
tccttatgtgggaaaataaagaggatagacagatttgatt

SEQ ID NO: 59 - DNA, 40 bp, synthetic construct
  source 1..40: mol_type = other DNA
agctgcgagtccctaacagacttccaggacagctgaaaaa

SEQ ID NO: 60 - DNA, 40 bp, synthetic construct
  source 1..40: mol_type = other DNA
aggacaagggagagacgcccacccgcctctgtcagggata

SEQ ID NO: 61 - DNA, 40 bp, synthetic construct
  source 1..40: mol_type = other DNA
aatccatgagggtgacatacacatccttactgttcccaca

SEQ ID NO: 62 - DNA, 40 bp, synthetic construct
  source 1..40: mol_type = other DNA
acttccttccctgagatgcccatcctttgattctgggatt

SEQ ID NO: 63 - DNA, 40 bp, synthetic construct
  source 1..40: mol_type = other DNA
gctcccggataaattaattaccgtgaccctgagctgcttc

SEQ ID NO: 64 - DNA, 40 bp, synthetic construct
  source 1..40: mol_type = other DNA
tagactaagagaatctaatttgtggcaaagatcttgagtg

SEQ ID NO: 65 - DNA, 40 bp, synthetic construct
  source 1..40: mol_type = other DNA
tgaaggatgactaagagcttccctataaaccccatactgg

SEQ ID NO: 66 - DNA, 40 bp, synthetic construct
  source 1..40: mol_type = other DNA
agccaggactatagagtttcagaaaagggagaaaattcta

SEQ ID NO: 67 - DNA, 40 bp, synthetic construct
  source 1..40: mol_type = other DNA
tgctgctaatttaagtttctggcaagtcaaaataaatctc

SEQ ID NO: 68 - DNA, 40 bp, synthetic construct
  source 1..40: mol_type = other DNA
cgaaaaccatcaattaactagaatgatcaggaaattgcgt

SEQ ID NO: 69 - DNA, 40 bp, synthetic construct
  source 1..40: mol_type = other DNA
tttatttagtccccagggtgtatgaagtgctcttccaggc

SEQ ID NO: 70 - DNA, 40 bp, synthetic construct
  source 1..40: mol_type = other DNA
ggtccttcttggtaccgatattgccatattggctggacat

SEQ ID NO: 71 - DNA, 40 bp, synthetic construct
  source 1..40: mol_type = other DNA
tggcttggtaggatgcactcacatgggctgtagtaatact

SEQ ID NO: 72 - DNA, 40 bp, synthetic construct
  source 1..40: mol_type = other DNA
tatcaccagcataacttgtggttcttcagccagtaatttc

SEQ ID NO: 73 - DNA, 40 bp, synthetic construct
  source 1..40: mol_type = other DNA
gaacaactgggtatctacaggcaaagaaatgaaccttgac

SEQ ID NO: 74 - DNA, 40 bp, synthetic construct
  source 1..40: mol_type = other DNA
taggtactgttgtgtccctatatatttgacttggtaataa

SEQ ID NO: 75 - DNA, 40 bp, synthetic construct
  source 1..40: mol_type = other DNA
tatgtgaacatcggtgaatatcataatttattatgcaaac

SEQ ID NO: 76 - DNA, 40 bp, synthetic construct
  source 1..40: mol_type = other DNA
agctgaacactctttgtggtcctcttgaagcctagaatta

SEQ ID NO: 77 - DNA, 40 bp, synthetic construct
  source 1..40: mol_type = other DNA
ccccacctcactgccccccagttctgactcacggtgtccc

SEQ ID NO: 78 - DNA, 40 bp, synthetic construct
  source 1..40: mol_type = other DNA
actcccatcacctggccagcttggctgtcccctgacccac

SEQ ID NO: 79 - DNA, 40 bp, synthetic construct
  source 1..40: mol_type = other DNA
ggctgcccagctgcccagcagcaaaactgcataggaactc

SEQ ID NO: 80 - DNA, 40 bp, synthetic construct
  source 1..40: mol_type = other DNA
gcccaggacgccaagtgtcaccaccctctccccaggcagg

SEQ ID NO: 81 - DNA, 40 bp, synthetic construct
  source 1..40: mol_type = other DNA
cacaaggtcagctccacccgtgggtcagtgtgccccagat

SEQ ID NO: 82 - DNA, 40 bp, synthetic construct
  source 1..40: mol_type = other DNA
ggagacaaaacgggcacccagcccagtcatgcccgtgcct

SEQ ID NO: 83 - DNA, 40 bp, synthetic construct
  source 1..40: mol_type = other DNA
ctgaaatcagtcagcagtttcggtgagtctgcagctgaca

SEQ ID NO: 84 - DNA, 40 bp, synthetic construct
  source 1..40: mol_type = other DNA
cgccacatttggggctgggagagatgtcacaggggctgac

SEQ ID NO: 85 - DNA, 40 bp, synthetic construct
  source 1..40: mol_type = other DNA
cacatgttctctgcataggtttttaagcagccagcagctg

SEQ ID NO: 86 - DNA, 40 bp, synthetic construct
  source 1..40: mol_type = other DNA
tttaaaatgaaaacccacacttccaaaatagcacttgagt

SEQ ID NO: 87 - DNA, 40 bp, synthetic construct
  source 1..40: mol_type = other DNA
aacatgtttgtgtaattaagcattttaaaatcataaccat

SEQ ID NO: 88 - DNA, 40 bp, synthetic construct
  source 1..40: mol_type = other DNA
tgcttatctgtgctttttatgttccacccccccaccacca

SEQ ID NO: 89 - DNA, 40 bp, synthetic construct
  source 1..40: mol_type = other DNA
attaataataattctgtgtttatggggattgcagatacat

SEQ ID NO: 90 - DNA, 40 bp, synthetic construct
  source 1..40: mol_type = other DNA
ccagctttgtgtcttcatgacccaactggagtaagaatgg

SEQ ID NO: 91 - DNA, 40 bp, synthetic construct
  source 1..40: mol_type = other DNA
aaagacctcatttgcagcatggttagcagtgtcaaacatt

SEQ ID NO: 92 - DNA, 40 bp, synthetic construct
  source 1..40: mol_type = other DNA
tctcgtagcactggctgcagccggcctgtgtgtgcccacc

SEQ ID NO: 93 - DNA, 40 bp, synthetic construct
  source 1..40: mol_type = other DNA
gccttcatcctgaacggctgaccagcggaaacaaaagatc

SEQ ID NO: 94 - DNA, 40 bp, synthetic construct
  source 1..40: mol_type = other DNA
atggccagataacagtgtttagacatgtctttgatgtttt

SEQ ID NO: 95 - DNA, 40 bp, synthetic construct
  source 1..40: mol_type = other DNA
ccctgactgtgtaaggggtctctctccatggggaatagag

SEQ ID NO: 96 - DNA, 40 bp, synthetic construct
  source 1..40: mol_type = other DNA
ctgagcttagcttctactgtgctgttaatttcaggcaaga

SEQ ID NO: 97 - DNA, 40 bp, synthetic construct
  source 1..40: mol_type = other DNA
agatcaataatatttgcattagctacttacatcagtctct

SEQ ID NO: 98 - DNA, 40 bp, synthetic construct
  source 1..40: mol_type = other DNA
taattgcagaaaacttataaagcatggaagaatacaaaac

SEQ ID NO: 99 - DNA, 40 bp, synthetic construct
  source 1..40: mol_type = other DNA
aaacaaattcctctacctggacatgactgttgttagcatt

SEQ ID NO: 100 - DNA, 40 bp, synthetic construct
  source 1..40: mol_type = other DNA
gggagattcttcatatccttttaatgtagatatgcacatt

SEQ ID NO: 101 - DNA, 40 bp, synthetic construct
  source 1..40: mol_type = other DNA
acaaaaaaggctatcatattgtacatataactttgctgta

SEQ ID NO: 102 - DNA, 40 bp, synthetic construct
  source 1..40: mol_type = other DNA
tctgctaggaacctgtacccatgtcattactgtaagcatt

SEQ ID NO: 103 - DNA, 40 bp, synthetic construct
  source 1..40: mol_type = other DNA
actactcaaattttagtatctgcagatatcagatatcctt

SEQ ID NO: 104 - DNA, 40 bp, synthetic construct
  source 1..40: mol_type = other DNA
tgaaatggtattgttgccctttctgattagtaaagtatac

SEQ ID NO: 105 - DNA, 40 bp, synthetic construct
  source 1..40: mol_type = other DNA
ttataatctagcaaggttagagatcatggatcactttcag

SEQ ID NO: 106 - DNA, 40 bp, synthetic construct
  source 1..40: mol_type = other DNA
acagcttgcctccgataagccagaattccagagcttctgg

SEQ ID NO: 107 - DNA, 40 bp, synthetic construct
  source 1..40: mol_type = other DNA
tcaatcaacctgatagcttaggggataaactaatttgaag

SEQ ID NO: 108 - DNA, 40 bp, synthetic construct
  source 1..40: mol_type = other DNA
gatcatgaaggatgaaagaatttcaccaatattataataa

SEQ ID NO: 109 - DNA, 40 bp, synthetic construct
  source 1..40: mol_type = other DNA
tttagccatctgtatcaatgagcagatataagctttacac

SEQ ID NO: 110 - DNA, 40 bp, synthetic construct
  source 1..40: mol_type = other DNA
aggggtagattatttatgctgcccatttttagaccataaa

SEQ ID NO: 111 - DNA, 40 bp, synthetic construct
  source 1..40: mol_type = other DNA
cactaccatttcacaattcgcactttctttctttgtcctt

SEQ ID NO: 112 - DNA, 40 bp, synthetic construct
  source 1..40: mol_type = other DNA
gctccatcaaatcataaaggacccacttcaaatgccatca

SEQ ID NO: 113 - DNA, 40 bp, synthetic construct
  source 1..40: mol_type = other DNA
tcctactttcaggaacttctttctccaaacgtcttctgcc

SEQ ID NO: 114 - DNA, 40 bp, synthetic construct
  source 1..40: mol_type = other DNA
aattctattttttcttcaacgtactttaggcttgtaatgt

SEQ ID NO: 115 - DNA, 40 bp, synthetic construct
  source 1..40: mol_type = other DNA
taagatgcaaatagtaagcctgagcccttctgtctaactt

SEQ ID NO: 116 - DNA, 40 bp, synthetic construct
  source 1..40: mol_type = other DNA
ctgtgtttcagaataaaataccaactctactactctcatc

SEQ ID NO: 117 - DNA, 40 bp, synthetic construct
  source 1..40: mol_type = other DNA
gaaaccatgtttatctcaggtttacaaatctccacttgtc

SEQ ID NO: 118 - DNA, 40 bp, synthetic construct
  source 1..40: mol_type = other DNA
ctttggaaaagtaatcaggtttagaggagctcatgagagc

SEQ ID NO: 119 - DNA, 40 bp, synthetic construct
  source 1..40: mol_type = other DNA
gctgaatccccaactcccaattggctccatttgtggggga

SEQ ID NO: 120 - DNA, 40 bp, synthetic construct
  source 1..40: mol_type = other DNA
ggtgttatgaacttaacgcttgtgtctccagaaaattcac

SEQ ID NO: 121 - DNA, 40 bp, synthetic construct
  source 1..40: mol_type = other DNA
agttaatgcacgttaataagcaagagtttagtttaatgtg

SEQ ID NO: 122 - DNA, 40 bp, synthetic construct
  source 1..40: mol_type = other DNA
taattgagaaggcagattcactggagttcttatataattg

SEQ ID NO: 123 - DNA, 40 bp, synthetic construct
  source 1..40: mol_type = other DNA
cacggtcagatgaaaatatagtgtgaagaatttgtataac

SEQ ID NO: 124 - DNA, 40 bp, synthetic construct
  source 1..40: mol_type = other DNA
cacaagtcagcatcagcgtgtcatgtctcagcagcagaac

SEQ ID NO: 125 - DNA, 40 bp, synthetic construct
  source 1..40: mol_type = other DNA
ggaggtggggacttaggtgaaggaaatgagccagcagaag

SEQ ID NO: 126 - DNA, 40 bp, synthetic construct
  source 1..40: mol_type = other DNA
gtcacagcatttcaaggaggagacctcattgtaagcttct

SEQ ID NO: 127 - DNA, 40 bp, synthetic construct
  source 1..40: mol_type = other DNA
aaagaggtgaaattaatcccatacccttaagtctacagac

SEQ ID NO: 128 - DNA, 40 bp, synthetic construct
  source 1..40: mol_type = other DNA
ctttactaaggaacttttcattttaagtgttgacgcatgc

SEQ ID NO: 129 - DNA, 40 bp, synthetic construct
  source 1..40: mol_type = other DNA
caggtttttctttccacggtaactacaatgaagtgatcct

SEQ ID NO: 130 - DNA, 40 bp, synthetic construct
  source 1..40: mol_type = other DNA
gctctacagggaggttgaggtgttagagatcagagcagga

SEQ ID NO: 131 - DNA, 40 bp, synthetic construct
  source 1..40: mol_type = other DNA
tactatttccaacggcatctggcttttctcagcccttgtg

SEQ ID NO: 132 - DNA, 40 bp, synthetic construct
  source 1..40: mol_type = other DNA
aaggtttaggcagggatagccattctattttattaggggc

SEQ ID NO: 133 - DNA, 40 bp, synthetic construct
  source 1..40: mol_type = other DNA
aggggctcaacgaagaaaaagtgttccaagctttaggaag

SEQ ID NO: 134 - DNA, 40 bp, synthetic construct
  source 1..40: mol_type = other DNA
gggctgaacccccttccctggattgcagcacagcagcgag

SEQ ID NO: 135 - DNA, 40 bp, synthetic construct
  source 1..40: mol_type = other DNA
ctgacgtcataatctaccaaggtcatggatcgagttcaga

SEQ ID NO: 136 - DNA, 40 bp, synthetic construct
  source 1..40: mol_type = other DNA
gaaggtagagctctcctccaataagccagatttccagagt

SEQ ID NO: 137 - DNA, 40 bp, synthetic construct
  source 1..40: mol_type = other DNA
caccaatattattataattcctatcaacctgataggttag

SEQ ID NO: 138 - DNA, 40 bp, synthetic construct
  source 1..40: mol_type = other DNA
agatataagccttacacaggattatgaagtctgaaaggat

SEQ ID NO: 139 - DNA, 40 bp, synthetic construct
  source 1..40: mol_type = other DNA
acatgtatctttctggtcttttagccgcctaacactttga

SEQ ID NO: 140 - DNA, 40 bp, synthetic construct
  source 1..40: mol_type = other DNA
caaagaacaagtgcaatatgtgcagctttgttgcgcaggt

SEQ ID NO: 141 - DNA, 40 bp, synthetic construct
  source 1..40: mol_type = other DNA
tattattatgtgagtaactggaagatactgataagttgac

SEQ ID NO: 142 - DNA, 40 bp, synthetic construct
  source 1..40: mol_type = other DNA
taaaaatctttctcacccatccttagattgagagaagtca

SEQ ID NO: 143 - DNA, 40 bp, synthetic construct
  source 1..40: mol_type = other DNA
ttgggttcacctcagtctctataatctgtaccagcatacc

SEQ ID NO: 144 - DNA, 40 bp, synthetic construct
  source 1..40: mol_type = other DNA
cacacccatctcacagatcccctatcttaaagagacccta

SEQ ID NO: 145 - DNA, 40 bp, synthetic construct
  source 1..40: mol_type = other DNA
atggaacccaaccagactctcagatatggccaaagatcta

SEQ ID NO: 146 - DNA, 40 bp, synthetic construct
  source 1..40: mol_type = other DNA
gacaccagtctctgacacattcttaaaggtcaggctctac

SEQ ID NO: 147 - DNA, 40 bp, synthetic construct
  source 1..40: mol_type = other DNA
agagattcaaaagattcacttgtttaggccttagcgggct

SEQ ID NO: 148 - DNA, 40 bp, synthetic construct
  source 1..40: mol_type = other DNA
tccttagtctgaggaggagcaattaagattcacttgttta

SEQ ID NO: 149 - DNA, 40 bp, synthetic construct
  source 1..40: mol_type = other DNA
taaatggggaagttgtttgaaaacaggagggatcctagat

SEQ ID NO: 150 - DNA, 40 bp, synthetic construct
  source 1..40: mol_type = other DNA
gggtttatacatgacttttagaacactgccttggtttttg

SEQ ID NO: 151 - DNA, 40 bp, synthetic construct
  source 1..40: mol_type = other DNA
aactcttaaaagatattgcctcaaaagcataagaggaaat

SEQ ID NO: 152 - DNA, 40 bp, synthetic construct
  source 1..40: mol_type = other DNA
aaatcgaggaataagacagttatggataaggagaaatcaa

SEQ ID NO: 153 - DNA, 40 bp, synthetic construct
  source 1..40: mol_type = other DNA
tcagttaggatttaatcaatgtcagaagcaatgatatagg

SEQ ID NO: 154 - DNA, 40 bp, synthetic construct
  source 1..40: mol_type = other DNA
cttgaaaacacttgaaattgcttgtgtaaagaaacagttt

SEQ ID NO: 155 - DNA, 40 bp, synthetic construct
  source 1..40: mol_type = other DNA
ataatcttcagaggaaagttttattctctgacttatttaa

SEQ ID NO: 156 - DNA, 40 bp, synthetic construct
  source 1..40: mol_type = other DNA
agattccttctgtcattttgcctctgttcgaatactttct

SEQ ID NO: 157 - DNA, 40 bp, synthetic construct
  source 1..40: mol_type = other DNA
atttcagcttctaaactttatttggcaatgccttcccatg

SEQ ID NO: 158 - DNA, 40 bp, synthetic construct
  source 1..40: mol_type = other DNA
gcaggagtttgttttcttctgcttcagagctttgaattta

SEQ ID NO: 159 - DNA, 40 bp, synthetic construct
  source 1..40: mol_type = other DNA
acatatcaacggcactggttctttatctaactctctggca

SEQ ID NO: 160 - DNA, 40 bp, synthetic construct
  source 1..40: mol_type = other DNA
ttatgcttccctgaaacaataccacctgctattctccact

SEQ ID NO: 161 - DNA, 40 bp, synthetic construct
  source 1..40: mol_type = other DNA
ttctcactccctaccactgaggacaagtttatgtccttag

SEQ ID NO: 162 - DNA, 40 bp, synthetic construct
  source 1..40: mol_type = other DNA
ttagagattatgtcattaccagagttaaaattctataatg

SEQ ID NO: 163 - DNA, 40 bp, synthetic construct
  source 1..40: mol_type = other DNA
ggtcattcttagaatagtaatccagccaatagtacaggtt

SEQ ID NO: 164 - DNA, 40 bp, synthetic construct
  source 1..40: mol_type = other DNA
caggcaataagggctttttaagcaaaacagttgtgataaa

SEQ ID NO: 165 - DNA, 40 bp, synthetic construct
  source 1..40: mol_type = other DNA
atgatgggcactgaaggttaaaacttgagtctgtcaactt

SEQ ID NO: 166 - DNA, 40 bp, synthetic construct
  source 1..40: mol_type = other DNA
aactcataaatatcccattttccgctgaaatatagcttta

SEQ ID NO: 167 - DNA, 40 bp, synthetic construct
  source 1..40: mol_type = other DNA
cctggtttctttgaccttttgggaccttgagtaagtaaag

SEQ ID NO: 168 - DNA, 40 bp, synthetic construct
  source 1..40: mol_type = other DNA
cttcatttattttcatgattaaaattctaagaaattcttg

SEQ ID NO: 169 - DNA, 40 bp, synthetic construct
  source 1..40: mol_type = other DNA
tttttaattaaattgcattgcctaatgtatttatgaacta

SEQ ID NO: 170 - DNA, 40 bp, synthetic construct
  source 1..40: mol_type = other DNA
catagaaataaaacaatactctgaagtagttcagaatgtg

SEQ ID NO: 171 - DNA, 40 bp, synthetic construct
  source 1..40: mol_type = other DNA
caatttatataaagagttaattcaaatgagactattttaa

SEQ ID NO: 172 - DNA, 40 bp, synthetic construct
  source 1..40: mol_type = other DNA
agggctttgaatcttatgtctagaaattttgaaaaacctc

SEQ ID NO: 173 - DNA, 40 bp, synthetic construct
  source 1..40: mol_type = other DNA
tatatgctaagattccacctctagtgctagaactgagaag

SEQ ID NO: 174 - DNA, 40 bp, synthetic construct
  source 1..40: mol_type = other DNA
tgacttggtgatcttttttaaattctgaaacaacagcaac

SEQ ID NO: 175 - DNA, 40 bp, synthetic construct
  source 1..40: mol_type = other DNA
agctaaggactttttcttgcctatgcatgctatcttcagt

SEQ ID NO: 176 - DNA, 40 bp, synthetic construct
  source 1..40: mol_type = other DNA
tgattatttagtattgaaactataacatagtatgtttcct

SEQ ID NO: 177 - DNA, 40 bp, synthetic construct
  source 1..40: mol_type = other DNA
aaaaaatgtgtatttctctggagaaggttaaaactgagga

SEQ ID NO: 178 - DNA, 40 bp, synthetic construct
  source 1..40: mol_type = other DNA
caagtgagcaaggcttaaatggaagaagcaatgatctcgt

SEQ ID NO: 179 - DNA, 40 bp, synthetic construct
  source 1..40: mol_type = other DNA
ccaccttcattaacgagatcatccatcatgaggaaatatg

SEQ ID NO: 180 - DNA, 40 bp, synthetic construct
  source 1..40: mol_type = other DNA
accaggccccctctgttttgtgtcactaagggtgaggatg

SEQ ID NO: 181 - DNA, 40 bp, synthetic construct
  source 1..40: mol_type = other DNA
atgatttttccctcccccgggcttcttttagccatcaata

SEQ ID NO: 182 - DNA, 40 bp, synthetic construct
  source 1..40: mol_type = other DNA
tagccccacaggagtttgttctgaaagtaaacttccacaa

SEQ ID NO: 183 - DNA, 40 bp, synthetic construct
  source 1..40: mol_type = other DNA
aagcttattgaggctaaggcatctgtgaaggaaagaaaca

SEQ ID NO: 184 - DNA, 40 bp, synthetic construct
  source 1..40: mol_type = other DNA
ctctaaaccactatgctgctagagcctcttttctgtactc

SEQ ID NO: 185 - DNA, 40 bp, synthetic construct
  source 1..40: mol_type = other DNA
ctcattcagacactagtgtcaccagtctcctcatatacct

SEQ ID NO: 186 - DNA, 40 bp, synthetic construct
  source 1..40: mol_type = other DNA
tattttcttcttcttgctggtttagtcatgttttctggga

SEQ ID NO: 187 - DNA, 40 bp, synthetic construct
  source 1..40: mol_type = other DNA
ggcaaacccattatttttttctttagacttgggatggtga

SEQ ID NO: 188 - DNA, 40 bp, synthetic construct
  source 1..40: mol_type = other DNA
tgggcagcgtcagaaactgtgtgtggatatagataagagc

SEQ ID NO: 189 - DNA, 40 bp, synthetic construct
  source 1..40: mol_type = other DNA
gactatgctgagctgtgatgagggaggggcctagctaaag

SEQ ID NO: 190 - DNA, 40 bp, synthetic construct
  source 1..40: mol_type = other DNA
tgagagtcagaatgctcctgctattgccttctcagtcccc

SEQ ID NO: 191 - DNA, 40 bp, synthetic construct
  source 1..40: mol_type = other DNA
ttggtttctacacaagtagatacatagaaaaggctatagg

SEQ ID NO: 192 - DNA, 40 bp, synthetic construct
  source 1..40: mol_type = other DNA
tgtttgagagtcctgcatgattagttgctcagaaatgccc

SEQ ID NO: 193 - DNA, 40 bp, synthetic construct
  source 1..40: mol_type = other DNA
ttacaaatatgtgattatcatcaaaacgtgagggctaaag

SEQ ID NO: 194 - DNA, 40 bp, synthetic construct
  source 1..40: mol_type = other DNA
cagataacttgcaagtcctaggataccaggaaaataaatt

SEQ ID NO: 195 - DNA, 40 bp, synthetic construct
  source 1..40: mol_type = other DNA
agcattatgtctgtctgtcattgtttttcatcctcttgta

SEQ ID NO: 196 - DNA, 40 bp, synthetic construct
  source 1..40: mol_type = other DNA
ttcacagttacccacacaggtgaacccttttagctctcct

SEQ ID NO: 197 - DNA, 40 bp, synthetic construct
  source 1..40: mol_type = other DNA
gaatgtttctttcctctcaggatcagagttgcctacatct

SEQ ID NO: 198 - DNA, 40 bp, synthetic construct
  source 1..40: mol_type = other DNA
aatgcaccaagactggcctgagatgtatccttaagatgag

SEQ ID NO: 199 - DNA, 40 bp, synthetic construct
  source 1..40: mol_type = other DNA
tcccagtagcaccccaagtcagatctgaccccgtatgtga

SEQ ID NO: 200 - DNA, 40 bp, synthetic construct
  source 1..40: mol_type = other DNA
gtgtcctctaacagcacaggccttttgccacctagctgtc

SEQ ID NO: 201 - DNA, 40 bp, synthetic construct
  source 1..40: mol_type = other DNA
ggcaaacaaggtttgttttcttttcctgttttcatgcctt

SEQ ID NO: 202 - DNA, 40 bp, synthetic construct
  source 1..40: mol_type = other DNA
ttccatatccttgtttcatattaatacatgtgtatagatc

SEQ ID NO: 203 - DNA, 40 bp, synthetic construct
  source 1..40: mol_type = other DNA
aaatctatacacatgtattaataaagcctgattctgccgc

SEQ ID NO: 204 - DNA, 40 bp, synthetic construct
  source 1..40: mol_type = other DNA
aggtatagaggccacctgcaagataaatatttgattcaca

SEQ ID NO: 205 - DNA, 40 bp, synthetic construct
  source 1..40: mol_type = other DNA
ctaatcattctatggcaattgataacaacaaatatatata

SEQ ID NO: 206 - DNA, 40 bp, synthetic construct
  source 1..40: mol_type = other DNA
ataatatattctagaatatgtcacattctgtctcaggcat

SEQ ID NO: 207 - DNA, 40 bp, synthetic construct
  source 1..40: mol_type = other DNA
tttctttatgatgccgtttgaggtggagttttagtcaggt

SEQ ID NO: 208 - DNA, 40 bp, synthetic construct
  source 1..40: mol_type = other DNA
agcttctccttttttttgccatctgccctgtaagcatcct

SEQ ID NO: 209 - DNA, 40 bp, synthetic construct
  source 1..40: mol_type = other DNA
gggacccagataggagtcatcactctaggctgagaacatc

SEQ ID NO: 210 - DNA, 40 bp, synthetic construct
  source 1..40: mol_type = other DNA
cacacaccctaagcctcagcatgactcatcatgactcagc

SEQ ID NO: 211 - DNA, 40 bp, synthetic construct
  source 1..40: mol_type = other DNA
ctgtgcttgagccagaaggtttgcttagaaggttacacag

SEQ ID NO: 212 - DNA, 40 bp, synthetic construct
  source 1..40: mol_type = other DNA
aactgctcatgcttggactatgggaggtcactaatggaga

SEQ ID NO: 213 - DNA, 40 bp, synthetic construct
  source 1..40: mol_type = other DNA
cagaaatgtaacaggaactaaggaaaaactgaagcttatt

SEQ ID NO: 214 - DNA, 40 bp, synthetic construct
  source 1..40: mol_type = other DNA
cagagatgaggatgctggaagggatagagggagctgagct

SEQ ID NO: 215 - DNA, 40 bp, synthetic construct
  source 1..40: mol_type = other DNA
aaaagtatagtaatcattcagcaaatggttttgaagcacc

SEQ ID NO: 216 - DNA, 40 bp, synthetic construct
  source 1..40: mol_type = other DNA
gtatcttattccccacaagagtccaagtaaaaaataacag

SEQ ID NO: 217 - DNA, 40 bp, synthetic construct
  source 1..40: mol_type = other DNA
gaaaagaatgtttctctcactgtggattattttagagagt

SEQ ID NO: 218 - DNA, 40 bp, synthetic construct
  source 1..40: mol_type = other DNA
aatggtcaagatttttttaaaaattaagaaaacataagtt

SEQ ID NO: 219 - DNA, 40 bp, synthetic construct
  source 1..40: mol_type = other DNA
cttgagaaatgaaaatttatttttttgttggaggataccc

SEQ ID NO: 220 - DNA, 40 bp, synthetic construct
  source 1..40: mol_type = other DNA
tctatctcccatcagggcaagctgtaaggaactggctaag

SEQ ID NO: 221 - DNA, 40 bp, synthetic construct
  source 1..40: mol_type = other DNA
agtgagacagagtgacttagtcttagaggccccactggta

SEQ ID NO: 222 - DNA, 40 bp, synthetic construct
  source 1..40: mol_type = other DNA
gatgagaaggcaccttcatcactcatcacagtcagctctg

SEQ ID NO: 223 - DNA, 40 bp, synthetic construct
  source 1..40: mol_type = other DNA
tctcctctctcctttctcatcagaaatttcataagtctac

SEQ ID NO: 224 - DNA, 40 bp, synthetic construct
  source 1..40: mol_type = other DNA
gtcaggcagatcacataagaaaagaggatgccagttaagg

SEQ ID NO: 225 - DNA, 40 bp, synthetic construct
  source 1..40: mol_type = other DNA
gttgctgttagacaatttcatctgtgccctgcttaggagc

SEQ ID NO: 226 - DNA, 40 bp, synthetic construct
  source 1..40: mol_type = other DNA
tctttaatgaaagctaagctttcattaaaaaaagtctaac

SEQ ID NO: 227 - DNA, 40 bp, synthetic construct
  source 1..40: mol_type = other DNA
tgcattcgactttgactgcagcagctggttagaaggttct

SEQ ID NO: 228 - DNA, 40 bp, synthetic construct
  source 1..40: mol_type = other DNA
gaggagggtcccagcccattgctaaattaacatcaggctc

SEQ ID NO: 229 - DNA, 40 bp, synthetic construct
  source 1..40: mol_type = other DNA
actggcagtatatctctaacagtggttgatgctatcttct

SEQ ID NO: 230 - DNA, 40 bp, synthetic construct
  source 1..40: mol_type = other DNA
cttgcctgctacattgagaccactgacccatacataggaa

SEQ ID NO: 231 - DNA, 40 bp, synthetic construct
  source 1..40: mol_type = other DNA
atagctctgtcctgaactgttaggccactggtccagagag

SEQ ID NO: 232 - DNA, 40 bp, synthetic construct
  source 1..40: mol_type = other DNA
catctcctttgatcctcataataaccctatgagatagaca

SEQ ID NO: 233 - DNA, 40 bp, synthetic construct
  source 1..40: mol_type = other DNA
tattactcttactttatagatgatgatcctgaaaacatag

SEQ ID NO: 234 - DNA, 40 bp, synthetic construct
  source 1..40: mol_type = other DNA
caaggcacttgcccctagctgggggtataggggagcagtc

SEQ ID NO: 235 - DNA, 40 bp, synthetic construct
  source 1..40: mol_type = other DNA
gtagtagtagaatgaaaaatgctgctatgctgtgcctccc

SEQ ID NO: 236 - DNA, 40 bp, synthetic construct
  source 1..40: mol_type = other DNA
ctttcccatgtctgccctctactcatggtctatctctcct

SEQ ID NO: 237 - DNA, 40 bp, synthetic construct
  source 1..40: mol_type = other DNA
cctgggagtcatggactccacccagcaccaccaacctgac

SEQ ID NO: 238 - DNA, 40 bp, synthetic construct
  source 1..40: mol_type = other DNA
ccacctatctgagcctgccagcctataacccatctgggcc

SEQ ID NO: 239 - DNA, 40 bp, synthetic construct
  source 1..40: mol_type = other DNA
tagctggtggccagccctgaccccaccccaccctccctgg

SEQ ID NO: 240 - DNA, 40 bp, synthetic construct
  source 1..40: mol_type = other DNA
tctgatagacacatctggcacaccagctcgcaaagtcacc

SEQ ID NO: 241 - DNA, 40 bp, synthetic construct
  source 1..40: mol_type = other DNA
gggtcttgtgtttgctgagtcaaaattccttgaaatccaa

SEQ ID NO: 242 - DNA, 40 bp, synthetic construct
  source 1..40: mol_type = other DNA
ttagagactcctgctcccaaatttacagtcatagacttct

SEQ ID NO: 243 - DNA, 40 bp, synthetic construct
  source 1..40: mol_type = other DNA
ggctgtctcctttatccacagaatgattcctttgcttcat

SEQ ID NO: 244 - DNA, 40 bp, synthetic construct
  source 1..40: mol_type = other DNA
ccatccatctgatcctcctcatcagtgcagcacagggccc

SEQ ID NO: 245 - DNA, 40 bp, synthetic construct
  source 1..40: mol_type = other DNA
gcagtagctgcagagtctcacataggtctggcactgcctc

SEQ ID NO: 246 - DNA, 40 bp, synthetic construct
  source 1..40: mol_type = other DNA
atgtccgaccttaggcaaatgcttgactcttctgagctca

SEQ ID NO: 247 - DNA, 40 bp, synthetic construct
  source 1..40: mol_type = other DNA
tgtcatggcaaaataaagataataatagtgtttttttatg

SEQ ID NO: 248 - DNA, 40 bp, synthetic construct
  source 1..40: mol_type = other DNA
tagcgtgaggatggaaaacaatagcaaaattgattagact

SEQ ID NO: 249 - DNA, 40 bp, synthetic construct
  source 1..40: mol_type = other DNA
aaggtctcaacaaatagtagtagattttatcgtccattaa

SEQ ID NO: 250 - DNA, 40 bp, synthetic construct
  source 1..40: mol_type = other DNA
tccctctcctctcttactcatcccatcacgtatgcctctt

SEQ ID NO: 251 - DNA, 40 bp, synthetic construct
  source 1..40: mol_type = other DNA
ttcccttacctataataagagttattcctcttattatatt

SEQ ID NO: 252 - DNA, 40 bp, synthetic construct
  source 1..40: mol_type = other DNA
ttatagtgattctggatattaaagtgggaatgaggggcag

SEQ ID NO: 253 - DNA, 40 bp, synthetic construct
  source 1..40: mol_type = other DNA
ctaacgaagaagatgtttctcaaagaagccattctcccca

SEQ ID NO: 254 - DNA, 40 bp, synthetic construct
  source 1..40: mol_type = other DNA
gatcatctcagcagggttcaggaagataaaggaggatcaa

SEQ ID NO: 255 - DNA, 40 bp, synthetic construct
  source 1..40: mol_type = other DNA
tgttgaggtgggaggaccgcttgagcctgggaagtgcaag

SEQ ID NO: 256 - DNA, 40 bp, synthetic construct
  source 1..40: mol_type = other DNA
agtgagccgagattttgccactacactcccatttgggtga

SEQ ID NO: 257 - DNA, 40 bp, synthetic construct
  source 1..40: mol_type = other DNA
gtgagaccctttctcaaaaacaaactaattaaaaaaccct

SEQ ID NO: 258 - DNA, 40 bp, synthetic construct
  source 1..40: mol_type = other DNA
tttacagatgaagaaactgagtcatacaactactaagaga

SEQ ID NO: 259 - DNA, 40 bp, synthetic construct
  source 1..40: mol_type = other DNA
gagtcactaatcactcaggtggtctggctccagcatctgt

SEQ ID NO: 260 - DNA, 40 bp, synthetic construct
  source 1..40: mol_type = other DNA
ttaatctctgctctatactgcccaagacttttataaagtc

SEQ ID NO: 261 - DNA, 40 bp, synthetic construct
  source 1..40: mol_type = other DNA
gttgagtcactgaaatgagttattgggatggctgtgtggg

SEQ ID NO: 262 - DNA, 40 bp, synthetic construct
  source 1..40: mol_type = other DNA
gtgctaagttctttcctaaaggtatgtgagaatacaaagg

SEQ ID NO: 263 - DNA, 40 bp, synthetic construct
  source 1..40: mol_type = other DNA
aagcatcctcctttttacacacgtgaactagtgcatgcaa

SEQ ID NO: 264 - DNA, 40 bp, synthetic construct
  source 1..40: mol_type = other DNA
gacactcagtgggcctgggtgaaggtgagaattttattgc

SEQ ID NO: 265 - DNA, 40 bp, synthetic construct
  source 1..40: mol_type = other DNA
tgagagcctctggggacatcttgccagtcaatgagtctca

SEQ ID NO: 266 - DNA, 40 bp, synthetic construct
  source 1..40: mol_type = other DNA
caatttccttctcagtcttggagtaacagaagctcatgca

SEQ ID NO: 267 - DNA, 40 bp, synthetic construct
  source 1..40: mol_type = other DNA
ataaacggaaattttgtattgaaatgagagccattggaaa

SEQ ID NO: 268 - DNA, 40 bp, synthetic construct
  source 1..40: mol_type = other DNA
ttactccagactcctacttataaaaagagaaactgaggct

SEQ ID NO: 269 - DNA, 40 bp, synthetic construct
  source 1..40: mol_type = other DNA
gaagggtggggactttctcagtatgacatggaaatgatca

SEQ ID NO: 270 - DNA, 40 bp, synthetic construct
  source 1..40: mol_type = other DNA
tggattcaaagctcctgactttctgtctagtgtatgtgca

SEQ ID NO: 271 - DNA, 40 bp, synthetic construct
  source 1..40: mol_type = other DNA
gccccttttcctctaactgaaagaaggaaaaaaaaatgga

SEQ ID NO: 272 - DNA, 40 bp, synthetic construct
  source 1..40: mol_type = other DNA
aaaatattctacatagtttccatgtcacagccagggctgg

SEQ ID NO: 273 - DNA, 40 bp, synthetic construct
  source 1..40: mol_type = other DNA
tctcctgttatttcttttaaaataaatatatcatttaaat

SEQ ID NO: 274 - DNA, 40 bp, synthetic construct
  source 1..40: mol_type = other DNA
aaataagcaaaccctgctcgggaatgggagggagagtctc

SEQ ID NO: 275 - DNA, 40 bp, synthetic construct
  source 1..40: mol_type = other DNA
gtccaccccttctcggccctggctctgcagatagtgctat

SEQ ID NO: 276 - DNA, 40 bp, synthetic construct
  source 1..40: mol_type = other DNA
gccctgacagagccctgcccattgctgggccttggagtga

SEQ ID NO: 277 - DNA, 40 bp, synthetic construct
  source 1..40: mol_type = other DNA
gcctagtagagaggcagggcaagccatctcatagctgctg

SEQ ID NO: 278 - DNA, 40 bp, synthetic construct
  source 1..40: mol_type = other DNA
ggagagagaaaagggctcattgtctataaactcaggtcat

SEQ ID NO: 279 - DNA, 40 bp, synthetic construct
  source 1..40: mol_type = other DNA
attcttattctcacactaagaaaaagaatgagatgtctac

SEQ ID NO: 280 - DNA, 40 bp, synthetic construct
  source 1..40: mol_type = other DNA
accctgcgtcccctcttgtgtactggggtccccaagagct

SEQ ID NO: 281 - DNA, 40 bp, synthetic construct
  source 1..40: mol_type = other DNA
aaaagtgatggcaaagtcattgcgctagatgccatcccat

SEQ ID NO: 282 - DNA, 40 bp, synthetic construct
  source 1..40: mol_type = other DNA
tataaacctgcatttgtctccacacaccagtcatggacaa

SEQ ID NO: 283 - DNA, 40 bp, synthetic construct
  source 1..40: mol_type = other DNA
cctcctcccaggtccacgtgcttgtctttgtataatactc

SEQ ID NO: 284 - DNA, 40 bp, synthetic construct
  source 1..40: mol_type = other DNA
aatttcggaaaatgtattctttcaatcttgttctgttatt

SEQ ID NO: 285 - DNA, 40 bp, synthetic construct
  source 1..40: mol_type = other DNA
tttcaatggcttagtagaaaaagtacatacttgttttccc

SEQ ID NO: 286 - DNA, 40 bp, synthetic construct
  source 1..40: mol_type = other DNA
attgacaatagacaatttcacatcaatgtctatatgggtc

SEQ ID NO: 287 - DNA, 40 bp, synthetic construct
  source 1..40: mol_type = other DNA
tgtttgctgtgtttgcaaaaactcacaataactttatatt

SEQ ID NO: 288 - DNA, 40 bp, synthetic construct
  source 1..40: mol_type = other DNA
ctactctaagaaagttacaacatggtgaatacaagagaaa

SEQ ID NO: 289 - DNA, 40 bp, synthetic construct
  source 1..40: mol_type = other DNA
ttacaagtccagaaaataaaagttatcatcttgaggcctc

SEQ ID NO: 290 - DNA, 40 bp, synthetic construct
  source 1..40: mol_type = other DNA
ttctaggaataatatcaatattacaaaattaatctaacaa

SEQ ID NO: 291 - DNA, 40 bp, synthetic construct
  source 1..40: mol_type = other DNA
gaacagcaatgagataatgtgtacaaagtacccagaccta

SEQ ID NO: 292 - DNA, 40 bp, synthetic construct
  source 1..40: mol_type = other DNA
gtagagcatcaaggaagcgcattgcggagcagttttttgt

SEQ ID NO: 293 - DNA, 40 bp, synthetic construct
  source 1..40: mol_type = other DNA
ttgtttttgtattctgtttcgtgaggcaaggtttcactct

SEQ ID NO: 294 - DNA, 40 bp, synthetic construct
  source 1..40: mol_type = other DNA
tccaggctggagtgcagtggcaagatcatgtctcactgca

SEQ ID NO: 295 - DNA, 40 bp, synthetic construct
  source 1..40: mol_type = other DNA
tgacctcctgagctcaagggatcctcccatttcggcctcc

SEQ ID NO: 296 - DNA, 40 bp, synthetic construct
  source 1..40: mol_type = other DNA
tagctgggactacaggtgtacatcacatgcctggctaatt

SEQ ID NO: 297 - DNA, 40 bp, synthetic construct
  source 1..40: mol_type = other DNA
ttttttttttaagtagagacgaggtcttgctatgttgtcc

SEQ ID NO: 298 - DNA, 40 bp, synthetic construct
  source 1..40: mol_type = other DNA
taatatcaaactcttgagctcaagcagtcctcccacttct

SEQ ID NO: 299 - DNA, 40 bp, synthetic construct
  source 1..40: mol_type = other DNA
tggaggtatccagtatgaaatttagataatacctgccttc

SEQ ID NO: 300 - DNA, 40 bp, synthetic construct
  source 1..40: mol_type = other DNA
gttgaaattagaacttaatgatataatgcatcaatgaact

SEQ ID NO: 301 - DNA, 40 bp, synthetic construct
  source 1..40: mol_type = other DNA
atagttcctagcacaaagtaagaatcctttcaatgtgtgt

SEQ ID NO: 302 - DNA, 40 bp, synthetic construct
  source 1..40: mol_type = other DNA
gtgtatgtatttatctgttattaataggaatcttatgggc

SEQ ID NO: 303 - DNA, 40 bp, synthetic construct
  source 1..40: mol_type = other DNA
tctcacttaatccttattaataactatgaagcaggtattt

SEQ ID NO: 304 - DNA, 40 bp, synthetic construct
  source 1..40: mol_type = other DNA
gagttttccaagtgagttaagtatagcttgtaatacttaa

SEQ ID NO: 305 - DNA, 40 bp, synthetic construct
  source 1..40: mol_type = other DNA
atatccacaggttacatagctagtatataactgagaaata

SEQ ID NO: 306 - DNA, 40 bp, synthetic construct
  source 1..40: mol_type = other DNA
tatttatattataaaacattctaacaatacagatgtatat

SEQ ID NO: 307 - DNA, 40 bp, synthetic construct
  source 1..40: mol_type = other DNA
taaaaaactgaaagggctcatgcaaccctaccttctcaat

SEQ ID NO: 308 - DNA, 40 bp, synthetic construct
  source 1..40: mol_type = other DNA
cttcttcacttagaaaaaaccagccttagctgtctgctat

SEQ ID NO: 309 - DNA, 40 bp, synthetic construct
  source 1..40: mol_type = other DNA
cctttcaaaatatacttctgagaaatgagagagagaaatg

SEQ ID NO: 310 - DNA, 40 bp, synthetic construct
  source 1..40: mol_type = other DNA
gggtagaaggaaggaagatagggtaagagacagggaagga

SEQ ID NO: 311 - DNA, 40 bp, synthetic construct
  source 1..40: mol_type = other DNA
tggggaaagaaattaaattattcttttctctgtctcttga

SEQ ID NO: 312 - DNA, 40 bp, synthetic construct
  source 1..40: mol_type = other DNA
gctctttccattacattgaatcaaaggtaatgttgccatt

SEQ ID NO: 313 - DNA, 40 bp, synthetic construct
  source 1..40: mol_type = other DNA
gactcttgaaataaagaaagaccgatgtatgaaataattt

SEQ ID NO: 314 - DNA, 40 bp, synthetic construct
  source 1..40: mol_type = other DNA
agtctatggcattttcaaaatgcaaggtgatgtcttacta

SEQ ID NO: 315 - DNA, 40 bp, synthetic construct
  source 1..40: mol_type = other DNA
gcctttgctttattattagaaatggggaagtgagtataga

SEQ ID NO: 316 - DNA, 40 bp, synthetic construct
  source 1..40: mol_type = other DNA
ttatcaggagatatattaggaaaaagggaaactggagaaa

SEQ ID NO: 317 - DNA, 40 bp, synthetic construct
  source 1..40: mol_type = other DNA
gaggagtatccagatgtcctgtccctgtaaggtgggggca

SEQ ID NO: 318 - DNA, 40 bp, synthetic construct
  source 1..40: mol_type = other DNA
ccttcaatcaaaagggctccttaacaacttccttgcttgg

SEQ ID NO: 319 - DNA, 40 bp, synthetic construct
  source 1..40: mol_type = other DNA
ccaccatcttggaccattagctccacaggtatcttcttcc

SEQ ID NO: 320 - DNA, 40 bp, synthetic construct
  source 1..40: mol_type = other DNA
agtggtcataacagcagcttcagctacctctctaaagagt

SEQ ID NO: 321 - DNA, 40 bp, synthetic construct
  source 1..40: mol_type = other DNA
ccagatataggtcaggaaatataatccactaataaaaaga

SEQ ID NO: 322 - DNA, 40 bp, synthetic construct
  source 1..40: mol_type = other DNA
cattttgactgtagttgtttgttttttgtcattgtgacta

SEQ ID NO: 323 - DNA, 40 bp, synthetic construct
  source 1..40: mol_type = other DNA
taacattctcactctttcatcagtaatcactcaggttatt

SEQ ID NO: 324 - DNA, 40 bp, synthetic construct
  source 1..40: mol_type = other DNA
gaccaacagactgtgggaaaaatcagagaaggaggcatcc

SEQ ID NO: 325 - DNA, 40 bp, synthetic construct
  source 1..40: mol_type = other DNA
gcttactagcctaaactgaaattgctatagcagagtgaac

SEQ ID NO: 326 - DNA, 40 bp, synthetic construct
  source 1..40: mol_type = other DNA
aggtttacagatattttccacaaagagtaaaaggattgaa

SEQ ID NO: 327 - DNA, 40 bp, synthetic construct
  source 1..40: mol_type = other DNA
tctccagatcaatgcataggaaataataatggaccataaa

SEQ ID NO: 328 - DNA, 40 bp, synthetic construct
  source 1..40: mol_type = other DNA
atattatgacgaacaacattaggataagtccatatcaatt

SEQ ID NO: 329 - DNA, 40 bp, synthetic construct
  source 1..40: mol_type = other DNA
atccagtcataagcacagactacgtgaagcacgtccaagt

SEQ ID NO: 330 - DNA, 40 bp, synthetic construct
  source 1..40: mol_type = other DNA
gcaggagaaatgagaggagcaagaaagaggagccatttga

SEQ ID NO: 331 - DNA, 40 bp, synthetic construct
  source 1..40: mol_type = other DNA
gaatagcagaaaaaggaaaggcaagtcatattaacaaatg

SEQ ID NO: 332 - DNA, 40 bp, synthetic construct
  source 1..40: mol_type = other DNA
tcatgccaacagtacagataactctgctaataaaggtaga

SEQ ID NO: 333 - DNA, 40 bp, synthetic construct
  source 1..40: mol_type = other DNA
taatacaggtagtagcagatatctacatagtagttaaagg

SEQ ID NO: 334 - DNA, 40 bp, synthetic construct
  source 1..40: mol_type = other DNA
ggccatcagtacagaagattccataaaggagaacctaaag

SEQ ID NO: 335 - DNA, 40 bp, synthetic construct
  source 1..40: mol_type = other DNA
agaataatttgtcagaagcttaaaagctgaactctgaggc

SEQ ID NO: 336 - DNA, 40 bp, synthetic construct
  source 1..40: mol_type = other DNA
aactacaatatccttttgactgtggaaagggtggtgaaag

SEQ ID NO: 337 - DNA, 40 bp, synthetic construct
  source 1..40: mol_type = other DNA
gttcaaggacatttgagccaacatagagaggaacattggc

SEQ ID NO: 338 - DNA, 40 bp, synthetic construct
  source 1..40: mol_type = other DNA
tgagggatatctgtcctgatgttgtccaggatggtgatga

SEQ ID NO: 339 - DNA, 40 bp, synthetic construct
  source 1..40: mol_type = other DNA
catataaataacgtagagaaaacaggaggggatagagatc

SEQ ID NO: 340 - DNA, 40 bp, synthetic construct
  source 1..40: mol_type = other DNA
caaagaggcatcaaagatagggatgtttgtaaggatgaaa

SEQ ID NO: 341 - DNA, 40 bp, synthetic construct
  source 1..40: mol_type = other DNA
ctgttcttctctgagtagccaagctcagcttggttcaagc

SEQ ID NO: 342 - DNA, 40 bp, synthetic construct
  source 1..40: mol_type = other DNA
catactgtggatctgtagcaaattccccctgaaaacccag

SEQ ID NO: 343 - DNA, 40 bp, synthetic construct
  source 1..40: mol_type = other DNA
tctgaccctcacattcaagttctgaggaagggccactgcc

SEQ ID NO: 344 - DNA, 40 bp, synthetic construct
  source 1..40: mol_type = other DNA
gccttgagatacctggtccttattccttggactttggcaa

SEQ ID NO: 345 - DNA, 40 bp, synthetic construct
  source 1..40: mol_type = other DNA
atagggcttgttttagggagaaacctgttctccaaactct

SEQ ID NO: 346 - DNA, 40 bp, synthetic construct
  source 1..40: mol_type = other DNA
ctggtgtccatactctgaatgggaagaatgatgggattac

SEQ ID NO: 347 - DNA, 40 bp, synthetic construct
  source 1..40: mol_type = other DNA
agcaggagaggatcaaccccatactctgaatctaagagaa

SEQ ID NO: 348 - DNA, 40 bp, synthetic construct
  source 1..40: mol_type = other DNA
tcagatccctggatgcaagccaggtctggaaccataggca

SEQ ID NO: 349 - DNA, 40 bp, synthetic construct
  source 1..40: mol_type = other DNA
ctcctccctaccacctttagccataaggaaacatggaatg

SEQ ID NO: 350 - DNA, 40 bp, synthetic construct
  source 1..40: mol_type = other DNA
gacacaaacctgggcctttcaatgctataacctttcttga

SEQ ID NO: 351 - DNA, 40 bp, synthetic construct
  source 1..40: mol_type = other DNA
ctacctgacttctgagtcaggatttataagccttgttact

SEQ ID NO: 352 - DNA, 40 bp, synthetic construct
  source 1..40: mol_type = other DNA
tgaaccaacaagcatcgaagcaataatgagactgcccgca

SEQ ID NO: 353 - DNA, 40 bp, synthetic construct
  source 1..40: mol_type = other DNA
gaaaagcaataatccatttttcatggtatctcatatgata

SEQ ID NO: 354 - DNA, 40 bp, synthetic construct
  source 1..40: mol_type = other DNA
taacacttatctctctgaactttgggcttttaatatagga

SEQ ID NO: 355 - DNA, 40 bp, synthetic construct
  source 1..40: mol_type = other DNA
ttttctgactgtctaatctttctgatctatcctggatggc

SEQ ID NO: 356 - DNA, 40 bp, synthetic construct
  source 1..40: mol_type = other DNA
atcttcatcgaatttgggtgtttctttctaaaagtccttt

SEQ ID NO: 357 - DNA, 40 bp, synthetic construct
  source 1..40: mol_type = other DNA
gaaattacaaatgctaaagcaaacccaaacaggcaggaat

SEQ ID NO: 358 - DNA, 40 bp, synthetic construct
  source 1..40: mol_type = other DNA
attaggcatcttacagtttttagaatcctgcatagaactt

SEQ ID NO: 359 - DNA, 40 bp, synthetic construct
  source 1..40: mol_type = other DNA
tacaatatttgactcttcaggttaaacatatgtcataaat

SEQ ID NO: 360 - DNA, 40 bp, synthetic construct
  source 1..40: mol_type = other DNA
aacattcagtgaagtgaagggcctactttacttaacaaga

SEQ ID NO: 361 - DNA, 40 bp, synthetic construct
  source 1..40: mol_type = other DNA
tcttttcctatcagtggtttacaagccttgtttatatttt

SEQ ID NO: 362 - DNA, 40 bp, synthetic construct
  source 1..40: mol_type = other DNA
tatttttgttctgagaatatagatttagatacataatgga

SEQ ID NO: 363 - DNA, 40 bp, synthetic construct
  source 1..40: mol_type = other DNA
caaaatctaacacaaaatctagtagaatcatttgcttaca

SEQ ID NO: 364 - DNA, 40 bp, synthetic construct
  source 1..40: mol_type = other DNA
agaatttatgacttgtgatatccaagtcattcctggataa

SEQ ID NO: 365 - DNA, 40 bp, synthetic construct
  source 1..40: mol_type = other DNA
ttacactagaaaatagccacaggcttcctgcaaggcagcc

SEQ ID NO: 366 - DNA, 40 bp, synthetic construct
  source 1..40: mol_type = other DNA
agtttgaacacttgttatggtctattctctcattctttac

SEQ ID NO: 367 - DNA, 40 bp, synthetic construct
  source 1..40: mol_type = other DNA
acttcgtgagagatgaggcagaggtacactacgaaagcaa

SEQ ID NO: 368 - DNA, 40 bp, synthetic construct
  source 1..40: mol_type = other DNA
tcttgagaatgagcctcagccctggctcaaactcacctgc

SEQ ID NO: 369 - DNA, 40 bp, synthetic construct
  source 1..40: mol_type = other DNA
aataggatgtctgtgctccaagttgccagagagagagatt

SEQ ID NO: 370 - DNA, 40 bp, synthetic construct
  source 1..40: mol_type = other DNA
attaaagatccctcctgcttaattaacattcacaagtaac

SEQ ID NO: 371 - DNA, 40 bp, synthetic construct
  source 1..40: mol_type = other DNA
acttaaagtagcgataccctttcaccctgtcctaatcaca

SEQ ID NO: 372 - DNA, 40 bp, synthetic construct
  source 1..40: mol_type = other DNA
tctcaggtgttaactttatagtgaggactttcctgccata

SEQ ID NO: 373 - DNA, 40 bp, synthetic construct
  source 1..40: mol_type = other DNA
atagtttcatataaatgggttcctcatcatctatgggtac

SEQ ID NO: 374 - DNA, 40 bp, synthetic construct
  source 1..40: mol_type = other DNA
ggtatttacatttgccattccctatgccctaaatatttaa

SEQ ID NO: 375 - DNA, 40 bp, synthetic construct
  source 1..40: mol_type = other DNA
tattgatattccttgaaaattctaagcatcttacatcttt

SEQ ID NO: 376 - DNA, 40 bp, synthetic construct
  source 1..40: mol_type = other DNA
cttttattctccccttcaccgaatctcatcctacattggc

SEQ ID NO: 377 - DNA, 40 bp, synthetic construct
  source 1..40: mol_type = other DNA
tagtgtcccaaattttataatttaggacttctatgatctc

SEQ ID NO: 378 - DNA, 40 bp, synthetic construct
  source 1..40: mol_type = other DNA
atatggtcacctctttgttcaaagtcttctgatagtttcc

SEQ ID NO: 379 - DNA, 40 bp, synthetic construct
  source 1..40: mol_type = other DNA
acaatcttcctgcttctaccactgccccactacaatttct

SEQ ID NO: 380 - DNA, 40 bp, synthetic construct
  source 1..40: mol_type = other DNA
agtcactgtcaccaccacctaaattatagctgttgactca

SEQ ID NO: 381 - DNA, 40 bp, synthetic construct
  source 1..40: mol_type = other DNA
ctgaccccttgccttcacctccaatgctaccactctggtc

SEQ ID NO: 382 - DNA, 40 bp, synthetic construct
  source 1..40: mol_type = other DNA
agaaaatcctgttggtttttcgtgaaaggatgttttcaga

SEQ ID NO: 383 - DNA, 40 bp, synthetic construct
  source 1..40: mol_type = other DNA
acatatactcacagccagaaattagcatgcactagagtgt

SEQ ID NO: 384 - DNA, 40 bp, synthetic construct
  source 1..40: mol_type = other DNA
acccaaagactcactttgcctagcttcaaaatccttactc

SEQ ID NO: 385 - DNA, 40 bp, synthetic construct
  source 1..40: mol_type = other DNA
tgaggtagagactgtgatgaacaaacaccttgacaaaatt

SEQ ID NO: 386 - DNA, 40 bp, synthetic construct
  source 1..40: mol_type = other DNA
tccatatccacccacccagctttccaattttaaagccaat

SEQ ID NO: 387 - DNA, 40 bp, synthetic construct
  source 1..40: mol_type = other DNA
aaggtatgatgtgtagacaagctccagagatggtttctca

SEQ ID NO: 388 - DNA, 40 bp, synthetic construct
  source 1..40: mol_type = other DNA
ctctggtcagcatccaagaaatacttgatgtcactttggc

SEQ ID NO: 389 - DNA, 40 bp, synthetic construct
  source 1..40: mol_type = other DNA
aactgtgaacttccttcagctagaggggcctggctcagaa

SEQ ID NO: 390 - DNA, 40 bp, synthetic construct
  source 1..40: mol_type = other DNA
tgattgttctctgacttatctaccattttccctccttaaa

SEQ ID NO: 391 - DNA, 40 bp, synthetic construct
  source 1..40: mol_type = other DNA
aaacaaaacccatcaaattccctgaccgaacagaattctg

SEQ ID NO: 392 - DNA, 40 bp, synthetic construct
  source 1..40: mol_type = other DNA
cagaggtcacagcctaaacatcaaattccttgaggtgcgg

SEQ ID NO: 393 - DNA, 40 bp, synthetic construct
  source 1..40: mol_type = other DNA
gaaggcaggtgtggctctgcagtgtgattgggtacttgca

SEQ ID NO: 394 - DNA, 40 bp, synthetic construct
  source 1..40: mol_type = other DNA
catggaggaaaaactcatcagggatggaggcacgcctcta

SEQ ID NO: 395 - DNA, 40 bp, synthetic construct
  source 1..40: mol_type = other DNA
agcttgttaaattgaattctatccttcttattcaattcta

SEQ ID NO: 396 - DNA, 40 bp, synthetic construct
  source 1..40: mol_type = other DNA
catagttgtcagcacaatgcctaggctataggaagtactc

SEQ ID NO: 397 - DNA, 40 bp, synthetic construct
  source 1..40: mol_type = other DNA
gcagatatagcttgatggccccatgcttggtttaacatcc

SEQ ID NO: 398 - DNA, 40 bp, synthetic construct
  source 1..40: mol_type = other DNA
ctaaataactagaatactctttattttttcgtatcatgaa

SEQ ID NO: 399 - DNA, 40 bp, synthetic construct
  source 1..40: mol_type = other DNA
agtgtttaaagggtgatatcagactaaacttgaaatatgt

SEQ ID NO: 400 - DNA, 40 bp, synthetic construct
  source 1..40: mol_type = other DNA
ggatgggtctagaaagactagcattgttttaggttgagtg

SEQ ID NO: 401 - DNA, 40 bp, synthetic construct
  source 1..40: mol_type = other DNA
tgctgccaacattaacagtcaagaaatacctccgaataac

SEQ ID NO: 402 - DNA, 40 bp, synthetic construct
  source 1..40: mol_type = other DNA
tattgtgagaggtctgaatagtgttgtaaaataagctgaa

SEQ ID NO: 403 - DNA, 40 bp, synthetic construct
  source 1..40: mol_type = other DNA
ttacaacatgatggcttgttgtctaaatatctcctaggga

SEQ ID NO: 404 - DNA, 40 bp, synthetic construct
  source 1..40: mol_type = other DNA
ctaagtagaagggtactttcacaggaacagagagcaaaag

SEQ ID NO: 405 - DNA, 40 bp, synthetic construct
  source 1..40: mol_type = other DNA
gtcttgtattgcccagtgacatgcacactggtcaaaagta

SEQ ID NO: 406 - DNA, 40 bp, synthetic construct
  source 1..40: mol_type = other DNA
ccctatgtcttccctgatgggctagagttcctctttctca

SEQ ID NO: 407 - DNA, 40 bp, synthetic construct
  source 1..40: mol_type = other DNA
aaagtttccccaaattttaccaatgcaagccatttctcca

SEQ ID NO: 408 - DNA, 40 bp, synthetic construct
  source 1..40: mol_type = other DNA
aactgcagattctctgcatctccctttgccgggtctgaca

SEQ ID NO: 409 - DNA, 40 bp, synthetic construct
  source 1..40: mol_type = other DNA
tagtgctgtggtgctgtgataggtacacaagaaatgagaa

SEQ ID NO: 410 - DNA, 40 bp, synthetic construct
  source 1..40: mol_type = other DNA
taactagcgtcaagaactgagggccctaaactatgctagg

SEQ ID NO: 411 - DNA, 40 bp, synthetic construct
  source 1..40: mol_type = other DNA
cattggctccgtcttcatcctgcagtgacctcagtgcctc

SEQ ID NO: 412 - DNA, 40 bp, synthetic construct
  source 1..40: mol_type = other DNA
tgtttatgtgttatagtgttcatttactcttctggtctaa

SEQ ID NO: 413 - DNA, 40 bp, synthetic construct
  source 1..40: mol_type = other DNA
cctttgaccccttggtcaagctgcaactttggttaaaggg

SEQ ID NO: 414 - DNA, 40 bp, synthetic construct
  source 1..40: mol_type = other DNA
ttctcttgggttacagagattgtcatatgacaaattataa

SEQ ID NO: 415 - DNA, 40 bp, synthetic construct
  source 1..40: mol_type = other DNA
tggaagttgtggtccaagccacagttgcagaccatacttc

SEQ ID NO: 416 - DNA, 40 bp, synthetic construct
  source 1..40: mol_type = other DNA
ctgccctgtggcccttgcttcttacttttacttcttgtcg

SEQ ID NO: 417 - DNA, 40 bp, synthetic construct
  source 1..40: mol_type = other DNA
aactcagatattgtggatgcgagaaattagaagtagatat

SEQ ID NO: 418 - DNA, 40 bp, synthetic construct
  source 1..40: mol_type = other DNA
tacagaaccaccaagtagtaaggctaggatgtagacccag

SEQ ID NO: 419 - DNA, 40 bp, synthetic construct
  source 1..40: mol_type = other DNA
tgagctctcctactgtctacattacatgagctcttattaa

SEQ ID NO: 420 - DNA, 40 bp, synthetic construct
  source 1..40: mol_type = other DNA
aagctaataagtagacaattagtaattagaagtcagatgg

SEQ ID NO: 421 - DNA, 40 bp, synthetic construct
  source 1..40: mol_type = other DNA
agcccaatgtacttgtagtgtagatcaacttattgaaagc

SEQ ID NO: 422 - DNA, 40 bp, synthetic construct
  source 1..40: mol_type = other DNA
ccaatactcagaagtagattattacctcatttattgatga

SEQ ID NO: 423 - DNA, 40 bp, synthetic construct
  source 1..40: mol_type = other DNA
gctagaatcaaatttaagtttatcatatgaggccgggcac

SEQ ID NO: 424 - DNA, 40 bp, synthetic construct
  source 1..40: mol_type = other DNA
taatactaatgataagtaacacctcttgagtacttagtat

SEQ ID NO: 425 - DNA, 40 bp, synthetic construct
  source 1..40: mol_type = other DNA
atggtaattctgtgagatatgtattattgaacatactata

SEQ ID NO: 426 - DNA, 40 bp, synthetic construct
  source 1..40: mol_type = other DNA
tgaaagagaagtgggaattaatacttactgaaatctttct

SEQ ID NO: 427 - DNA, 40 bp, synthetic construct
  source 1..40: mol_type = other DNA
gagagacacgaggaaatagtgtagatttaggctggaggta

SEQ ID NO: 428 - DNA, 40 bp, synthetic construct
  source 1..40: mol_type = other DNA
gttgagagggaaacaagatggtgaagggactagaaaccac

SEQ ID NO: 429 - DNA, 40 bp, synthetic construct
  source 1..40: mol_type = other DNA
caaggttctgaacatgagaaatttttaggaatctgcacag

SEQ ID NO: 430 - DNA, 40 bp, synthetic construct
  source 1..40: mol_type = other DNA
tgccatctaaaaaaatctgacttcactggaaacatggaag

SEQ ID NO: 431 - DNA, 40 bp, synthetic construct
  source 1..40: mol_type = other DNA
gggatcctctcttaagtgtttcctgctggaatctcctcac

SEQ ID NO: 432 - DNA, 40 bp, synthetic construct
  source 1..40: mol_type = other DNA
gtttccttcatgtgacagggagcctcctgccccgaacttc

SEQ ID NO: 433 - DNA, 40 bp, synthetic construct
  source 1..40: mol_type = other DNA
ttggataagagtagggaagaacctagagcctacgctgagc

SEQ ID NO: 434 - DNA, 40 bp, synthetic construct
  source 1..40: mol_type = other DNA
atctggggctttgtgaagactggcttaaaatcagaagccc

SEQ ID NO: 435 - DNA, 40 bp, synthetic construct
  source 1..40: mol_type = other DNA
accgcaatgcttcctgcccattcagggctccagcatgtag

SEQ ID NO: 436 - DNA, 40 bp, synthetic construct
  source 1..40: mol_type = other DNA
tatggggaagcagggtatgaaagagctctgaatgaaatgg

SEQ ID NO: 437 - DNA, 40 bp, synthetic construct
  source 1..40: mol_type = other DNA
ggttgcatgaatcagattatcaacagaaatgttgagacaa

SEQ ID NO: 438 - DNA, 40 bp, synthetic construct
  source 1..40: mol_type = other DNA
aatgcaggcctaggcatgactgaaggctctctcataattc

SEQ ID NO: 439 - DNA, 40 bp, synthetic construct
  source 1..40: mol_type = other DNA
taacgttttcttgtctgctaccccatcatatgcacaacaa

SEQ ID NO: 440 - DNA, 40 bp, synthetic construct
  source 1..40: mol_type = other DNA
ttaattcccaaactcatatagctctgagaaagtctatgct

SEQ ID NO: 441 - DNA, 40 bp, synthetic construct
  source 1..40: mol_type = other DNA
ccctataggggatttctaccctgagcaaaaggctggtctt

SEQ ID NO: 442 - DNA, 40 bp, synthetic construct
  source 1..40: mol_type = other DNA
tcctcaccatatagaaagcttttaacccatcattgaataa

SEQ ID NO: 443 - DNA, 40 bp, synthetic construct
  source 1..40: mol_type = other DNA
taagctgtctagcaaaagcaagggcttggaaaatctgtga

SEQ ID NO: 444 - DNA, 40 bp, synthetic construct
  source 1..40: mol_type = other DNA
aggattagaagattcttctgtgtgtaagaatttcataaac

SEQ ID NO: 445 - DNA, 40 bp, synthetic construct
  source 1..40: mol_type = other DNA
attatcttctggaatagggaatcaagttatattatgtaac

SEQ ID NO: 446 - DNA, 40 bp, synthetic construct
  source 1..40: mol_type = other DNA
ctctctggttgactgttagagttctggcacttgtcactat

SEQ ID NO: 447 - DNA, 40 bp, synthetic construct
  source 1..40: mol_type = other DNA
tcttcagttagatggttaactttgtgaagttgaaaactgt

SEQ ID NO: 448 - DNA, 40 bp, synthetic construct
  source 1..40: mol_type = other DNA
ctacaccatgtggagaaggggtggtggttttgattgctgc

SEQ ID NO: 449 - DNA, 40 bp, synthetic construct
  source 1..40: mol_type = other DNA
actttcctaacctgagcctaacatccctgacatcaggaaa

SEQ ID NO: 450 - DNA, 40 bp, synthetic construct
  source 1..40: mol_type = other DNA
tacactttattcgtctgtgtcctgctctgggatgatagtc

SEQ ID NO: 451 - DNA, 40 bp, synthetic construct
  source 1..40: mol_type = other DNA
tactctttgcattccactgtttttcctaagtgactaaaaa

SEQ ID NO: 452 - DNA, 40 bp, synthetic construct
  source 1..40: mol_type = other DNA
aaaggcctcccaggccaagttatccattcagaaagcattt

SEQ ID NO: 453 - DNA, 40 bp, synthetic construct
  source 1..40: mol_type = other DNA
tattgacatgtacttcttggcagtctgtatgctggatgct

SEQ ID NO: 454 - DNA, 40 bp, synthetic construct
  source 1..40: mol_type = other DNA
tttggtcctaattatgtctttgctcactatccaataaata

SEQ ID NO: 455 - DNA, 40 bp, synthetic construct
  source 1..40: mol_type = other DNA
gttaaaaaaactacctctcaacttgctcaagcatacactc

SEQ ID NO: 456 - DNA, 40 bp, synthetic construct
  source 1..40: mol_type = other DNA
taattagtgctttgcataattaatcatatttaatactctt

SEQ ID NO: 457 - DNA, 40 bp, synthetic construct
  source 1..40: mol_type = other DNA
actagtgttctgtactttatgcccattcatctttaactgt

SEQ ID NO: 458 - DNA, 40 bp, synthetic construct
  source 1..40: mol_type = other DNA
gtattttttgtttaactgcaatcattcttgctgcaggtga

SEQ ID NO: 459 - DNA, 40 bp, synthetic construct
  source 1..40: mol_type = other DNA
gcagtgacttataaatgctaactactctagaaatgtttgc

SEQ ID NO: 460 - DNA, 40 bp, synthetic construct
  source 1..40: mol_type = other DNA
ttataagcatgattacaggagttttaacaggctcataaga

SEQ ID NO: 461 - DNA, 40 bp, synthetic construct
  source 1..40: mol_type = other DNA
agtatccctcaagtagtgtcaggaattagtcatttaaata

SEQ ID NO: 462 - DNA, 40 bp, synthetic construct
  source 1..40: mol_type = other DNA
agtcacccatttggtatattaaagatgtgttgtctactgt

SEQ ID NO: 463 - DNA, 40 bp, synthetic construct
  source 1..40: mol_type = other DNA
tggtcataaaacattgaattctaatctccctctcaaccct

SEQ ID NO: 464 - DNA, 40 bp, synthetic construct
  source 1..40: mol_type = other DNA
acagttgaaaagacctaagcttgtgcctgatttaagcctt

SEQ ID NO: 465 - DNA, 40 bp, synthetic construct
  source 1..40: mol_type = other DNA
caactacagggccttgaactgcacactttcagtccggtcc

SEQ ID NO: 466 - DNA, 40 bp, synthetic construct
  source 1..40: mol_type = other DNA
gtggttctttgaagagacttccacctgggaacagttaaac

SEQ ID NO: 467 - DNA, 40 bp, synthetic construct
  source 1..40: mol_type = other DNA
tggaggaaatatttatccccaggtagttccctttttgcac

SEQ ID NO: 468 - DNA, 40 bp, synthetic construct
  source 1..40: mol_type = other DNA
gcctggtgcttttggtaggggagcttgcactttccccctt

SEQ ID NO: 469 - DNA, 40 bp, synthetic construct
  source 1..40: mol_type = other DNA
tctcatttctttgagaacttcagggaaaatagacaaggac

SEQ ID NO: 470 - DNA, 40 bp, synthetic construct
  source 1..40: mol_type = other DNA
caaacttttcaagccttctctaatcttaaaggtaaacaag

SEQ ID NO: 471 - DNA, 40 bp, synthetic construct
  source 1..40: mol_type = other DNA
tcaacaaaggagaaaagtttgttggcctccaaaggcacag

SEQ ID NO: 472 - DNA, 40 bp, synthetic construct
  source 1..40: mol_type = other DNA
gatgcaacagaccttggaagcatacaggagagctgaactt

SEQ ID NO: 473 - DNA, 40 bp, synthetic construct
  source 1..40: mol_type = other DNA
catctgagatcccagcttctaagaccttcaattctcactc

SEQ ID NO: 474 - DNA, 40 bp, synthetic construct
  source 1..40: mol_type = other DNA
tatcttaacagtgagtgaacaggaaatctcctcttttccc

SEQ ID NO: 475 - DNA, 40 bp, synthetic construct
  source 1..40: mol_type = other DNA
aactcatgctttgtagatgactagatcaaaaaatttcagc

SEQ ID NO: 476 - DNA, 40 bp, synthetic construct
  source 1..40: mol_type = other DNA
tcaaaggaagtcaaaagatgtgaaaaacaatttctgaccc

SEQ ID NO: 477 - DNA, 40 bp, synthetic construct
  source 1..40: mol_type = other DNA
tgccttcacttaagtaatcaattcctaggttatattctga

SEQ ID NO: 478 - DNA, 40 bp, synthetic construct
  source 1..40: mol_type = other DNA
ccctaccttgttcaaaatgttcctgtccagaccaaagtac

SEQ ID NO: 479 - DNA, 40 bp, synthetic construct
  source 1..40: mol_type = other DNA
gcacttacaaattatactacgctctatactttttgtttaa

SEQ ID NO: 480 - DNA, 40 bp, synthetic construct
  source 1..40: mol_type = other DNA
ctttagtttcatttcaaacaatccatacacacacagccct

SEQ ID NO: 481 - DNA, 40 bp, synthetic construct
  source 1..40: mol_type = other DNA
tagggaccacagggttaagggggcagtagaattatactcc

SEQ ID NO: 482 - DNA, 40 bp, synthetic construct
  source 1..40: mol_type = other DNA
ctcacaattaagctaagcagctaagagtcttgcagggtag

SEQ ID NO: 483 - DNA, 40 bp, synthetic construct
  source 1..40: mol_type = other DNA
gttgaaagacagagaggatggggtgctatgccccaaatca

SEQ ID NO: 484 - DNA, 40 bp, synthetic construct
  source 1..40: mol_type = other DNA
gcttgtctaattttatatatcaccctactgaacatgaccc

SEQ ID NO: 485 - DNA, 40 bp, synthetic construct
  source 1..40: mol_type = other DNA
aatattgtacacgtacaccaaagcatcatgttgtacccca

SEQ ID NO: 486 - DNA, 40 bp, synthetic construct
  source 1..40: mol_type = other DNA
tgtgaagtggtggatttgttaattagccttatttaaccat

SEQ ID NO: 487 - DNA, 40 bp, synthetic construct
  source 1..40: mol_type = other DNA
tgacacatatgacattttaactatgttccagatttttgaa

SEQ ID NO: 488 - DNA, 40 bp, synthetic construct
  source 1..40: mol_type = other DNA
gcaaggaatcattcaatgttttctaaatctattactgcat

SEQ ID NO: 489 - DNA, 40 bp, synthetic construct
  source 1..40: mol_type = other DNA
cattttcataggttttcctcgattgatcattattcatgat

SEQ ID NO: 490 - DNA, 40 bp, synthetic construct
  source 1..40: mol_type = other DNA
aaagtgatcaagatatttttagttcaggctccaaaatttt

SEQ ID NO: 491 - DNA, 40 bp, synthetic construct
  source 1..40: mol_type = other DNA
ctttacaggccgagaaaaatgaatctgaattcctgacctc

SEQ ID NO: 492 - DNA, 40 bp, synthetic construct
  source 1..40: mol_type = other DNA
tccactcaaggcctacattctgctataatgcaatttcaag

SEQ ID NO: 493 - DNA, 40 bp, synthetic construct
  source 1..40: mol_type = other DNA
aactgcttaaaattaatggcacaagtcatgtttttgatgt

SEQ ID NO: 494 - DNA, 40 bp, synthetic construct
  source 1..40: mol_type = other DNA
ctgactgtgacgtagcaataaagaaacccacgtttcatat

SEQ ID NO: 495 - DNA, 40 bp, synthetic construct
  source 1..40: mol_type = other DNA
ctggcccactgcttggaggagagcactcaggaccatgaac

SEQ ID NO: 496 - DNA, 40 bp, synthetic construct
  source 1..40: mol_type = other DNA
ttctgaaatgataaagtcaatcacaggaaggcacctggac

SEQ ID NO: 497 - DNA, 40 bp, synthetic construct
  source 1..40: mol_type = other DNA
atcattctctttcccttcctctatgtggcagaaagtaaaa

SEQ ID NO: 498 - DNA, 40 bp, synthetic construct
  source 1..40: mol_type = other DNA
ggagataataatgtgttactccctaaggcagagtgccctt

SEQ ID NO: 499 - DNA, 40 bp, synthetic construct
  source 1..40: mol_type = other DNA
caattaacttggccatgtgactggttgtgactaaaataat

SEQ ID NO: 500 - DNA, 40 bp, synthetic construct
  source 1..40: mol_type = other DNA
cactaaatcaatatacttctcaacaatttccaacagccct

SEQ ID NO: 501 - DNA, 40 bp, synthetic construct
  source 1..40: mol_type = other DNA
ctaggctcctgagtttgctggggatgcgaagaacccttat

SEQ ID NO: 502 - DNA, 40 bp, synthetic construct
  source 1..40: mol_type = other DNA
ccgaggaccccgcactcggagccgccagccggccccaccg

SEQ ID NO: 503 - DNA, 40 bp, synthetic construct
  source 1..40: mol_type = other DNA
ttggaagcacagggtgtgggataatgctaattactagtga

SEQ ID NO: 504 - DNA, 40 bp, synthetic construct
  source 1..40: mol_type = other DNA
gttcagtatgcctttgattttacaataatattcctgttat

SEQ ID NO: 505 - DNA, 40 bp, synthetic construct
  source 1..40: mol_type = other DNA
agattccatgaagtattacagcatttggtagtctttttgc

SEQ ID NO: 506 - DNA, 40 bp, synthetic construct
  source 1..40: mol_type = other DNA
tatttgctctgaaataagacataatttggggtgagaaagc

SEQ ID NO: 507 - DNA, 40 bp, synthetic construct
  source 1..40: mol_type = other DNA
actcatgatatttggctctagaatacatgctctgaatcat

SEQ ID NO: 508 - DNA, 40 bp, synthetic construct
  source 1..40: mol_type = other DNA
tccaagatgaagtggctactaactgacagagggcataatt

SEQ ID NO: 509 - DNA, 40 bp, synthetic construct
  source 1..40: mol_type = other DNA
tattcacagtaactctgtgcctcaagtactattgtaatac

SEQ ID NO: 510 - DNA, 40 bp, synthetic construct
  source 1..40: mol_type = other DNA
acatcctcaatctacacactaggatagtataaaagtaata

SEQ ID NO: 511 - DNA, 40 bp, synthetic construct
  source 1..40: mol_type = other DNA
gtctacccatatgtgaccttcatgtctttgctctaagccc

SEQ ID NO: 512 - DNA, 40 bp, synthetic construct
  source 1..40: mol_type = other DNA
cgtgtaatccttgacaatgtcatctcatctatttattccc

SEQ ID NO: 513 - DNA, 40 bp, synthetic construct
  source 1..40: mol_type = other DNA
tctgaaagagactaaccttccctcgctttgcagagaaaga

SEQ ID NO: 514 - DNA, 40 bp, synthetic construct
  source 1..40: mol_type = other DNA
atgcatggattctcttgaaaaaatgtttctgccatgatgt

SEQ ID NO: 515 - DNA, 40 bp, synthetic construct
  source 1..40: mol_type = other DNA
tagttgaagacctactgtgttcagggccgtgagccagggc

SEQ ID NO: 516 - DNA, 40 bp, synthetic construct
  source 1..40: mol_type = other DNA
caacgtggagagctgtcctggcaccatttcttcctgctgt

SEQ ID NO: 517 - DNA, 40 bp, synthetic construct
  source 1..40: mol_type = other DNA
atcctcaaaggagcctggcttgggctaacaaggaagaact

SEQ ID NO: 518 - DNA, 40 bp, synthetic construct
  source 1..40: mol_type = other DNA
tgcctgggaccctgccccaagcaaagtaataatctgaatg

SEQ ID NO: 519 - DNA, 40 bp, synthetic construct
  source 1..40: mol_type = other DNA
ctggtgtgtccagtgtgatccctgcacccatgcccggagc

SEQ ID NO: 520 - DNA, 40 bp, synthetic construct
  source 1..40: mol_type = other DNA
ctgccccctgcagcagggaaggggctctggaagggtctga

SEQ ID NO: 521 - DNA, 40 bp, synthetic construct
  source 1..40: mol_type = other DNA
tagctgctgccccactatgcaccatcgcttatctgttctt

SEQ ID NO: 522 - DNA, 40 bp, synthetic construct
  source 1..40: mol_type = other DNA
gaaacccgaaaaatgtcctggtcctcttcttaagtctggg

SEQ ID NO: 523 - DNA, 40 bp, synthetic construct
  source 1..40: mol_type = other DNA
gctgagaacatgactctgcttggcgttccatttaattgac

SEQ ID NO: 524 - DNA, 40 bp, synthetic construct
  source 1..40: mol_type = other DNA
gagagggtgtgcatttgaagtatagatttgttaaacatag

SEQ ID NO: 525 - DNA, 40 bp, synthetic construct
  source 1..40: mol_type = other DNA
catcaggcaaaaatacttcgatgggactgtgttctttcag

SEQ ID NO: 526 - DNA, 40 bp, synthetic construct
  source 1..40: mol_type = other DNA
tctaaagtgatgtaatgttgccacggaaattctaatccct

SEQ ID NO: 527 - DNA, 40 bp, synthetic construct
  source 1..40: mol_type = other DNA
cgtgcagaaccagctctgtcttcccagacactgtcgcttt

SEQ ID NO: 528 - DNA, 40 bp, synthetic construct
  source 1..40: mol_type = other DNA
acccctgagcacctcagtgtccgtgactgtggagcggagg

SEQ ID NO: 529 - DNA, 40 bp, synthetic construct
  source 1..40: mol_type = other DNA
ctgcctgggacacgtacggctgcccagtgatcctgagcgc

SEQ ID NO: 530 - DNA, 40 bp, synthetic construct
  source 1..40: mol_type = other DNA
cacagccggatggtgtgggagctggcactgccggggctcc

SEQ ID NO: 531 - DNA, 40 bp, synthetic construct
  source 1..40: mol_type = other DNA
cgtcttggcagaggctccctgtcatcaaggacctgaggtt

SEQ ID NO: 532 - DNA, 40 bp, synthetic construct
  source 1..40: mol_type = other DNA
gaccccacaaagatgagcgggtccccttcccaattttcgg

SEQ ID NO: 533 - DNA, 40 bp, synthetic construct
  source 1..40: mol_type = other DNA
tcaggaagccggtgctcagcaaacttatctgaagctcttg

SEQ ID NO: 534 - DNA, 40 bp, synthetic construct
  source 1..40: mol_type = other DNA
gaggctgcagaggaacatcgtttggtcaaatgtgaaatgt

SEQ ID NO: 535 - DNA, 40 bp, synthetic construct
  source 1..40: mol_type = other DNA
ctagcttctagaaagtgctgccaatttggggaccaaggga

SEQ ID NO: 536 - DNA, 40 bp, synthetic construct
  source 1..40: mol_type = other DNA
ggaaacacttctttttcccttgacaaaggacatcctctgc

SEQ ID NO: 537 - DNA, 40 bp, synthetic construct
  source 1..40: mol_type = other DNA
gcatgtgcataaacactcgtgtgtgtgtccttttatccca

SEQ ID NO: 538 - DNA, 40 bp, synthetic construct
  source 1..40: mol_type = other DNA
ccaaatctctatacatgtccatagagagaggcagacgtat

SEQ ID NO: 539 - DNA, 40 bp, synthetic construct
  source 1..40: mol_type = other DNA
gggttgaagacaaggggctcagagcttgctttttatacac

SEQ ID NO: 540 - DNA, 40 bp, synthetic construct
  source 1..40: mol_type = other DNA
agattcatcttcatggcaggacttcaggcaagagaggccc

SEQ ID NO: 541 - DNA, 40 bp, synthetic construct
  source 1..40: mol_type = other DNA
ctcaccccttagcaggaccctgacggaactgggtacaggc

SEQ ID NO: 542 - DNA, 40 bp, synthetic construct
  source 1..40: mol_type = other DNA
ggttgggagacaatgggtggcccctcggtgtggtgtcctc

SEQ ID NO: 543 - DNA, 40 bp, synthetic construct
  source 1..40: mol_type = other DNA
agagtctagagggcccgtggggacgggagtcctgggaacc

SEQ ID NO: 544 - DNA, 40 bp, synthetic construct
  source 1..40: mol_type = other DNA
gcggcatgtccggcttcaccctgcccagaatcacagcctc

SEQ ID NO: 545 - DNA, 40 bp, synthetic construct
  source 1..40: mol_type = other DNA
atggttaaaaaattctcctacttaagactcccagacccct

SEQ ID NO: 546 - DNA, 40 bp, synthetic construct; identical to SEQ ID NO: 539
  source 1..40: mol_type = other DNA

SEQ ID NO: 547 - DNA, 40 bp, synthetic construct; identical to SEQ ID NO: 540
  source 1..40: mol_type = other DNA

SEQ ID NO: 548 - DNA, 40 bp, synthetic construct; identical to SEQ ID NO: 541
  source 1..40: mol_type = other DNA

SEQ ID NO: 549 - DNA, 40 bp, synthetic construct; identical to SEQ ID NO: 542
  source 1..40: mol_type = other DNA

SEQ ID NO: 550 - DNA, 40 bp, synthetic construct; identical to SEQ ID NO: 543
  source 1..40: mol_type = other DNA

SEQ ID NO: 551 - DNA, 40 bp, synthetic construct; identical to SEQ ID NO: 544
  source 1..40: mol_type = other DNA

SEQ ID NO: 552 - DNA, 40 bp, synthetic construct
  source 1..40: mol_type = other DNA
tgagattccagggctggttccacaacggccggcatcggcc

SEQ ID NO: 553 - DNA, 40 bp, synthetic construct
  source 1..40: mol_type = other DNA
ctgagtcactaacaaagctcaggcctgaccacaggacatt

SEQ ID NO: 554 - DNA, 40 bp, synthetic construct
  source 1..40: mol_type = other DNA
ggctggcctacctgccacggggccagggctgggtgctttc

SEQ ID NO: 555 - DNA, 40 bp, synthetic construct
  source 1..40: mol_type = other DNA
gggctctggacgctggaggcctgaggctgcaccccaggtt

SEQ ID NO: 556 - DNA, 40 bp, synthetic construct
  source 1..40: mol_type = other DNA
acagtggccactcacccactgggcccacatccccacaggc

SEQ ID NO: 557 - DNA, 40 bp, synthetic construct
  source 1..40: mol_type = other DNA
actctgccagcctttgatgcctcgctgagacagagggtct

SEQ ID NO: 558 - DNA, 40 bp, synthetic construct
  source 1..40: mol_type = other DNA
agccggggctctggccccatccaggggctcccccagcagc

SEQ ID NO: 559 - DNA, 40 bp, synthetic construct
  source 1..40: mol_type = other DNA
ccttggaagtcagtcagcaggtcaggacacagttcagccc

SEQ ID NO: 560 - DNA, 40 bp, synthetic construct
  source 1..40: mol_type = other DNA
ttacatgcagttggtcttctcctgtgaatggggaaactga

SEQ ID NO: 561 - DNA, 40 bp, synthetic construct
  source 1..40: mol_type = other DNA
ctgcatcacagaacagctgcatttctaatgtcaggcttct

SEQ ID NO: 562 - DNA, 40 bp, synthetic construct
  source 1..40: mol_type = other DNA
cagcctgggaggcttgtcaacctcctttgacaagcacgcc

SEQ ID NO: 563 - DNA, 40 bp, synthetic construct
  source 1..40: mol_type = other DNA
agaaactggggctccagggcatggaggctgcctgtggcca

SEQ ID NO: 564 - DNA, 40 bp, synthetic construct
  source 1..40: mol_type = other DNA
tcccggcctggaggaagtcttattagcctcatttcatgga

SEQ ID NO: 565 - DNA, 40 bp, synthetic construct
  source 1..40: mol_type = other DNA
tcctgccagccccctcacgctcacgaattcagtcccaggg

SEQ ID NO: 566 - DNA, 40 bp, synthetic construct
  source 1..40: mol_type = other DNA
aattctaaaggtgaagggacgtctacacccccaacaaaac

SEQ ID NO: 567 - DNA, 40 bp, synthetic construct
  source 1..40: mol_type = other DNA
ggaaatattagtcccctctgcctgggacaagaccaccgaa

SEQ ID NO: 568 - DNA, 40 bp, synthetic construct
  source 1..40: mol_type = other DNA
aaacacacctctgaatggaaagctgagaaacagtgatctc

SEQ ID NO: 569 - DNA, 40 bp, synthetic construct
  source 1..40: mol_type = other DNA
actgcaccccctcccttcccgtgccggcaatttaaccggg

SEQ ID NO: 570 - DNA, 40 bp, synthetic construct
  source 1..40: mol_type = other DNA
tgccttcctaccttgaccagtcggtccttgcgggggtccc

SEQ ID NO: 571 - DNA, 40 bp, synthetic construct
  source 1..40: mol_type = other DNA
atttccttcatcttgtccttctagcctggagactcttcgg

SEQ ID NO: 572 - DNA, 40 bp, synthetic construct
  source 1..40: mol_type = other DNA
aatgcccgaaaattccagcagcagcccaagatggtggcca

SEQ ID NO: 573 - DNA, 40 bp, synthetic construct
  source 1..40: mol_type = other DNA
cgttgcaaatgcccaagggggtaaccctaaaagttaaagg

SEQ ID NO: 574 - DNA, 40 bp, synthetic construct
  source 1..40: mol_type = other DNA
acacaacccctgtgcaagtttcattccggcgcacaggggc

SEQ ID NO: 575 - DNA, 40 bp, synthetic construct
  source 1..40: mol_type = other DNA
tgcaagaactaatttagcatgcaaggacggggaggaccgg

SEQ ID NO: 576 - DNA, 40 bp, synthetic construct
  source 1..40: mol_type = other DNA
gccacgagggcacccacgggcggacagacggccaaagaat

SEQ ID NO: 577 - DNA, 40 bp, synthetic construct
  source 1..40: mol_type = other DNA
accccatatccaagccggcagaatgggcgcatttccaaga

SEQ ID NO: 578 - DNA, 40 bp, synthetic construct
  source 1..40: mol_type = other DNA
gcctggggagaccacgagaaggggtgactggggcgcggcg

SEQ ID NO: 579 - DNA, 40 bp, synthetic construct
  source 1..40: mol_type = other DNA
ctgcagtaggggacaactaggaaggccggcaggccacacg

SEQ ID NO: 580 - DNA, 40 bp, synthetic construct
  source 1..40: mol_type = other DNA
gagtgggtcccccgggatttagggggtgaggtggaggtgg

SEQ ID NO: 581 - DNA, 40 bp, synthetic construct
  source 1..40: mol_type = other DNA
tccccgccagggaagaggggtgcagggggccccgtccgcc

SEQ ID NO: 582 - DNA, 40 bp, synthetic construct
  source 1..40: mol_type = other DNA
tgaggcgccgcgcctgccctgcggcggagttgcccctgta

SEQ ID NO: 583 - DNA, 40 bp, synthetic construct
  source 1..40: mol_type = other DNA
aaacgccgggagcagcgaggggcagagcccaaaagccatc

SEQ ID NO: 584 - DNA, 40 bp, synthetic construct
  source 1..40: mol_type = other DNA
ttgttaagcaaagatcaaagcccggcagagaatgggagcg

SEQ ID NO: 585 - DNA, 40 bp, synthetic construct
  source 1..40: mol_type = other DNA
caacttcaacaaaactcccctgtagtccgtgtgacgttac

SEQ ID NO: 586 - DNA, 40 bp, synthetic construct
  source 1..40: mol_type = other DNA
ctgctactgcgccgacagccctctggaggctccaggactt

SEQ ID NO: 587 - DNA, 40 bp, synthetic construct
  source 1..40: mol_type = other DNA
gctcttctgcccctcgccggagcgtgcggactctgctgct

SEQ ID NO: 588 - DNA, 40 bp, synthetic construct
  source 1..40: mol_type = other DNA
tccgcgctcggctctcgcttctgctgccccgcgctccctc

SEQ ID NO: 589 - DNA, 40 bp, synthetic construct
  source 1..40: mol_type = other DNA
tttccacttcgcagcacaggagctggtgttccatggctgg

SEQ ID NO: 590 - DNA, 40 bp, synthetic construct
  source 1..40: mol_type = other DNA
ggtcgttgaggaggttggcatcggggtacgcgcggcggat

SEQ ID NO: 591 - DNA, 40 bp, synthetic construct
  source 1..40: mol_type = other DNA
tgtcctacttcaaatgtgtgcagaaggaggtcctgccgtc

SEQ ID NO: 592 - DNA, 40 bp, synthetic construct
  source 1..40: mol_type = other DNA
tcgggcggctctcttaagacttccctgcaacttgttgccc

SEQ ID NO: 593 - DNA, 40 bp, synthetic construct
  source 1..40: mol_type = other DNA
acccacgtttctttgctactcacccccctcccttctctcc

SEQ ID NO: 594 - DNA, 40 bp, synthetic construct
  source 1..40: mol_type = other DNA
ctagaactttgaagtttgccgtggtgtttctagggatccg

SEQ ID NO: 595 - DNA, 40 bp, synthetic construct
  source 1..40: mol_type = other DNA
agaagggggtccgggaggggtgccttcgggagaagccagt

SEQ ID NO: 596 - DNA, 40 bp, synthetic construct
  source 1..40: mol_type = other DNA
caggggcaccccaatgggcccgagggtgcgggctggcagg

SEQ ID NO: 597 - DNA, 40 bp, synthetic construct
  source 1..40: mol_type = other DNA
gggtgcgctttgtgtcccccgcctgcgccccagcccggct

SEQ ID NO: 598 - DNA, 40 bp, synthetic construct
  source 1..40: mol_type = other DNA
gcctcagcggccgggagccgccaactccggggggaggggg

SEQ ID NO: 599 - DNA, 40 bp, synthetic construct
  source 1..40: mol_type = other DNA
aaagtgcagtaatacccttgatcagagttgatgacttgaa

SEQ ID NO: 600 - DNA, 40 bp, synthetic construct
  source 1..40: mol_type = other DNA
gagagaaataaagtagttgctctatttgtaaattgaaaag

SEQ ID NO: 601 - DNA, 40 bp, synthetic construct
  source 1..40: mol_type = other DNA
ggtagcagtgattgctgtatatttgtgaaaaggaggcaag

SEQ ID NO: 602 - DNA, 40 bp, synthetic construct
  source 1..40: mol_type = other DNA
tgctgataatggaagtgcagtgggttagctttgtttccat

SEQ ID NO: 603 - DNA, 40 bp, synthetic construct
  source 1..40: mol_type = other DNA
ccgttctaccgtgactagtatggaattgtgggaaccagaa

SEQ ID NO: 604 - DNA, 40 bp, synthetic construct
  source 1..40: mol_type = other DNA
ttaacatcagtgtcaactgcagtgttgtttctgagtaata

SEQ ID NO: 605 - DNA, 40 bp, synthetic construct
  source 1..40: mol_type = other DNA
cataactccatgctctcaaaccaatcactccttcattcat

SEQ ID NO: 606 - DNA, 40 bp, synthetic construct
  source 1..40: mol_type = other DNA
ttctcctatgctgcaccagaaagggttttgtgggttatca

SEQ ID NO: 607 - DNA, 40 bp, synthetic construct
  source 1..40: mol_type = other DNA
atcgttcagcatctttaggaaatatccagagactgcattg

SEQ ID NO: 608 - DNA, 40 bp, synthetic construct
  source 1..40: mol_type = other DNA
tttattaagagcaaaaaaagcctgtttcgttagccagtca

SEQ ID NO: 609 - DNA, 40 bp, synthetic construct
  source 1..40: mol_type = other DNA
ttgttcatatgcctaacttaataaattcttcatacagaaa

SEQ ID NO: 610 - DNA, 40 bp, synthetic construct
  source 1..40: mol_type = other DNA
ataacttttaaacccaaacacctagagatttcattatgta

SEQ ID NO: 611 - DNA, 40 bp, synthetic construct
  source 1..40: mol_type = other DNA
ttcttaccattaagtcttccaaatgataatttattataaa

SEQ ID NO: 612 - DNA, 40 bp, synthetic construct
  source 1..40: mol_type = other DNA
tatgtaaggacaacttcattatatgcttgaagaaattgtt

SEQ ID NO: 613 - DNA, 40 bp, synthetic construct
  source 1..40: mol_type = other DNA
aatcttaaaagtgacactagtcacattccacacggttaaa

SEQ ID NO: 614 - DNA, 40 bp, synthetic construct
  source 1..40: mol_type = other DNA
attttgaaaactattcctttatctggaatgaatgtaaacc

SEQ ID NO: 615 - DNA, 40 bp, synthetic construct
  source 1..40: mol_type = other DNA
ttgcattaagggcaccagaaacttatagaaaaccaaaaag

SEQ ID NO: 616 - DNA, 40 bp, synthetic construct
  source 1..40: mol_type = other DNA
taaaagacagtgaactgaacagtaattaacattacatcca

SEQ ID NO: 617 - DNA, 40 bp, synthetic construct
  source 1..40: mol_type = other DNA
caaaaaactgtgtttatcatataccaaacattttcaagtt

SEQ ID NO: 618 - DNA, 40 bp, synthetic construct
  source 1..40: mol_type = other DNA
tctcaggatattttgttctctgacacaaatacaccagtca

SEQ ID NO: 619 - DNA, 40 bp, synthetic construct
  source 1..40: mol_type = other DNA
tagctttacatctcagaatgaatcaatgtgggggcagaaa

SEQ ID NO: 620 - DNA, 40 bp, synthetic construct
  source 1..40: mol_type = other DNA
agacctatatacctatagtgcctaatagacaataagccac

SEQ ID NO: 621 - DNA, 40 bp, synthetic construct
  source 1..40: mol_type = other DNA
tctctcccctgcctagactaaggtaagtgggtcttacctt

SEQ ID NO: 622 - DNA, 40 bp, synthetic construct
  source 1..40: mol_type = other DNA
catcctgcttttaaaacccttagtgctcagcggcttgtct

SEQ ID NO: 623 - DNA, 40 bp, synthetic construct
  source 1..40: mol_type = other DNA
agcttataaacttcagagtaatgtagcacaaatgtctgtc

SEQ ID NO: 624 - DNA, 40 bp, synthetic construct
  source 1..40: mol_type = other DNA
aacttgaaataaaactttaaacgttgattgattctttccc

SEQ ID NO: 625 - DNA, 40 bp, synthetic construct
  source 1..40: mol_type = other DNA
gacaggcttagagtccataacaaacaatcttagctggaaa

SEQ ID NO: 626 - DNA, 40 bp, synthetic construct
  source 1..40: mol_type = other DNA
tgctcaacaacacttgtggaagagcagggcaagctatttc

SEQ ID NO: 627 - DNA, 40 bp, synthetic construct
  source 1..40: mol_type = other DNA
ttacaacatcactgtagacattacttttacccacagtgcc

SEQ ID NO: 628 - DNA, 40 bp, synthetic construct
  source 1..40: mol_type = other DNA
atcctagttgtatatacttcttggataaagtatcttcgta

SEQ ID NO: 629 - DNA, 40 bp, synthetic construct
  source 1..40: mol_type = other DNA
atttttggggagtgccattcctgcaggtcttgaagacagg

SEQ ID NO: 630 - DNA, 40 bp, synthetic construct
  source 1..40: mol_type = other DNA
cacacagccaatgaaactgacagagccaatgcaaccaaaa

SEQ ID NO: 631 - DNA, 40 bp, synthetic construct
  source 1..40: mol_type = other DNA
acgacttcaatcaagagaaacaggcaggtcagagtgtgaa

SEQ ID NO: 632 - DNA, 40 bp, synthetic construct
  source 1..40: mol_type = other DNA
ctggttatcagggttcatagcacataggtttgacaaccac

SEQ ID NO: 633 - DNA, 40 bp, synthetic construct
  source 1..40: mol_type = other DNA
tttattattcagctgggtaagccaagtgacagtcttcccc

SEQ ID NO: 634 - DNA, 40 bp, synthetic construct
  source 1..40: mol_type = other DNA
gttttattctaggaatcaactgctttctaaaaatgtctaa

SEQ ID NO: 635 - DNA, 40 bp, synthetic construct
  source 1..40: mol_type = other DNA
tttactgatggtacttattcccccaattattgattattga

SEQ ID NO: 636 - DNA, 40 bp, synthetic construct
  source 1..40: mol_type = other DNA
gcatttaggaatattcaatattgatactaaggtcatcttt

SEQ ID NO: 637 - DNA, 40 bp, synthetic construct
  source 1..40: mol_type = other DNA
tactctgtaatgtagtaatctttatgaagaaataaatttg

SEQ ID NO: 638 - DNA, 40 bp, synthetic construct
  source 1..40: mol_type = other DNA
attttgaaaaaatgtttcactgcattttactatacaagct

SEQ ID NO: 639 - DNA, 40 bp, synthetic construct
  source 1..40: mol_type = other DNA
accacacattcatcaaaaaatacctcaaagaaaattctgc

SEQ ID NO: 640 - DNA, 40 bp, synthetic construct
  source 1..40: mol_type = other DNA
gttgtcacaataaactcagtactgagtaaaatatcacaaa

SEQ ID NO: 641 - DNA, 40 bp, synthetic construct
  source 1..40: mol_type = other DNA
gagtatatattgtattacttacctgatgcgcaaagaccca

SEQ ID NO: 642 - DNA, 40 bp, synthetic construct
  source 1..40: mol_type = other DNA
aaaatgacagcaacataggtgccacctgaggtccacatct

SEQ ID NO: 643 - DNA, 40 bp, synthetic construct
  source 1..40: mol_type = other DNA
tggagagagtggggttaatctgttactacactttgctact

SEQ ID NO: 644 - DNA, 40 bp, synthetic construct
  source 1..40: mol_type = other DNA
atttccatcattttgtctttcagtaagcatgtacgaagta

SEQ ID NO: 645 - DNA, 40 bp, synthetic construct
  source 1..40: mol_type = other DNA
gagatgaagatggtacatcagtagggagcccctctactgg

SEQ ID NO: 646 - DNA, 40 bp, synthetic construct
  source 1..40: mol_type = other DNA
tctaattcatcaaagtattctgggttgattccaggtacgt

SEQ ID NO: 647 - DNA, 40 bp, synthetic construct
  source 1..40: mol_type = other DNA
acaaactcgttttgtacagagaggaaaatattaaaacacc

SEQ ID NO: 648 - DNA, 40 bp, synthetic construct
  source 1..40: mol_type = other DNA
atgttaattataaacactgttataagttttacaaatgtaa

SEQ ID NO: 649 - DNA, 40 bp, synthetic construct
  source 1..40: mol_type = other DNA
tccactggcagagagaatatatgtttccattacggtccca

SEQ ID NO: 650 - DNA, 40 bp, synthetic construct
  source 1..40: mol_type = other DNA
tcaaaggttttctatcacgttttctattatttactcacat

SEQ ID NO: 651 - DNA, 40 bp, synthetic construct
  source 1..40: mol_type = other DNA
aaaaacaagagtcacacaacctatgctccacaatatctgc

SEQ ID NO: 652 - DNA, 40 bp, synthetic construct
  source 1..40: mol_type = other DNA
ataggttattctacaatcgacaccaactatcagcggcttt

SEQ ID NO: 653 - DNA, 40 bp, synthetic construct
  source 1..40: mol_type = other DNA
attgaattaaatgatggcttgattatccaggaatcagcca

SEQ ID NO: 654 - DNA, 40 bp, synthetic construct
  source 1..40: mol_type = other DNA
cttaccataacagagtaatctctagcttattccaaggata

SEQ ID NO: 655 - DNA, 40 bp, synthetic construct
  source 1..40: mol_type = other DNA
acctaaaatttaactagaatcacttttcaatgaagctgct

SEQ ID NO: 656 - DNA, 40 bp, synthetic construct
  source 1..40: mol_type = other DNA
taaactaagagcctttgatcttgccttattctgataaaat

SEQ ID NO: 657 - DNA, 40 bp, synthetic construct
  source 1..40: mol_type = other DNA
aaataataattcacaaggaaatccttattgtttatttaaa

SEQ ID NO: 658 - DNA, 40 bp, synthetic construct
  source 1..40: mol_type = other DNA
gtaatatgtaggttaaacagaaatgttggttgaatcatgt

SEQ ID NO: 659 - DNA, 40 bp, synthetic construct
  source 1..40: mol_type = other DNA
tgcagacactaatcaaaccaaacagggccaattaaaattg

SEQ ID NO: 660 - DNA, 40 bp, synthetic construct
  source 1..40: mol_type = other DNA
taaagtgcaatgggacagagcaacttcattttcacaaaca

SEQ ID NO: 661 - DNA, 40 bp, synthetic construct
  source 1..40: mol_type = other DNA
taatctaattgccagaaatgcttgcccattgcaatgggag

SEQ ID NO: 662 - DNA, 40 bp, synthetic construct
  source 1..40: mol_type = other DNA
agttgacaatgactgcttagtttagggttttgaagtaaac

SEQ ID NO: 663 - DNA, 40 bp, synthetic construct
  source 1..40: mol_type = other DNA
cagatggcaggtattctgtgaattaacactgatgcttctg

SEQ ID NO: 664 - DNA, 40 bp, synthetic construct
  source 1..40: mol_type = other DNA
agtcaagttcagaaatgatctgttatgaccccatgaaacg

SEQ ID NO: 665 - DNA, 40 bp, synthetic construct
  source 1..40: mol_type = other DNA
gggatgctctgatacatcattcagtaaaatgatagaaaaa

SEQ ID NO: 666 - DNA, 40 bp, synthetic construct
  source 1..40: mol_type = other DNA
tagctgtattgcttgatagcttcatagcttgataaccatt

SEQ ID NO: 667 - DNA, 40 bp, synthetic construct
  source 1..40: mol_type = other DNA
ttttagcagggaattaacacaggtatataaatgaagaaaa

SEQ ID NO: 668 - DNA, 40 bp, synthetic construct
  source 1..40: mol_type = other DNA
ttgattgtttatgaagctgagattgtttactggtttcgag

SEQ ID NO: 669 - DNA, 40 bp, synthetic construct
  source 1..40: mol_type = other DNA
tctgtgtttttatgtttgggaacatgagggaatcagttct

SEQ ID NO: 670 - DNA, 40 bp, synthetic construct
  source 1..40: mol_type = other DNA
ttcttaagctttcatttttccagtggtgaatgtagagaga

SEQ ID NO: 671 - DNA, 40 bp, synthetic construct
  source 1..40: mol_type = other DNA
acggtaactgaataaacttaagaactgaggtaaagttttc

SEQ ID NO: 672 - DNA, 40 bp, synthetic construct
  source 1..40: mol_type = other DNA
tcaatatgtaaaattgatcaattcagacacctttatatgg

SEQ ID NO: 673 - DNA, 40 bp, synthetic construct
  source 1..40: mol_type = other DNA
tgtctctttcatgctgtaaatagagcattgcatgaaagat

SEQ ID NO: 674 - DNA, 40 bp, synthetic construct
  source 1..40: mol_type = other DNA
ttcatagcacagtttataaacctaagaaagcaaagatgaa

SEQ ID NO: 675 - DNA, 40 bp, synthetic construct
  source 1..40: mol_type = other DNA
aaccaagcaggattctatgactaaaaaagtgtatttgtat

SEQ ID NO: 676 - DNA, 40 bp, synthetic construct
  source 1..40: mol_type = other DNA
agatagagaatttcaaagaaaccatctttatcagctgcac

SEQ ID NO: 677 - DNA, 40 bp, synthetic construct
  source 1..40: mol_type = other DNA
ccaagaatgaaaagatgcactaattcgactgaaagccaag

SEQ ID NO: 678 - DNA, 40 bp, synthetic construct
  source 1..40: mol_type = other DNA
tcatagttgagacatataacaaccataaaggtccgcatat

SEQ ID NO: 679 - DNA, 40 bp, synthetic construct
  source 1..40: mol_type = other DNA
aggaaagggtggaaaggcaagcagcggggagtgttggctg

SEQ ID NO: 680 - DNA, 40 bp, synthetic construct
  source 1..40: mol_type = other DNA
ctataaattgacctatcctgtaaaaaaggatgtcacagca

SEQ ID NO: 681 - DNA, 40 bp, synthetic construct
  source 1..40: mol_type = other DNA
acaattgacctaagactgtaaattgtaaattgactataaa

SEQ ID NO: 682 - DNA, 40 bp, synthetic construct
  source 1..40: mol_type = other DNA
gcaagactgggtatactattaataggaaaaaatgaacttc

SEQ ID NO: 683 - DNA, 40 bp, synthetic construct
  source 1..40: mol_type = other DNA
attgctttgatattgattgaatcacagagaaaatcctaag

SEQ ID NO: 684 - DNA, 40 bp, synthetic construct
  source 1..40: mol_type = other DNA
tagattatgctggcaaatctcagtgatcagagaattatat

SEQ ID NO: 685 - DNA, 40 bp, synthetic construct
  source 1..40: mol_type = other DNA
attcagaaatggaataggaagatatttatgtgccatcctg

SEQ ID NO: 686 - DNA, 40 bp, synthetic construct
  source 1..40: mol_type = other DNA
gtttgaattattattcaaacagtgtatgtttgtttgtact

SEQ ID NO: 687 - DNA, 40 bp, synthetic construct
  source 1..40: mol_type = other DNA
aatgcaacagagacaggtatttatagcatctgttttccat

SEQ ID NO: 688 - DNA, 40 bp, synthetic construct
  source 1..40: mol_type = other DNA
tttaatatccaaatatgtatggacacatacaattgtacat

SEQ ID NO: 689 - DNA, 40 bp, synthetic construct
  source 1..40: mol_type = other DNA
acgtctaccgtcattttcgtaattattcggtttccctgtc

SEQ ID NO: 690 - DNA, 40 bp, synthetic construct
  source 1..40: mol_type = other DNA
ggagcgctcctgcgcgccttgttcgttaggatttattttt

SEQ ID NO: 691 - DNA, 40 bp, synthetic construct
  source 1..40: mol_type = other DNA
ggtggctccctaatgcctgctcgtttcaggtctcagctct

SEQ ID NO: 692 - DNA, 40 bp, synthetic construct
  source 1..40: mol_type = other DNA
ccttagtgtgttgaggacgctgcagaaggtacagaggaga

SEQ ID NO: 693 - DNA, 40 bp, synthetic construct
  source 1..40: mol_type = other DNA
gaccagatggtaggacagtcattctcctctgcgtctccgc

SEQ ID NO: 694 - DNA, 40 bp, synthetic construct
  source 1..40: mol_type = other DNA
cgtgaggcatggagtttttgtcctgcccctgcctggttag

SEQ ID NO: 695 - DNA, 40 bp, synthetic construct
  source 1..40: mol_type = other DNA
tttaagtctctggcaccgtgcatagcagaattggttggga

SEQ ID NO: 696 - DNA, 40 bp, synthetic construct
  source 1..40: mol_type = other DNA
tctttctccaagtgcctctatgttggcacatctctgaaat

SEQ ID NO: 697 - DNA, 40 bp, synthetic construct
  source 1..40: mol_type = other DNA
tgcgtcccggccaggtaagcagcttccctctcagctgcct

SEQ ID NO: 698 - DNA, 40 bp, synthetic construct
  source 1..40: mol_type = other DNA
gggtgtatgtagctggcagaagtgggacttggtcgcaacc

SEQ ID NO: 699 - DNA, 40 bp, synthetic construct
  source 1..40: mol_type = other DNA
cgtggcgagtgggcggtagctgctcgtagagcgtgtgaaa

SEQ ID NO: 700 - DNA, 40 bp, synthetic construct
  source 1..40: mol_type = other DNA
gttggccctaaaagttatcattcatgctagtttgaccaat

SEQ ID NO: 701 - DNA, 40 bp, synthetic construct
  source 1..40: mol_type = other DNA
aagtgggaggagctgggcaagaaagtccacccctttttct

SEQ ID NO: 702 - DNA, 40 bp, synthetic construct
  source 1..40: mol_type = other DNA
gccgagccgaagtcatctgccaatcaaaacagccacaggg

SEQ ID NO: 703 - DNA, 40 bp, synthetic construct
  source 1..40: mol_type = other DNA
cgcgtacctaatgggagacagacaggtgcctttaaagcgg

SEQ ID NO: 704 - DNA, 40 bp, synthetic construct
  source 1..40: mol_type = other DNA
tggggaaagcggaggaaggcatggagtgtgggcgttaggg

SEQ ID NO: 705 - DNA, 40 bp, synthetic construct
  source 1..40: mol_type = other DNA
gcatattctgccttgaagtcattggttggtcctggaagtg

SEQ ID NO: 706 - DNA, 40 bp, synthetic construct
  source 1..40: mol_type = other DNA
aattggtctgggggaggagctacgacagtccaggggcggg

SEQ ID NO: 707 - DNA, 40 bp, synthetic construct
  source 1..40: mol_type = other DNA
gtgtcgtgctgattggatgtatccgcccccctctcttaaa

SEQ ID NO: 708 - DNA, 40 bp, synthetic construct
  source 1..40: mol_type = other DNA
caacacgccagcgcgaggacccgaacgtcaatcaagagac

SEQ ID NO: 709 - DNA, 40 bp, synthetic construct
  source 1..40: mol_type = other DNA
gcgttcgattggcctcccgcgcaggctgctaggattggct

SEQ ID NO: 710 - DNA, 40 bp, synthetic construct
  source 1..40: mol_type = other DNA
ccctgccccctttcgcggattgggtgatcgctccaaggcg

SEQ ID NO: 711 - DNA, 40 bp, synthetic construct
  source 1..40: mol_type = other DNA
ctgacccttggaggctttctattggttcctggcagggatg

SEQ ID NO: 712 - DNA, 40 bp, synthetic construct
  source 1..40: mol_type = other DNA
tcccgaatataggccagtcattgctcctgctgaacgtcgc

SEQ ID NO: 713 - DNA, 40 bp, synthetic construct
  source 1..40: mol_type = other DNA
cccctcctctcttctcgtctctggcgccgacccgcccccg

SEQ ID NO: 714 - DNA, 40 bp, synthetic construct
  source 1..40: mol_type = other DNA
gctcaagggaggccgcggcgtctgccgatggctccgcgga

SEQ ID NO: 715 - DNA, 40 bp, synthetic construct
  source 1..40: mol_type = other DNA
tgggggagtgggcccggggttgttctgacgacgggggtcg

SEQ ID NO: 716 - DNA, 40 bp, synthetic construct
  source 1..40: mol_type = other DNA
cccgggcgctatcgcgatagcggcgcgaagcggaagtggg

SEQ ID NO: 717 - DNA, 40 bp, synthetic construct
  source 1..40: mol_type = other DNA
cgggggaggcgagcgcccgccgcctttttctcgcgccccg

SEQ ID NO: 718 - DNA, 40 bp, synthetic construct
  source 1..40: mol_type = other DNA
cacaggagctggcgccgccgctgaggagcgtatcgcgaca

SEQ ID NO: 719 - DNA, 40 bp, synthetic construct
  source 1..40: mol_type = other DNA
gttgccgactcgcgctctcggcttctgctccggggcttct

SEQ ID NO: 720 - DNA, 40 bp, synthetic construct
  source 1..40: mol_type = other DNA
actcggagctcggatcccagtgtggacctggactcgaatc

SEQ ID NO: 721 - DNA, 40 bp, synthetic construct
  source 1..40: mol_type = other DNA
ggctcctccttgttccgagcccgaaggcccgccccttcac

SEQ ID NO: 722 - DNA, 40 bp, synthetic construct
  source 1..40: mol_type = other DNA
ctttccggagcccgtctgttccccttcgggtccaaagctt

SEQ ID NO: 723 - DNA, 40 bp, synthetic construct
  source 1..40: mol_type = other DNA
gaccccgcctcattcctcacggcgagctccagaccccgcc

SEQ ID NO: 724 - DNA, 40 bp, synthetic construct
  source 1..40: mol_type = other DNA
agaactcaagctcccgattgtgcccgaaggaacccgaagg

SEQ ID NO: 725 - DNA, 40 bp, synthetic construct
  source 1..40: mol_type = other DNA
actattgccgaagtgagccgaagtttgtggccccgcttcc

SEQ ID NO: 726 - DNA, 40 bp, synthetic construct
  source 1..40: mol_type = other DNA
acatgtggctccgcccacactggcctcagctctccgttct

SEQ ID NO: 727 - DNA, 40 bp, synthetic construct
  source 1..40: mol_type = other DNA
acagtgaccctaaggactcgactacctccgaagaaagccg

SEQ ID NO: 728 - DNA, 40 bp, synthetic construct
  source 1..40: mol_type = other DNA
cttgtacccaactatctacgaagtaaaccgaagcttgtgg

SEQ ID NO: 729 - DNA, 40 bp, synthetic construct
  source 1..40: mol_type = other DNA
tatctggcgaacctgttgactccgcctatcatcctagcgt

SEQ ID NO: 730 - DNA, 40 bp, synthetic construct
  source 1..40: mol_type = other DNA
ggcaagtcgctttcgccccgcccccttgtaaatactcatg

SEQ ID NO: 731 - DNA, 40 bp, synthetic construct
  source 1..40: mol_type = other DNA
ctcctctacttgggaacttgaggatcgtcaccctggcccg

SEQ ID NO: 732 - DNA, 40 bp, synthetic construct
  source 1..40: mol_type = other DNA
ttggctccgccccactgagcgcacctccctctgccgcttc

SEQ ID NO: 733 - DNA, 40 bp, synthetic construct
  source 1..40: mol_type = other DNA
tccttgctccaccccctcatgccgacaccctcgtcaactt

SEQ ID NO: 734 - DNA, 40 bp, synthetic construct
  source 1..40: mol_type = other DNA
tccaccgatagaaccagcgagtcacctcataaacagtaat

SEQ ID NO: 735 - DNA, 40 bp, synthetic construct
  source 1..40: mol_type = other DNA
cgctcagtccgcctccttgcctcccttcagaatgtcccac

SEQ ID NO: 736 - DNA, 40 bp, synthetic construct
  source 1..40: mol_type = other DNA
gccgtccactctccgctcgggcgggctcaccccaattggg

SEQ ID NO: 737 - DNA, 40 bp, synthetic construct
  source 1..40: mol_type = other DNA
cgaccgaaccccacagccgaaagccccgccccctggacac

SEQ ID NO: 738 - DNA, 40 bp, synthetic construct
  source 1..40: mol_type = other DNA
ctccgagcgccagcgcaccccagttggggagttcccgccc

SEQ ID NO: 739 - DNA, 40 bp, synthetic construct
  source 1..40: mol_type = other DNA
agccccgcctcctcccggacgcaataggttcggcgttcgg

SEQ ID NO: 740 - DNA, 40 bp, synthetic construct
  source 1..40: mol_type = other DNA
agcaatttgacgttcgggtgttctcggctcggccgaatcc

SEQ ID NO: 741 - DNA, 40 bp, synthetic construct
  source 1..40: mol_type = other DNA
tgccccctcccgagcacaggaagttcggcgttcgggcgtc

SEQ ID NO: 742 - DNA, 40 bp, synthetic construct
  source 1..40: mol_type = other DNA
tttcggacctcctcgctctcagactcccacagtacaaaac

SEQ ID NO: 743 - DNA, 40 bp, synthetic construct
  source 1..40: mol_type = other DNA
cgagccttcgctcctcctctttccgaacgactgtgattcg

SEQ ID NO: 744 - DNA, 40 bp, synthetic construct
  source 1..40: mol_type = other DNA
gaggctaaggcaccgccgaggccacaccctcttccggacg

SEQ ID NO: 745 - DNA, 40 bp, synthetic construct
  source 1..40: mol_type = other DNA
gcgtcccccttcgggtgttcccgtcagcggtcagaagctc

SEQ ID NO: 746 - DNA, 40 bp, synthetic construct
  source 1..40: mol_type = other DNA
ccttacaaaggtccattttggcaccaccctcttgcaaagt

SEQ ID NO: 747 - DNA, 40 bp, synthetic construct
  source 1..40: mol_type = other DNA
ggagcgtgaaaaacaaacctccgcaagcgcggcgacacgc

SEQ ID NO: 748 - DNA, 40 bp, synthetic construct
  source 1..40: mol_type = other DNA
acccgctctgtgcccgcactgccgtacctaccattgcgcc

SEQ ID NO: 749 - DNA, 40 bp, synthetic construct
  source 1..40: mol_type = other DNA
ggtcctcagcatctgcatatgtagcccctcccgctggtca

SEQ ID NO: 750 - DNA, 40 bp, synthetic construct
  source 1..40: mol_type = other DNA
cccaacccctacccccaatccatcttagagctgattctct

SEQ ID NO: 751 - DNA, 40 bp, synthetic construct
  source 1..40: mol_type = other DNA
actccagtgattcttccttatgctagggactcgaggaccc

SEQ ID NO: 752 - DNA, 40 bp, synthetic construct
  source 1..40: mol_type = other DNA
gagaattgagaagtcagtgtgggaggggatgtcccagtac

SEQ ID NO: 753 - DNA, 40 bp, synthetic construct
  source 1..40: mol_type = other DNA
tttctggttcgcgttggctgcattgtggagctgagggatg

SEQ ID NO: 754 - DNA, 40 bp, synthetic construct
  source 1..40: mol_type = other DNA
tagcttcttaatctccttctttaggtcagcctcatacttt

SEQ ID NO: 755 - DNA, 40 bp, synthetic construct
  source 1..40: mol_type = other DNA
ttctccctgggacccagcagtccactctcccagttccctc

SEQ ID NO: 756 - DNA, 40 bp, synthetic construct
  source 1..40: mol_type = other DNA
aaagtcagacctcaggacccaggaactggggcccacagct

SEQ ID NO: 757 - DNA, 40 bp, synthetic construct
  source 1..40: mol_type = other DNA
tcttgatttggtccctcagccgctgcagatgggaaaagca

SEQ ID NO: 758 - DNA, 40 bp, synthetic construct
  source 1..40: mol_type = other DNA
taagctgcctcttgtccttgatctcgttggacgctaccca

SEQ ID NO: 759 - DNA, 40 bp, synthetic construct
  source 1..40: mol_type = other DNA
ggctctgggctcctaccgtctcaatgagcttgcggttgtc

SEQ ID NO: 760 - DNA, 40 bp, synthetic construct
  source 1..40: mol_type = other DNA
tgaggacctctggggtctggccgctctgcctccgcccctt

SEQ ID NO: 761 - DNA, 40 bp, synthetic construct
  source 1..40: mol_type = other DNA
ctgcctcttcacttcccttaggtgcagaaaccttacttct

SEQ ID NO: 762 - DNA, 40 bp, synthetic construct
  source 1..40: mol_type = other DNA
cgacctgagcctcgtgaccctactttctgagctctgagtc

SEQ ID NO: 763 - DNA, 40 bp, synthetic construct
  source 1..40: mol_type = other DNA
tcaaaggtgggaaaggagctgactaagggccagcagacac

SEQ ID NO: 764 - DNA, 40 bp, synthetic construct
  source 1..40: mol_type = other DNA
ccgttccatttgctgtagagagtgcagttggcaggggggc

SEQ ID NO: 765 - DNA, 40 bp, synthetic construct
  source 1..40: mol_type = other DNA
gctgtaagctttggttttggtctctcgttccacaactttg

SEQ ID NO: 766 - DNA, 40 bp, synthetic construct
  source 1..40: mol_type = other DNA
ccaactcaccgtgagccactggccaacctcttccttctcc

SEQ ID NO: 767 - DNA, 40 bp, synthetic construct
  source 1..40: mol_type = other DNA
ccagggctcaggatcctcagagttcacctcctcttctcta

SEQ ID NO: 768 - DNA, 40 bp, synthetic construct
  source 1..40: mol_type = other DNA
gtccacctgcatgttgagcgtgtcgatggtattctagggg

SEQ ID NO: 769 - DNA, 40 bp, synthetic construct
  source 1..40: mol_type = other DNA
gcgtgtctgcactgacagtgactccacttcactctcaaac

SEQ ID NO: 770 - DNA, 40 bp, synthetic construct
  source 1..40: mol_type = other DNA
tgtcgggtctccctcactcacatccttgtcgcccttcttc

SEQ ID NO: 771 - DNA, 40 bp, synthetic construct
  source 1..40: mol_type = other DNA
ctgctggccagcccattcccatgcccatccccatcccaaa

SEQ ID NO: 772 - DNA, 40 bp, synthetic construct
  source 1..40: mol_type = other DNA
gaatccaggccccaactcccaggagcataaatgactggcc

SEQ ID NO: 773 - DNA, 40 bp, synthetic construct
  source 1..40: mol_type = other DNA
tctcaaatccctaatcccggctgttggccctgtccgcctg

SEQ ID NO: 774 - DNA, 40 bp, synthetic construct
  source 1..40: mol_type = other DNA
cctgccccacgcgtgcagctgctaagccctcccaatcctg

SEQ ID NO: 775 - DNA, 40 bp, synthetic construct
  source 1..40: mol_type = other DNA
cccagacacccaggggaccctgagattctgtctgacctcc

SEQ ID NO: 776 - DNA, 40 bp, synthetic construct
  source 1..40: mol_type = other DNA
cttcccccaagtcgctcctcttcacaaaggccccacggtc

SEQ ID NO: 777 - DNA, 40 bp, synthetic construct
  source 1..40: mol_type = other DNA
cctctgggtgccaggaggcctcttgccatgggtgtccttc

SEQ ID NO: 778 - DNA, 40 bp, synthetic construct
  source 1..40: mol_type = other DNA
ctgccttgtctctacccactgtgctctccctaggaccagg

SEQ ID NO: 779 - DNA, 40 bp, synthetic construct
  source 1..40: mol_type = other DNA
ggcgagggggaggtcctgcagctgctcgcgtgggctgccc

SEQ ID NO: 780 - DNA, 40 bp, synthetic construct
  source 1..40: mol_type = other DNA
tgcgctcgatctcatccttcagttcgtagcccacctgggg

SEQ ID NO: 781 - DNA, 40 bp, synthetic construct
  source 1..40: mol_type = other DNA
tcacctgcttcacaggcggcggctcctgccacttgtcgaa

SEQ ID NO: 782 - DNA, 40 bp, synthetic construct
  source 1..40: mol_type = other DNA
ctcgcttcttccgctgtccatccaggggcgcaggcagcgg

SEQ ID NO: 783 - DNA, 40 bp, synthetic construct
  source 1..40: mol_type = other DNA
cccatgcctaccggacccccagggcccctcacctgcggcc

SEQ ID NO: 784 - DNA, 40 bp, synthetic construct
  source 1..40: mol_type = other DNA
agtcggctgggaggaggacgccggcttctcccctccatga

SEQ ID NO: 785 - DNA, 40 bp, synthetic construct
  source 1..40: mol_type = other DNA
atcttgcggtacctggggacgggtgggtgggcggcgccag

SEQ ID NO: 786 - DNA, 40 bp, synthetic construct
  source 1..40: mol_type = other DNA
ttggcctgcttccggatctccgtcagccccagccgctcct

SEQ ID NO: 787 - DNA, 40 bp, synthetic construct
  source 1..40: mol_type = other DNA
ggagggcgctctgggagtctgacctctccgaagctcatac

SEQ ID NO: 788 - DNA, 40 bp, synthetic construct
  source 1..40: mol_type = other DNA
aggaggcagagggcggtggcggctggctggctgtggggtt

SEQ ID NO: 789 - DNA, 40 bp, synthetic construct
  source 1..40: mol_type = other DNA
agacatgagccagggccacaggacgagaggaggggcggtg

SEQ ID NO: 790 - DNA, 40 bp, synthetic construct
  source 1..40: mol_type = other DNA
ccaagggccgcgagggtcgctttggggctgaatggatgga

SEQ ID NO: 791 - DNA, 40 bp, synthetic construct
  source 1..40: mol_type = other DNA
gatgggaagccgcgggggctctaagcagcggagacacagg

SEQ ID NO: 792 - DNA, 40 bp, synthetic construct
  source 1..40: mol_type = other DNA
ggagcctctgggcagggaggaaccggccaaggagcccggg

SEQ ID NO: 793 - DNA, 40 bp, synthetic construct
  source 1..40: mol_type = other DNA
ggcggggcccagggacggggcggccgtgcagcagggcact

SEQ ID NO: 794 - DNA, 40 bp, synthetic construct
  source 1..40: mol_type = other DNA
ctgcaggaccaaggggatgacgctgggataacagaggaga

SEQ ID NO: 795 - DNA, 40 bp, synthetic construct
  source 1..40: mol_type = other DNA
cagaacaggtttaataggatgaggtggcctctgagttcgg

SEQ ID NO: 796 - DNA, 40 bp, synthetic construct
  source 1..40: mol_type = other DNA
ccattccttccttactcgtgtgggtcgggggatgtcagga

SEQ ID NO: 797 - DNA, 40 bp, synthetic construct
  source 1..40: mol_type = other DNA
ggcccggtcccagcactgctctgtgagctcagagttggga

SEQ ID NO: 798 - DNA, 40 bp, synthetic construct
  source 1..40: mol_type = other DNA
tgggggcccacacacgcgggggatgccggggagcctgaga

SEQ ID NO: 799 - DNA, 40 bp, synthetic construct
  source 1..40: mol_type = other DNA
cacgggcacctgctccggtacccactcggcccggctgagg

SEQ ID NO: 800 - DNA, 40 bp, synthetic construct
  source 1..40: mol_type = other DNA
ctccaccagccggaagcccagcggtcaccagccggccggt

SEQ ID NO: 801 - DNA, 40 bp, synthetic construct
  source 1..40: mol_type = other DNA
aggcgtcctcctcgatctagggggaagaggaggcgccctg

SEQ ID NO: 802 - DNA, 40 bp, synthetic construct
  source 1..40: mol_type = other DNA
acttgcccaggtggcccaggctgaatcccaggtcctcctg

SEQ ID NO: 803 - DNA, 40 bp, synthetic construct
  source 1..40: mol_type = other DNA
tggcctcgtttacctgtgtctgccgcacacgcccactgcc

SEQ ID NO: 804 - DNA, 40 bp, synthetic construct
  source 1..40: mol_type = other DNA
gtctggcccatacctgcagcgtcttggagatcctggcctt

SEQ ID NO: 805 - DNA, 40 bp, synthetic construct
  source 1..40: mol_type = other DNA
gctccccccaccttgtgtccctcggtccccagccccacct

SEQ ID NO: 806 - DNA, 40 bp, synthetic construct
  source 1..40: mol_type = other DNA
tgcagggtccgctgtggggaggacagggaggctgcgatct

SEQ ID NO: 807 - DNA, 40 bp, synthetic construct
  source 1..40: mol_type = other DNA
tcgcggatggtggacttcccgccatatacgacgctctgct

SEQ ID NO: 808 - DNA, 40 bp, synthetic construct
  source 1..40: mol_type = other DNA
agtggggtgaaggccacgctggaggccgtgcccgaggagc

SEQ ID NO: 809 - DNA, 40 bp, synthetic construct
  source 1..40: mol_type = other DNA
cggctgctgagcctaaccacctcctgggcttctttccagc

SEQ ID NO: 810 - DNA, 40 bp, synthetic construct
  source 1..40: mol_type = other DNA
gctcatggtatccctaccgcaggcaatctgtggacagcac

SEQ ID NO: 811 - DNA, 40 bp, synthetic construct
  source 1..40: mol_type = other DNA
ctgaatgtcacctgaagggtcacagaagctactcacaggg

SEQ ID NO: 812 - DNA, 40 bp, synthetic construct
  source 1..40: mol_type = other DNA
ttaagtgttctcaatatgagattagctggagccgcctaat

SEQ ID NO: 813 - DNA, 40 bp, synthetic construct
  source 1..40: mol_type = other DNA
gaagatccatctgttggaagccagaggactagtgggaaac

SEQ ID NO: 814 - DNA, 40 bp, synthetic construct
  source 1..40: mol_type = other DNA
cccccacagggatctgacacacaacttaggttgtcagcca

SEQ ID NO: 815 - DNA, 40 bp, synthetic construct
  source 1..40: mol_type = other DNA
gcccagcttcccaagtcctgcctggacaccgccccatgga

SEQ ID NO: 816 - DNA, 40 bp, synthetic construct
  source 1..40: mol_type = other DNA
aatcaccttcatgcttaaaacactcacactgatttccagc

SEQ ID NO: 817 - DNA, 40 bp, synthetic construct
  source 1..40: mol_type = other DNA
cctcttggggacctgggtgaccttactcaccctcatggct

SEQ ID NO: 818 - DNA, 40 bp, synthetic construct
  source 1..40: mol_type = other DNA
gttgctgtggacaggcttggagccgtttttggctggagac

SEQ ID NO: 819 - DNA, 40 bp, synthetic construct
  source 1..40: mol_type = other DNA
ggaggggtaggtgggcggcacagctggggactgagggtgc

SEQ ID NO: 820 - DNA, 40 bp, synthetic construct
  source 1..40: mol_type = other DNA
gccaggagtggtgctcaaggcagaggcagcaggcgggggg

SEQ ID NO: 821 - DNA, 40 bp, synthetic construct
  source 1..40: mol_type = other DNA
cagggcacttgggggtgctgcgggggcggggaccccattg

SEQ ID NO: 822 - DNA, 40 bp, synthetic construct
  source 1..40: mol_type = other DNA
ggtgcccgagttgtggctgggagctggactggccttgggg

SEQ ID NO: 823 - DNA, 40 bp, synthetic construct
  source 1..40: mol_type = other DNA
ctgcttgccagcccctccaccggcactgctgttactactg

SEQ ID NO: 824 - DNA, 40 bp, synthetic construct
  source 1..40: mol_type = other DNA
gccccccaccccgctgcctcctcactcactggtggcgcca

SEQ ID NO: 825 - DNA, 40 bp, synthetic construct
  source 1..40: mol_type = other DNA
cgggctgtctgccacaactgagctgtaacctgggaacaaa

SEQ ID NO: 826 - DNA, 40 bp, synthetic construct
  source 1..40: mol_type = other DNA
gctggcattgttgcccccactgctgctcaaagccacctct

SEQ ID NO: 827 - DNA, 40 bp, synthetic construct
  source 1..40: mol_type = other DNA
aggtgggttgtgggggccggaaggggggcccaaggcctgg

SEQ ID NO: 828 - DNA, 40 bp, synthetic construct
  source 1..40: mol_type = other DNA
tcccaaccctgccgatggccgagacactcacgaggtgctg

SEQ ID NO: 829 - DNA, 40 bp, synthetic construct
  source 1..40: mol_type = other DNA
gggggtgaggcgcctgcgcctctctgtttcaaaaggctgc

SEQ ID NO: 830 - DNA, 40 bp, synthetic construct
  source 1..40: mol_type = other DNA
attcccagcagcaagggcggggggttcagaacccaccgat

SEQ ID NO: 831 - DNA, 40 bp, synthetic construct
  source 1..40: mol_type = other DNA
gggggtgtaacacccgagggagatggaggatagcgcttgg

SEQ ID NO: 832 - DNA, 40 bp, synthetic construct
  source 1..40: mol_type = other DNA
caaagcagggaggctgatgtagtttccttgctggaaagaa

SEQ ID NO: 833 - DNA, 40 bp, synthetic construct
  source 1..40: mol_type = other DNA
cttccacttagatgagaacgtattttagaatgttctgaag

SEQ ID NO: 834 - DNA, 40 bp, synthetic construct
  source 1..40: mol_type = other DNA
taacagaaatggggaggaaagggtatggggctcttgagaa

SEQ ID NO: 835 - DNA, 40 bp, synthetic construct
  source 1..40: mol_type = other DNA
aaacagtgaccctccggtggcagtcaattggcctcaggca

SEQ ID NO: 836 - DNA, 40 bp, synthetic construct
  source 1..40: mol_type = other DNA
gcagaggaataaggacttcgggacaattcactttgaaaag

SEQ ID NO: 837 - DNA, 40 bp, synthetic construct
  source 1..40: mol_type = other DNA
gacccagtggaatggtctgagctaagatttgaaggagtgg

SEQ ID NO: 838 - DNA, 40 bp, synthetic construct
  source 1..40: mol_type = other DNA
tgcacactgatctttcttagggcattcttcgggaaacagg

SEQ ID NO: 839 - DNA, 40 bp, synthetic construct
  source 1..40: mol_type = other DNA
ggctcaggatgaacagcaacaggggttgggatgatcactg

SEQ ID NO: 840 - DNA, 40 bp, synthetic construct
  source 1..40: mol_type = other DNA
gatcatggagatgtgatctagggaacaaagccagagaagg

SEQ ID NO: 841 - DNA, 40 bp, synthetic construct
  source 1..40: mol_type = other DNA
aggcattcccacggtgtgaggtcagattgggcagggccta

SEQ ID NO: 842 - DNA, 40 bp, synthetic construct
  source 1..40: mol_type = other DNA
agagccagcacttgctgttccacacatactagatcagtct

SEQ ID NO: 843 - DNA, 40 bp, synthetic construct
  source 1..40: mol_type = other DNA
tggacaaccccctcccacacccagagctgtggaaggggag

SEQ ID NO: 844 - DNA, 40 bp, synthetic construct
  source 1..40: mol_type = other DNA
cacctagatgctgaccaaggccctccccatgctgctggag

SEQ ID NO: 845 - DNA, 40 bp, synthetic construct
  source 1..40: mol_type = other DNA
ataaagccttcattctccaggaccccgcccttgccctgtt

SEQ ID NO: 846 - DNA, 40 bp, synthetic construct
  source 1..40: mol_type = other DNA
aggtggtgagtttggggctggggggcctccctgaggagcc

SEQ ID NO: 847 - DNA, 40 bp, synthetic construct
  source 1..40: mol_type = other DNA
gagagaaccaggtcccacatgctgacacaggtgtccacgg

SEQ ID NO: 848 - DNA, 40 bp, synthetic construct
  source 1..40: mol_type = other DNA
atccccccaatctcaccagtgcaccccacagacaaggcga

SEQ ID NO: 849 - DNA, 40 bp, synthetic construct
  source 1..40: mol_type = other DNA
aagggcttcagcataagagtcagaacccgccccccttcct

SEQ ID NO: 850 - DNA, 40 bp, synthetic construct
  source 1..40: mol_type = other DNA
tgtgggctgaagggacgaggctggggcactgggtgggagg

SEQ ID NO: 851 - DNA, 40 bp, synthetic construct
  source 1..40: mol_type = other DNA
ttgcaatgtggaagagtcaggggcacattgtctgggctga

SEQ ID NO: 852 - DNA, 40 bp, synthetic construct
  source 1..40: mol_type = other DNA
taagtgggagggagcggggacctagtgtgggcatgaggac

SEQ ID NO: 853 - DNA, 40 bp, synthetic construct
  source 1..40: mol_type = other DNA
ggagcagggatttggctgggcaatggagagaaaggtctga

SEQ ID NO: 854 - DNA, 40 bp, synthetic construct
  source 1..40: mol_type = other DNA
acacagagatgcccaggaacttgctctttagtaaagcagc

SEQ ID NO: 855 - DNA, 40 bp, synthetic construct
  source 1..40: mol_type = other DNA
tggagagaggtccttgaaaggttttgaaccccataaagag

SEQ ID NO: 856 - DNA, 40 bp, synthetic construct
  source 1..40: mol_type = other DNA
tcaggaggcagcccagtgatagggtccaaggaaccagtgg

SEQ ID NO: 857 - DNA, 40 bp, synthetic construct
  source 1..40: mol_type = other DNA
acagtctactgacttttcctattcagctgtgagcattcaa

SEQ ID NO: 858 - DNA, 40 bp, synthetic construct
  source 1..40: mol_type = other DNA
ctgtcccctggaccttgacacctggctccccaaccctgtc

SEQ ID NO: 859 - DNA, 40 bp, synthetic construct
  source 1..40: mol_type = other DNA
aggaaacccagattccaccagacacttccttcttcccccc

SEQ ID NO: 860 - DNA, 40 bp, synthetic construct
  source 1..40: mol_type = other DNA
ggctatctggcctgagacaacaaatgctgcctcccaccct

SEQ ID NO: 861 - DNA, 40 bp, synthetic construct
  source 1..40: mol_type = other DNA
gtctggcactgggactttcagaactcctccttccctgact

SEQ ID NO: 862 - DNA, 40 bp, synthetic construct
  source 1..40: mol_type = other DNA
ttgccccagacccgtcattcaatggctagctttttccatg

SEQ ID NO: 863 - DNA, 40 bp, synthetic construct
  source 1..40: mol_type = other DNA
aaaaacacgagcacccccaaccacaacggccagttctctg

SEQ ID NO: 864 - DNA, 40 bp, synthetic construct
  source 1..40: mol_type = other DNA
ttaaccttggacatggtaaaccatccaaaaccttcctctc

SEQ ID NO: 865 - DNA, 40 bp, synthetic construct
  source 1..40: mol_type = other DNA
agcaactaaacctctccactgggcacttatccttggtttc

SEQ ID NO: 866 - DNA, 40 bp, synthetic construct
  source 1..40: mol_type = other DNA
gaacctcttattctcttagaacccacagctgccaccacag

SEQ ID NO: 867 - DNA, 40 bp, synthetic construct
  source 1..40: mol_type = other DNA
tcccttctcccagtgtaagaccccaaatcactccaaatga

SEQ ID NO: 868 - DNA, 40 bp, synthetic construct
  source 1..40: mol_type = other DNA
caacccccaacccgatgcctgcttcagatgtttcccatgt

SEQ ID NO: 869 - DNA, 40 bp, synthetic construct
  source 1..40: mol_type = other DNA
cataaacctggctcctaaaggctaaatattttgttggaga

SEQ ID NO: 870 - DNA, 40 bp, synthetic construct
  source 1..40: mol_type = other DNA
ctgctgacctgccctcccaggtcagaatcatcctcatgca

SEQ ID NO: 871 - DNA, 40 bp, synthetic construct
  source 1..40: mol_type = other DNA
tgttctccagacctgtgcactctatctgtgcaacagagat

SEQ ID NO: 872 - DNA, 40 bp, synthetic construct
  source 1..40: mol_type = other DNA
cgtgcagcaaacaatgtggaattccaataaccccccactc

SEQ ID NO: 873 - DNA, 40 bp, synthetic construct
  source 1..40: mol_type = other DNA
aaatatgagtctcccaaagttccctagcatttcaaaatcc

SEQ ID NO: 874 - DNA, 40 bp, synthetic construct
  source 1..40: mol_type = other DNA
catcataaaaagatcttgtggtccacagatcctctagccc

SEQ ID NO: 875 - DNA, 40 bp, synthetic construct
  source 1..40: mol_type = other DNA
ctcccaacccagaatccagctccacagatacattgctact

SEQ ID NO: 876 - DNA, 40 bp, synthetic construct
  source 1..40: mol_type = other DNA
cactctgagaccagaaactagaacttttattcctcatgct

SEQ ID NO: 877 - DNA, 40 bp, synthetic construct
  source 1..40: mol_type = other DNA
caccagcactcaggagattgtgagactccctgatccctgc

SEQ ID NO: 878 - DNA, 40 bp, synthetic construct
  source 1..40: mol_type = other DNA
tgcctagatcctttgcactccaagacccagtgtgccctaa

SEQ ID NO: 879 - DNA, 40 bp, synthetic construct
  source 1..40: mol_type = other DNA
gggggtgggtacgatccccgattcttcatacaaagcctca

SEQ ID NO: 880 - DNA, 40 bp, synthetic construct
  source 1..40: mol_type = other DNA
ggacaaaggcagaggagacacgcccaggatgaaacagaaa

SEQ ID NO: 881 - DNA, 40 bp, synthetic construct
  source 1..40: mol_type = other DNA
tggatgcaccaggccctgtagctcatggagacttcatcta

SEQ ID NO: 882 - DNA, 40 bp, synthetic construct
  source 1..40: mol_type = other DNA
gggagagctagcacttgctgttctgcaattactagatcac

SEQ ID NO: 883 - DNA, 40 bp, synthetic construct
  source 1..40: mol_type = other DNA
ggctggacaaccccctcccacacccagagctgtggaaggg

SEQ ID NO: 884 - DNA, 40 bp, synthetic construct
  source 1..40: mol_type = other DNA
tggcacccagaggctgaccaaggccctccccatgctgctg

SEQ ID NO: 885 - DNA, 40 bp, synthetic construct
  source 1..40: mol_type = other DNA
cctataaaaccttcattccccaggactccgcccctgccct

SEQ ID NO: 886 - DNA, 40 bp, synthetic construct
  source 1..40: mol_type = other DNA
tgcaggtggtaagcttggggctggggagcctcccccagga

SEQ ID NO: 887 - DNA, 40 bp, synthetic construct
  source 1..40: mol_type = other DNA
aggaagacaaccgggacccacatggtgacacagctctccg

SEQ ID NO: 888 - DNA, 40 bp, synthetic construct
  source 1..40: mol_type = other DNA
caaccatggcccctctcaccaatccacgtcacggacaggg

SEQ ID NO: 889 - DNA, 40 bp, synthetic construct
  source 1..40: mol_type = other DNA
tcagcttgacagtcagggctggctccctctcctgcatccc

SEQ ID NO: 890 - DNA, 40 bp, synthetic construct
  source 1..40: mol_type = other DNA
tccctgtctgggctggggtgctgggttgggggggaaagag

SEQ ID NO: 891 - DNA, 40 bp, synthetic construct
  source 1..40: mol_type = other DNA
tgtgggagtgaggactgttgcaatatggaggggctggggg

SEQ ID NO: 892 - DNA, 40 bp, synthetic construct
  source 1..40: mol_type = other DNA
gggagaaagttctggggtaagtgggagggagcggggacct

SEQ ID NO: 893 - DNA, 40 bp, synthetic construct
  source 1..40: mol_type = other DNA
ttgtggggctcaaaacctccaaggacctctctcaatgcca

SEQ ID NO: 894 - DNA, 40 bp, synthetic construct
  source 1..40: mol_type = other DNA
tgcccaaccctatcccagagaccttgatgcttggcctccc

SEQ ID NO: 895 - DNA, 40 bp, synthetic construct
  source 1..40: mol_type = other DNA
tcttgccctaggatacccagatgccaaccagacacctcct

SEQ ID NO: 896 - DNA, 40 bp, synthetic construct
  source 1..40: mol_type = other DNA
ttcctagccaggctatctggcctgagacaacaaatgggtc

SEQ ID NO: 897 - DNA, 40 bp, synthetic construct
  source 1..40: mol_type = other DNA
tcttagccccagactcttcattcagtggcccacattttcc

SEQ ID NO: 898 - DNA, 40 bp, synthetic construct
  source 1..40: mol_type = other DNA
aggaaaaacatgagcatccccagccacaactgccagctct

SEQ ID NO: 899 - DNA, 40 bp, synthetic construct
  source 1..40: mol_type = other DNA
ccccttcagagttactgacaaacaggtgggcactgagact

SEQ ID NO: 900 - DNA, 40 bp, synthetic construct
  source 1..40: mol_type = other DNA
tggaaagttagcttatttgtttgcaagtcagtaaaatgtc

SEQ ID NO: 901 - DNA, 40 bp, synthetic construct
  source 1..40: mol_type = other DNA
gactcaggagtctcatggactctgccagcattcacaaaac

SEQ ID NO: 902 - DNA, 40 bp, synthetic construct
  source 1..40: mol_type = other DNA
atgctgtctgctaagctgtgagcagtaaaagcctttgcct

SEQ ID NO: 903 - DNA, 40 bp, synthetic construct
  source 1..40: mol_type = other DNA
gatttggggggggcaaggtgtactaatgtgaacatgaacc

SEQ ID NO: 904 - DNA, 40 bp, synthetic construct
  source 1..40: mol_type = other DNA
gtgtgcacagcatccacctagactgctctggtcaccctac

SEQ ID NO: 905 - DNA, 40 bp, synthetic construct
  source 1..40: mol_type = other DNA
aggattcctaatctcaggtttctcaccagtggcacaaacc

SEQ ID NO: 906 - DNA, 40 bp, synthetic construct
  source 1..40: mol_type = other DNA
caaaggctgagcaggtttgcaagttgtcccagtataagat

SEQ ID NO: 907 - DNA, 40 bp, synthetic construct
  source 1..40: mol_type = other DNA
gtcaaggacaatcgatacaatatgttcctccagagtaggt

SEQ ID NO: 908 - DNA, 40 bp, synthetic construct
  source 1..40: mol_type = other DNA
gcaagatgatatctctctcagatccaggcttgcttactgt

SEQ ID NO: 909 - DNA, 40 bp, synthetic construct
  source 1..40: mol_type = other DNA
tctgtgtgtcttctgagcaaagacagcaacaccttttttt

SEQ ID NO: 910 - DNA, 40 bp, synthetic construct
  source 1..40: mol_type = other DNA
aacgttgagactgtcctgcagacaagggtggaaggctctg

SEQ ID NO: 911 - DNA, 40 bp, synthetic construct
  source 1..40: mol_type = other DNA
cataaataagcaggatgtgacagaagaagtatttaatggt

SEQ ID NO: 912 - DNA, 40 bp, synthetic construct
  source 1..40: mol_type = other DNA
gctgccagacacagtcgatcgggacctagaaccttggtta

SEQ ID NO: 913 - DNA, 40 bp, synthetic construct
  source 1..40: mol_type = other DNA
gggatcctgagcgctgccttattctgggtttggcagtgga

SEQ ID NO: 914 - DNA, 40 bp, synthetic construct
  source 1..40: mol_type = other DNA
tcactcaaacccagaagttctgatccccagccatgcccct

SEQ ID NO: 915 - DNA, 40 bp, synthetic construct
  source 1..40: mol_type = other DNA
agcctcttcctcctttgaaattcaagagggtggacccact

SEQ ID NO: 916 - DNA, 40 bp, synthetic construct
  source 1..40: mol_type = other DNA
ggagctgggaccttaccagtctcctccctcattgacctaa

SEQ ID NO: 917 - DNA, 40 bp, synthetic construct
  source 1..40: mol_type = other DNA
gaggatatgagattcttaggccattcccacatcagtacct

SEQ ID NO: 918 - DNA, 40 bp, synthetic construct
  source 1..40: mol_type = other DNA
tacccagaactctacccctcaggattccagcaccttcttc

SEQ ID NO: 919 - DNA, 40 bp, synthetic construct
  source 1..40: mol_type = other DNA
gcctctgcccttcaggggccaaagagccttaagccacaaa

SEQ ID NO: 920 - DNA, 40 bp, synthetic construct
  source 1..40: mol_type = other DNA
atcccattactatcaccccaaaccctggacctaatggttc

SEQ ID NO: 921 - DNA, 40 bp, synthetic construct
  source 1..40: mol_type = other DNA
aatgggcaaccctcgatcctcagactcttgaggaatcaag

SEQ ID NO: 922 - DNA, 40 bp, synthetic construct
  source 1..40: mol_type = other DNA
gataccctcaagtggagtaaggattaggtggcaagatgga

SEQ ID NO: 923 - DNA, 40 bp, synthetic construct
  source 1..40: mol_type = other DNA
gtgcttgcccaggggcaccttcatggagctagaagggctg

SEQ ID NO: 924 - DNA, 40 bp, synthetic construct
  source 1..40: mol_type = other DNA
gatgacacccaaggcctctggggcatctttcatgctcaga

SEQ ID NO: 925 - DNA, 40 bp, synthetic construct
  source 1..40: mol_type = other DNA
tgctggccacaccctcagagtgtggatgctggatgatgag

SEQ ID NO: 926 - DNA, 40 bp, synthetic construct
  source 1..40: mol_type = other DNA
gaggcacgctgcagggatagtcacagcaacatgacgtcat

SEQ ID NO: 927 - DNA, 40 bp, synthetic construct
  source 1..40: mol_type = other DNA
agaggaggatgtcggcagctctacggttggcaggtggctg

SEQ ID NO: 928 - DNA, 40 bp, synthetic construct
  source 1..40: mol_type = other DNA
gacactaggcctcagcctggcaccatgcaggccactccca

SEQ ID NO: 929 - DNA, 40 bp, synthetic construct
  source 1..40: mol_type = other DNA
acttttgagtcctggatccctatgattccaggctccctgt

SEQ ID NO: 930 - DNA, 40 bp, synthetic construct
  source 1..40: mol_type = other DNA
ccttgagatttcatggatggtgacatatggccattctcta

SEQ ID NO: 931 - DNA, 40 bp, synthetic construct
  source 1..40: mol_type = other DNA
aaaacccataagttcaggtccctgtgccctccacccagaa

SEQ ID NO: 932 - DNA, 40 bp, synthetic construct
  source 1..40: mol_type = other DNA
tcgtatctgggagactcacttgggagagcaatagacttgg

SEQ ID NO: 933 - DNA, 40 bp, synthetic construct
  source 1..40: mol_type = other DNA
tacaagatgtggtggagataaggctgatgctggcacagtg

SEQ ID NO: 934 - DNA, 40 bp, synthetic construct
  source 1..40: mol_type = other DNA
gtacacaccatggtgttcatcagggccctgggtagtccct

SEQ ID NO: 935 - DNA, 40 bp, synthetic construct
  source 1..40: mol_type = other DNA
gctgtgacctcacaggagtccgtgcctccaccccctactc

SEQ ID NO: 936 - DNA, 16 bp, synthetic construct
  source 1..16: mol_type = other DNA
cgtcgcaccaagaaat

SEQ ID NO: 937 - DNA, 16 bp, synthetic construct
  source 1..16: mol_type = other DNA
tagacttgccatacgt

SEQ ID NO: 938 - DNA, 16 bp, synthetic construct
  source 1..16: mol_type = other DNA
aattcttgagaccagg

SEQ ID NO: 939 - DNA, 16 bp, synthetic construct
  source 1..16: mol_type = other DNA
atctgcccaaactcca

SEQ ID NO: 940 - DNA, 16 bp, synthetic construct
  source 1..16: mol_type = other DNA
ttcccaagcgtcatct

SEQ ID NO: 941 - DNA, 16 bp, synthetic construct
  source 1..16: mol_type = other DNA
tctatcggacgctgta

SEQ ID NO: 942 - DNA, 26 bp, synthetic construct
  source 1..26: mol_type = other DNA
aaaaaaaaaaatttcttggtgcgacg

SEQ ID NO: 943 - DNA, 26 bp, synthetic construct
  source 1..26: mol_type = other DNA
aaaaaaaaaaacgtatggcaagtcta

SEQ ID NO: 944 - DNA, 26 bp, synthetic construct
  source 1..26: mol_type = other DNA
aaaaaaaaaacctggtctcaagaatt

SEQ ID NO: 945 - DNA, 26 bp, synthetic construct
  source 1..26: mol_type = other DNA
aaaaaaaaaatggagtttgggcagat

SEQ ID NO: 946 - DNA, 26 bp, synthetic construct
  source 1..26: mol_type = other DNA
aaaaaaaaaaagatgacgcttgggaa

SEQ ID NO: 947 - DNA, 26 bp, synthetic construct
  source 1..26: mol_type = other DNA
aaaaaaaaaatacagcgtccgataga

SEQ ID NO: 948 - DNA, 41 bp, synthetic construct
  source 1..41: mol_type = other DNA
atttatagaaaccgggggcgaaaaaacgtatggcaagtcta

SEQ ID NO: 949 - DNA, 41 bp, synthetic construct
  source 1..41: mol_type = other DNA
cgaacaggaggagcagagagaaaaaacgtatggcaagtcta

SEQ ID NO: 950 - DNA, 41 bp, synthetic construct
  source 1..41: mol_type = other DNA
gctggcgacgcaaaagaagaaaaaaacgtatggcaagtcta

SEQ ID NO: 951 - DNA, 41 bp, synthetic construct
  source 1..41: mol_type = other DNA
catggtgtctgagcgatgtgaaaaaacgtatggcaagtcta

SEQ ID NO: 952 - DNA, 41 bp, synthetic construct
  source 1..41: mol_type = other DNA
tacgaccaaatccgttgactaaaaaacgtatggcaagtcta

SEQ ID NO: 953 - DNA, 41 bp, synthetic construct
  source 1..41: mol_type = other DNA
cagagttaaaagcagccctgaaaaaacgtatggcaagtcta

SEQ ID NO: 954 - DNA, 41 bp, synthetic construct
  source 1..41: mol_type = other DNA
gggtcattgatggcaacaataaaaaacgtatggcaagtcta

SEQ ID NO: 955 - DNA, 41 bp, synthetic construct
  source 1..41: mol_type = other DNA
aaccatgtagttgaggtcaaaaaaaacgtatggcaagtcta

SEQ ID NO: 956 - DNA, 41 bp, synthetic construct
  source 1..41: mol_type = other DNA
gggtggaatcatattggaacaaaaaacgtatggcaagtcta

SEQ ID NO: 957 - DNA, 41 bp, synthetic construct
  source 1..41: mol_type = other DNA
ttgacggtgccatggaatttaaaaaacgtatggcaagtcta

SEQ ID NO: 958 - DNA, 41 bp, synthetic construct
  source 1..41: mol_type = other DNA
cattgatgacaagcttcccgaaaaaacgtatggcaagtcta

SEQ ID NO: 959 - DNA, 41 bp, synthetic construct
  source 1..41: mol_type = other DNA
tcctggaagatggtgatgggaaaaaacgtatggcaagtcta

SEQ ID NO: 960 - DNA, 41 bp, synthetic construct
  source 1..41: mol_type = other DNA
ccacttgattttggagggataaaaaacgtatggcaagtcta

SEQ ID NO: 961 - DNA, 41 bp, synthetic construct
  source 1..41: mol_type = other DNA
ggactccacgacgtactcagaaaaaacgtatggcaagtcta

SEQ ID NO: 962 - DNA, 41 bp, synthetic construct
  source 1..41: mol_type = other DNA
ttctccatggtggtgaagacaaaaaacgtatggcaagtcta

SEQ ID NO: 963 - DNA, 41 bp, synthetic construct
  source 1..41: mol_type = other DNA
agagatgatgacccttttggaaaaaacgtatggcaagtcta

SEQ ID NO: 964 - DNA, 41 bp, synthetic construct
  source 1..41: mol_type = other DNA
gacgaacatgggggcatcagaaaaaacgtatggcaagtcta

SEQ ID NO: 965 - DNA, 41 bp, synthetic construct
  source 1..41: mol_type = other DNA
catacttctcatggttcacaaaaaaacgtatggcaagtcta

SEQ ID NO: 966 - DNA, 41 bp, synthetic construct
  source 1..41: mol_type = other DNA
attgctgatgatcttgaggcaaaaaacgtatggcaagtcta

SEQ ID NO: 967 - DNA, 41 bp, synthetic construct
  source 1..41: mol_type = other DNA
ctaagcagttggtggtgcagaaaaaacgtatggcaagtcta

SEQ ID NO: 968 - DNA, 41 bp, synthetic construct
  source 1..41: mol_type = other DNA
ccacgataccaaagttgtcaaaaaaacgtatggcaagtcta

SEQ ID NO: 969 - DNA, 41 bp, synthetic construct
  source 1..41: mol_type = other DNA
tcttctgggtggcagtgatgaaaaaacgtatggcaagtcta

SEQ ID NO: 970 - DNA, 41 bp, synthetic construct
  source 1..41: mol_type = other DNA
tagaggcagggatgatgttcaaaaaacgtatggcaagtcta

SEQ ID NO: 971 - DNA, 41 bp, synthetic construct
  source 1..41: mol_type = other DNA
tcagctcagggatgaccttgaaaaaacgtatggcaagtcta

SEQ ID NO: 972 - DNA, 41 bp, synthetic construct
  source 1..41: mol_type = other DNA
cactgacacgttggcagtggaaaaaacgtatggcaagtcta

SEQ ID NO: 973 - DNA, 41 bp, synthetic construct
  source 1..41: mol_type = other DNA
caggtttttctagacggcagaaaaaacgtatggcaagtcta

SEQ ID NO: 974 - DNA, 41 bp, synthetic construct
  source 1..41: mol_type = other DNA
caccttcttgatgtcatcataaaaaacgtatggcaagtcta

SEQ ID NO: 975 - DNA, 41 bp, synthetic construct
  source 1..41: mol_type = other DNA
gctgttgaagtcagaggagaaaaaaacgtatggcaagtcta

SEQ ID NO: 976 - DNA, 41 bp, synthetic construct
  source 1..41: mol_type = other DNA
cgtcaaaggtggaggagtggaaaaaacgtatggcaagtcta

SEQ ID NO: 977 - DNA, 41 bp, synthetic construct
  source 1..41: mol_type = other DNA
agtggtcgttgagggcaatgaaaaaacgtatggcaagtcta

SEQ ID NO: 978 - DNA, 41 bp, synthetic construct
  source 1..41: mol_type = other DNA
tcataccaggaaatgagcttaaaaaacgtatggcaagtcta

SEQ ID NO: 979 - DNA, 41 bp, synthetic construct
  source 1..41: mol_type = other DNA
cctgttgctgtagccaaattaaaaaacgtatggcaagtcta

SEQ ID NO: 980 - DNA, 41 bp, synthetic construct
  source 1..41: mol_type = other DNA
tgaggaggggagattcagtgaaaaaacgtatggcaagtcta

SEQ ID NO: 981 - DNA, 41 bp, synthetic construct
  source 1..41: mol_type = other DNA
ctcttcaaggggtctacatgaaaaaacgtatggcaagtcta

SEQ ID NO: 982 - DNA, 41 bp, synthetic construct
  source 1..41: mol_type = other DNA
tacatgacaaggtgcggctcaaaaaacgtatggcaagtcta

SEQ ID NO: 983 - DNA, 41 bp, synthetic construct
  source 1..41: mol_type = other DNA
tgagcacagggtactttattaaaaaacgtatggcaagtcta

SEQ ID NO: 984 - DNA, 41 bp, synthetic construct
  source 1..41: mol_type = other DNA
aggagtatgattgtcaacccaaaaatggagtttgggcagat

SEQ ID NO: 985 - DNA, 40 bp, synthetic construct
  source 1..40: mol_type = other DNA
cctatataaggcgaagcacaaaaatggagtttgggcagat

SEQ ID NO: 986 - DNA, 41 bp, synthetic construct
  source 1..41: mol_type = other DNA
gagtgactcgagagagctacaaaaatggagtttgggcagat

SEQ ID NO: 987 - DNA, 41 bp, synthetic construct
  source 1..41: mol_type = other DNA
atagtgagcaacgtaggcttaaaaatggagtttgggcagat

SEQ ID NO: 988 - DNA, 41 bp, synthetic construct
  source 1..41: mol_type = other DNA
ggacatgagttgggcaatttaaaaatggagtttgggcagat

SEQ ID NO: 989 - DNA, 41 bp, synthetic construct
  source 1..41: mol_type = other DNA
gagatcaactcccgaaggaaaaaaatggagtttgggcagat

SEQ ID NO: 990 - DNA, 41 bp, synthetic construct
  source 1..41: mol_type = other DNA
aatcttgtccaaggcatcagaaaaatggagtttgggcagat

SEQ ID NO: 991 - DNA, 41 bp, synthetic construct
  source 1..41: mol_type = other DNA
aacttcgaagggtctgtcagaaaaatggagtttgggcagat

SEQ ID NO: 992 - DNA, 41 bp, synthetic construct
  source 1..41: mol_type = other DNA
ggttggggatgatgtcaattaaaaatggagtttgggcagat

SEQ ID NO: 993 - DNA, 41 bp, synthetic construct
  source 1..41: mol_type = other DNA
taccaaagtcagggtacgttaaaaatggagtttgggcagat

SEQ ID NO: 994 - DNA, 41 bp, synthetic construct
  source 1..41: mol_type = other DNA
tgagatcagctttggtcatgaaaaatggagtttgggcagat

SEQ ID NO: 995 - DNA, 41 bp, synthetic construct
  source 1..41: mol_type = other DNA
ttggcaatggttcccaaattaaaaatggagtttgggcagat

SEQ ID NO: 996 - DNA, 41 bp, synthetic construct
  source 1..41: mol_type = other DNA
ctgaagagcctccatgaatgaaaaatggagtttgggcagat

SEQ ID NO: 997 - DNA, 41 bp, synthetic construct
  source 1..41: mol_type = other DNA
ccaccaagtaggcagaataaaaaaatggagtttgggcagat

SEQ ID NO: 998 - DNA, 41 bp, synthetic construct
  source 1..41: mol_type = other DNA
tgctttgtgatcacaaccacaaaaatggagtttgggcagat

SEQ ID NO: 999 - DNA, 41 bp, synthetic construct
  source 1..41: mol_type = other DNA
cagaagactcccaagcatacaaaaatggagtttgggcagat

SEQ ID NO: 1000 - DNA, 41 bp, synthetic construct
  source 1..41: mol_type = other DNA
agcacgcacagtgaaggaacaaaaatggagtttgggcagat

SEQ ID NO: 1001 - DNA, 41 bp, synthetic construct
  source 1..41: mol_type = other DNA
tctaggtactctgtctgatcaaaaatggagtttgggcagat

SEQ ID NO: 1002 - DNA, 41 bp, synthetic construct
  source 1..41: mol_type = other DNA
taaagggtgatgggatagccaaaaatggagtttgggcagat

SEQ ID NO: 1003 - DNA, 41 bp, synthetic construct
  source 1..41: mol_type = other DNA
tgtttagttcttcctgatcaaaaaatggagtttgggcagat

SEQ ID NO: 1004 - DNA, 41 bp, synthetic construct
  source 1..41: mol_type = other DNA
agggtttctggtccaaatagaaaaatggagtttgggcagat

SEQ ID NO: 1005 - DNA, 41 bp, synthetic construct
  source 1..41: mol_type = other DNA
tcattagtgaggctcttgtaaaaaatggagtttgggcagat

SEQ ID NO: 1006 - DNA, 41 bp, synthetic construct
  source 1..41: mol_type = other DNA
aaagtgcttgactgccaagtaaaaatggagtttgggcagat

SEQ ID NO: 1007 - DNA, 41 bp, synthetic construct
  source 1..41: mol_type = other DNA
tgaattccaactgaccttctaaaaatggagtttgggcagat

SEQ ID NO: 1008 - DNA, 41 bp, synthetic construct
  source 1..41: mol_type = other DNA
gagcccgacgaggaataaataaaaatggagtttgggcagat

SEQ ID NO: 1009 - DNA, 41 bp, synthetic construct
  source 1..41: mol_type = other DNA
tgaacacacggcggacatagaaaaatggagtttgggcagat

SEQ ID NO: 1010 - DNA, 41 bp, synthetic construct
  source 1..41: mol_type = other DNA
atcaactcatcacagctgtcaaaaatggagtttgggcagat

SEQ ID NO: 1011 - DNA, 41 bp, synthetic construct
  source 1..41: mol_type = other DNA
aagattttgctctgctggagaaaaatggagtttgggcagat

SEQ ID NO: 1012 - DNA, 41 bp, synthetic construct
  source 1..41: mol_type = other DNA
agagaagagctcaaggcactaaaaatggagtttgggcagat

SEQ ID NO: 1013 - DNA, 41 bp, synthetic construct
  source 1..41: mol_type = other DNA
gtggattccaagcttgagataaaaatggagtttgggcagat

SEQ ID NO: 1014 - DNA, 41 bp, synthetic construct
  source 1..41: mol_type = other DNA
agactgggaggtatgatagcaaaaatggagtttgggcagat

SEQ ID NO: 1015 - DNA, 41 bp, synthetic construct
  source 1..41: mol_type = other DNA
ctctgacagagatgtcatctaaaaatggagtttgggcagat

SEQ ID NO: 1016 - DNA, 41 bp, synthetic construct
  source 1..41: mol_type = other DNA
tagatggacttctgtgtctcaaaaatggagtttgggcagat

SEQ ID NO: 1017 - DNA, 41 bp, synthetic construct
  source 1..41: mol_type = other DNA
gctccacaaaagctgagttgaaaaatggagtttgggcagat

SEQ ID NO: 1018 - DNA, 41 bp, synthetic construct
  source 1..41: mol_type = other DNA
catatataccacctcgaagcaaaaatggagtttgggcagat

SEQ ID NO: 1019 - DNA, 41 bp, synthetic construct
  source 1..41: mol_type = other DNA
acacagtactcgtcaatgggaaaaatggagtttgggcagat

SEQ ID NO: 1020 - DNA, 41 bp, synthetic construct
  source 1..41: mol_type = other DNA
ttcccatcaaattccttgagaaaaatggagtttgggcagat

SEQ ID NO: 1021 - DNA, 41 bp, synthetic construct
  source 1..41: mol_type = other DNA
gagattgtcaccttctcaacaaaaatggagtttgggcagat

SEQ ID NO: 1022 - DNA, 41 bp, synthetic construct
  source 1..41: mol_type = other DNA
tgcagcaaggtgaagacacaaaaaatggagtttgggcagat

SEQ ID NO: 1023 - DNA, 41 bp, synthetic construct
  source 1..41: mol_type = other DNA
gctttttggccatcatatagaaaaatggagtttgggcagat

SEQ ID NO: 1024 - DNA, 41 bp, synthetic construct
  source 1..41: mol_type = other DNA
aactgccttatcattcttgtaaaaatggagtttgggcagat

SEQ ID NO: 1025 - DNA, 41 bp, synthetic construct
  source 1..41: mol_type = other DNA
atcctcaagggaaaagccagaaaaatggagtttgggcagat

SEQ ID NO: 1026 - DNA, 41 bp, synthetic construct
  source 1..41: mol_type = other DNA
tgatcatgcgatagatgcggaaaaatggagtttgggcagat

SEQ ID NO: 1027 - DNA, 41 bp, synthetic construct
  source 1..41: mol_type = other DNA
catcaggaactgcagcattgaaaaatggagtttgggcagat

SEQ ID NO: 1028 - DNA, 41 bp, synthetic construct
  source 1..41: mol_type = other DNA
caagggcacaagttttccaaaaaaatggagtttgggcagat

SEQ ID NO: 1029 - DNA, 41 bp, synthetic construct
  source 1..41: mol_type = other DNA
tactgccttcaacacaaggaaaaaatggagtttgggcagat

SEQ ID NO: 1030 - DNA, 41 bp, synthetic construct
  source 1..41: mol_type = other DNA
agagtagagagggaatggggaaaaatggagtttgggcagat

SEQ ID NO: 1031 - DNA, 41 bp, synthetic construct
  source 1..41: mol_type = other DNA
tacacaacatccaatcctgcaaaaatggagtttgggcagat

SEQ ID NO: 1032 - DNA, 16 bp, synthetic construct
  source 1..16: mol_type = other DNA
agatgacgcttgggaa

SEQ ID NO: 1033 - DNA, 22 bp, synthetic construct
  source 1..22: mol_type = other DNA
tcgctctctgctcctcctgttc

SEQ ID NO: 1034 - DNA, 20 bp, synthetic construct
  source 1..20: mol_type = other DNA
cgcccaatacgaccaaatcc

SEQ ID NO: 1035 - DNA, 21 bp, synthetic construct
  source 1..21: mol_type = other DNA
gtcaaccccaccgtgttcttc

SEQ ID NO: 1036 - DNA, 23 bp, synthetic construct
  source 1..23: mol_type = other DNA
tttctgctgtctttgggaccttg

SEQ ID NO: 1037 - DNA, 10 bp, synthetic construct
  source 1..10: mol_type = other DNA
aaaaaaaaaa